(19) United States
(12) Patent Application Publication
(10) Pub. No.: US 2025/0367173 A1
(43) Pub. Date: Dec. 4, 2025

(54) ECTONUCLEOTIDE PYROPHOSPHATASE-PHOSPHODIESTERASE-1 INHIBITORS AND PHARMACEUTICAL COMPOSITIONS COMPRISING THE SAME

(71) Applicant: LigaChem Biosciences Inc., Daejeon (KR)

(72) Inventors: Ju Hyeon Kim, Daejeon (KR); Eun Mi Jung, Daejeon (KR); Yeon Hee Lee, Daejeon (KR); Jeong Hyun Kim, Daejeon (KR); Dae Yon Lee, Daejeon (KR); Pyoung Oh Yoon, Daejeon (KR); Young Cheol Lee, Daejeon (KR); Eun Ji Son, Daejeon (KR); A-Ram Lee, Daejeon (KR)

(21) Appl. No.: 18/873,569

(22) PCT Filed: Jun. 14, 2023
(86) PCT No.: PCT/IB2023/000356

§ 371 Date: Dec. 10, 2024

(30) Foreign Application Priority Data
Jun. 14, 2022 (KR) 10-2022-0072041

(51) Int. Cl. A61K 31/437; C07D 471/04; C07D 471/14; C07D 487/04

(52) U.S. Cl. CPC A61K 31/437; C07D 471/04; C07D 471/14; C07D 487/04

(57) ABSTRACT

The present disclosure relates compounds for inhibiting ectonucleotide pyrophosphatase/phosphodiesterase-1 (ENPP-1), or a pharmaceutically acceptable salts thereof.

### RELATED APPLICATIONS

This application claims the benefit of priority to Korean Patent Application 10-2022-0072041, filed Jun. 14, 2022; the contents of which are hereby incorporated by reference in their entirety.

### BACKGROUND

Ectonucleotide pyrophosphatase/phosphodiesterase-1 (i.e., ENPP-1), is a type II transmembrane glycoprotein with pyrophosphatase and phosphodiesterase activities and is highly expressed in bone and cartilage.

ATP is a substrate of ENPP-1 and is hydrolyzed to AMP and PPi, and AMP is converted to adenosine and inorganic phosphate (Pi) by CD73. ENPP-1 hydrolysis of nucleotide pyrophosphate bonds (for example, in ATP) and phosphodiester bonds (for example, in oligonucleotides) as part of functions the mediated by nucleotide pyrophosphatases/phosphodiesterases (NPPs) is necessary in a wide range of cellular processes, including nucleotide pyrophosphate recycling, purinergic receptor signaling and ATP-mediated apoptosis.

Recently, ENPP-1 has been found to play a key role in immunological responses to various stimuli through the cyclic GMP-AMP synthase (cGAS)-stimulator of interferon genes (STING) pathway. Damage associated molecular patterns (DAMPs) as well as pathogen associated molecular patterns (PAMPs) activate the immune system through STING. cGAS senses cytoplasmic DNA and catalyzes the conversion of GTP and ATP to cyclic GMP-AMP (cGAMP). Subsequently, 2,3′-cGAMP initiates an inflammatory response through the TANK-binding kinase 1 (TBK1)-interferon regulatory factor (IRF) 3 pathway to produce type 1 interferons (IFNs) and other cytokines. A link between the cGAS-STING pathway and ENPP-1 has emerged, whereby hydrolysis of cGAMP by ENPP-1 attenuates cGAS-STING signaling. ENPP-1 plays a regulatory function in immune cells such as neutrophils, macrophages, dendritic cells, natural killer cells, and B lymphocytes. Expression of ENPP-1 increases in M2 macrophages in the presence of cancer and promotes tumor growth and spread. The role of ENPP-1 in cancer is exemplified by the observation of enhanced tumor metastasis from breast cancer to bone, for example, over-expression of ENPP-1.

Recent reports suggest that cyclic dinucleotides, which are substrates of ENPP-1, stimulate innate immunity through STING-dependent activation of interferon genes. ENPP-1 inhibition of STING pathway activation is critical for tumor control, similar to that of checkpoint inhibitors such as anti-PD-1 or PD-L1, which are promising immunotherapeutic agents for various cancers.

Since STING activation is a promising therapeutic strategy for treating cancer, more and more compounds that activate the STING pathway have been reported. ENPP-1 is a highly potent cGAMP-degrading enzyme, and ENPP-1 inhibitors are applied for anti-tumor therapy.

ADP-ribosylation is a conserved post-translational modification of proteins, which plays a role in all major cellular processes, particularly DNA repair, transcription, translation, stress response and cell death. Poly ADP-ribosylation (PARylation) mediated by poly ADP-ribose polymerase (PARP) plays a key role in DNA damage repair. Inhibition of poly ADP-ribosylation by PARP inhibitors inhibits DNA damage repair and induces death of DNA repair-deficient tumor cells. Thus, PARP inhibitors have been approved by the US FDA for the treatment of various types of cancer.

However, recent studies suggest that de-poly ADP-ribosylation (dePARylation) also plays a key role in DNA damage repair. De-poly ADP-ribosylation acts as a downstream step of poly ADP-ribosylation in DNA damage repair, instead of antagonizing poly ADP-ribosylation. Protein poly ADP-ribosylation may be reversed by macrodomains including the proteins PARG, TARG1, MacroD1, MacroD2 and ENPP-1. Recently, ENPP-1 has been known to play a role in the de-poly ADP-ribosylation process by hydrolyzing the ester bonds known to link proteins to ADP-ribose. De-poly ADP-ribosylation inhibitors represent a new class of inhibitors that replace PARP inhibition, and may overcome the anticancer drug resistance of PARPi. Thus, ENPP-1 inhibitors may play a key role in the DNA damage repair process.

Based on the above principles, there is an urgent and growing need for a class of antitumor compounds capable of inhibiting ENPP-1 and treating various types of cancer.

### SUMMARY OF THE INVENTION

The present disclosure relates to inhibitors of ectonucleotide pyrophosphatase/phosphodiesterase-1 (ENPP-1) and pharmaceutically acceptable salts thereof.

In certain aspects, the present disclosure provides compound having a structure represented by Formula I or a pharmaceutically acceptable salt thereof:

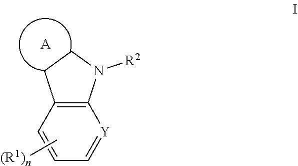

wherein
each R¹ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
A is an aryl, heteroaryl, or heterocyclyl;
Y is CH, CR¹, or N;
R² is aralkyl, heteroaralkyl, heterocyclylalkyl, or cycloalkylalkyl; and
n is 0, 1, 2, 3, or 4.

In certain aspects, the present disclosure provides methods of treating cancer in a subject in need thereof comprising administering a compound disclosed herein, or a pharmaceutically acceptable salt thereof, to the subject.

In certain aspects, the present disclosure provides methods of treating a disease or disorder characterized by ENPP-1 in a subject in need thereof comprising administering a compound disclosed herein, or a pharmaceutically acceptable salt thereof, to the subject.

### DETAILED DESCRIPTION OF THE INVENTION

Hereinafter, the present disclosure will be described in more detail.

The compounds according to the present disclosure is a are novel exhibit high inhibitory activity against ENPP-1, while at the same time, activating the STING pathway, and thus may be usefully used to treat, prevent and alleviate diseases caused by abnormal cell growth mediated by ENPP-1, such as cancer.

In certain aspects, the present disclosure provides compound having a structure represented by Formula I or a pharmaceutically acceptable salt thereof:

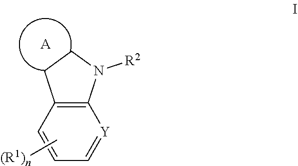

wherein
each R¹ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
A is an aryl, heteroaryl, or heterocyclyl;
Y is CH, CR¹, or N;
R² is aralkyl, heteroaralkyl, heterocyclylalkyl, or cycloalkylalkyl; and
n is 0, 1, 2, 3, or 4.

In certain embodiments, A is a heteroaryl (e.g., pyridinyl, pyrimidinyl, or pyridazinyl, preferably pyridinyl). In other embodiments, A is a heterocyclyl (e.g., piperidinyl, piperidonyl, tetrahydropyridazinonyl,

In certain preferred embodiments, Y is CH. In other embodiments, Y is N.

In certain embodiments, the compound has a structure represented by Formula Ia or a pharmaceutically acceptable salt thereof:

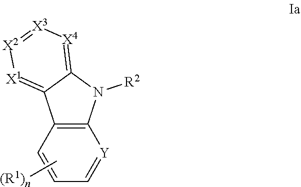

wherein,
X¹ is CR³ or N;
X² is CR⁴ or N;
X³ is CR⁵ or N;
X⁴ is CR⁶ or N; and
R³, R⁴, R⁵, and R⁶ are each independently selected from H, alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, and sulfonamido.

In certain preferred embodiments, X¹ is N. In other embodiments, X¹ is CR³.

In certain embodiments, X¹ is N. In other embodiments, X¹ is CR³.

In certain embodiments, X² is N. In other embodiments, X² is CR⁴.

In certain embodiments, X³ is N. In other embodiments, X³ is CR⁵.

In certain embodiments, X⁴ is N. In other embodiments, X⁴ is CR⁶.

In certain embodiments, R³ is H.

In certain preferred embodiments, R⁴ is H.

In certain preferred embodiments, R⁵ is H.

In certain preferred embodiments, R⁶ is H.

In certain embodiments, n is 1. In other embodiments, n is 2.

In certain embodiments, the compound has a structure represented by Formula Ib or a pharmaceutically acceptable salt thereof:

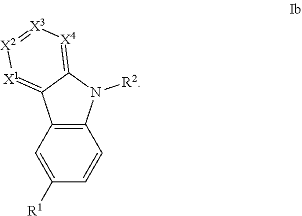

In certain embodiments, the compound has a structure represented by Formula Ic or a pharmaceutically acceptable salt thereof:

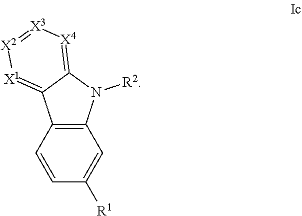

In certain embodiments, the compound has a structure represented by Formula Id or a pharmaceutically acceptable salt thereof:

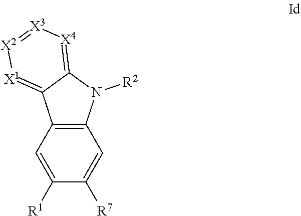

wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.

In certain embodiments, the compound has a structure represented by Formula Ie or a pharmaceutically acceptable salt thereof:

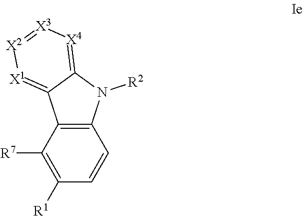

wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.

In certain embodiments, the compound has a structure represented by Formula If or a pharmaceutically acceptable salt thereof:

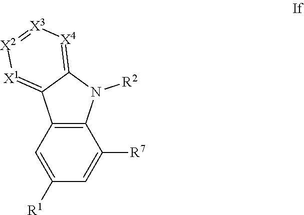

wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.

In certain preferred embodiments, R² is benzyl, and is preferably substituted by iminosulfanonyl (e.g., (imino)(methyl) sulfanonyl).

In certain embodiments, the compound has a structure represented by Formula IIa or a pharmaceutically acceptable salt thereof:

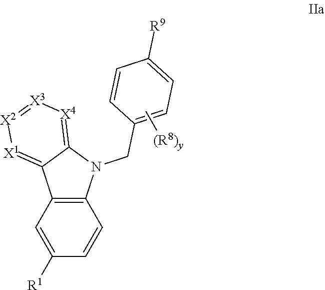

wherein,
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

In certain embodiments, the compound has a structure represented by Formula IIb or a pharmaceutically acceptable salt thereof:

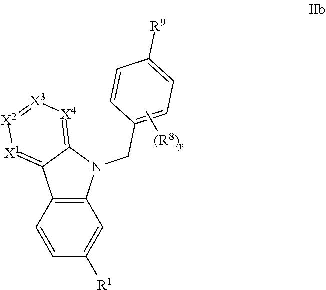

wherein,
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

In certain embodiments, the compound has a structure represented by Formula IIc or a pharmaceutically acceptable salt thereof:

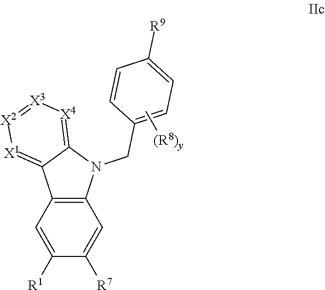

wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

In certain embodiments, the compound has a structure represented by Formula IId or a pharmaceutically acceptable salt thereof:

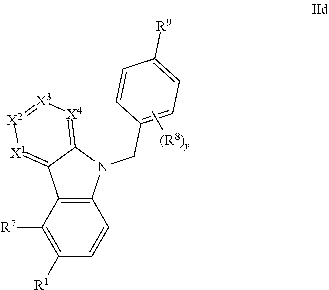

wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclic, alkylsulfoxidyl, alkylsulfonyl, or sulfonamido.
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

In certain embodiments, the compound has a structure represented by Formula Ile or a pharmaceutically acceptable salt thereof:

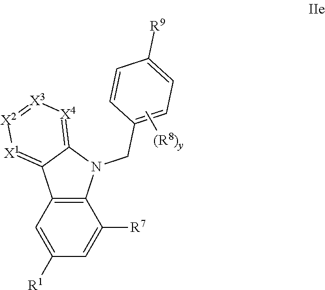

wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 0, 1, 2, 3, or 4.

In certain embodiments, R¹ is alkyl (e.g., methyl). In certain preferred embodiments, R¹ is alkoxy (e.g., methoxy, trifluoromethoxy, or ethoxy, preferably methoxy). In certain embodiments, R¹ is halo (e.g., fluoro or chloro).

In certain embodiments, R⁷ is alkyl (e.g., methyl). In certain embodiments, R⁷ is alkoxy (e.g., methoxy). In certain embodiments, R⁷ is halo (e.g., fluoro or chloro).

In certain embodiments, y is 0. In other embodiments, y is 1.

In certain embodiments, R⁸ is halo (e.g., fluoro or chloro). In certain embodiments, R⁸ is alkyl (e.g., methyl or trifluoromethyl). In certain embodiments, R⁸ is alkoxy (e.g., methoxy).

In certain preferred embodiments, R⁹ is iminosulfanonyl (e.g., (imino)(methyl) sulfanonyl, (imino) (ethyl) sulfanonyl, (imino) (benzyl) sulfanonyl, or imino) (cyclopropyl) sulfanonyl). In other embodiments, R⁹ is sulfonamido (e.g., methylsulfonamido). In yet other embodiments, R⁹ is phosphoryl. In yet other embodiments, R⁹ is alkyl (e.g., phosphorylalkyl, such as phosphorylmethyl or phosphoryldifluoromethyl). In yet other embodiments, R⁹ is phosphoramidityl. In yet other embodiments, R⁹ is phosphoramidatyl.

In certain embodiments, R¹¹ is

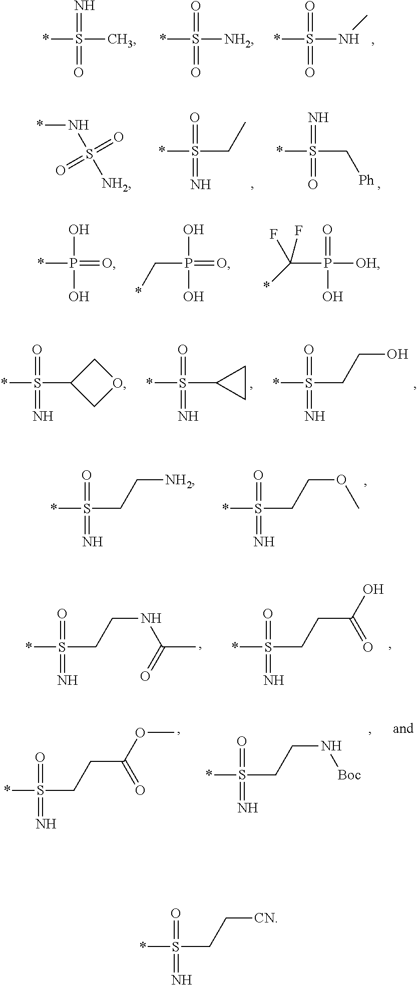

In certain embodiments, the compound is selected from:

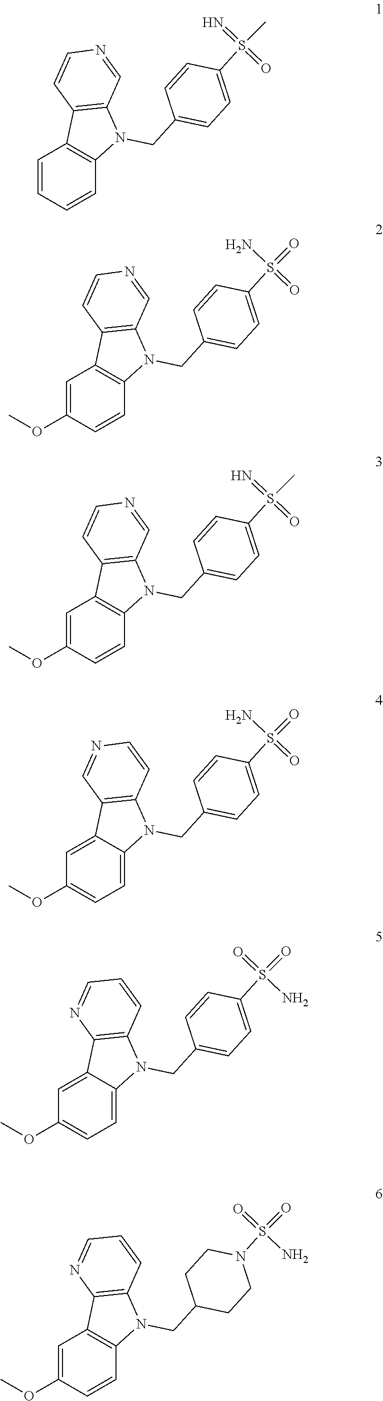 [SMILES: CS(=N)(=O)c1ccc(Cn2c3ccccc3c3ccncc32)cc1 ; COc1ccc2c(c1)c1ccncc1n2Cc1ccc(S(N)(=O)=O)cc1 ; COc1ccc2c(c1)c1ccncc1n2Cc1ccc(S(C)(=N)=O)cc1 ; COc1ccc2c(c1)c1cnccc1n2Cc1ccc(S(N)(=O)=O)cc1 ; COc1ccc2c(c1)c1ncccc1n2Cc1ccc(S(N)(=O)=O)cc1 ; COc1ccc2c(c1)c1ncccc1n2CC1CCN(S(N)(=O)=O)CC1]

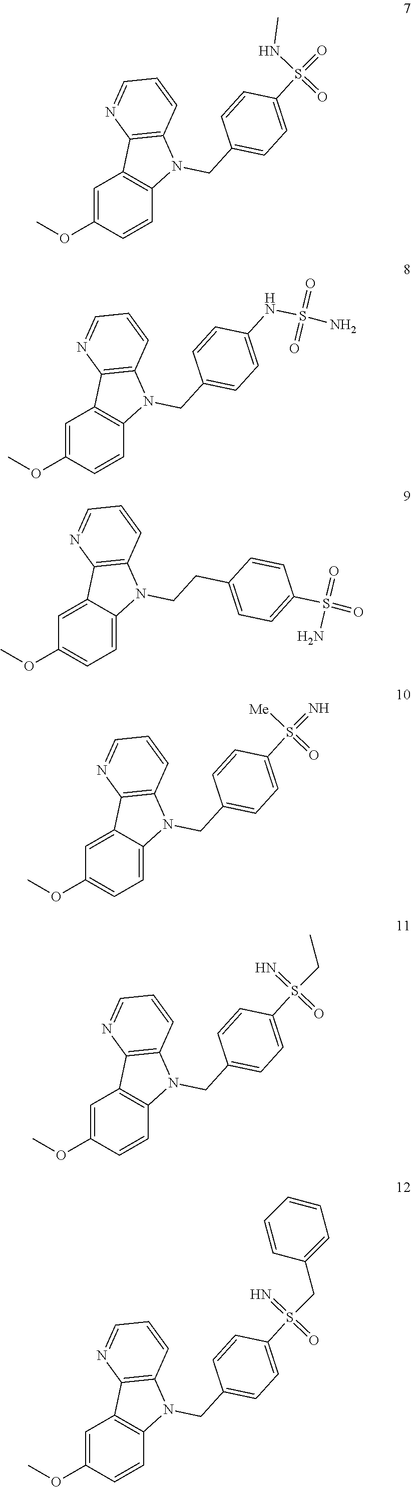

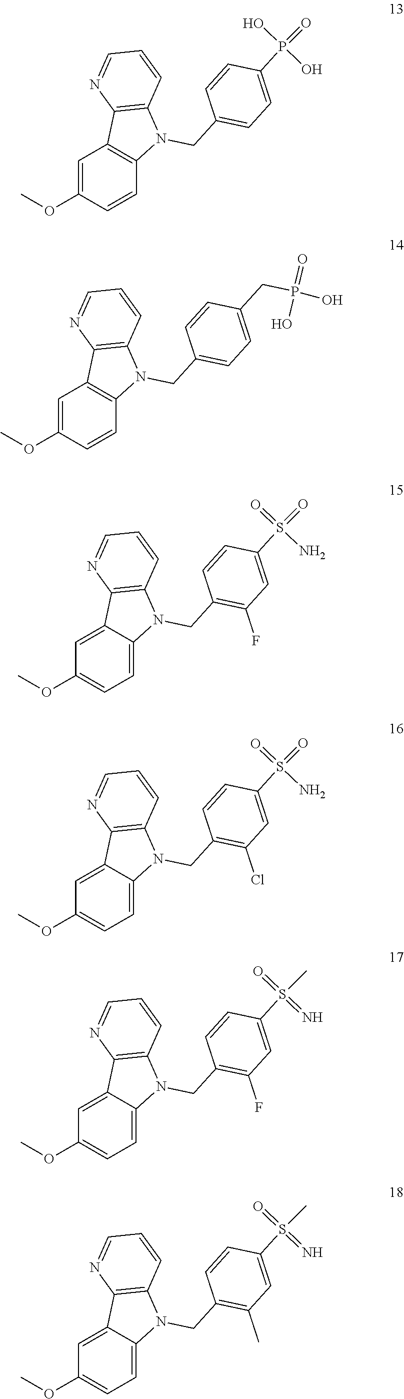 [SMILES: COc1ccc2c(c1)c1ncccc1n2Cc1ccc(P(=O)(O)O)cc1 ; COc1ccc2c(c1)c1ncccc1n2Cc1ccc(CP(=O)(O)O)cc1 ; COc1ccc2c(c1)c1ncccc1n2Cc1ccc(S(N)(=O)=O)cc1F ; COc1ccc2c(c1)c1ncccc1n2Cc1ccc(S(N)(=O)=O)cc1Cl ; COc1ccc2c(c1)c1ncccc1n2Cc1ccc(S(C)(=N)=O)cc1F ; COc1ccc2c(c1)c1ncccc1n2Cc1ccc(S(C)(=N)=O)cc1C]

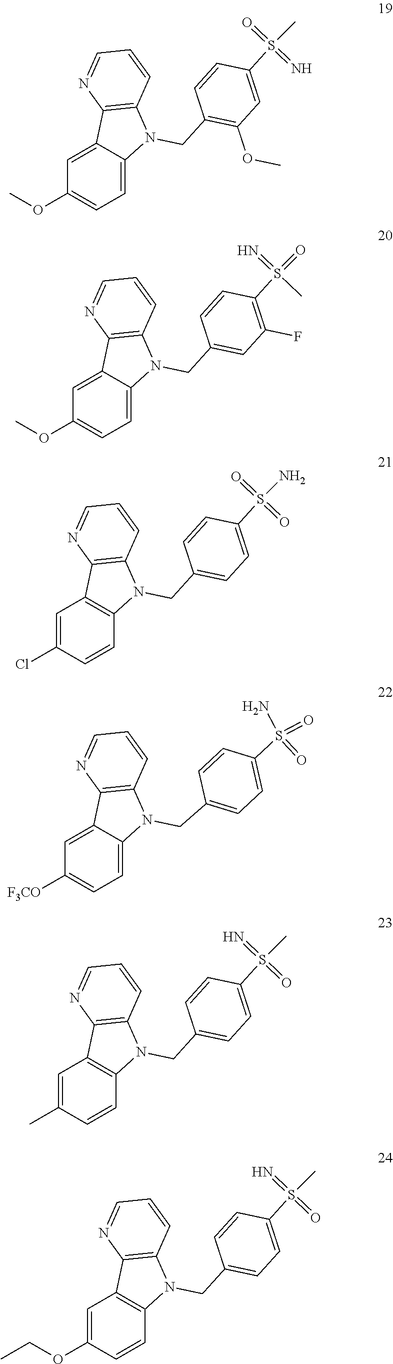

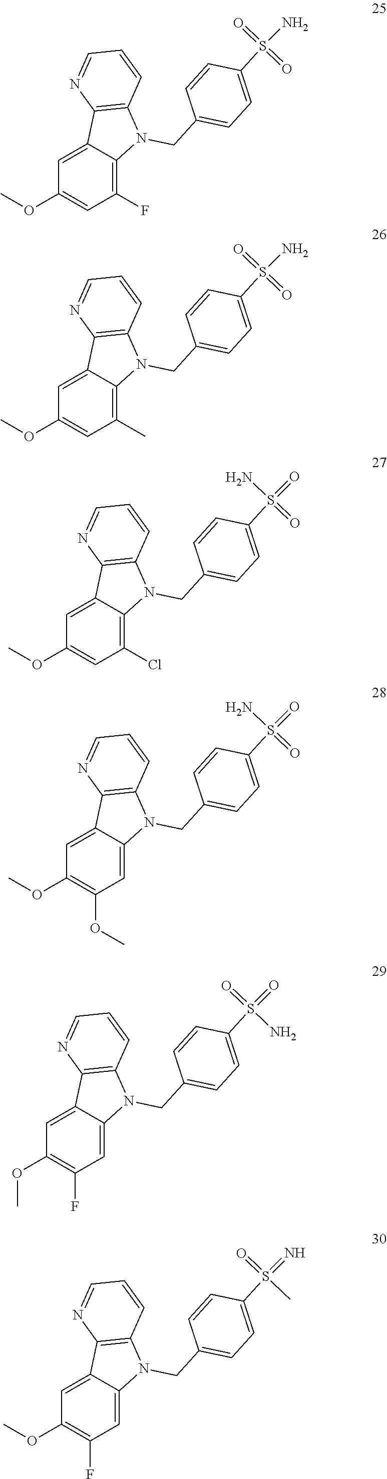

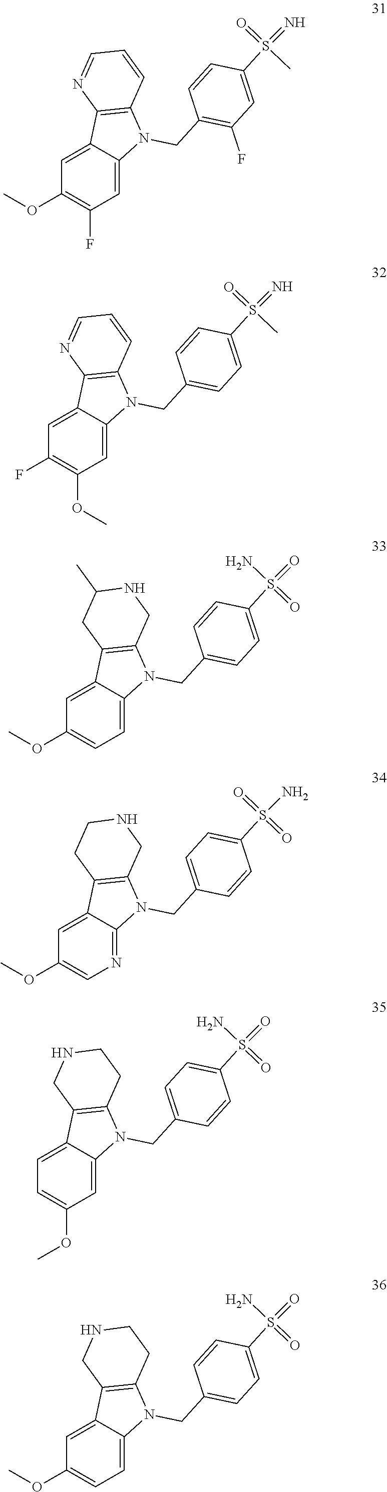

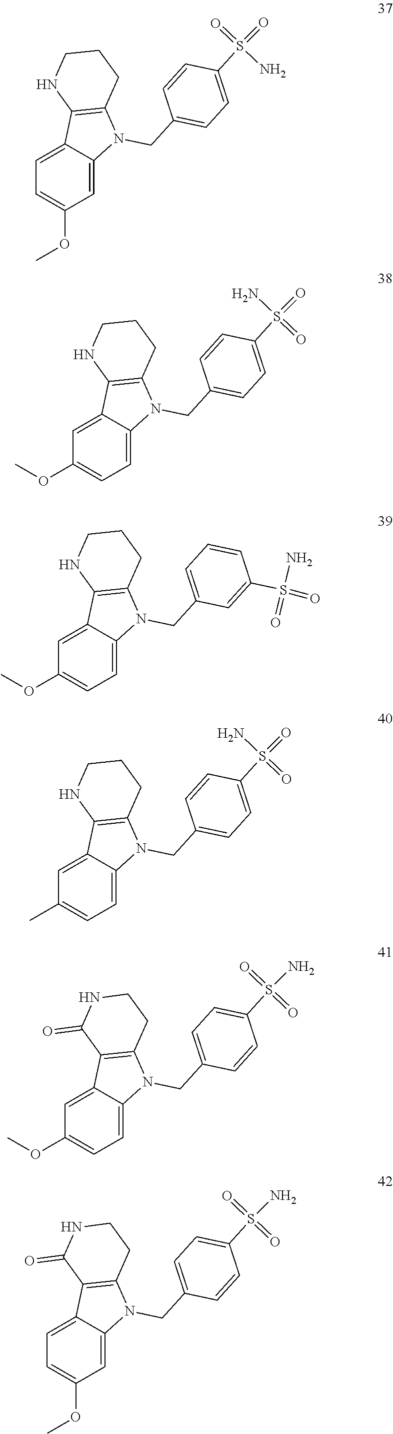 [SMILES: COc1ccc2c3c(n(Cc4ccc(S(N)(=O)=O)cc4)c2c1)CCCN3 ; COc1ccc2c(c1)c1c(n2Cc2ccc(S(N)(=O)=O)cc2)CCCN1 ; COc1ccc2c(c1)c1c(n2Cc2cccc(S(N)(=O)=O)c2)CCCN1 ; Cc1ccc2c(c1)c1c(n2Cc2ccc(S(N)(=O)=O)cc2)CCCN1 ; COc1ccc2c(c1)c1c(n2Cc2ccc(S(N)(=O)=O)cc2)CCNC1=O ; COc1ccc2c3c(n(Cc4ccc(S(N)(=O)=O)cc4)c2c1)CCNC3=O]

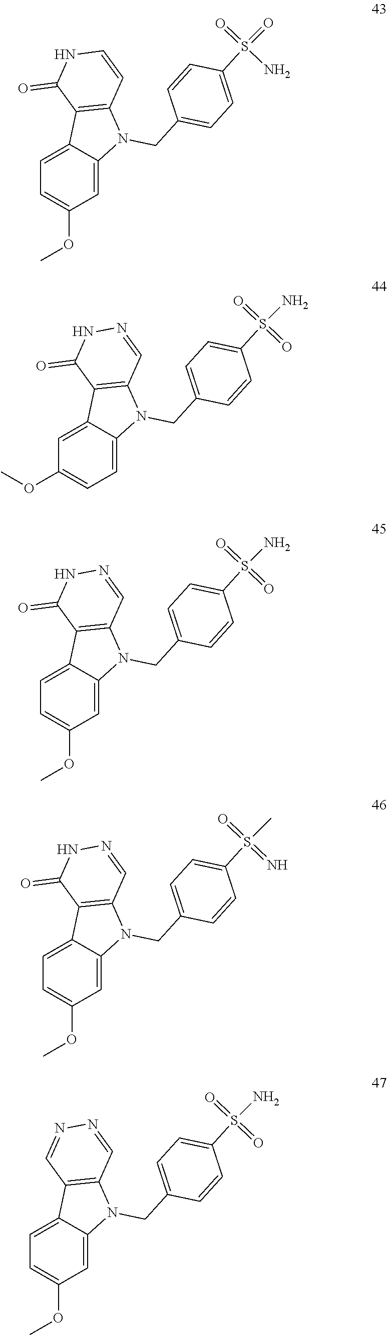 [SMILES: COc1ccc2c3c(=O)[nH]ccc3n(Cc3ccc(S(N)(=O)=O)cc3)c2c1 ; COc1ccc2c(c1)c1c(=O)[nH]ncc1n2Cc1ccc(S(N)(=O)=O)cc1 ; COc1ccc2c3c(=O)[nH]ncc3n(Cc3ccc(S(N)(=O)=O)cc3)c2c1 ; COc1ccc2c3c(=O)[nH]ncc3n(Cc3ccc(S(C)(=N)=O)cc3)c2c1 ; COc1ccc2c3cnncc3n(Cc3ccc(S(N)(=O)=O)cc3)c2c1]

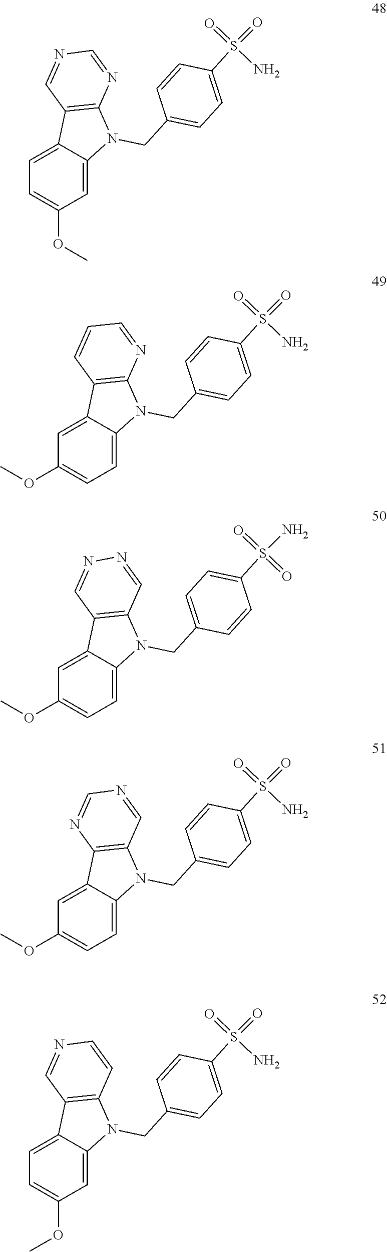 [SMILES: COc1ccc2c3cncnc3n(Cc3ccc(S(N)(=O)=O)cc3)c2c1 ; COc1ccc2c(c1)c1cccnc1n2Cc1ccc(S(N)(=O)=O)cc1 ; COc1ccc2c(c1)c1cnncc1n2Cc1ccc(S(N)(=O)=O)cc1 ; COc1ccc2c(c1)c1ncncc1n2Cc1ccc(S(N)(=O)=O)cc1 ; COc1ccc2c3cnccc3n(Cc3ccc(S(N)(=O)=O)cc3)c2c1]

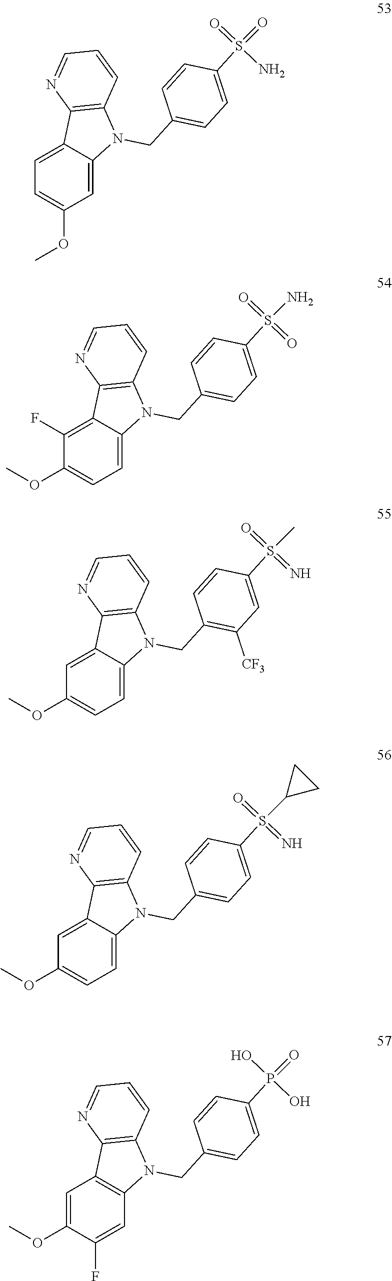 [SMILES: COc1ccc2c3ncccc3n(Cc3ccc(S(N)(=O)=O)cc3)c2c1 ; COc1ccc2c(c1F)c1ncccc1n2Cc1ccc(S(N)(=O)=O)cc1 ; COc1ccc2c(c1)c1ncccc1n2Cc1ccc(S(C)(=N)=O)cc1C(F)(F)F ; COc1ccc2c(c1)c1ncccc1n2Cc1ccc(S(=N)(=O)C2CC2)cc1 ; COc1cc2c3ncccc3n(Cc3ccc(P(=O)(O)O)cc3)c2cc1F]

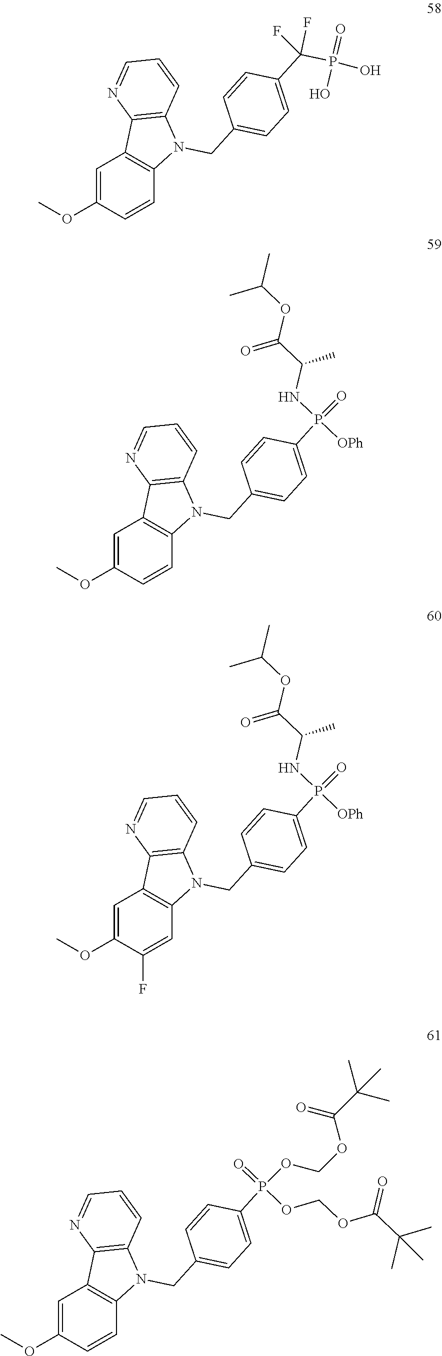 [SMILES: COc1ccc2c(c1)c1ncccc1n2Cc1ccc(C(F)(F)P(=O)(O)O)cc1 ; COc1ccc2c(c1)c1ncccc1n2Cc1ccc(P(=O)(N[C@@H](C)C(=O)OC(C)C)Oc2ccccc2)cc1 ; COc1cc2c3ncccc3n(Cc3ccc(P(=O)(N[C@@H](C)C(=O)OC(C)C)Oc4ccccc4)cc3)c2cc1F ; COc1ccc2c(c1)c1ncccc1n2Cc1ccc(P(=O)(OCOC(=O)C(C)(C)C)OCOC(=O)C(C)(C)C)cc1]

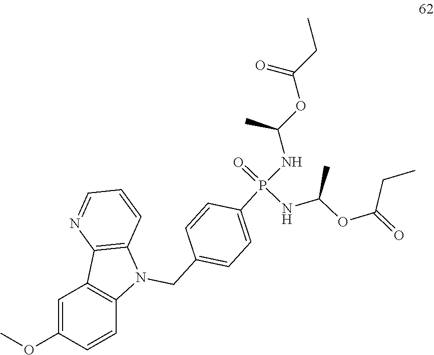 [SMILES: CCC(=O)O[C@H](C)NP(=O)(N[C@@H](C)OC(=O)CC)c1ccc(Cn2c3ccc(OC)cc3c3ncccc32)cc1]

or a pharmaceutically acceptable salt thereof.

Many of the compounds useful in the methods and compositions of this disclosure, such as those recited above, have at least one stereogenic center in their structure (e.g., due to the presence of a lone pair on a sulfur atom). This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

In other embodiments, the present disclosure provides a compound represented by following Formula 1, a hydrate thereof, a solvate thereof, an isomer thereof, or a pharmaceutically acceptable salt thereof:

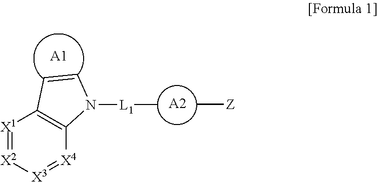

wherein,
A1 is a substituted or unsubstituted C3-30 cycloalkyl ring, a substituted or unsubstituted C2-30 heterocycloalkyl ring, a substituted or unsubstituted C6-30 aryl ring, or a substituted or unsubstituted C3-30 heteroaryl ring, fused to an adjacent ring;
A2 is a substituted or unsubstituted C3-20 cycloalkylene group, a substituted or unsubstituted C2-20 heterocycloalkylene group, a substituted or unsubstituted C6-30 arylene group, or a substituted or unsubstituted C3-30 heteroarylene group;
X₁ to X₄ are each independently CR or N;
R is hydrogen, a hydroxy group, a halogen group, a C1-10 alkyl group, a C1-10 alkoxy group, a C6-20 aryl group, a C3-20 heteroaryl group, a C3-10 cycloalkyl group, a C3-10 heterocycloalkyl group, an amino group, a nitro group, an amide group, a carboxyl acid group, a nitrile group, a urea group, or a sulfonamide group;
L₁ is a C1-10 alkylene group or a C2-10 alkenylene group;
Z is represented by

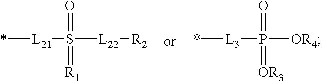

R₁ is O or NR₅;
R₂ is hydrogen, a hydroxy group, a cyano group, a C1-C10 alkyl group, a C1-10 alkoxy group, a C6-C12 aryl group, a C3-C10 cycloalkyl group, a C2-C12 heterocycloalkyl group, —C(═O)OR₆, or —NR₈R₉;
L₂₁, L₂₂ and L₃ are each independently a single bond, a substituted or unsubstituted C1-C5 alkylene group, or —NR₇—;
R₃ to R₇ are each independently hydrogen or a C1-C5 alkyl group;
R₈ and R₉ are each independently hydrogen, —C(═O)R₁₀ (with the proviso that R₁₀ is a C1-C5 alkyl group), or -Boc (tert-butoxycarbonyl); and
* is a point connected to A2 in Formula 1.

In certain embodiments, the compound represented by Formula 1 according to the present disclosure exhibits very high inhibitory activity against ENPP-1 and at the same time activates the STING pathway, and thus may be used to treat, prevent and alleviate cancer caused by abnormal cell growth mediated by ENPP-1.

In the compound according to an embodiment of the present disclosure, A1 in Formula 1 above may be represented by following Formula 2 or Formula 3:

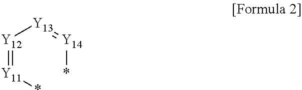

wherein,
Y₁₁ to Y₁₄ are each independently CRₐ or N;
Rₐ is hydrogen, a hydroxy group, a halogen group, a C1-10 alkyl group, a C1-10 alkoxy group, a C6-20 aryl group, a C3-20 heteroaryl group, a C3-10 cycloalkyl group, a C3-10 heterocycloalkyl group, an amino group, a nitro group, an amide group, a carboxyl acid group, a nitrile group, a urea group, or a sulfonamide group; and
* is a point connected to each of two adjacent carbon atoms on the fused ring to which A1 is attached, and

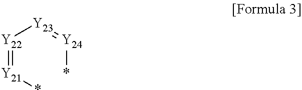

wherein:
Y₂₁ to Y₂₄ are each independently CR_{b}R_{c}, —C(═O)—, or NR_{d};
R_{b} to R_{d} are each independently hydrogen, a hydroxy group, a halogen group, a C1-10 alkyl group, a C1-10 alkoxy group, a C6-20 aryl group, a C3-20 heteroaryl group, a C3-10 cycloalkyl group, a C3-10 heterocycloalkyl group, an amino group, a nitro group, an amide group, a carboxyl acid group, a nitrile group, a urea group, or a sulfonamide group; and
* is a point connected to each of two adjacent carbon atoms on the fused ring to which A1 is attached.

In the compound according to an embodiment of the present disclosure, A1 in Formula 1 may be a substituted or unsubstituted C3-10 cycloalkyl ring, a substituted or unsubstituted C2-10 heterocycloalkyl ring, a substituted or unsubstituted C6-10 aryl ring, or a substituted or unsubstituted C3-10 heteroaryl ring, fused to an adjacent ring;
A2 may be a substituted or unsubstituted C3-10 cycloalkylene group, a substituted or unsubstituted C2-10 heterocycloalkylene group, a substituted or unsubstituted C6-10 arylene group, or a substituted or unsubstituted C3-10 heteroarylene group;
X₁ to X₄ may be each independently CR or N;
R may be hydrogen, a hydroxy group, a halogen group, a C1-10 alkyl group, a C1-10 alkoxy group, a C6-10 aryl group, a C3-10 heteroaryl group, a C3-10 cycloalkyl group, a C3-10 heterocycloalkyl group, an amino group, a nitro group, an amide group, a carboxyl acid group, a nitrile group, a urea group, or a sulfonamide group;
L₁ may be a C1-10 alkylene group.

In the compound according to an embodiment of the present disclosure, A1 in Formula 1 may be a substituted or unsubstituted C5 heterocycloalkyl ring, a substituted or unsubstituted C6 aryl ring, or a substituted or unsubstituted C4-5 heteroaryl ring, fused to an adjacent ring;
A2 may be a substituted or unsubstituted C5 heterocycloalkylene group, a substituted or unsubstituted C6 arylene group, or a substituted or unsubstituted C5 heteroarylene group;
X₁ to X₄ may be each independently CR or N;
R may be hydrogen, a halogen group, a C1-2 alkyl group, or a C1-2 alkoxy group; and
L₁ may be a C1-2 alkylene group.

In the compound according to an embodiment of the present disclosure, preferably, Y₁₁ to Y₁₄ in Formula 2 above may be each independently CRₐ or N; and Rₐ may be hydrogen, a hydroxy group, a C1-10 alkyl group, or a C1-10 alkoxy group.

In the compound according to an embodiment of the present disclosure, preferably, Y₂₁ to Y₂₄ in Formula 3 above may be each independently CR_{b}R_{c}, —C(═O)—, or NR_{d}; and R_{b} to R_{d} may be each independently hydrogen, a hydroxy group, a C1-10 alkyl group, or a C1-10 alkoxy group.

In the compound according to an embodiment of the present disclosure, A1 in the compound represented by Formula 1 above may be selected from the following structures:

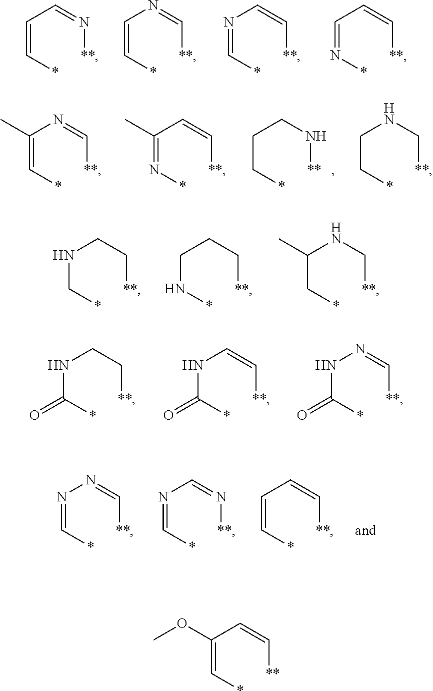

(with the proviso that * and ** in the above structures indicate points connected to each of two adjacent carbon atoms on the fused ring which A1 is attached in the compound represented by Formula 1, respectively).

In the compound according to an embodiment of the present disclosure, A2 in the compound represented by Formula 1 above may be selected from the following structures:

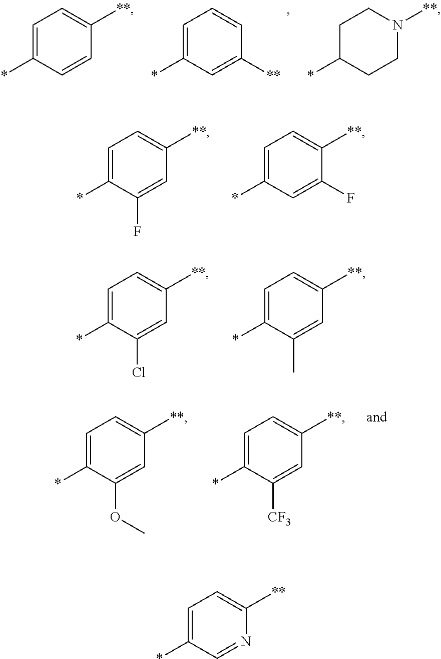

(with the proviso that * and ** in the above structures indicate points connected to L₁ and Z in the compound represented by Formula 1, respectively).

In the compound according to an embodiment of the present disclosure, Z in the compound represented by Formula 1 above may be selected from the following structures:

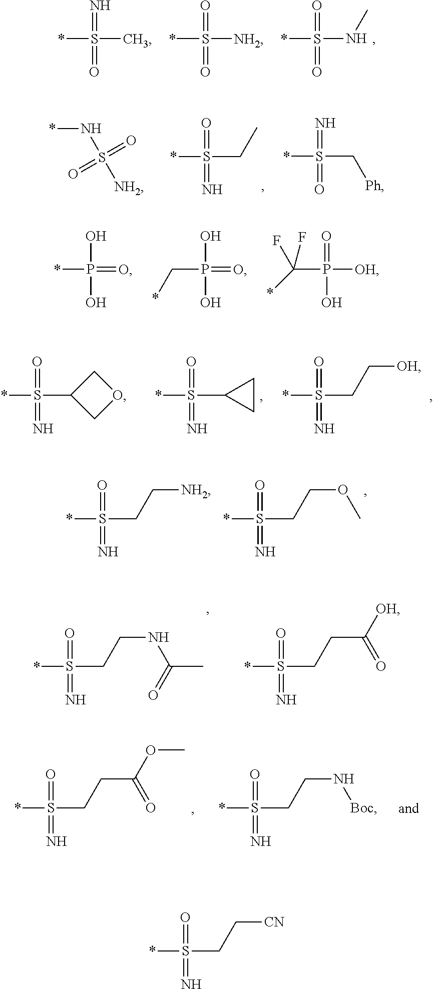

(with the proviso that * is a point connected to A2 in Formula 1).

Representative compounds of Formula 1 according to the present disclosure may include, but are not limited to, the following compounds:

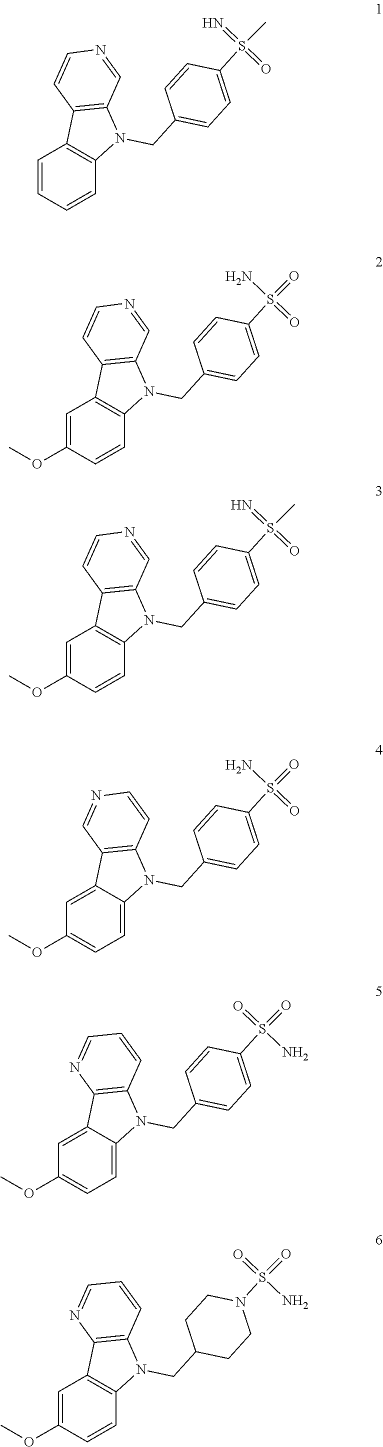 [SMILES: CS(=N)(=O)c1ccc(Cn2c3ccccc3c3ccncc32)cc1 ; COc1ccc2c(c1)c1ccncc1n2Cc1ccc(S(N)(=O)=O)cc1 ; COc1ccc2c(c1)c1ccncc1n2Cc1ccc(S(C)(=N)=O)cc1 ; COc1ccc2c(c1)c1cnccc1n2Cc1ccc(S(N)(=O)=O)cc1 ; COc1ccc2c(c1)c1ncccc1n2Cc1ccc(S(N)(=O)=O)cc1 ; COc1ccc2c(c1)c1ncccc1n2CC1CCN(S(N)(=O)=O)CC1]

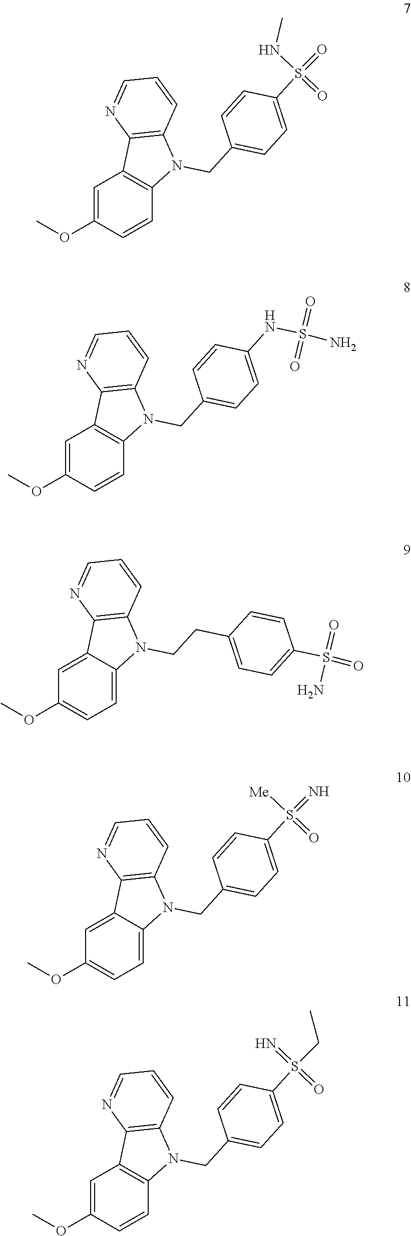 [SMILES: CNS(=O)(=O)c1ccc(Cn2c3ccc(OC)cc3c3ncccc32)cc1 ; COc1ccc2c(c1)c1ncccc1n2Cc1ccc(NS(N)(=O)=O)cc1 ; COc1ccc2c(c1)c1ncccc1n2CCc1ccc(S(N)(=O)=O)cc1 ; COc1ccc2c(c1)c1ncccc1n2Cc1ccc(S(C)(=N)=O)cc1 ; CCS(=N)(=O)c1ccc(Cn2c3ccc(OC)cc3c3ncccc32)cc1]

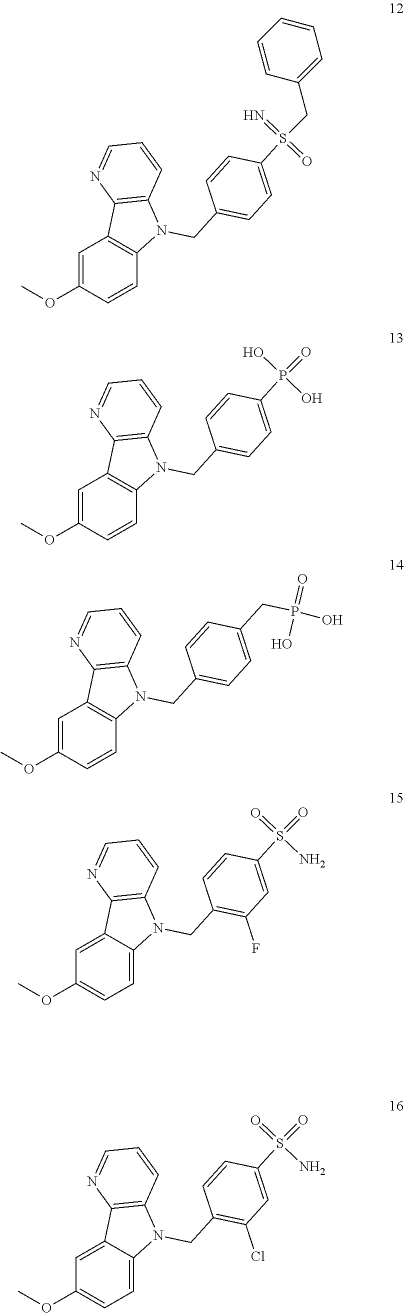 [SMILES: COc1ccc2c(c1)c1ncccc1n2Cc1ccc(S(=N)(=O)Cc2ccccc2)cc1 ; COc1ccc2c(c1)c1ncccc1n2Cc1ccc(P(=O)(O)O)cc1 ; COc1ccc2c(c1)c1ncccc1n2Cc1ccc(CP(=O)(O)O)cc1 ; COc1ccc2c(c1)c1ncccc1n2Cc1ccc(S(N)(=O)=O)cc1F ; COc1ccc2c(c1)c1ncccc1n2Cc1ccc(S(N)(=O)=O)cc1Cl]

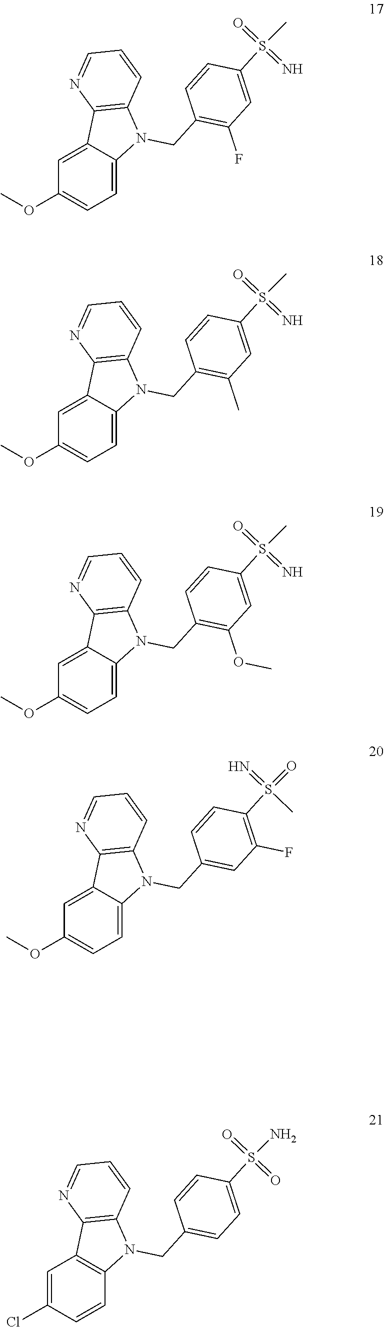

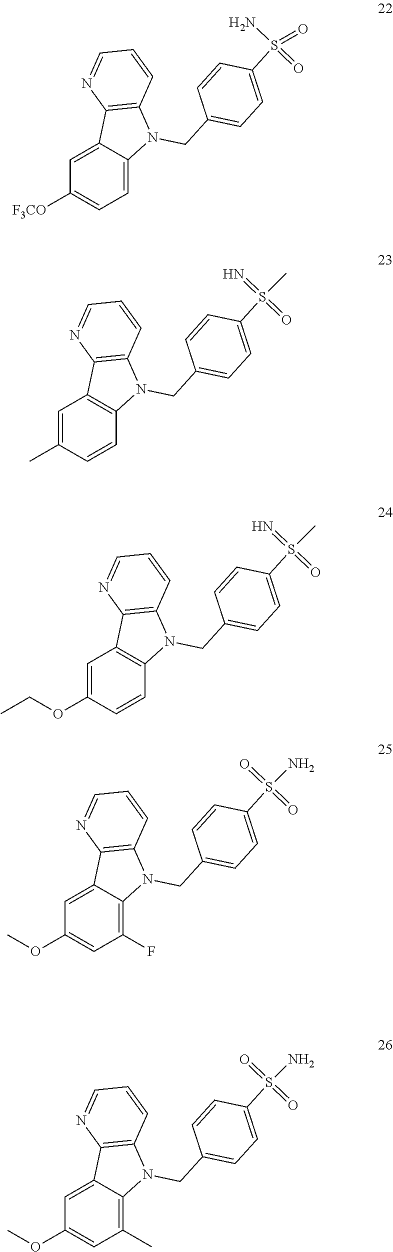 [SMILES: NS(=O)(=O)c1ccc(Cn2c3ccc(OC(F)(F)F)cc3c3ncccc32)cc1 ; Cc1ccc2c(c1)c1ncccc1n2Cc1ccc(S(C)(=N)=O)cc1 ; CCOc1ccc2c(c1)c1ncccc1n2Cc1ccc(S(C)(=N)=O)cc1 ; COc1cc(F)c2c(c1)c1ncccc1n2Cc1ccc(S(N)(=O)=O)cc1 ; COc1cc(C)c2c(c1)c1ncccc1n2Cc1ccc(S(N)(=O)=O)cc1]

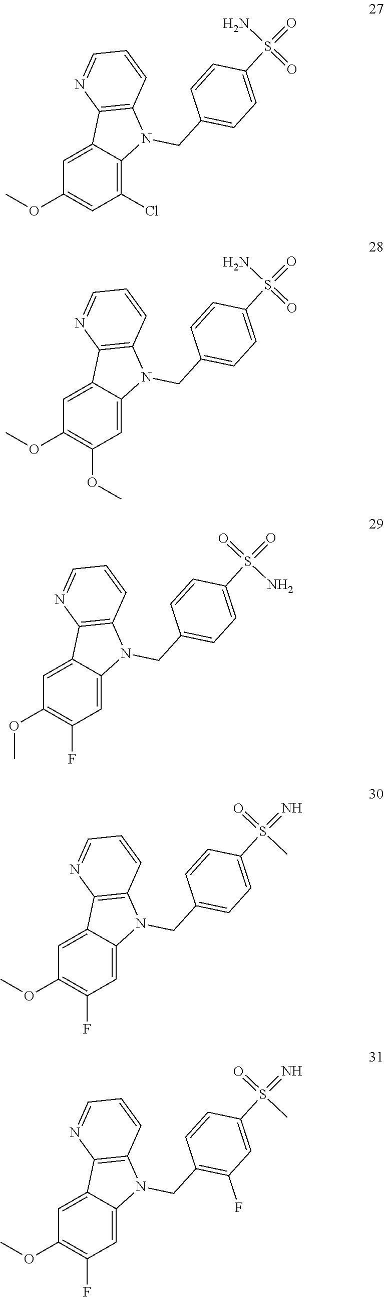

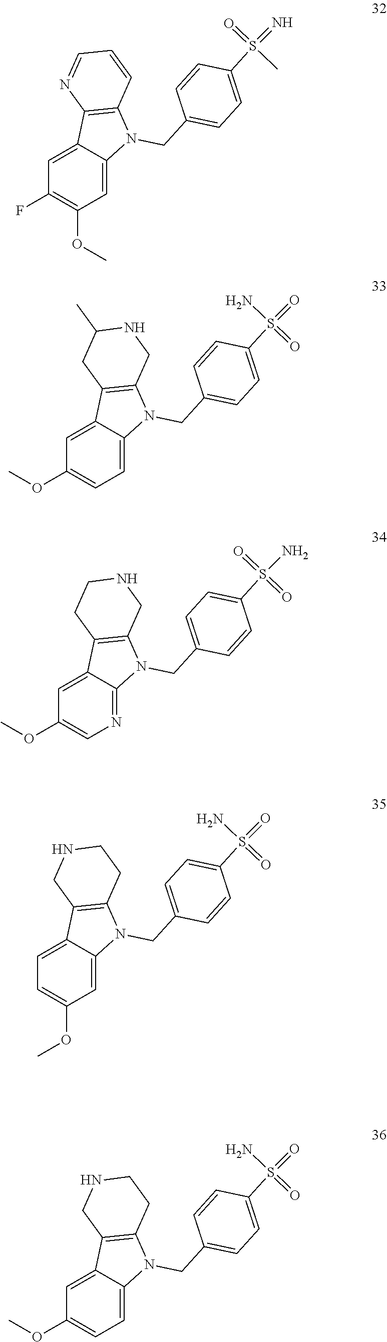

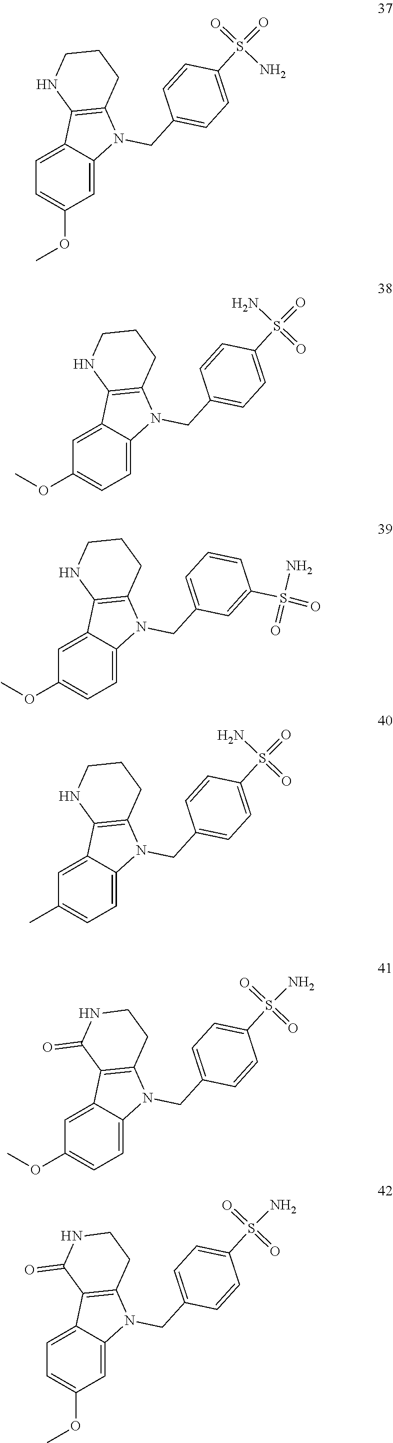 [SMILES: COc1ccc2c3c(n(Cc4ccc(S(N)(=O)=O)cc4)c2c1)CCCN3 ; COc1ccc2c(c1)c1c(n2Cc2ccc(S(N)(=O)=O)cc2)CCCN1 ; COc1ccc2c(c1)c1c(n2Cc2cccc(S(N)(=O)=O)c2)CCCN1 ; Cc1ccc2c(c1)c1c(n2Cc2ccc(S(N)(=O)=O)cc2)CCCN1 ; COc1ccc2c(c1)c1c(n2Cc2ccc(S(N)(=O)=O)cc2)CCNC1=O ; COc1ccc2c3c(n(Cc4ccc(S(N)(=O)=O)cc4)c2c1)CCNC3=O]

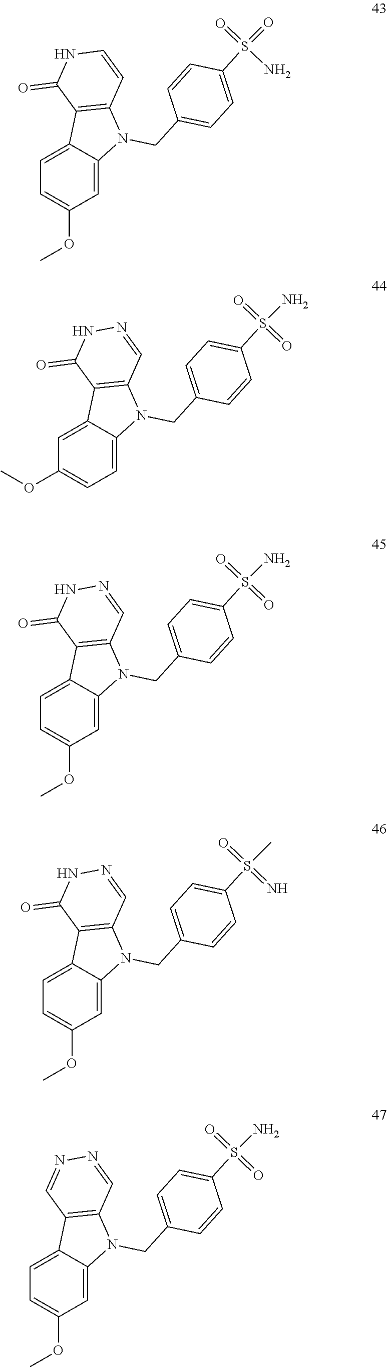 [SMILES: COc1ccc2c3c(=O)[nH]ccc3n(Cc3ccc(S(N)(=O)=O)cc3)c2c1 ; COc1ccc2c(c1)c1c(=O)[nH]ncc1n2Cc1ccc(S(N)(=O)=O)cc1 ; COc1ccc2c3c(=O)[nH]ncc3n(Cc3ccc(S(N)(=O)=O)cc3)c2c1 ; COc1ccc2c3c(=O)[nH]ncc3n(Cc3ccc(S(C)(=N)=O)cc3)c2c1 ; COc1ccc2c3cnncc3n(Cc3ccc(S(N)(=O)=O)cc3)c2c1]

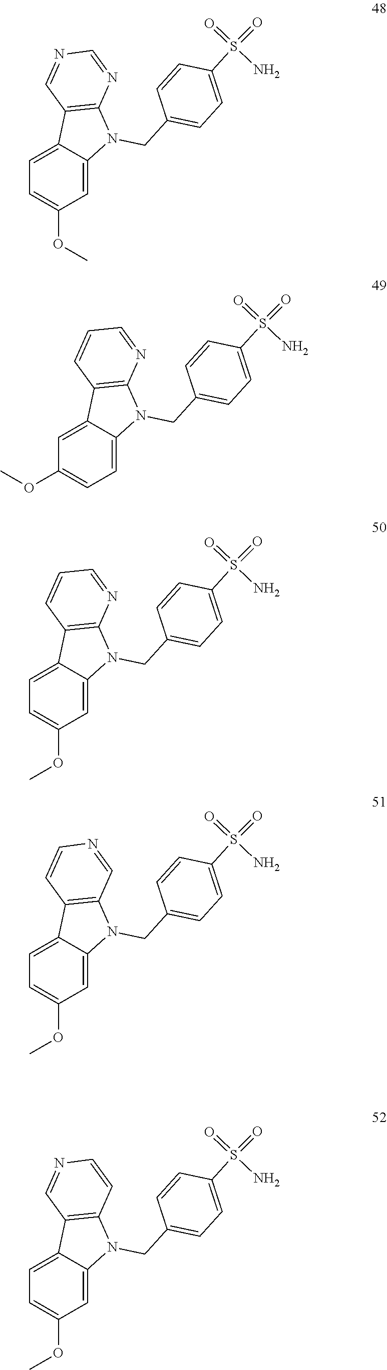 [SMILES: COc1ccc2c3cncnc3n(Cc3ccc(S(N)(=O)=O)cc3)c2c1 ; COc1ccc2c(c1)c1cccnc1n2Cc1ccc(S(N)(=O)=O)cc1 ; COc1ccc2c3cccnc3n(Cc3ccc(S(N)(=O)=O)cc3)c2c1 ; COc1ccc2c3ccncc3n(Cc3ccc(S(N)(=O)=O)cc3)c2c1 ; COc1ccc2c3cnccc3n(Cc3ccc(S(N)(=O)=O)cc3)c2c1]

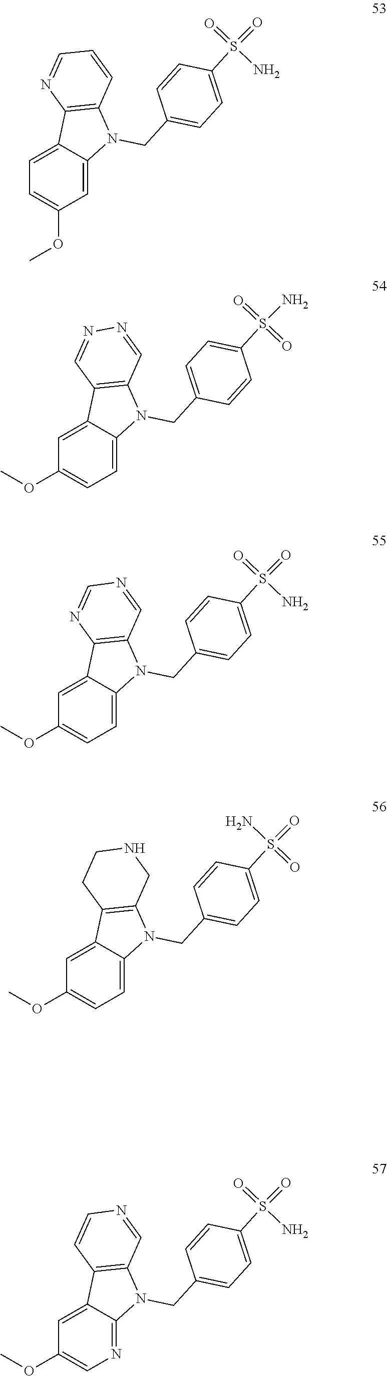 [SMILES: COc1ccc2c3ncccc3n(Cc3ccc(S(N)(=O)=O)cc3)c2c1 ; COc1ccc2c(c1)c1cnncc1n2Cc1ccc(S(N)(=O)=O)cc1 ; COc1ccc2c(c1)c1ncncc1n2Cc1ccc(S(N)(=O)=O)cc1 ; COc1ccc2c(c1)c1c(n2Cc2ccc(S(N)(=O)=O)cc2)CNCC1 ; COc1cnc2c(c1)c1ccncc1n2Cc1ccc(S(N)(=O)=O)cc1]

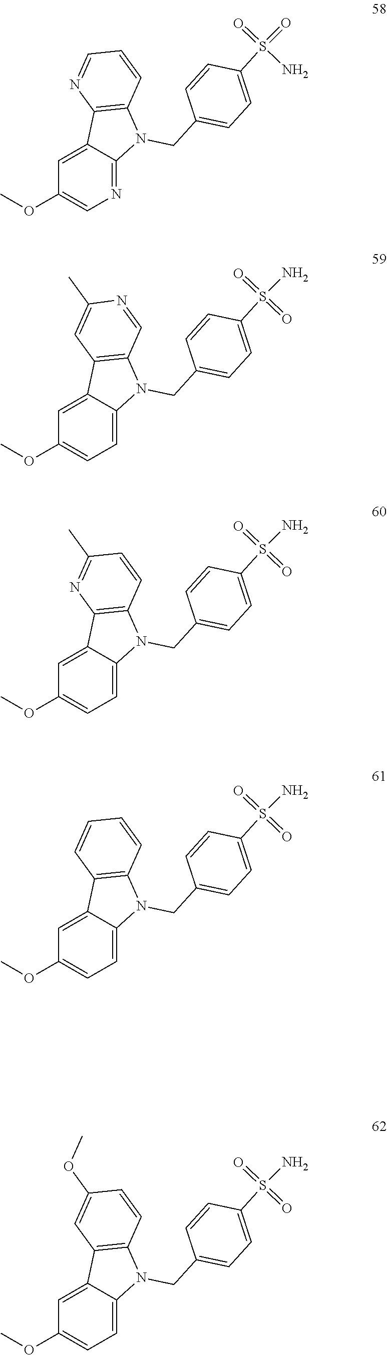 [SMILES: COc1cnc2c(c1)c1ncccc1n2Cc1ccc(S(N)(=O)=O)cc1 ; COc1ccc2c(c1)c1cc(C)ncc1n2Cc1ccc(S(N)(=O)=O)cc1 ; COc1ccc2c(c1)c1nc(C)ccc1n2Cc1ccc(S(N)(=O)=O)cc1 ; COc1ccc2c(c1)c1ccccc1n2Cc1ccc(S(N)(=O)=O)cc1 ; COc1ccc2c(c1)c1cc(OC)ccc1n2Cc1ccc(S(N)(=O)=O)cc1]

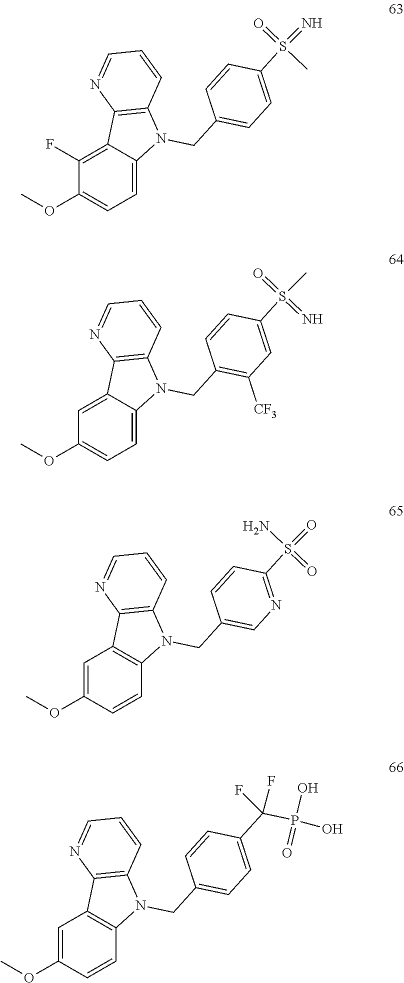

Salts, for example, pharmaceutically acceptable salts, of the compounds referred to herein are provided. The present disclosure also includes any or all stereochemical forms, including any enantiomeric or diastereomeric forms and any tautomeric or other forms of the compounds described.

In certain embodiments, compounds shown herein may exist as salts even when salts are not shown, and may include all salts and solvates of the compounds shown, as well as non-salt and non-solvate forms of the compounds.

In cases where tautomeric forms may exist for any of the compounds described herein, each and every tautomeric form is intended to be one or a portion of the tautomeric forms, even though they may be explicitly shown. A specifically shown tautomeric form may or may not be the form that predominates in solution when used in accordance with the methods described herein.

The novel compounds according to an embodiment of the present disclosure disclosure may have an asymmetric carbon center and may exist in the form of racemates or individual optical isomers. Of course, any form of isomers, including these optical isomers, may also belong to the category of the compound according to an embodiment. As used herein, the term “isomers” may collectively refer to different compounds having the same molecular formula, and “optical isomers” may collectively refer to any stereoisomers that may exist for the compound according to an embodiment, including same geometric isomers.

In the compounds represented by Formula 1 according to an embodiment of the present disclosure, it may be understood that each substituent may be attached to a chiral center of a carbon atom. In addition, any asymmetric carbon atom on the compound according to an embodiment may exist in any form of (R)-, (S)- or (R, S)-configuration, and may appropriately exist in either the (R)- or (S)-configuration, each in its separate form. In addition, the compound according to an embodiment may exist in any form of any possible isomer or mixture thereof and, for example, may exist in any form of pure geometric isomers, diastereomers, optical isomers, racemates, or mixtures thereof. In addition, when the compound according to an embodiment has a double bond, each substituent bonded to the double bond may be in the E or Z configuration. In addition, when the compound according to an embodiment contains a disubstituted cycloalkyl, each substituent of the cycloalkyl may have a cis or trans configuration.

Solvates and/or polymorphs of the compounds provided herein or pharmaceutically acceptable salts thereof are also contemplated. Solvates contain stoichiometric or non-stoichiometric amounts of a solvent, and are often formed during the crystallization process. Hydrates are formed when the solvent is water, or alcoholates are formed when the solvent is alcohol. Polymorphs include different crystal packing arrangements of compounds having the same elemental composition. Polymorphs usually have different X-ray diffraction patterns, infrared spectra, melting points, density, hardness, crystal shape, optical and electrical properties, stability, and/or solubility. A single crystal form may predominate by various factors, such as recrystallization solvent, rate of crystallization, and storage temperature.

In another aspect, the present disclosure provides a pharmaceutical composition for preventing, alleviating or treating cancer, comprising the compound represented by Formula 1 according to the present disclosure, a hydrate thereof, a solvate thereof, an isomer thereof or a pharmaceutically acceptable salt thereof as an active ingredient.

The pharmaceutical composition according to the present disclosure can be used to inhibit the activity of ENPP-1 in the body. Thus, the pharmaceutical composition of the present disclosure may be used for the purpose of treating, preventing and alleviating cancer diseases, and the cancer diseases may include, but are not limited to, gastric cancer, lung cancer, liver cancer, colorectal cancer, small intestine cancer, pancreatic cancer, brain cancer, bone cancer, melanoma, breast cancer, sclerosing adenosis, uterine cancer, cervical cancer, head and neck cancer, esophageal cancer, thyroid cancer, parathyroid cancer, kidney cancer, sarcoma, prostate cancer, urethral cancer, bladder cancer, hematologic malignancy (including leukemia, multiple myeloma, and myelodysplastic syndrome), lymphoma (including Hodgkin's disease and non-Hodgkin's lymphoma), psoriasis, fibroadenoma, and the like, and any cancer disease caused by abnormal cell growth may be prevented, treated or alleviated by treatment with the pharmaceutical composition according to the present disclosure.

In another aspect, the present disclosure provides an ENPP-1 inhibitor comprising the compound represented by Formula 1 according to the present disclosure, a hydrate thereof, a solvate thereof, an isomer thereof or a pharmaceutically acceptable salt thereof as an active ingredient.

The amount of the compound represented by Formula 1 above, a hydrate thereof, a solvate thereof, an isomer thereof or a pharmaceutically acceptable salt thereof as an active ingredient may be appropriately adjusted by the selection of those skilled in the art, depending on the aspect and method of using the pharmaceutical composition according to the present disclosure.

For example, the pharmaceutical composition according to the present disclosure comprises the compound represented by Formula 1 above, a hydrate thereof, a solvate thereof, an isomer thereof or a pharmaceutically acceptable salt thereof in an amount of 0.1 to 10% by weight, or 0.1 to 5% by weight based on the total weight of the total composition.

The compound represented by Formula 1 above, a hydrate thereof, a solvate thereof, an isomer thereof or a pharmaceutically acceptable salt thereof may be included alone in the pharmaceutical composition, or may also be included in combination with other pharmacologically acceptable carriers, excipients, diluents or auxiliary ingredients.

Examples of the pharmacologically acceptable carrier, excipient or diluent may be, but is not limited to, one or more selected from the group consisting of lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil, propylhydroxybenzoate, talc, magnesium stearate and mineral oil, dextrin, calcium carbonate, propylene glycol, liquid paraffin and physiological saline, and all conventional carriers, excipients or diluents may be used. In addition, the pharmaceutical composition may further comprise conventional fillers, extenders, binders, disintegrants, anti-coagulants, lubricants, wetting agents, pH adjusters, nutrients, vitamins, electrolytes, alginic acid and salts thereof, pectic acid and salts thereof, protective colorides, glycerin, flavorings, emulsifiers, preservatives, or the like.

The compound represented by Formula 1 according to the present disclosure, a hydrate thereof, a solvate thereof, an isomer thereof or a pharmaceutically acceptable salt thereof may enhance the therapeutic effect of anticancer drugs by conjoint administration with other anticancer drugs for treating cancer or tumors.

The method of administering the pharmaceutical composition may be either oral or parenteral, and as an example, it may be administered through various routes including oral, percutaneous, subcutaneous, intravenous or intramuscular routes. In addition, the formulation of the composition may vary depending on the method of use, and may be formulated using a method well known in the art to provide rapid, sustained or delayed release of the active ingredient after administration to a mammal. In general, solid formulations for oral administration include tablets, troches, soft or hard capsules, pills, powders, granules, and the like, and such formulations may be prepared by mixing one or more excipients, such as starch, calcium carbonate, sucrose or lactose, gelatin, and the like. In addition to simple excipients, lubricants such as magnesium stearate and talc may also be used. Liquid formulations for oral administration include suspensions, solutions for internal use, emulsions, syrups, and the like, and may comprise various excipients, such as wetting agents, sweeteners, flavoring agents, preservatives, and the like, in addition to commonly used simple diluents such as water and liquid paraffin. Forms for parenteral administration may be in the form of creams, lotions, ointments, plasters, liquids and solutions, aerosols, fluidextracts, elixirs, infusions, sachets, patches, injections, or the like, and may preferably be in the form of an isotonic aqueous solution or suspension in the case of an injectable formulation.

In addition, the pharmaceutical composition may further comprise adjuvants such as sterilizers, preservatives, stabilizers, hydrating agents or emulsifying accelerators, salts and/or buffers for controlling osmotic pressure, and the like, and other therapeutically useful substances, and may be formulated according to conventional mixing, granulating or coating methods, and may also be formulated using appropriate methods known in the art.

In addition, the present disclosure provides a method for preparing the compound represented by Formula 1 above.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” it is meant the concentration of a compound that is sufficient to elicit the desired therapeutic effect. It is generally understood that the effective amount of the compound will vary according to the weight, sex, age, and medical history of the subject. Other factors which influences the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present disclosure. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N. Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In certain embodiments, a compound or an agent is administered orally, e.g., to a subject by ingestion. In certain embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, “alkyl” refers to a straight-chain or branched-chain aliphatic saturated hydrocarbon group, and may be preferably an alkyl having 1 to 6 carbon atoms, and more preferably an alkyl having 1 to 4 carbon atoms. Examples of such alkyls include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethyl butyl, 2,2-dimethylbutyl, 3,3-dimethylbutyl, and 2-ethylbutyl. The “alkyl” group may be optionally substituted. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O) NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

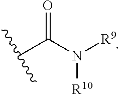

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

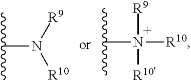

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

As used herein, “aryl” refers to a carbocyclic aromatic group that may be further fused with a second 5- or 6-membered carbocyclic group that may be aromatic, saturated or unsaturated, and examples of aryl may include, but are not limited to, phenyl, indanyl, 1-naphthyl, 2-naphthyl, tetrahydronapthyl, and the like. Aryl may be linked to other groups at appropriate positions on the aromatic ring. Preferably the ring is a 5- to 7-membered ring, more preferably a 6-membered ring. Aryl groups include benzene, naphthalene, phenanthrene, phenol, aniline, and the like.

The term “carbamate” is art-recognized and refers to a group

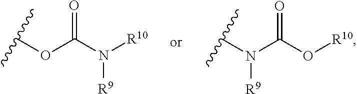

wherein R⁹ and R¹⁰ each independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group-C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

As used herein, “heteroaryl” refers to a heteroaromatic compound containing at least one heteroatom selected from the group consisting of N, O and S, unless otherwise specified, and preferably the heteroaryl group may include, but are not limited to, a pyridine group, a pyrazine group, a pyrimidine group, a pyridazine group, a pyrazole group, an imidazole group, a triazole group, an indole group, an oxadiazole group, a thiadiazole group, a quinoline group, an isoquinoline group, an isoxazole group, an oxazole group, a thiazole group, and pyrrole group. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

As used herein, “heterocycle” refers to an aromatic or non-aromatic ring containing a heteroatom selected from a nitrogen atom, a sulfur atom and an oxygen atom other than a carbon atom as a ring member atom, and preferably includes a 4- to 10-membered and more preferably a 5- to 9-membered aromatic or non-aromatic ring containing 1 to 4 of the above heteroatoms. Examples of such aromatic rings include thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl, and benzothiazolyl. In addition, examples of such non-aromatic rings include tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisoxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, and azepinyl. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls.

As used herein, “arylene” and “heteroarylene” refer to divalent radicals of an aromatic ring and a heteroaromatic ring.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “phosphoryl” is art-recognized and refers to the group represented by the general formula

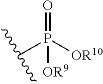

wherein R⁹ and R¹⁰ each independently represent a negative charge, hydrogen, or hydrocarbyl.

The term “phosphoramidityl” is art-recognized and refers to the group represented by the general formula

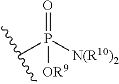

wherein R⁹ represents a negative charge, hydrogen, or hydrocarbyl; and each R¹⁰ independently represents hydrogen or hydrocarbyl.

The term “phosphoramidatyl” is art-recognized and refers to the group represented by the general formula

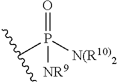

wherein each R⁹ and R¹⁰ independently represent hydrogen or hydrocarbyl.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

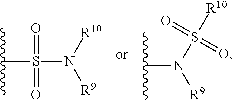

wherein each R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “iminosulfanonyl” is art-recognized and refers to the group represented by the general formula

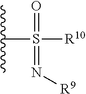

wherein R⁹ and R¹⁰ each independently represents hydrogen or hydrocarbyl.

In certain embodiments, as used herein, “substituent” includes a halogen group, a cyano group, a nitro group, a substituted or unsubstituted amino group including a substituted or unsubstituted alkyl group and a substituted or unsubstituted carboxyl group, a substituted or unsubstituted hydrocarbon group, substituted or unsubstituted heterocyclic group, acyl group, substituted or unsubstituted amino group, substituted or unsubstituted carbamoyl group, substituted or unsubstituted thiocarbamoyl group, substituted or unsubstituted sulfamoyl group, substituted or unsubstituted hydroxy group, a substituted or unsubstituted sulfonyl (SH) group, and a substituted or unsubstituted silyl group.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O) R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

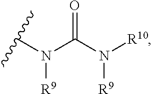

wherein R⁹ and R¹⁰ each independently represent hydrogen or a hydrocarbyl.

The term “modulate” as used herein includes the inhibition or suppression of a function or activity (such as cell proliferation) as well as the enhancement of a function or activity.

As used herein, the term “pharmaceutically acceptable salt” according to the present disclosure equally retains the biological effectiveness and properties of a compound disclosed herein according to an embodiment, and may collectively refer to any preferred salt in terms of pharmaceutical, biological or other properties. Non-limiting examples of these salts may be salts in which an inorganic base or an organic base is added to the compound disclosed herein, or acid addition salts. Examples of organic acids capable of forming such acid addition salts may be acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, toluenesulfonic acid, and the like, and examples of inorganic acids may be hydrochloric acid, hydrobromic acid, sulfuric acid, nitric acid, phosphoric acid, and the like.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present disclosure and are not intended to limit the invention.

### Example 1: Synthesis of Intermediates

### [Preparative Example 1] Preparation of 4-(bromomethyl)benzenesulfonamide (Intermediate 1)

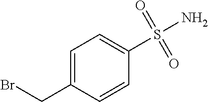 [SMILES: NS(=O)(=O)c1ccc(CBr)cc1]

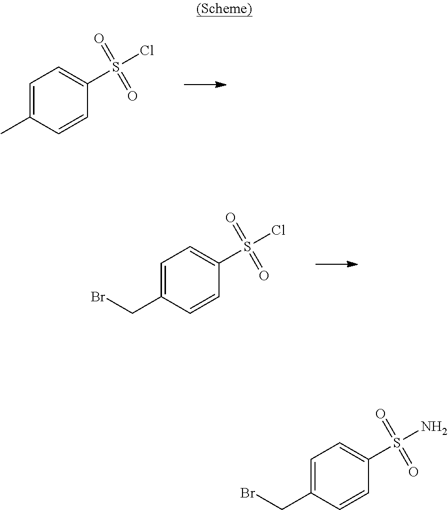

### (Step 1) Preparation of 4-(bromomethyl)benzenesulfonyl chloride

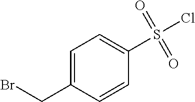 [SMILES: O=S(=O)(Cl)c1ccc(CBr)cc1]

p-Toluenesulfonyl chloride (5.00 g, 26.2 mmol) was dissolved in benzene (10.0 mL), and then N-bromosuccinimide (5.60 g, 31.5 mmol) and a,a′-azobis(isobutyronitrile) (4.70 g, 28.8 mmol) were added to the mixture sequentially. The reaction mixture was stirred at 100° C. for 12 hours under nitrogen. After cooling to room temperature, distilled water was added and extracted twice with ethyl acetate. The organic layers were dried over anhydrous sodium sulfate, and then concentrated under reduced pressure. The residue was purified by silica gel column chromatography (ethyl acetate:n-hexane=1:20) to obtain the title compound as a white solid. (5.12 g, 72%) MS m/z: 267 [M+1]⁺

### (Step 2) Preparation of 4-(bromomethyl)benzenesulfonamide (Intermediate 1)

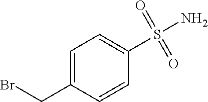 [SMILES: NS(=O)(=O)c1ccc(CBr)cc1]

The compound prepared in (Step 1) above (3.30 g, 12.2 mmol) was dissolved in tetrahydrofuran (34.0 mL), and then cooled to 0° C., and 28-30% aqueous ammonia (4.80 mL) was added to the mixture slowly. The reaction mixture was stirred at room temperature for 2 hours under nitrogen. After completion of the reaction, distilled water was added and extracted twice with ethyl acetate. The organic layers were washed with distilled water and a saturated saline solution, dried over anhydrous sodium sulfate, and then concentrated under reduced pressure. The title compound was obtained as a white solid without further purification. (2.84 g, 93%) MS m/z: 251 [M+1]^{+ 1}H NMR (400 MHz, CDCl₃) δ ppm: 7.81 (d, J=8.4 Hz, 2H), 7.63 (d, J=8.4 Hz, 2H), 7.39 (s, 2H), 4.76 (s, 2H).

### [Preparative Example 2] Preparation of tert-butyl ((4-(bromomethyl) piperidin-1-yl)sulfonyl)carbamate (Intermediate 2)

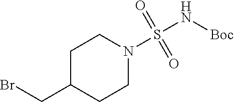 [SMILES: CC(C)(C)OC(=O)NS(=O)(=O)N1CCC(CBr)CC1]

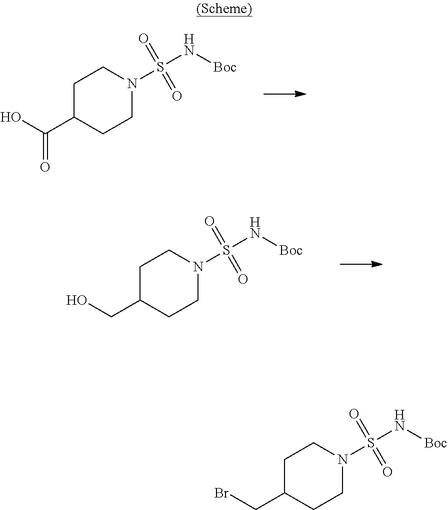 [SMILES: CC(C)(C)OC(=O)NS(=O)(=O)N1CCC(C(=O)O)CC1 ; CC(C)(C)OC(=O)NS(=O)(=O)N1CCC(CO)CC1 ; CC(C)(C)OC(=O)NS(=O)(=O)N1CCC(CBr)CC1]

### (Step 1) Preparation of tert-butyl ((4-(hydroxymethyl) piperidin-1-yl)sulfonyl)carbamate

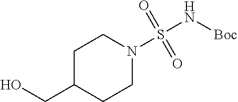 [SMILES: CC(C)(C)OC(=O)NS(=O)(=O)N1CCC(CO)CC1]

1-(tert-Butoxycarbonylsulfamoyl) piperidine-4-carboxylic acid (200 mg, 0.65 mmol) was dissolved in tetrahydrofuran (2.00 mL), and then cooled to 0° C., and lithium aluminum hydride (36.9 mg, 0.97 mmol) was added to the mixture. It was stirred at 0° C. for 2 hours under nitrogen. After completion of the reaction, a mixed solvent in a ratio of methanol:15% sodium carbonate solution:distilled water=1:1:3 was added, and then extracted twice with ethyl acetate. The organic layers were dried over anhydrous sodium sulfate, and then concentrated under reduced pressure. The residue was purified by silica gel column chromatography (ethyl acetate:n-hexane=1:3) to obtain the title compound as a colorless oil. (95.0 mg, 50%) MS m/z: 295 [M+1]⁺

### (Step 2) Preparation of tert-butyl ((4-(bromomethyl) piperidin-1-yl)sulfonyl)carbamate (Intermediate 2)

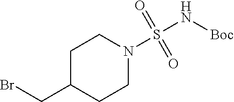 [SMILES: CC(C)(C)OC(=O)NS(=O)(=O)N1CCC(CBr)CC1]

The compound prepared in (Step 1) above (95.0 mg, 0.32 mmol) was dissolved in methylene chloride (1.00 mL), and then phosphorus tribromide (118 mg, 0.36 mmol) and triphenylphosphine (76.2 mg, 0.29 mmol) were added to the mixture slowly. The reaction mixture was stirred at room temperature for 5 hours under nitrogen. After completion of the reaction, distilled water was added thereto slowly, and extracted twice with ethyl acetate. The organic layers were collected, washed with distilled water and a saturated saline solution, dried over anhydrous sodium sulfate, and then concentrated under reduced pressure. The residue was purified by silica gel column chromatography (ethyl acetate:n-hexane=1:5) to obtain the title compound as a white solid. (56.0 mg, 49%) MS m/z: 358 [M+1]^{+ 1}H NMR (400 MHz, CDCl₃) δ 6.93 (s, 1H), 3.91 (d, J=12.6 Hz, 2H), 3.31 (d, J=6.3 Hz, 2H), 2.93 (t, J=12.5 Hz, 2H), 1.93 (d, J=13.2 Hz, 2H), 1.63-1.45 (m, 11H).

### [Preparative Example 3] Preparation of tert-butyl ((4-(bromomethyl)phenyl)sulfonyl)(methyl)carbamate (Intermediate 3)

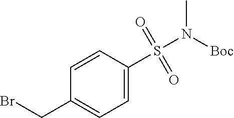 [SMILES: CN(C(=O)OC(C)(C)C)S(=O)(=O)c1ccc(CBr)cc1]

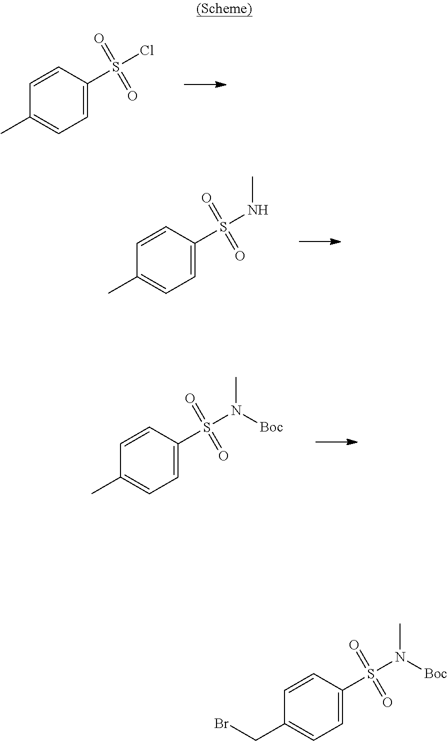

### (Step 1) Preparation of N,4-dimethylbenzenesulfonamide

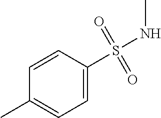

p-Toluene sulfonyl chloride (1.50 g, 7.87 mmol) was dissolved in methylene chloride (8.00 mL), and then cooled to 0° C., and an aqueous solution of methylamine (40 wt. % in H₂O, 2.04 mL, 23.6 mmol) and N,N-diisopropylethylamine (2.70 mL, 15.7 mmol) were added to the mixture slowly. The reaction mixture was stirred at room temperature for 2 hours. After completion of the reaction, distilled water and a saturated aqueous ammonium chloride solution were added, and extracted twice with methylene chloride. The organic layers were dried over anhydrous sodium sulfate, and then concentrated under reduced pressure. The residue was purified by silica gel column chromatography (ethyl acetate:n-hexane=1:4 to 1:1) to obtain the title compound as a white solid. (1.40 g, 97%) MS m/z: 186 [M+1]^{+ 1}H NMR (400 MHz, CDCl₃) δ ppm: 7.74 (d, J=8.5 Hz, 2H), 7.31 (d, J=8.5 Hz, 2H), 4.27 (d, J=4.5 Hz, 1H), 2.65 (d, J=5.5 Hz, 3H), 2.44 (s, 3H).

### (Step 2) Preparation of tert-butyl methyl(tosyl) carbamate

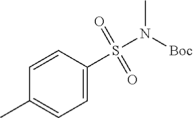 [SMILES: Cc1ccc(S(=O)(=O)N(C)C(=O)OC(C)(C)C)cc1]

The compound prepared in (Step 1) above (600 mg, 3.24 mmol) was dissolved in methylene chloride (16.0 mL), di-tert-butyl dicarbonate (0.90 mL, 3.89 mmol), N,N-diisopropylethylamine (1.70 mL, 9.72 mmol) and 4-dimethylaminopyridine (39.6 mg, 0.32 mmol) were slowly added to the mixture sequentially. The reaction mixture was stirred at room temperature for 16 hours under nitrogen. After completion of the reaction, a saturated aqueous ammonium chloride solution was added, and extracted twice with methylene chloride. The organic layers were dried over anhydrous sodium sulfate, and then concentrated under reduced pressure. The residue was purified by silica gel column chromatography (ethyl acetate:n-hexane=3:7) to obtain the title compound as a colorless liquid. (922 mg, 99%) MS m/z: 286 [M+1]^{+ 1}H NMR (400 MHz, CDCl₃) δ ppm: 7.77 (d, J=8.5 Hz, 2H), 7.30 (d, J=7.9 Hz 2H), 3.35 (s, 3H), 2.44 (s, 3H), 1.35 (s, 9H).

### (Step 3) Preparation of tert-butyl ((4-(bromomethyl)phenyl)sulfonyl)(methyl)carbamate (Intermediate 3)

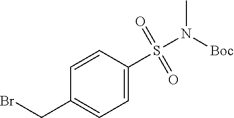 [SMILES: CN(C(=O)OC(C)(C)C)S(=O)(=O)c1ccc(CBr)cc1]

The compound prepared in (Step 2) above (920 mg, 3.22 mmol) was dissolved in carbon tetrachloride (5.00 mL), and then N-bromosuccinimide (545.1 mg, 3.06 mmol) and a,a′-azobis(isobutyronitrile) (37.1 mg, 0.23 mmol) were added to the mixture sequentially. The reaction mixture was stirred at 80° C. for 3 hours under nitrogen. After cooling to room temperature, it was concentrated under reduced pressure, and distilled water was added, and then extracted twice with ethyl acetate. The organic layers were washed with distilled water and dried over anhydrous sodium sulfate, and then concentrated under reduced pressure. The residue was purified by silica gel column chromatography (ethyl acetate:n-hexane=1:4) to obtain the title compound as a colorless liquid. (662 mg, 56%) MS m/z: 365 [M+1]^{+ 1}H NMR (400 MHz, CDCl₃) δ ppm: 7.86 (d, J=8.5 Hz, 2H), 7.53 (d, J=8.5 Hz, 2H), 4.50 (s, 2H), 3.36 (s, 3H), 1.35 (s, 9H).

### [Preparative Example 4] Preparation of tert-butyl (4-(bromomethyl)phenyl) carbamate (Intermediate 4)

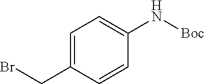 [SMILES: CC(C)(C)OC(=O)Nc1ccc(CBr)cc1]

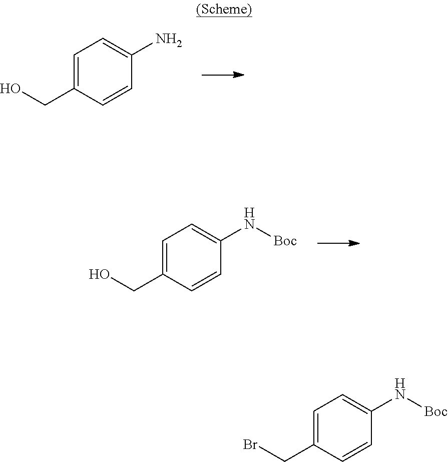 [SMILES: Nc1ccc(CO)cc1 ; CC(C)(C)OC(=O)Nc1ccc(CO)cc1 ; CC(C)(C)OC(=O)Nc1ccc(CBr)cc1]

### (Step 1) Preparation of tert-butyl (4-(hydroxymethyl)phenyl) carbamate

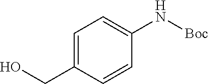 [SMILES: CC(C)(C)OC(=O)Nc1ccc(CO)cc1]

4-Aminobenzyl alcohol (300 mg, 2.43 mmol) was dissolved in tetrahydrofuran (5.00 mL), sodium carbonate (309 mg, 2.92 mmol) and di-tert-butyl dicarbonate (0.56 mL, 2.43 mmol) were slowly added to the mixture sequentially. The reaction mixture was stirred at room temperature for 24 hours under nitrogen. After completion of the reaction, 2N HCl (10.0 mL) was added and extracted twice with ethyl acetate. The organic layers were washed with distilled water and a saturated saline solution, dried over anhydrous sodium sulfate, and then concentrated under reduced pressure. The title compound was obtained as a yellow liquid without further purification. (542 mg, 99%) MS m/z: 224 [M+1]^{+ 1}H NMR (400 MHz, CDCl₃) δ 7.43-7.15 (m, 4H), 6.48 (s, 1H), 4.63 (d, J=2.3 Hz, 2H), 1.57-1.42 (m, 9H).

### (Step 2) Preparation of tert-butyl (4-(bromomethyl)phenyl) carbamate (Intermediate 4)

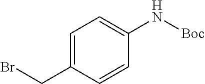 [SMILES: CC(C)(C)OC(=O)Nc1ccc(CBr)cc1]

The compound prepared in (Step 1) above (300 mg, 1.34 mmol) was dissolved in methylene chloride (6.00 mL), and then phosphorus tribromide (400 mg, 1.48 mmol) was added to the mixture slowly. The reaction mixture was stirred at room temperature for 5 hours under nitrogen. After completion of the reaction, distilled water was added, and extracted twice with ethyl acetate. The organic layers were washed with distilled water and a saturated saline solution, dried over anhydrous sodium sulfate, and then concentrated under reduced pressure. The title compound was obtained as a white solid without further purification. (146 mg, 38%) MS m/z: 287 [M+1]^{+ 1}H NMR (400 MHz, CDCl₃) δ 7.36-7.27 (m, 4H), 6.56 (d, J=15.4 Hz, 1H), 4.47 (s, 2H), 1.51 (s, 9H).

### [Preparative Example 5] Preparation of 4-vinylbenzenesulfonamide (Intermediate 5)

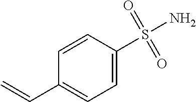

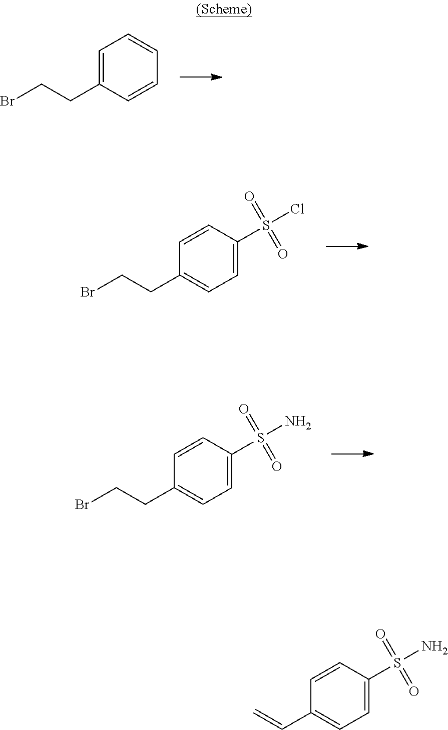

### (Step 1) Preparation of 4-(2-bromoethyl)benzenesulfonyl chloride

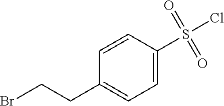 [SMILES: O=S(=O)(Cl)c1ccc(CCBr)cc1]

(2-Bromoethyl)benzene (2.00 g, 10.8 mmol) was dissolved in methylene chloride anhydride (10.8 mL), and then cooled to 0° C., and chlorosulfonic acid (2.20 mL, 32.4 mmol) was added to the mixture slowly. The reaction mixture was stirred at room temperature for 3 hours under nitrogen. After completion of the reaction, reaction mixture was slowly dropped into ice water, and then extracted twice with methylene chloride. The organic layers were collected, washed with distilled water and a saturated saline solution, dried over anhydrous sodium sulfate, and then concentrated under reduced pressure. The title compound was obtained as a white solid without further purification. (2.10 g, 67%) MS m/z: 284 [M+1]^{+ 1}H NMR (400 MHz, CDCl₃) δ ppm: 7.89 (d, J=5.6 Hz, 2H), 7.36 (s, 2H), 3.55 (s, 2H), 3.21 (s, 2H).

### (Step 2) Preparation of 4-(2-bromoethyl)benzenesulfonamide

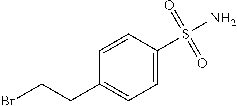 [SMILES: NS(=O)(=O)c1ccc(CCBr)cc1]

The compound prepared in (Step 1) above (2.10 g) was dissolved in tetrahydrofuran (6.00 mL), and then cooled to 0° C., and 28-30% aqueous ammonia (2.00 mL) was added to the mixture slowly. The reaction mixture was stirred at room temperature for 2 hours under nitrogen. The mixture was dissolved in distilled water, and then the resulting solid was filtered and dried to obtain the title compound as a white solid. (1.06 g, 55%) MS m/z: 265 [M+1]^{+ 1}H NMR (400 MHz, DMSO-d₆) δ ppm: 7.76 (d, J=5.5 Hz, 2H), 7.56-7.40 (m, 2H), 7.31 (s, 2H), 3.78 (s, 2H), 3.21 (s, 2H).

### (Step 3) Preparation of 4-vinylbenzenesulfonamide (Intermediate 5)

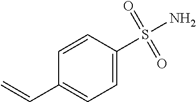

The compound prepared in (Step 2) above (300 mg, 1.14 mmol) was dissolved in tetrahydrofuran (5.00 mL), and then cooled to 0° C., and potassium-tert-butoxide (1.0 M tetrahydrofuran solution, 3.3 mL, 3.40 mmol) was added slowly. The reaction mixture was stirred at 60° C. for 3 hours under nitrogen. After completion of the reaction, distilled water was added slowly, and extracted twice with ethyl acetate. The organic layers were washed with distilled water and a saturated saline solution, dried over anhydrous sodium sulfate, and then concentrated under reduced pressure. The title compound was obtained as a white solid without further purification. (157 mg, 75%) MS m/z: 184 [M+1]^{+ 1}H NMR (400 MHz, CDCl₃) δ ppm: 7.87 (d, J=9.1 Hz, 2H), 7.52 (d, J=8.5 Hz, 2H), 6.79-6.68 (m, 1H), 5.87 (d, J=17.5 Hz, 1H), 5.43 (d, J=9.1 Hz, 1H), 4.77 (s, 2H).

### [Preparative Example 6] Preparation of 4-(bromomethyl)phenyl)(methyl) sulfane (Intermediate 6)

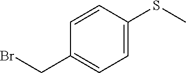

(4-Methylsulfanylphenyl) methanol (5.00 g, 32.4 mmol) was dissolved in methylene chloride (20.0 mL), and then tetrabromomethane (13.9 g, 42.1 mmol) and triphenylphosphine (9.30 g, 35.7 mmol) were added to the mixture at 0° C. The reaction mixture was stirred at room temperature for 16 hours under nitrogen. After completion of the reaction, distilled water was added slowly, and extracted twice with methylene chloride. The organic layers were dried over anhydrous sodium sulfate, and then concentrated under reduced pressure. The residue was purified by silica gel column chromatography (ethyl acetate:n-hexane=0:10 to 1:4) to obtain the title compound as a white solid. (5.40 g, 77%) MS m/z: 218 [M+1]^{+ 1}H NMR (400 MHz, CDCl₃) δ ppm: 7.30 (d, J=8.4 Hz, 2H), 7.2 (d, J=8.5 Hz, 2H), 4.48 (s, 2H), 2.48 (s, 3H).

### [Preparative Example 7] Preparation of (4-(bromomethyl)phenyl) (ethyl) sulfane (Intermediate 7)

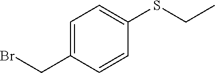

(4-Ethylsulfanylphenyl) methanol (0.50 g, 2.97 mmol) was dissolved in methylene chloride (10.0 mL), and then tetrabromomethane (1.30 g, 3.86 mmol) and triphenylphosphine (857.4 mg, 3.27 mmol) were added to the mixture at 0° C. The reaction mixture was stirred at room temperature for 16 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a colorless liquid. (232 mg, 34%) MS m/z: 232 [M+1]^{+ 1}H NMR (400 MHz, CDCl₃) δ ppm: 7.29 (d, J=8.5 Hz, 2H), 7.25 (d, J=8.6 Hz, 2H), 4.47 (s, 2H), 2.95 (q, J=7.4 Hz, 2H), 1.33 (t, J=7.4 Hz, 3H).

### [Preparative Example 8] Preparation of benzyl (4-(bromomethyl)phenyl) sulfane (Intermediate 8)

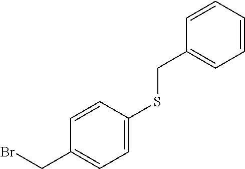 [SMILES: BrCc1ccc(SCc2ccccc2)cc1]

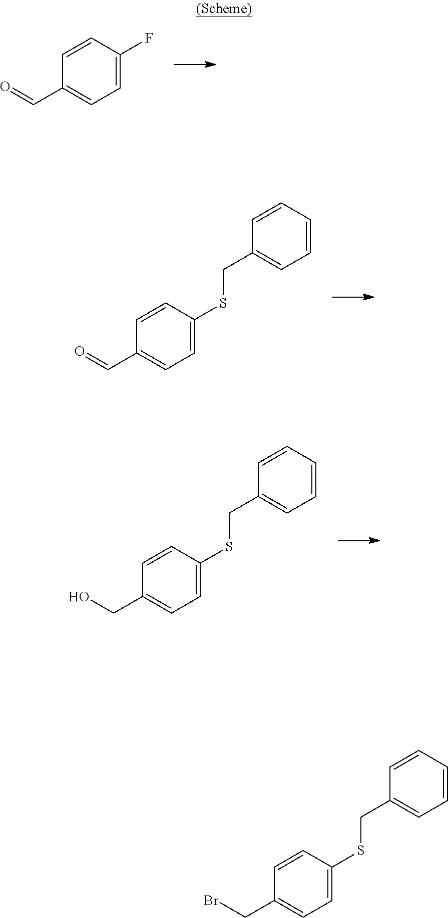 [SMILES: O=Cc1ccc(F)cc1 ; O=Cc1ccc(SCc2ccccc2)cc1 ; OCc1ccc(SCc2ccccc2)cc1 ; BrCc1ccc(SCc2ccccc2)cc1]

### (Step 1) Preparation of 4-(benzylthio)benzaldehyde

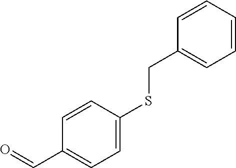 [SMILES: O=Cc1ccc(SCc2ccccc2)cc1]

4-Fluorobenzaldehyde (1.00 g, 8.06 mmol) was dissolved in N,N-dimethylformamide (4.00 mL), and then benzyl mercaptan (1.10 g, 8.86 mmol) and potassium carbonate (1.70 g, 12.1 mmol) was added to the mixture. The reaction mixture was stirred at 100° C. for 4 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a green solid. (1.10 g, 59%) MS m/z: 229 [M+1]^{+ 1}H NMR (400 MHz, CDCl₃) δ ppm: 9.92 (s, 1H), 7.73 (d, J=8.4 Hz, 2H), 7.39-7.36 (m, 4H), 7.31 (t, J=7.2 Hz, 2H), 7.27 (d, J=7.0 Hz, 1H), 4.24 (s, 2H).

### (Step 2) Preparation of (4-(benzylthio)phenyl) methanol

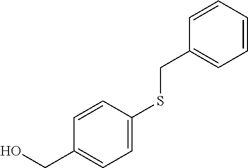 [SMILES: OCc1ccc(SCc2ccccc2)cc1]

The compound prepared in (Step 1) above (1.10 g, 4.77 mmol) was dissolved in methanol (7.00 mL), and then cooled to 0° C., and sodium borohydride (199 mg, 5.25 mmol) was added to the mixture and stirred for 3 hours. After completion of the reaction, distilled water was added slowly, and extracted twice with ethyl acetate. The organic layers were dried over anhydrous sodium sulfate, and then concentrated under reduced pressure. The title compound was obtained as a white solid without further purification. (978 mg, 89%) MS m/z: 231 [M+1]^{+ 1}H NMR (400 MHz, CDCl₃) δ ppm: 7.31-7.24 (m, 9H), 4.64 (d, J=5.9 Hz, 2H), 4.11 (s, 2H).

### (Step 3) Preparation of benzyl (4-(bromomethyl)phenyl) sulfane (Intermediate 8)

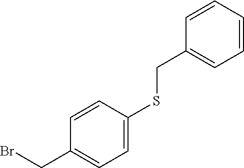 [SMILES: BrCc1ccc(SCc2ccccc2)cc1]

The compound prepared in (Step 2) above (0.50 g, 2.17 mmol) was dissolved in methylene chloride (10.0 mL), and then tetrabromomethane (936 mg, 2.82 mmol) and triphenylphosphine (626 mg, 2.39 mmol) were added to the mixture at 0° C. The reaction mixture was stirred at room temperature for 16 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a white solid. (468 mg, 74%) MS m/z: 294 [M+1]^{+ 1}H NMR (400 MHz, CDCl₃) δ ppm: 7.36-7.20 (m, 9H), 4.45 (s, 2H), 4.13 (s, 2H).

### [Preparative Example 9] Preparation of diethyl (4-(bromomethyl)benzyl)phosphonate (Intermediate 9)

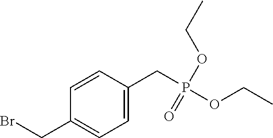 [SMILES: CCOP(=O)(Cc1ccc(CBr)cc1)OCC]

1,4-bis(bromomethyl)benzene (396 mg, 1.50 mmol) was dissolved in N,N-dimethylformamide (0.80 mL), and then triethyl phosphite (128 μl, 0.75 mmol) was added. The reaction mixture was stirred in a microwave reactor at 150° C. for 2 minutes. After completion of the reaction, distilled water was added to the mixture slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 10:0) to obtain the title compound as a white oil. (130 mg, 54%) MS m/z: 322 [M+1]^{+ 1}H NMR (400 MHz, CDCl₃) δ 7.34 (d, J=8.1 Hz, 2H), 7.30-7.26 (m, 2H), 4.48 (s, 2H), 4.08-3.96 (m, 4H), 3.16 (d, J=7.2 Hz, 1H), 3.11 (d, J=5.8 Hz, 1H), 1.29-1.17 (m, 6H).

### [Preparative Example 10] Preparation of 4-(bromomethyl)-3-fluorobenzenesulfonamide (Intermediate 10)

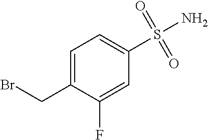 [SMILES: NS(=O)(=O)c1ccc(CBr)c(F)c1]

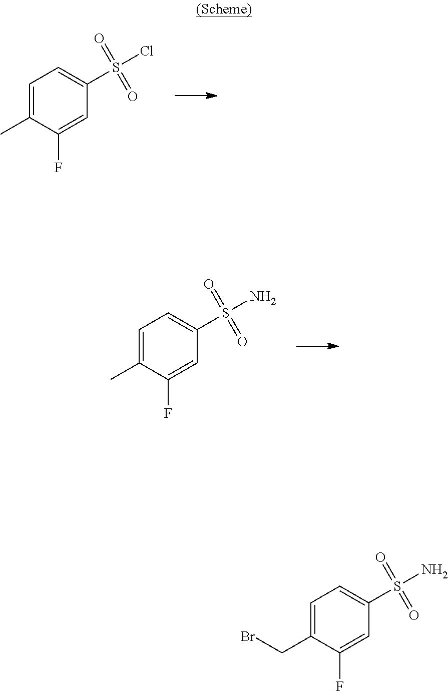

### (Step 1) Preparation of 3-Fluoro-4-methylbenzenesulfonamide

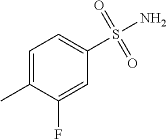

3-Fluoro-4-methyl-benzenesulfonyl chloride (500 mg, 2.40 mmol) was dissolved in 28% aqueous ammonia (10.0 mL), and then stirred at room temperature for 4 hours under nitrogen. After completion of the reaction, distilled water was added, and extracted with ethyl acetate. The organic layers were dried over anhydrous sodium sulfate, and then concentrated under reduced pressure. The title compound was obtained as a white solid without further purification. (387 mg, 85%) MS m/z: 190 [M+1]⁺

### (Step 2) Preparation of 4-(bromomethyl)-3-fluorobenzenesulfonamide (Intermediate 10)

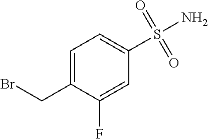 [SMILES: NS(=O)(=O)c1ccc(CBr)c(F)c1]

3-Fluoro-4-methyl-benzenesulfonamide (387 mg, 2.05 mmol) was dissolved in chloroform (16.0 mL), and then N-bromosuccinimide (546 mg, 3.07 mmol) and a,a′-azobis(isobutyronitrile) (33.6 mg, 0.20 mmol) were added to the mixture sequentially. The reaction mixture was stirred at 90° C. for 8 hours under nitrogen. After cooling to room temperature, distilled water was added, and extracted twice with ethyl acetate. The organic layers were washed with distilled water and a saturated saline solution, dried over anhydrous sodium sulfate, and then concentrated under reduced pressure. The residue was purified by silica gel column chromatography (ethyl acetate:n-hexane=1:8) to obtain the title compound as a white solid. (491 mg, 90%) MS m/z: 269 [M+1]⁺

### [Preparative Example 11] Preparation of 4-(bromomethyl)-3-chlorobenzenesulfonamide (Intermediate 11)

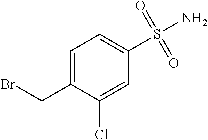 [SMILES: NS(=O)(=O)c1ccc(CBr)c(Cl)c1]

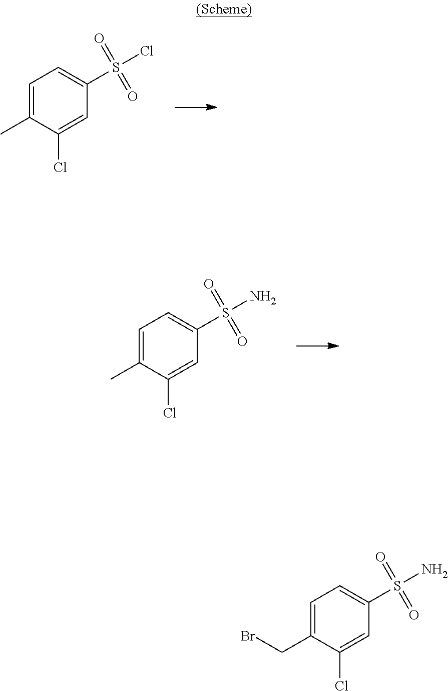

### (Step 1) Preparation of 3-chloro-4-methylbenzenesulfonamide

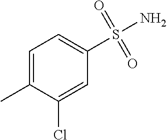

3-Chloro-4-methyl-benzenesulfonyl chloride (1.00 g, 4.44 mmol) was dissolved in 28% aqueous ammonia (20.0 mL), and then stirred at room temperature for 4 hours under nitrogen. After completion of the reaction, distilled water was added thereto, and extracted with ethyl acetate. The organic layers were collected, dried over anhydrous sodium sulfate, and then concentrated under reduced pressure. The title compound was obtained as a white solid without further purification. (745 mg, 82%) MS m/z: 206 [M+1]⁺

### (Step 2) Preparation of 4-(bromomethyl)-3-chlorobenzenesulfonamide (Intermediate 11)

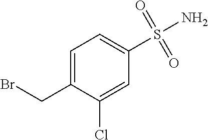 [SMILES: NS(=O)(=O)c1ccc(CBr)c(Cl)c1]

3-Chloro-4-methyl-benzenesulfonamide (500 mg, 2.43 mmol) was dissolved in chloroform (16.0 mL), and then N-bromosuccinimide (649 mg, 3.64 mmol) and a,a′-azobis(isobutyronitrile) (39.9 mg, 0.24 mmol) were added to the mixture sequentially. The reaction mixture was stirred at 90° C. for 8 hours under nitrogen. After cooling to room temperature, distilled water was added, and extracted twice with ethyl acetate. The organic layers were washed with distilled water and a saturated saline solution, dried over anhydrous sodium sulfate, and then concentrated under reduced pressure. The residue was purified by silica gel column chromatography (ethyl acetate:n-hexane=1:8) to obtain the title compound as a white solid. (499 mg, 72%) MS m/z: 285 [M+1]^{+ 1}H NMR (400 MHz, DMSO-d₆) δ ppm: 7.88 (s, 1H), 7.82 (d, J=7.6 Hz, 1H), 7.77 (d, J=7.6 Hz, 1H), 7.44 (s, 2H), 4.78 (s, 2H).

### [Preparative Example 12] Preparation of (4-(bromomethyl)-3-fluorophenyl)(methyl) sulfane (Intermediate 12)

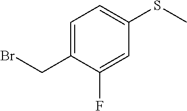

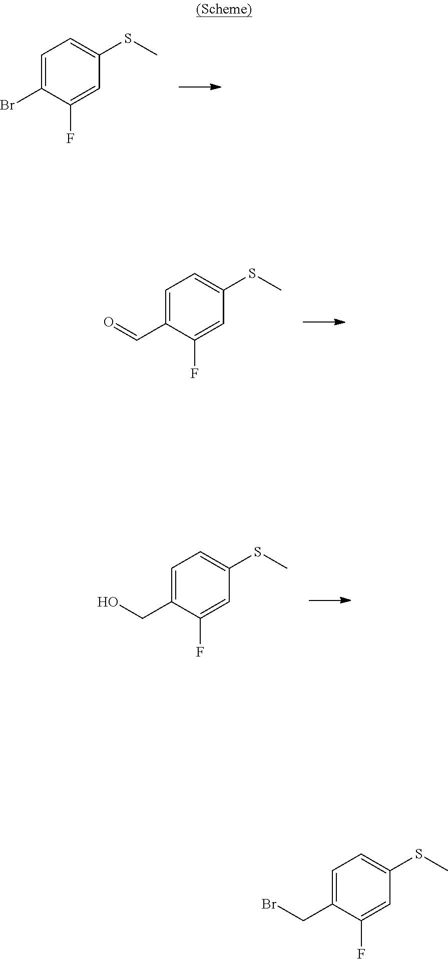

### (Step 1) Preparation of 2-fluoro-4-(methylthio)benzaldehyde

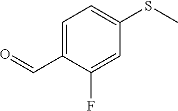

1-Bromo-2-fluoro-4-methylsulfanylbenzene (1.00 g, 4.52 mmol) was dissolved in tetrahydrofuran anhydride (11.0 mL), and then cooled to 0° C., and isopropyl magnesium chloride (2.0 M tetrahydrofuran solution, 2.60 mL, 5.20 mmol) was added and stirred for 30 minutes. N,N-Dimethylformamide anhydride (0.50 mL, 6.78 mmol) was added to the reaction mixture at 0° C., and then the reaction mixture was stirred at room temperature for 1 hour under nitrogen. After completion of the reaction, 1 N aqueous hydrogen chloride solution was slowly added at 0° C. and extracted twice with diethyl ether. The organic layers were dried over anhydrous sodium sulfate, and then concentrated under reduced pressure. The residue was purified by silica gel column chromatography (ethyl acetate:n-hexane=0:10 to 1:9) to obtain the title compound as a white solid. (483 mg, 63%) MS m/z: 171 [M+1]^{+ 1}H NMR (400 MHz, CDCl₃) δ ppm: 10.25 (s, 1H), 7.78-7.74 (m, 1H), 7.06 (d, J=8.4 Hz, 1H), 6.94 (d, J=11.3 Hz, 1H), 2.53 (s, 3H).

### (Step 2) Preparation of (2-fluoro-4-(methylthio)phenyl) methanol

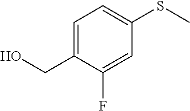

The compound prepared in (Step 1) above (483 mg, 2.84 mmol) was dissolved in methanol (7.00 mL), and then cooled to 0° C., and sodium borohydride (118 mg, 3.12 mmol) was added to the mixture and stirred for 3 hours. After completion of the reaction, distilled water was added slowly, and extracted twice with ethyl acetate. The organic layers were dried over anhydrous sodium sulfate, and then concentrated under reduced pressure to obtain the title compound as a colorless liquid. (473 mg, 97%) MS m/z: 173 [M+1]^{+ 1}H NMR (400 MHz, CDCl₃) δ ppm: 7.34-7.29 (m, 1H), 7.01 (d, J=9.7 Hz, 1H), 7.92 (d, J=10.7 Hz 1H), 4.70 (s, 2H), 2.48 (s, 3H).

### (Step 3) Preparation of (4-(bromomethyl)-3-fluorophenyl)(methyl) sulfane (Intermediate 12)

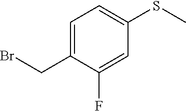

The compound prepared in (Step 2) above (473 mg, 2.75 mmol) was dissolved in methylene chloride (5.40 mL), and then tetrabromomethane (1.20 g, 3.57 mmol) and triphenylphosphine (792 mg, 3.02 mmol) were added to the mixture at 0° C. The reaction mixture was stirred at room temperature for 16 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a white solid. (552 mg, 85%) MS m/z: 236 [M+1]^{+ 1}H NMR (400 MHz, CDCl₃) δ ppm: 7.29-7.25 (m, 1H), 6.97 (d, J=8.7 Hz, 1H), 6.91 (d, J=10.2 Hz, 1H), 4.50 (s, 2H), 2.48 (s, 3H).

### [Preparative Example 13] Preparation of (4-(bromomethyl)-3-methoxyphenyl)(methyl) sulfane (Intermediate 13)

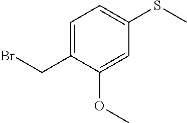

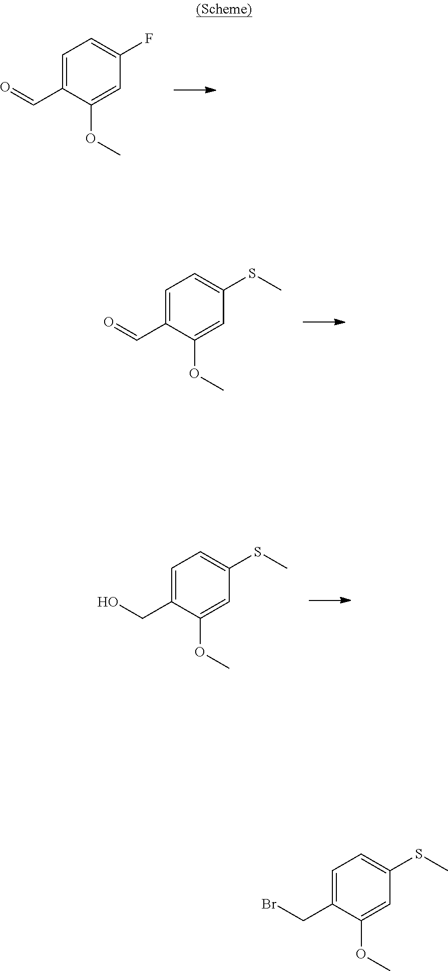

### (Step 1) Preparation of 2-methoxy-4-(methylthio)benzaldehyde

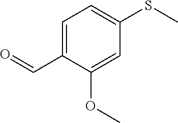

4-Fluoro-2-methoxy-benzaldehyde (500 mg, 3.24 mmol) was dissolved in acetonitrile anhydride (7.00 mL), and then sodium thiomethoxide (239 mg, 3.41 mmol) was added to the mixture. The reaction mixture was stirred at room temperature for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a white solid. (427 mg, 72%) MS m/z: 183 [M+1]^{+ 1}H NMR (400 MHz, CDCl₃) δ ppm: 10.35 (s, 1H), 7.74 (d, J=8.3 Hz, 1H), 6.83 (d, J=8.3 Hz, 1H), 6.79 (s, 1H), 3.93 (s, 3H), 2.54 (s, 3H).

### (Step 2) Preparation of (2-methoxy-4-(methylthio)phenyl) methanol

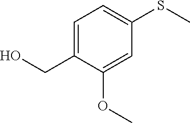

The compound prepared in (Step 1) above (427 mg, 2.34 mmol) was dissolved in methanol (7.00 mL), and then cooled to 0° C., and sodium borohydride (97.5 mg, 2.58 mmol) was added to the mixture and stirred for 3 hours. After completion of the reaction, distilled water was added slowly, and extracted twice with ethyl acetate. The organic layers were dried over anhydrous sodium sulfate, and then concentrated under reduced pressure to obtain the title compound as a colorless liquid. (429 mg, 99%) MS m/z: 185 [M+1]^{+ 1}H NMR (400 MHz, CDCl₃) δ ppm: 7.19 (d, J=7.8 Hz, 1H), 6.84-6.80 (m, 2H), 4.63 (d, J=6.3 Hz, 2H), 3.87 (s, 3H), 2.50 (s, 3H).

### (Step 3) Preparation of (4-(bromomethyl)-3-methoxyphenyl)(methyl) sulfane (Intermediate 13)

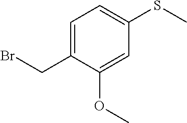

The compound prepared in (Step 2) above (429 mg, 2.33 mmol) was dissolved in methylene chloride (10.0 mL), and then tetrabromomethane (1.00 g, 3.03 mmol) and triphenylphosphine (672 mg, 2.56 mmol) were added to the mixture at 0° C. The reaction mixture was stirred at room temperature for 16 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a colorless liquid. (113 mg, 20%) MS m/z: 248 [M+1]^{+ 1}H NMR (400 MHz, CDCl₃) δ ppm: 7.23 (d, J=7.9 Hz, 1H), 6.80-6.77 (m, 2H), 4.54 (s, 2H), 3.89 (s, 3H), 2.49 (s, 3H).

### [Preparative Example 14] Preparation of (4-(bromomethyl)-3-methylphenyl)(methyl) sulfane (Intermediate 14)

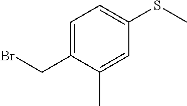

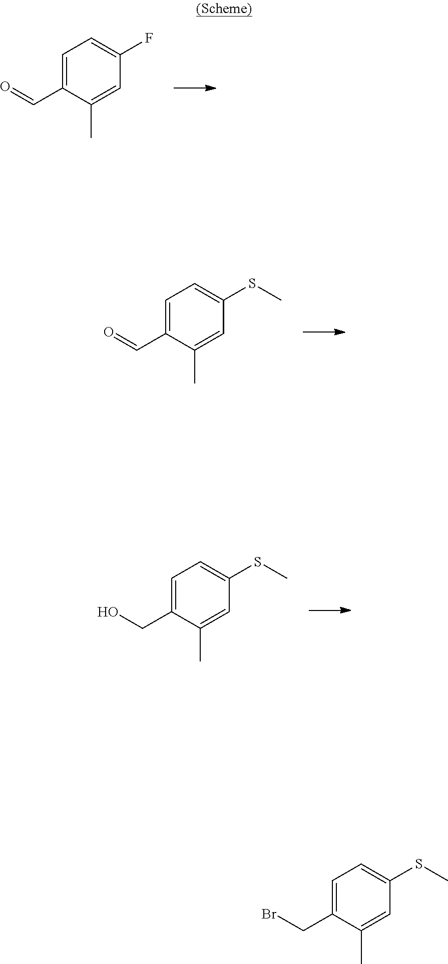

### (Step 1) Preparation of 2-methyl-4-(methylthio)benzaldehyde

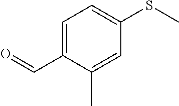

4-Fluoro-2-methyl-benzaldehyde (1.00 g, 7.23 mmol) was dissolved in acetonitrile anhydride (14.0 mL), and then sodium thiomethoxide (558 mg, 7.96 mmol) was added to the mixture. The reaction mixture was stirred at room temperature for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a colorless liquid. (1.03 g, 85%) MS m/z: 167 [M+1]^{+ 1}H NMR (400 MHz, CDCl₃) δ ppm: 10.16 (s, 1H), 7.69 (d, J=8.1 Hz, 1H), 7.15 (d, J=8.3 Hz, 1H), 7.05 (s, 1H), 2.64 (s, 3H), 2.52 (s, 3H).

### (Step 2) Preparation of (2-methyl-4-(methylthio)phenyl) methanol

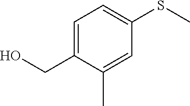

The compound prepared in (Step 1) above (1.00 g, 6.18 mmol) was dissolved in methanol (10.0 mL), and then cooled to 0° C., and sodium borohydride (257 mg, 6.80 mmol) was added to the mixture and stirred for 3 hours. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=3:7) to obtain the title compound as a colorless liquid. (972 mg, 93%) MS m/z: 169 [M+1]^{+ 1}H NMR (400 MHz, CDCl₃) δ ppm: 7.28 (s, 1H), 7.13-7.04 (m, 2H), 4.66 (s, 2H), 2.48 (s, 3H), 2.35 (s, 3H).

### (Step 3) Preparation of (4-(bromomethyl)-3-methylphenyl)(methyl) sulfane (Intermediate 14)

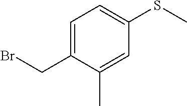

The compound prepared in (Step 2) above (472 mg, 2.81 mmol) was dissolved in methylene chloride (10.0 mL), and then tetrabromomethane (1.20 g, 3.65 mmol) and triphenylphosphine (809 mg, 3.08 mmol) were added to the mixture at 0° C. The reaction mixture was stirred at room temperature for 16 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:9) to obtain the title compound as a colorless liquid. (573 mg, 88%) MS m/z: 232 [M+1]^{+ 1}H NMR (400 MHz, CDCl₃) δ ppm: 7.21 (d, J=7.9 Hz, 1H), 7.06-7.03 (m, 2H), 4.50 (s, 2H), 2.47 (s, 3H), 2.39 (s, 3H).

### [Preparative Example 15] Preparation of (4-(bromomethyl)-2-fluorophenyl)(methyl) sulfane (Intermediate 15)

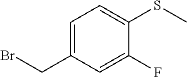

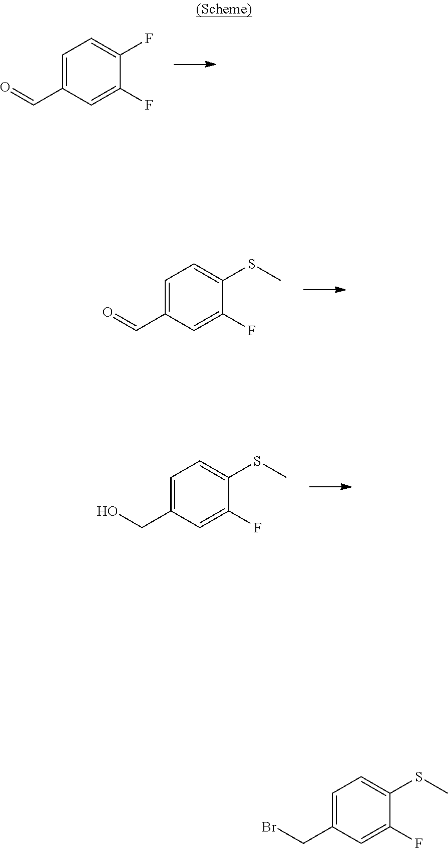

### (Step 1) Preparation of 3-fluoro-4-(methylthio)benzaldehyde

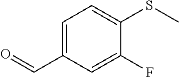

3,4-Difluorobenzaldehyde (800 mg, 5.63 mmol) was dissolved in acetonitrile anhydride (11.0 mL), and then sodium thiomethoxide (434 mg, 6.19 mmol) was added to the mixture. The reaction mixture was stirred at room temperature for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:3) to obtain the title compound as a colorless liquid. (616 mg, 64%) MS m/z: 171 [M+1]^{+ 1}H NMR (400 MHz, CDCl₃) δ ppm: 9.91 (s, 1H), 7.63 (d, J=8.2 Hz, 1H), 7.51 (d, J=10.0 Hz 1H), 7.35-7.29 (m, 1H), 2.54 (s, 3H).

### (Step 2) Preparation of (3-fluoro-4-(methylthio)phenyl) methanol

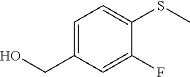

The compound prepared in (Step 1) above (616 mg, 3.62 mmol) was dissolved in methanol (7.00 mL), and then cooled to 0° C., and sodium borohydride (151 mg, 3.98 mmol) was added to the mixture and stirred for 3 hours. After completion of the reaction, distilled water was added slowly, and extracted twice with ethyl acetate. The organic layers were dried over anhydrous sodium sulfate, and then concentrated under reduced pressure. The title compound was obtained as a colorless liquid without further purification. (656 mg) MS m/z: 173 [M+1]^{+ 1}H NMR (400 MHz, CDCl₃) δ ppm: 7.26 (s, 1H), 7.12-7.06 (m, 2H), 4.67 (s, 2H), 2.46 (s, 3H).

### (Step 3) Preparation of (4-(bromomethyl)-2-fluorophenyl)(methyl) sulfane (Intermediate 15)

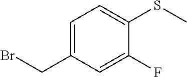

The compound prepared in (Step 2) above (656 mg, 3.62 mmol) was dissolved in methylene chloride (7.20 mL), and then tetrabromomethane (1.60 g, 4.71 mmol) and triphenylphosphine (1.00 g, 3.98 mmol) were added to the mixture at 0° C. The reaction mixture was stirred at room temperature for 16 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a colorless liquid. (825 mg, 97%) MS m/z: 236 [M+1]^{+ 1}H NMR (400 MHz, CDCl₃) δ ppm: 7.21-7.07 (m, 3H), 4.44 (s, 2H), 2.47 (s, 3H).

### [Preparative Example 16] Preparation of (4-(bromomethyl)phenyl) (cyclopropyl) sulfane (Intermediate 16)

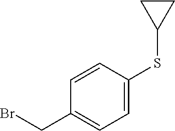 [SMILES: BrCc1ccc(SC2CC2)cc1]

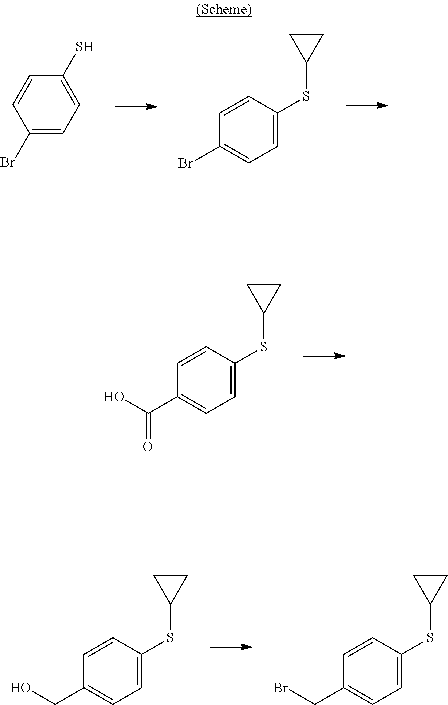 [SMILES: Sc1ccc(Br)cc1 ; Brc1ccc(SC2CC2)cc1 ; O=C(O)c1ccc(SC2CC2)cc1 ; OCc1ccc(SC2CC2)cc1 ; BrCc1ccc(SC2CC2)cc1]

### (Step 1) Preparation of (4-bromophenyl) (cyclopropyl) sulfane

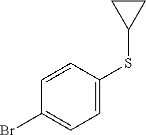 [SMILES: Brc1ccc(SC2CC2)cc1]

4-Bromobenzenethiol (1.50 g, 7.93 mmol) was dissolved in dimethylsulfoxide (10.0 mL), and then potassium tert-butoxide (979.3 mg, 8.73 mmol) and bromocyclopropane (1.58 mL, 19.83 mmol) were added to the mixture. The reaction mixture was stirred at 80° C. for 24 hours under reduced pressure. After completion of the reaction, the reaction mixture was cooled to room temperature. Distilled water was added slowly and adjusted to pH 2-3 with 1 M aqueous HCl solution, and then extracted with ethyl acetate three times. The organic layers were and dried over anhydrous sodium sulfate, and then concentrated under reduced pressure. The residue was purified by silica gel column chromatography (ethyl acetate:n-hexane=0:10 to 1:9) to obtain the title compound as a pale yellow liquid. (1.66 g, 92%). 1H NMR (400 MHz, CDCl₃) δ ppm: 7.39 (d, J=8.4 Hz, 2H), 7.22 (d, J=8.3 Hz, 2H), 2.16 (td, J=8.3, 3.5 Hz, 1H), 1.16-1.01 (m, 2H), 0.72-0.66 (dd, J=4.8, 2.0 Hz, 2H).

### (Step 2) Preparation of 4-(cyclopropylthio)benzoic acid

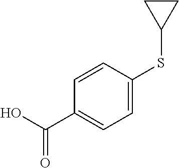 [SMILES: O=C(O)c1ccc(SC2CC2)cc1]

The compound (1.66 g, 7.24 mmol) prepared in (Step 1) above was dissolved in anhydrous tetrahydrofuran (25.0 mL), and then cooled to −78° C., and n-butyllithium (5.4 mL, 8.69 mmol, 1.6 M in hexane) was added to the mixture and stirred for 20 minutes. Carbon dioxide gas (CO2) was injected into the reaction mixture and stirred for 1 hour. After completion of the reaction, distilled water was added slowly and adjusted to pH 2-3 with 1 M aqueous HCl solution, and then extracted with ethyl acetate three times. The organic layers were dried over anhydrous sodium sulfate, and then concentrated under reduced pressure to obtain the title compound as a pale yellow solid. (1.48 g). MS m/z: 195 [M+1]+ 1H NMR (400 MHz, DMSO-d6) δ ppm: 7.74-7.63 (m, 2H), 7.27 (d, J=8.5 Hz, 2H), 2.14 (td, J=7.1, 4.4 Hz, 1H), 0.97 (dd, J=7.3, 2.3 Hz, 2H), 0.48-0.38 (m, 2H).

### (Step 3) Preparation of (4-(cyclopropylthio)phenyl) methanol

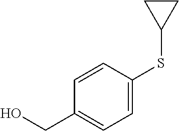 [SMILES: OCc1ccc(SC2CC2)cc1]

The compound (700 mg, 3.60 mmol) prepared in (Step 2) above was dissolved in anhydrous tetrahydrofuran (6.00 mL), and then lithium aluminum hydride (7.20 mL, 7.20 mmol, 1 M in THF) was added to the mixture at 0° C. The reaction mixture was stirred at 0° C. for 2 hours under nitrogen. After completion of the reaction, distilled water was added slowly, and 1 N aqueous sodium hydroxide solution (1.00 mL) was added thereto. The reaction mixture was filtered through Celite, and then washed with ethyl acetate. The filtrate was concentrated under reduced pressure, and then the mixture was purified by silica gel column chromatography (ethyl acetate:n-hexane=1:9 to 1:1) to obtain the title compound as a colorless liquid. (411 mg, 63%). MS m/z: 181 [M+1]+ 1H NMR (400 MHz, CDCl3,) δ ppm: 7.36 (d, J=8.4 Hz, 2H), 7.29 (d, J=8.3 Hz, 2H), 4.65 (s, 2H), 2.25-2.15 (m, 1H), 1.10-1.04 (m, J=5.9 Hz, 2H), 0.72-0.66 (q, J=4.6 Hz, 2H).

### (Step 4) Preparation of (4-(bromomethyl)phenyl) (cyclopropyl) sulfane

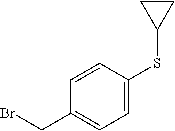 [SMILES: BrCc1ccc(SC2CC2)cc1]

The compound (411 mg, 2.28 mmol) prepared in (Step 3) above was dissolved in methylene chloride (10.0 mL), and then tetrabromomethane (982.9 mg, 2.96 mmol) and triphenylphosphine (657.8 mg, 2.51 mmol) were added to the mixture at 0° C. The reaction mixture was stirred at room temperature for 18 hours under nitrogen. After completion of the reaction, distilled water was added, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:4) to obtain the title compound as a colorless liquid. (460 mg, 83%). 1H NMR (400 MHz, CDCl3) δ ppm: 7.34-7.29 (m, 4H), 4.48 (s, 2H), 2.20-2.15 (m, 1H), 1.11-1.06 (m, 2H), 0.74-0.65 (m, 2H).

### [Preparative Example 17] Preparation of (4-(bromomethyl)-3-(trifluoromethyl)phenyl)(methyl) sulfane (Intermediate 17)

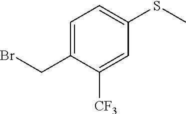

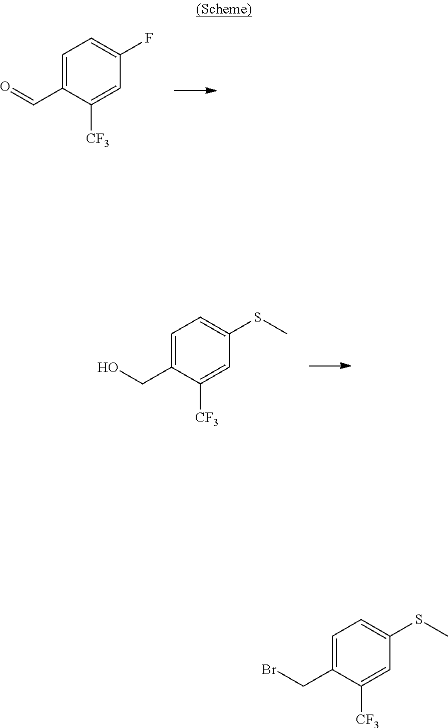

### (Step 1) Preparation of (4-(methylthio)-2-(trifluoromethyl)phenyl) methanol

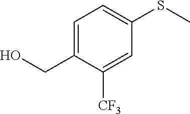

4-Fluoro-2-(trifluoromethyl)benzaldehyde (1.00 g, 5.21 mmol) was dissolved in tetrahydrofuran (10.0 mL), and then sodium thiomethoxide (547 mg, 7.81 mmol) was added to the mixture. The reaction mixture was stirred at 70° C. for 2 hours under reduced pressure. After completion of the reaction, the reaction mixture was cooled to room temperature. Distilled water was added slowly, and extracted with ethyl acetate three times. The organic layers were dried over anhydrous sodium sulfate, and then concentrated under reduced pressure. The residue was purified by silica gel column chromatography (ethyl acetate:ether=1:100 to 1:3) to obtain the title compound as a colorless liquid. (400 mg, 34%). 1H NMR (400 MHz, CDCl3) δ ppm: 7.61 (d, J=8.1 Hz, 1H), 7.49 (s, 1H), 7.42 (d, J=8.1 Hz, 1H), 4.82 (s, 2H), 2.52 (s, 3H).

### (Step 2) Preparation of (4-(bromomethyl)-3-(trifluoromethyl)phenyl)(methyl) sulfane (Intermediate 17)

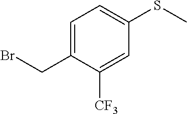

The compound (400 mg, 1.80 mmol) prepared in (Step 1) above was dissolved in methylene chloride (5.00 mL), and then thionyl bromide (1.87 g, 9.00 mmol) was added to the mixture. The reaction mixture was stirred at room temperature for 3 hours. After confirming the completion of the reaction, the reaction mixture was concentrated under reduced pressure. The title compound was obtained as a colorless liquid without further purification. (350 mg, 68%). 1H NMR (400 MHz, CDCl3) δ ppm: 7.46-7.51 (m, 2H), 7.38 (dd, J=8.2, 1.7 Hz, 1H), 4.61 (s, 2H), 2.53 (s, 3H).

### [Preparative Example 18] Preparation of diethyl ((4-(bromomethyl)phenyl)difluoromethyl)phosphonate (Intermediate 18)

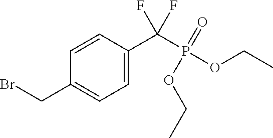 [SMILES: CCOP(=O)(OCC)C(F)(F)c1ccc(CBr)cc1]

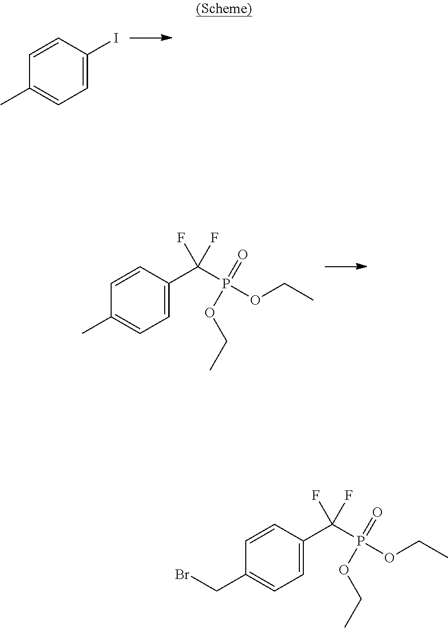

### (Step 1) Preparation of diethyl (difluoro (p-tolyl)methyl)phosphonate

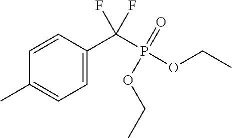 [SMILES: CCOP(=O)(OCC)C(F)(F)c1ccc(C)cc1]

Diethyl (bromodifluoromethyl)phosphonate (1.47 g, 5.5 mmol) was dissolved in N,N-dimethylamide (5.0 mL), and then zinc (359 mg, 5.5 mmol) was added to the mixture. The reaction mixture was stirred at 55° C. for 30 minutes under nitrogen. The mixture was sonicated for 3 hours, copper bromide (I) (770 mg, 5.37 mmol) was added, and further stirred at room temperature for 30 minutes. 4-Iodotoluene (300 mg, 1.38 mmol) dissolved in N,N-dimethylformacetamide (5.0 mL) was slowly added to the mixture, and stirred at 25° C. for 18 hours under nitrogen. After completion of the reaction, distilled water was added slowly, and extracted twice with diethyl ether. The organic layers were dried over anhydrous sodium sulfate, and then concentrated under reduced pressure. The residue was purified by silica gel column chromatography (ethyl acetate:n-hexane=1:19 to 10:0) to obtain the title compound as a colorless oil. (147.0 mg, 38%). MS m/z: 279 [M+1]+ 1H NMR (400 MHz, CDCl3) δ 7.50 (d, J=7.9 Hz, 2H), 7.24 (s, 2H), 4.23-4.11 (m, 4H), 2.40 (d, J=5.0 Hz, 3H), 1.31 (t, J=7.1 Hz, 6H).

### (Step 2) Preparation of diethyl ((4-(bromomethyl)phenyl)difluoromethyl)phosphonate (Intermediate 18)

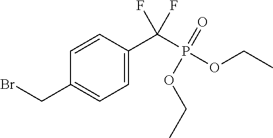 [SMILES: CCOP(=O)(OCC)C(F)(F)c1ccc(CBr)cc1]

The compound (70.0 mg, 0.25 mmol) prepared in (Step 1) above was dissolved in carbon tetrachloride (1.00 mL), and then N-bromosuccinimide (53.4 mg, 0.30 mmol) and a,a′-azobis(isobutyronitrile) (1.00 mg) were added to the mixture. The mixture was stirred at 80° C. for 4 hours under nitrogen. After completion of the reaction, the mixture was concentrated under reduced pressure, and the residue was purified by silica gel column chromatography (ethyl acetate:n-hexane=1:4 to 10:0) to obtain the title compound as a white solid in the form of a mixture. (41.0 mg, 46%). MS m/z: 358 [M+1]+

### Example 2: Synthesis of Examples

### [Example 1] Preparation of (4-((9H-pyrido[3,4-b]indol-9-yl)methyl)phenyl)(imino)(methyl)-l6-sulfanone (Compound 1)

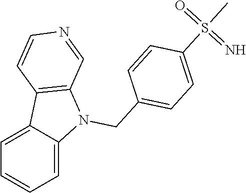 [SMILES: CS(=N)(=O)c1ccc(Cn2c3ccccc3c3ccncc32)cc1]

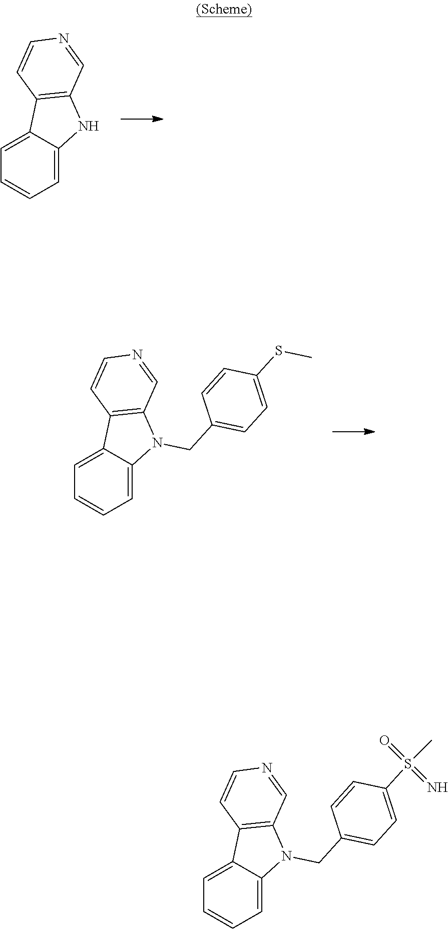 [SMILES: c1ccc2c(c1)[nH]c1cnccc12 ; CSc1ccc(Cn2c3ccccc3c3ccncc32)cc1 ; CS(=N)(=O)c1ccc(Cn2c3ccccc3c3ccncc32)cc1]

### (Step 1) Preparation of 9-(4-(methylthio)benzyl)-9H-pyrido[3,4-b]indole

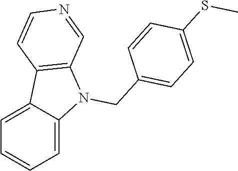 [SMILES: CSc1ccc(Cn2c3ccccc3c3ccncc32)cc1]

9H-Pyrido[3,4-b]indole (50.0 mg, 0.30 mmol) was dissolved in N,N-dimethylformamide (1.00 mL), and then cooled to 0° C., and 60% sodium hydride (35.6 mg, 0.89 mmol) was added to the mixture and stirred for 30 minutes. Intermediate 6 (77.4 mg, 0.36 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (32.0 mg, 35%) MS m/z: 305 [M+1]⁺

### (Step 2) Preparation of (4-((9H-pyrido[3,4-b]indol-9-yl)methyl)phenyl)(imino)(methyl)-l6-sulfanone (Compound 1)

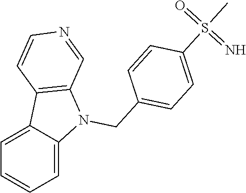 [SMILES: CS(=N)(=O)c1ccc(Cn2c3ccccc3c3ccncc32)cc1]

The compound prepared in (Step 1) above (32.0 mg, 0.11 mmol) was dissolved in ethanol (1.00 mL), and then iodobenzene diacetate (98.1 mg, 0.30 mmol) and ammonium acetate (30.8 mg, 0.40 mmol) were added to the mixture. The reaction mixture was stirred at room temperature for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (5.80 mg, 16%) MS m/z: 336 [M+1]^{+ 1}H NMR (400 MHz, CD₃OD) δ 9.37 (s, 1H), 8.81 (d, J=6.3 Hz, 1H), 8.58 (d, J=6.3 Hz, 1H), 8.55 (d, J=8.0 Hz, 1H), 8.04-7.99 (m, 2H), 7.86 (ddd, J=8.4, 7.0, 1.3 Hz, 1H), 7.78 (d, J=8.5 Hz, 1H), 7.57 (ddd, J=8.1, 6.9, 1.1 Hz, 1H), 7.49 (dd, J=8.7, 2.1 Hz, 2H), 6.07 (s, 2H), 3.36 (s, 3H).

### [Example 2] Preparation of 4-((6-methoxy-9H-pyrido[3,4-b]indol-9-yl)methyl)benzenesulfonamide (Compound 2)

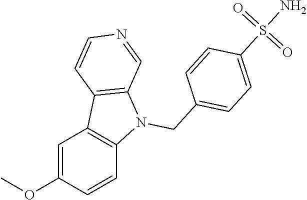 [SMILES: COc1ccc2c(c1)c1ccncc1n2Cc1ccc(S(N)(=O)=O)cc1]

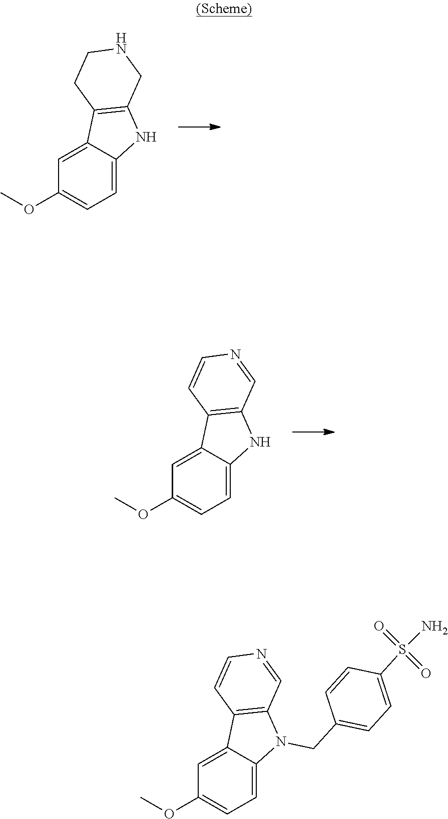 [SMILES: COc1ccc2[nH]c3c(c2c1)CCNC3 ; COc1ccc2[nH]c3cnccc3c2c1 ; COc1ccc2c(c1)c1ccncc1n2Cc1ccc(S(N)(=O)=O)cc1]

### (Step 1) Preparation of 6-methoxy-9H-pyrido[3,4-b]indole

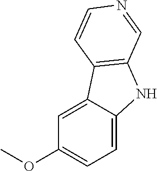

6-Methoxy-2,3,4,9-tetrahydro-1H-pyrido[3,4-b]indole (1.8 g, 8.90 mmol) was dissolved in cumene (150 mL), and then Pd/C (10 wt %, 1.00 g) was added to the mixture. The reaction mixture was stirred at 110° C. for 16 hours under nitrogen. Precipitation was removed by filtration using Celite, and then the filtrate was concentrated under reduced pressure. The residue was purified by medium pressure liquid chromatography (ethyl acetate:methanol=10:0 to 9:1) to obtain the title compound as a yellow solid. (1.0 g, 56%) MS m/z: 199 [M+1]^{+ 1}H NMR (400 MHz, CDCl₃) δ 8.92 (d, J=0.8 Hz, 1H), 8.46 (d, J=5.6 Hz, 1H), 7.93 (d, J=5.2 Hz, 1H), 7.59 (d, J=2.4 Hz, 1H), 7.45 (d, J=8.8 Hz, 1H), 7.24 (d, J=6.4 Hz, 1H), 3.95 (s, 3H).

### (Step 2) Preparation of 4-((6-methoxy-9H-pyrido[3,4-b]indol-9-yl)methyl)benzenesulfonamide (Compound 2)

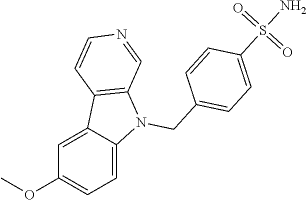 [SMILES: COc1ccc2c(c1)c1ccncc1n2Cc1ccc(S(N)(=O)=O)cc1]

The compound prepared in (Step 1) above (50.0 mg, 0.25 mmol) was dissolved in N,N-dimethylformamide (2.50 mL), and then cooled to 0° C., and 60% sodium hydride (20.2 mg, 0.50 mmol) was added to the mixture and stirred for 30 minutes. Intermediate 1 (75.7 mg, 0.30 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, acetonitrile:distilled water=5:95 to 100:0) to obtain the title compound as a yellow solid. (45.4 mg, 49.0%) MS m/z: 368 [M+1]^{+ 1}H NMR (400 MHz, DMSO-d₆) δ 9.48 (s, 1H), 8.75 (d, J=6.0 Hz, 1H), 8.60 (d, J=6.0 Hz, 1H), 8.11 (d, J=2.4 Hz, 1H), 7.75-7.71 (m, 3H), 7.44 (dd, J=2.6, 9.0 Hz, 1H), 7.34-7.30 (m, 4H), 5.97 (s, 2H), 3.90 (s, 3H).

### [Example 3] Preparation of imino (4-((6-methoxy-9H-pyrido[3,4-b]indol-9-yl)methyl)phenyl)(methyl)-l6-sulfanone (Compound 3)

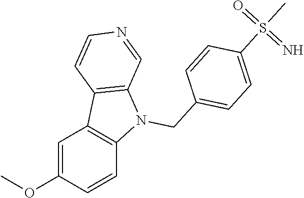 [SMILES: COc1ccc2c(c1)c1ccncc1n2Cc1ccc(S(C)(=N)=O)cc1]

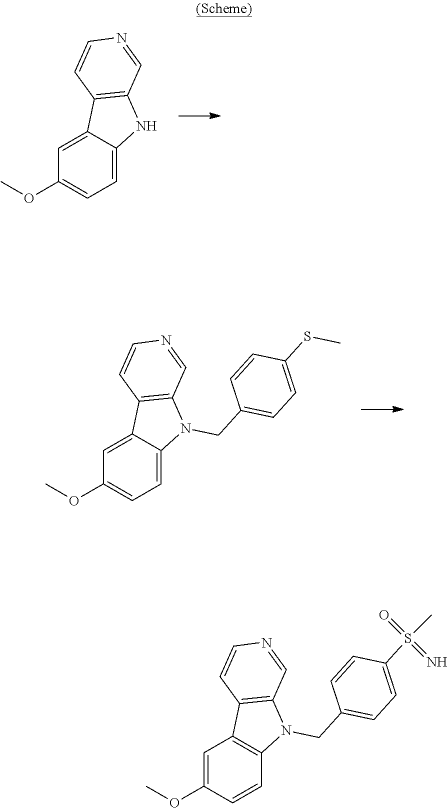

### (Step 1) Preparation of 6-methoxy-9-(4-(methylthio)benzyl)-9H-pyrido[3,4-b]indole

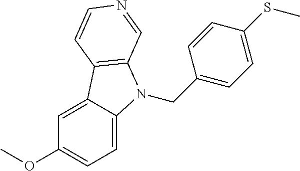

The compound prepared in (Step 1) of Example 2 (50.0 mg, 0.252 mmol) was dissolved in N,N-dimethylformamide (2.50 mL), and then cooled to 0° C., and 60% sodium hydride (20.2 mg, 0.50 mmol) was added to the mixture and stirred for 30 minutes. Intermediate 6 (65.7 mg, 0.30 mmol) dissolved in N,N-dimethylformamide (0.50 mL) was slowly added to the reaction mixture, and then stirred at 80° C. for 15 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (64.4 mg, 76%) MS m/z: 335 [M+1]⁺

### (Step 2) Preparation of imino (4-((6-methoxy-9H-pyrido[3,4-b]indol-9-yl)methyl)phenyl)(methyl)-l6-sulfanone (Compound 3)

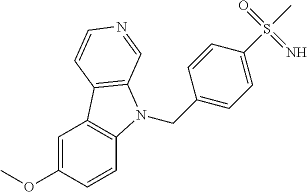 [SMILES: COc1ccc2c(c1)c1ccncc1n2Cc1ccc(S(C)(=N)=O)cc1]

The compound prepared in (Step 1) above (64.4 mg, 0.19 mmol) was dissolved in ethanol (1.90 mL), and then iodobenzene diacetate (180 mg, 0.56 mmol) and ammonium acetate (56.4 mg, 0.73 mmol) were added to the mixture. The reaction mixture was stirred at room temperature for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (22.6 mg, 32%) MS m/z: 366 [M+1]^{+ 1}H NMR (400 MHz, DMSO-d₆) δ 9.54 (s, 1H), 8.81 (d, J=6.0 Hz, 1H), 8.64 (d, J=5.6 Hz, 1H), 8.15 (d, J=2.0 Hz, 1H), 7.87 (d, J=8.4 Hz, 1H), 7.79 (d, J=9.2 Hz, 2H), 7.48 (dd, J=2.0, 9.2 Hz, 1H), 7.39 (d, J=8.0 Hz, 2H), 6.03 (s, 2H), 3.92 (s, 3H), 3.10 (s, 3H).

### [Example 4] Preparation of 4-((8-methoxy-5H-pyrido[4,3-b]indol-5-yl)methyl)benzenesulfonamide (Compound 4)

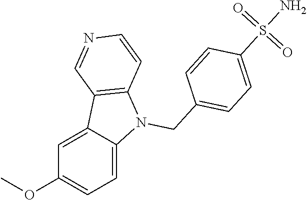 [SMILES: COc1ccc2c(c1)c1cnccc1n2Cc1ccc(S(N)(=O)=O)cc1]

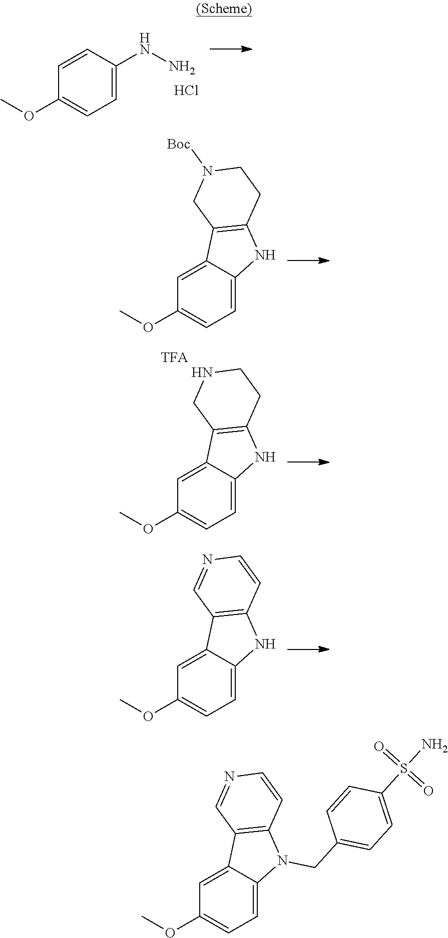

### (Step 1) Preparation of tert-butyl 8-methoxy-1,3,4,5-tetrahydro-2H-pyrido[4,3-b]indole-2-carboxylate

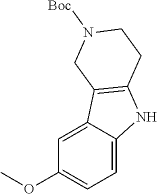 [SMILES: COc1ccc2[nH]c3c(c2c1)CN(C(=O)OC(C)(C)C)CC3]

1,3-Dimethylurea (5.30 g, 60.2 mmol) and L-(+)-tartaric acid (2.26 g, 15.0 mmol) were mixed, and then the reaction mixture was stirred at 80° C. for 2 hours under nitrogen. 4-Methoxyphenylhydrazine-hydrochloride (1.05 g, 6.02 mmol) and tert-butyl-4-oxopiperidine-1-carboxylate (1.00 g, 5.01 mmol) were sequentially added to the reaction mixture, and then stirred at 80° C. for 2 hours. After cooling to room temperature, distilled water and an aqueous sodium bicarbonate solution were added, and extracted twice with methylene chloride. The organic layers were dried over anhydrous sodium sulfate, and then concentrated under reduced pressure. The residue was purified by silica gel column chromatography (ethyl acetate:n-hexane=0:10 to 3:7) to obtain the title compound as a yellow solid. (391 mg, 26%) MS m/z: 303 [M+1]^{+ 1}H NMR (400 MHz, DMSO-d₆) δ 10.73 (s, 1H), 7.26-7.14 (m, 1H), 6.91 (s, 1H), 6.68 (dd, J=8.7, 2.5 Hz, 1H), 4.51 (s, 2H), 3.77 (d, J=1.3 Hz, 3H), 3.71 (t, J=5.8 Hz, 2H), 2.76 (s, 2H), 1.57-1.39 (m, 9H).

### (Step 2) Preparation of 8-methoxy-2,3,4,5-tetrahydro-1H-pyrido[4,3-b]indole-trifluoroacetic acid

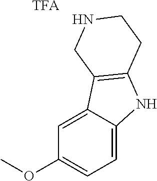

The compound prepared in (Step 1) above (105 mg, 0.35 mmol) was dissolved in methylene chloride (3.00 mL), and then trifluoroacetic acid (1.00 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (110 mg, 100%) MS m/z: 203 [M+1]⁺

### (Step 3) Preparation of 8-methoxy-5H-pyrido[4,3-b]indole

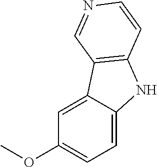

The compound prepared in (Step 2) above (110 mg, 0.35 mmol) was dissolved in dimethyl sulfoxide (0.60 mL), and then iodine (22.1 mg, 0.09 mmol) and an aqueous hydrogen peroxide solution (11.0 μl, 0.35 mmol) were added to the mixture. The reaction mixture was stirred at 100° C. for 6 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, acetonitrile:distilled water=5:95 to 100:0) to obtain the title compound as a yellow solid. (5.30 mg, 8%) MS m/z: 199 [M+1]⁺

### Step 4) Preparation of 4-((8-methoxy-5H-pyrido[4,3-b]indol-5-yl)methyl)benzenesulfonamide (Compound 4)

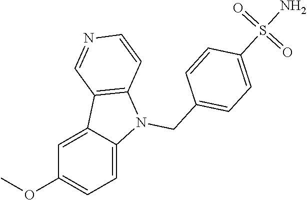 [SMILES: COc1ccc2c(c1)c1cnccc1n2Cc1ccc(S(N)(=O)=O)cc1]

The compound prepared in (Step 3) above (5.30 mg, 0.03 mmol) was dissolved in N,N-dimethylformamide (0.30 mL), and then cooled to 0° C., and 60% sodium hydride (2.10 mg, 0.05 mmol) was added to the mixture and stirred for 30 minutes. Intermediate 1 (8.00 mg, 0.03 mmol) dissolved in N,N-dimethylformamide (0.10 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (1.30 mg, 21%) MS m/z: 368 [M+1]^{+ 1}H NMR (400 MHz, CD₃OD) δ 9.60 (d, J=2.9 Hz, 1H), 8.58-8.49 (m, 1H), 8.09 (s, 1H), 7.99 (s, 1H), 7.83 (d, J=7.8 Hz, 2H), 7.71-7.56 (m, 2H), 7.40-7.28 (m, 2H), 5.93 (s, 2H), 3.96 (s, 3H).

### [Example 5] Preparation of 4-((8-methoxy-5H-pyrido[3,2-b]indol-5-yl)methyl)benzenesulfonamide (Compound 5)

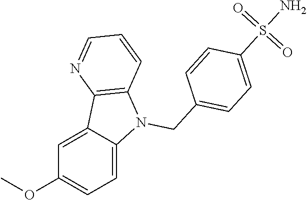 [SMILES: COc1ccc2c(c1)c1ncccc1n2Cc1ccc(S(N)(=O)=O)cc1]

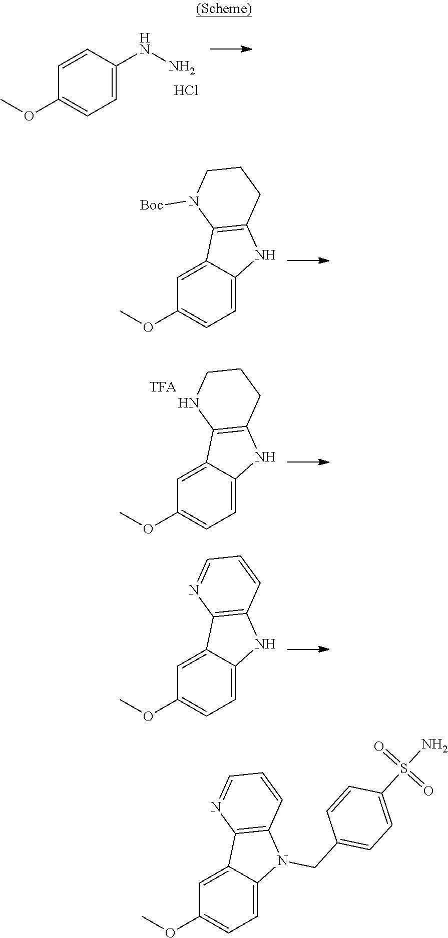

### (Step 1) Preparation of tert-butyl 8-methoxy-2,3,4,5-tetrahydro-1H-pyrido[3,2-b]indole-1-carboxylate

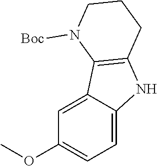 [SMILES: COc1ccc2[nH]c3c(c2c1)N(C(=O)OC(C)(C)C)CCC3]

4-Methoxyphenylhydrazine-hydrochloride (5.00 g, 28.6 mmol) was dissolved in toluene (95.4 mL), and then tert-butyl-3-oxopiperidine-1-carboxylate (6.85 g, 34.4 mmol) and propanephosphonic anhydride (2.90 mL, 5.73 mmol) were added to the mixture sequentially. The reaction mixture was stirred at 90° C. for 6 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (4.20 g, 48%) MS m/z: 303 [M+1]⁺

### (Step 2) Preparation of 8-methoxy-2,3,4,5-tetrahydro-1H-pyrido[3,2-b]indole-trifluoroacetic acid

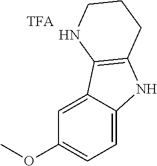

The compound prepared in (Step 1) above (8.30 g, 27.5 mmol) was dissolved in methylene chloride (109 mL), and then trifluoroacetic acid (10.0 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (8.70 g, 100%)

MS m/z: 203 [M+1]⁺

### (Step 3) Preparation of 8-methoxy-5H-pyrido[3,2-b]indole

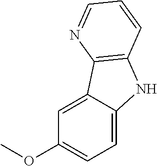

The compound prepared in (Step 2) above (4.00 g, 12.6 mmol) was dissolved in toluene (126 mL), and then Pd/C (10 wt %, 2.00 g) was added to the mixture. The reaction mixture was stirred at 90° C. for 36 hours under nitrogen. Precipitation was removed by filtration using Celite, and then the filtrate was concentrated under reduced pressure. The residue was purified by medium pressure liquid chromatography (ethyl acetate:methanol=10:0 to 9:1) to obtain the title compound as a yellow solid. (1.23 g, 49%) MS m/z: 199 [M+1]^{+ 1}H NMR (400 MHz, DMSO-d₆) δ 11.2 (s, 1H), 8.41 (d, J=4.4 Hz, 1H), 7.84 (d, J=8.0 Hz, 1H), 7.66 (d, J=2.4 Hz, 1H), 7.47 (d, J=8.8 Hz, 1H), 7.35 (dd, J=4.6, 8.2 Hz, 1H), 7.14 (dd, J=2.4, 8.8 Hz, 1H), 3.86 (s, 3H).

### (Step 4) Preparation of ((8-methoxy-5H-pyrido[3,2-b]indol-5-yl)methyl)benzenesulfonamide (Compound 5)

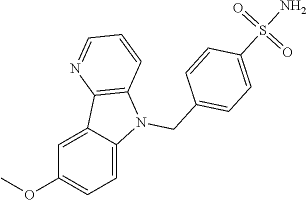 [SMILES: COc1ccc2c(c1)c1ncccc1n2Cc1ccc(S(N)(=O)=O)cc1]

The compound prepared in (Step 3) above (100 mg, 0.51 mmol) was dissolved in N,N-dimethylformamide (5.00 mL), and then cooled to 0° C., and 60% sodium hydride (40.4 mg, 1.01 mmol) was added to the mixture and stirred for 30 minutes. Intermediate 1 (151 mg, 0.61 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (23.2 mg, 13%) MS m/z: 368 [M+1]^{+ 1}H NMR (400 MHz, CD₃OD) δ 8.53 (d, J=4.4 Hz, 1H), 8.44 (d, J=8.4 Hz, 1H), 7.77 (s, 1H), 7.72-7.70 (m, 3H), 7.56 (d, J=9.2 Hz, 1H), 7.31 (d, J=9.2 Hz, 1H), 7.20 (d, J=8.0 Hz, 2H), 5.77 (s, 2H), 3.85 (s, 3H).

### [Example 6] Preparation of 4-((8-methoxy-5H-pyrido[3,2-b]indol-5-yl)methyl) piperidine-1-sulfonamide (Compound 6)

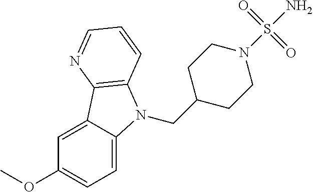 [SMILES: COc1ccc2c(c1)c1ncccc1n2CC1CCN(S(N)(=O)=O)CC1]

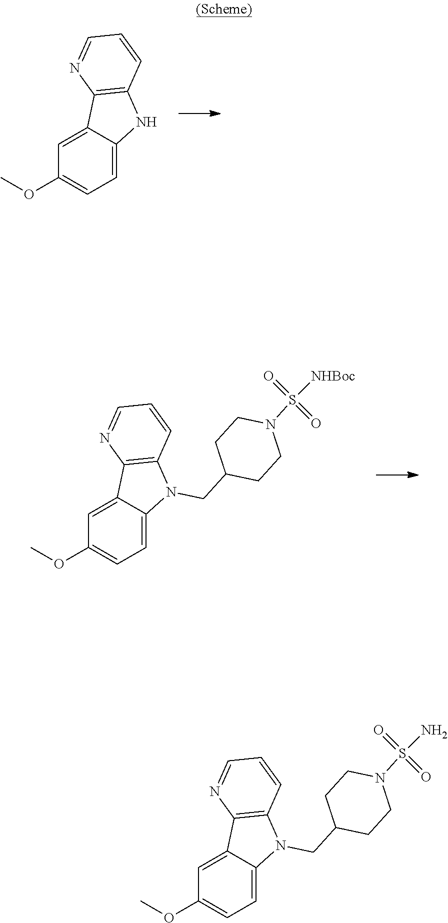 [SMILES: COc1ccc2[nH]c3cccnc3c2c1 ; COc1ccc2c(c1)c1ncccc1n2CC1CCN(S(=O)(=O)NC(=O)OC(C)(C)C)CC1 ; COc1ccc2c(c1)c1ncccc1n2CC1CCN(S(N)(=O)=O)CC1]

### (Step 1) Preparation of tert-butyl ((4-((8-methoxy-5H-pyrido[3,2-b]indol-5-yl)methyl) piperidin-1-yl)sulfonyl)carbamate

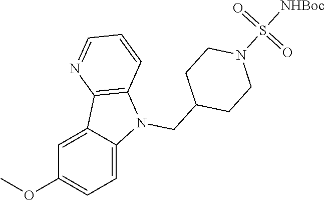 [SMILES: COc1ccc2c(c1)c1ncccc1n2CC1CCN(S(=O)(=O)NC(=O)OC(C)(C)C)CC1]

The compound prepared in (Step 3) of Example 5 (20.0 mg, 0.10 mmol) was dissolved in N,N-dimethylformamide (1.00 mL), and then cooled to 0° C., and 60% sodium hydride (8.00 mg, 0.20 mmol) was added to the mixture and stirred for 30 minutes. Intermediate 2 (39.0 mg, 0.11 mmol) dissolved in N,N-dimethylformamide (0.50 mL) was slowly added to the reaction mixture, and then stirred at 80° C. for 15 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, acetonitrile:distilled water=5:95 to 100:0) to obtain the title compound as a yellow solid. (17.0 mg, 35%) MS m/z: 475 [M+1]⁺

### Step 2) Preparation of 4-((8-methoxy-5H-pyrido[3,2-b]indol-5-yl)methyl) piperidine-1-sulfonamide (Compound 6)

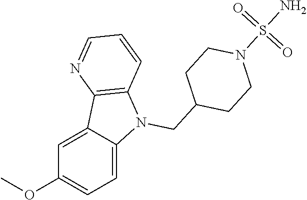 [SMILES: COc1ccc2c(c1)c1ncccc1n2CC1CCN(S(N)(=O)=O)CC1]

The compound prepared in (Step 1) above (17.0 mg, 0.04 mmol) was dissolved in methylene chloride (1.00 mL), and then trifluoroacetic acid (1.00 mL) was added to the mixture. The reaction mixture was stirred at room temperature for 2 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (1.20 mg, 9%) MS m/z: 375 [M+1]^{+ 1} H NMR (400 MHz, CD₃OD) δ 8.04 (s, 1H), 7.88 (s, 1H), 7.83 (s, 1H), 7.60 (s, 1H), 7.51 (s, 1H), 7.24 (s, 1H), 5.55 (s, 2H), 4.53 (s, 1H), 4.34 (s, 2H), 3.90 (s, 3H), 3.58 (s, 2H), 3.41 (s, 2H), 3.10 (s, 2H).

### [Example 7] Preparation of 4-((8-methoxy-5H-pyrido[3,2-b]indol-5-yl)methyl)-N-methylbenzenesulfonamide (Compound 7)

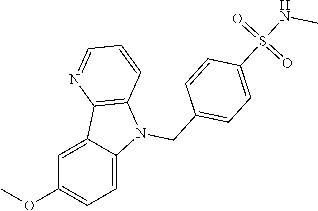

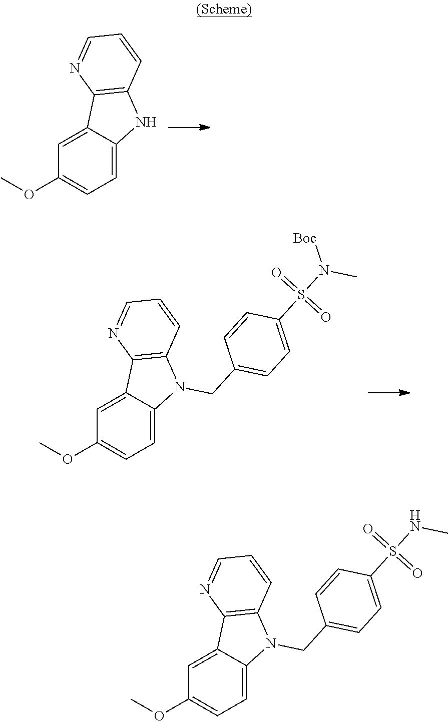

### (Step 1) Preparation of tert-butyl ((4-((8-methoxy-5H-pyrido[3,2-b]indol-5-yl)methyl)phenyl)sulfonyl)(methyl)carbamate

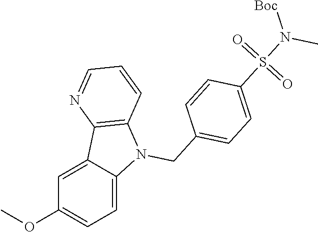

The compound prepared in (Step 3) of Example 5 (30.0 mg, 0.15 mmol) was dissolved in N,N-dimethylformamide (1.00 mL), and then cooled to 0° C., and 60% sodium hydride (7.20 mg, 0.18 mmol) was added to the mixture and stirred for 30 minutes. Intermediate 3 (66.2 mg, 0.18 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 3:2) to obtain the title compound as a yellow solid. (46.0 mg, 63%) MS m/z: 482 [M+1]^{+ 1}H NMR (400 MHz, CDCl₃) δ ppm: 8.56 (dd, J=4.7, 1.3 Hz, 1H), 7.90 (d, J=2.4 Hz, 1H), 7.79 (d, J=8.6 Hz, 2H), 7.55 (dd, J=8.3, 1.4 Hz, 1H), 7.30 (dd, J=8.3, 4.8 Hz, 1H), 7.24-7.19 (m, 3H), 7.15 (dd, J=8.9, 2.5 Hz, 1H), 5.56 (s, 2H), 3.95 (s, 3H), 3.31 (s, 3H), 1.29 (s, 9H).

### (Step 2) Preparation of 4-((8-methoxy-5H-pyrido[3,2-b]indol-5-yl)methyl)-N-methylbenzenesulfonamide (Compound 7)

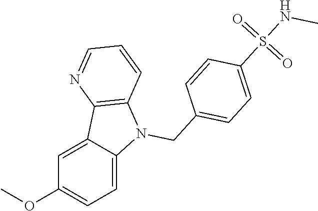

The compound prepared in (Step 1) above (46.0 mg, 0.10 mmol) was dissolved in methylene chloride (1.00 mL), and then trifluoroacetic acid (1.00 mL) was added to the mixture. The reaction mixture was stirred at room temperature for 2 hours under nitrogen. After completion of the reaction, distilled water and an aqueous sodium hydrogen carbonate solution were added slowly to neutralize, and extracted twice with methylene chloride. The organic layers were dried over anhydrous sodium sulfate, and then concentrated under reduced pressure. The residue was purified by silica gel column chromatography (ethyl acetate:n-hexane=3:7 to 9:1) to obtain the title compound as a yellow solid. (12.0 mg, 32%) MS m/z: 382 [M+1]^{+ 1}H NMR (400 MHz, DMSO-d₆) δ ppm: 8.47 (dd, J=4.6, 1.4 Hz, 1H), 8.02 (dd, J=8.4, 1.4 Hz, 1H), 7.73 (d, J=2.5 Hz, 1H), 7.67 (d, J=8.5 Hz, 2H), 7.60 (d, J=9.0 Hz, 1H), 7.41 (dd, J=8.4, 4.6 Hz, 1H), 7.37 (d, J=4.8 Hz, 1H), 7.30 (d, J=8.5 Hz, 2H), 7.18 (dd, J=9.0, 2.6 Hz, 1H), 5.78 (s, 2H), 3.88 (s, 3H), 2.35 (d, J=4.4 Hz, 3H).

### [Example 8] Preparation of N-(4-((8-methoxy-5H-pyrido[3,2-b]indol-5-yl)methyl)phenyl) sulfamide (Compound 8)

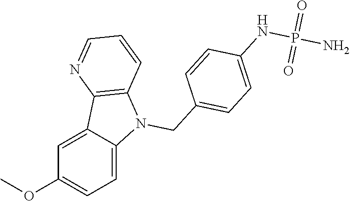

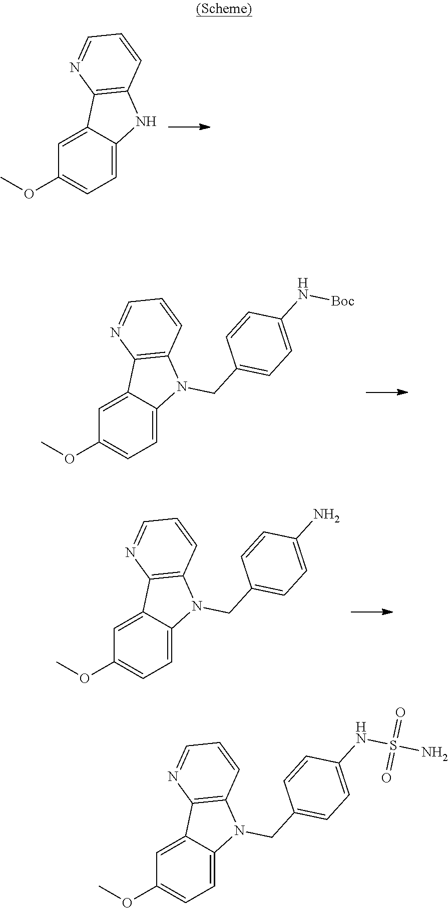 [SMILES: COc1ccc2[nH]c3cccnc3c2c1 ; COc1ccc2c(c1)c1ncccc1n2Cc1ccc(NC(=O)OC(C)(C)C)cc1 ; COc1ccc2c(c1)c1ncccc1n2Cc1ccc(N)cc1 ; COc1ccc2c(c1)c1ncccc1n2Cc1ccc(NS(N)(=O)=O)cc1]

### (Step 1) Preparation of tert-butyl (4-((8-methoxy-5H-pyrido[3,2-b]indol-5-yl)methyl)phenyl) carbamate

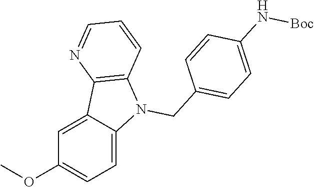 [SMILES: COc1ccc2c(c1)c1ncccc1n2Cc1ccc(NC(=O)OC(C)(C)C)cc1]

The compound prepared in (Step 3) of Example 5 (50.0 mg, 0.17 mmol) was dissolved in N,N-dimethylformamide (1.00 mL), and then cooled to 0° C., and 60% sodium hydride (14.0 mg, 0.34 mmol) was added to the mixture and stirred for 30 minutes. Intermediate 4 (38.1 mg, 0.19 mmol) dissolved in N,N-dimethylformamide (0.50 mL) was slowly added to the reaction mixture, and then stirred at 0° C. for 2 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (36.0 mg, 51%) MS m/z: 404 [M+1]⁺

### (Step 2) Preparation of 4-((8-methoxy-5H-pyrido[3,2-b]indol-5-yl)methyl) aniline

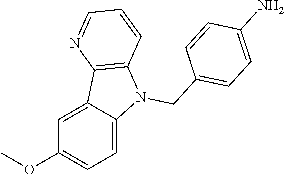 [SMILES: COc1ccc2c(c1)c1ncccc1n2Cc1ccc(N)cc1]

The compound prepared in (Step 1) above (36.0 mg, 0.09 mmol) was dissolved in methylene chloride (0.50 mL), and then trifluoroacetic acid (0.50 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a red solid. (27.0 mg, 99%) MS m/z: 304 [M+1]⁺

### (Step 3) Preparation of N-(4-((8-methoxy-5H-pyrido[3,2-b]indol-5-yl)methyl)phenyl) sulfamide (Compound 8)

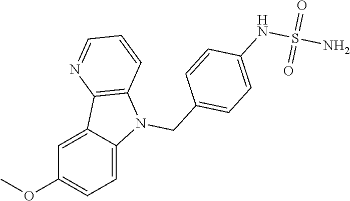 [SMILES: COc1ccc2c(c1)c1ncccc1n2Cc1ccc(NS(N)(=O)=O)cc1]

The compound prepared in (Step 2) above (31.0 mg, 0.10 mmol) was dissolved in acetonitrile (1.00 mL), and then N,N-diisopropylethylamine (37.0 μl, 0.20 mmol) and (4-nitrophenyl) sulfamate (223 mg, 1.01 mmol) were added to the mixture sequentially. The reaction mixture was stirred at room temperature for 3 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a white solid. (2.30 mg, 6%) MS m/z: 383 [M+1]^{+ 1}H NMR (400 MHz, CD₃OD) δ 8.41 (dd, J=4.8, 1.2 Hz, 1H), 7.95 (dd, J=8.4, 1.2 Hz, 1H), 7.86 (d, J=2.5 Hz, 1H), 7.51 (d, J=9.0 Hz, 1H), 7.43 (dd, J=8.4, 4.8 Hz, 1H), 7.20 (dd, J=9.0, 2.5 Hz, 1H), 7.15-7.07 (m, 4H), 5.57 (s, 2H), 3.92 (s, 3H).

### [Example 9] Preparation of 4-(2-(8-methoxy-5H-pyrido[3,2-b]indol-5-yl)ethyl)benzenesulfonamide (Compound 9)

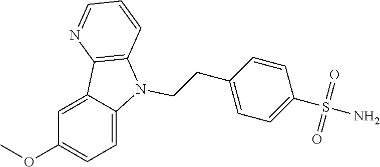 [SMILES: COc1ccc2c(c1)c1ncccc1n2CCc1ccc(S(N)(=O)=O)cc1]

The compound prepared in (Step 3) of Example 5 (25.0 mg, 0.13 mmol) and Intermediate 5 (23.0 mg, 0.13 mmol) were dissolved in dimethyl sulfoxide (1.00 mL), and then potassium hydroxide (14.0 mg, 0.25 mmol) was added to the mixture and stirred at 110° C. for 48 hours. After cooling to room temperature, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=3:7 to 9:1) to obtain the title compound as a white solid. (6.70 mg, 14%) MS m/z: 382 [M+1]^{+ 1}H NMR (400 MHz, DMSO-d₆) δ ppm: 8.40 (dd, J=4.6, 1.4 Hz, 1H), 7.91 (dd, J=8.3, 1.4 Hz, 1H), 7.70-7.61 (m, 4H), 7.42 (d, J=8.4 Hz, 2H), 7.34 (dd, J=8.3, 4.6 Hz, 1H), 7.26 (s, 2H), 7.17 (dd, J=8.9, 2.6 Hz, 1H), 4.64 (t, J=7.4 Hz, 2H), 3.87 (s, 3H), 3.12 (t, J=7.3 Hz, 2H).

### [Example 10] Preparation of imino (4-((8-methoxy-5H-pyrido[3,2-b]indol-5-yl)methyl)phenyl)(methyl)-l6-sulfanone (Compound 10)

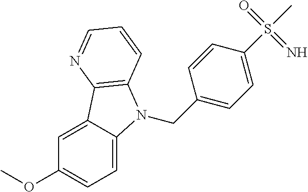 [SMILES: COc1ccc2c(c1)c1ncccc1n2Cc1ccc(S(C)(=N)=O)cc1]

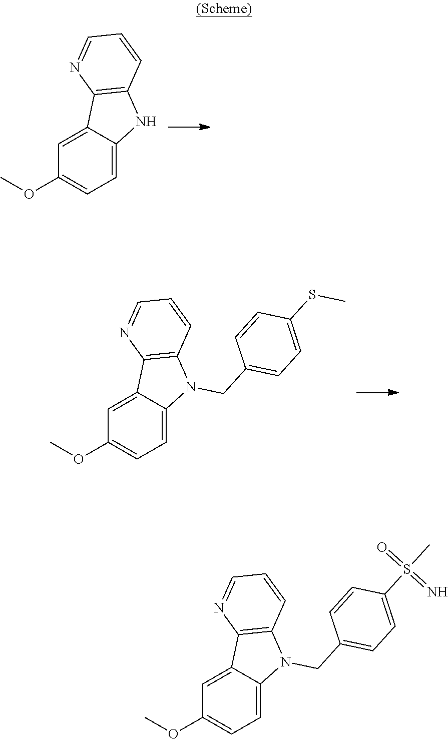

### (Step 1) Preparation of 8-methoxy-5-(4-(methylthio)benzyl)-5H-pyrido[3,2-b]indole

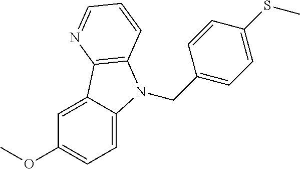

The compound prepared in (Step 3) of Example 5 (140 mg, 0.71 mmol) was dissolved in N,N-dimethylformamide (3.00 mL), and then cooled to 0° C., and 60% sodium hydride (84.8 mg, 2.12 mmol) was added to the mixture and stirred for 30 minutes. Intermediate 6 (184 mg, 0.85 mmol) dissolved in N,N-dimethylformamide (1 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:1) to obtain the title compound as a yellow solid. (95.0 mg, 40%) MS m/z: 335 [M+1]⁺

### (Step 2) Preparation of imino (4-((8-methoxy-5H-pyrido[3,2-b]indol-5-yl)methyl)phenyl)(methyl)-l6-sulfanone (Compound 10)

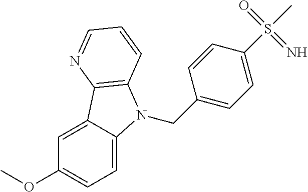 [SMILES: COc1ccc2c(c1)c1ncccc1n2Cc1ccc(S(C)(=N)=O)cc1]

The compound prepared in (Step 1) above (95.0 mg, 0.28 mmol) was dissolved in ethanol (1.00 mL), and then iodobenzene diacetate (265 mg, 0.82 mmol) and ammonium acetate (83.2 mg, 1.08 mmol) were added to the mixture. The reaction mixture was stirred at room temperature for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (90.0 mg, 86%) Compound 10 was purified by preparative high performance liquid chromatography (prep-HPLC, chiralpak (250*30 mm, 5 μm), hexane:methanol:ethanol=40:30:30) to obtain 10a and 10b, which are white optical isomers, respectively. 3a: [α]D24+6° (c 0.14, CHCl3), 3b: [α]D24−6° (c 0.13, CHCl3). MS m/z: 366 [M+1]^{+ 1}H NMR (400 MHz, CD₃OD) δ 8.69 (d, J=4.9 Hz, 1H), 8.47 (d, J=8.3 Hz, 1H), 7.96 (d, J=8.0 Hz, 2H), 7.89 (s, 1H), 7.73 (d, J=8.3 Hz, 2H), 7.45 (d, J=8.0 Hz, 2H), 7.34 (d, J=9.1 Hz, 1H), 5.97 (s, 2H), 3.89 (s, 3H), 3.53 (s, 3H).

### [Example 11] Preparation of ethyl (imino) (4-((8-methoxy-5H-pyrido[3,2-b]indol-5-yl)methyl)phenyl)-l6-sulfanone (Compound 11)

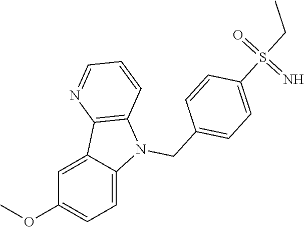 [SMILES: CCS(=N)(=O)c1ccc(Cn2c3ccc(OC)cc3c3ncccc32)cc1]

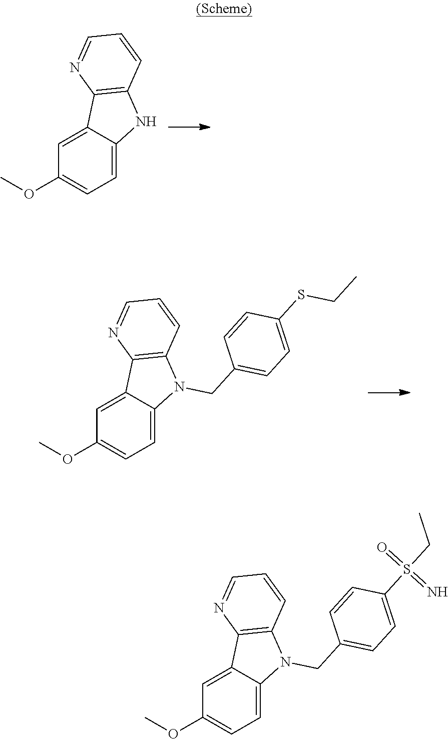

### (Step 1) Preparation of 5-(4-(ethylthio)benzyl)-8-methoxy-5H-pyrido[3,2-b]indole

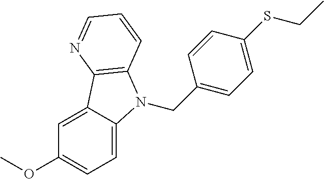 [SMILES: CCSc1ccc(Cn2c3ccc(OC)cc3c3ncccc32)cc1]

The compound prepared in (Step 3) of Example 5 (30.0 mg, 0.15 mmol) was dissolved in N,N-dimethylformamide (1.00 mL), and then cooled to 0° C., and 60% sodium hydride (9.10 mg, 0.23 mmol) was added to the mixture and stirred for 30 minutes. Intermediate 7 (45.5 mg, 0.20 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow solid. (16.3 mg, 31%) MS m/z: 349 [M+1]^{+ 1}H NMR (400 MHz, CDCl₃) δ ppm: 8.54 (d, J=4.6 Hz, 1H), 7.89 (d, J=2.6 Hz, 1H), 7.60 (d, J=9.8 Hz, 1H), 7.29 (dd, J=8.3, 4.6 Hz, 2H), 7.20 (d, J=8.4 Hz, 2H), 7.16 (d, J=8.9 Hz, 1H), 7.01 (d, J=8.3 Hz, 2H), 5.45 (s, 2H), 3.95 (s, 3H), 2.88 (q, J=7.4 Hz, 2H), 1.31-1.24 (m, 3H).

### (Step 2) Preparation of ethyl(imino)(4-((8-methoxy-5H-pyrido[3,2-b]indol-5-yl)methyl)phenyl)-l6-sulfanone (Compound 11)

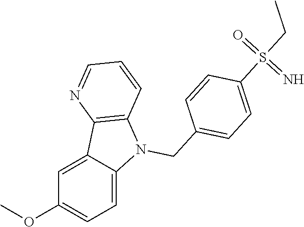 [SMILES: CCS(=N)(=O)c1ccc(Cn2c3ccc(OC)cc3c3ncccc32)cc1]

The compound prepared in (Step 1) above (16.5 mg, 0.05 mmol) was dissolved in ethanol (2.00 mL), and then iodobenzene diacetate (43.7 mg, 0.14 mmol) and ammonium acetate (13.7 mg, 0.18 mmol) were added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (8.20 mg, 46%) MS m/z: 380 [M+1]^{+ 1}H NMR (400 MHz, DMSO-d₆) δ ppm: 8.56 (d, J=3.6 Hz, 1H), 8.22 (d, J=7.9 Hz, 1H), 7.82 (d, J=8.4 Hz, 2H), 7.79 (d, J=2.5 Hz, 1H), 7.65 (d, J=9.0 Hz, 1H), 7.54 (dd, J=8.3, 4.8 Hz, 1H), 7.36 (d, J=8.4 Hz, 2H), 7.24 (dd, J=8.9, 2.6 Hz, 1H), 5.87 (s, 2H), 3.88 (s, 3H), 3.29 (q, J=7.2 Hz, 2H), 1.04 (t, J=7.3 Hz, 3H).

### [Example 12] Preparation of benzyl(imino)(4-((8-methoxy-5H-pyrido[3,2-b]indol-5-yl)methyl)phenyl)-l6-sulfanone (Compound 12)

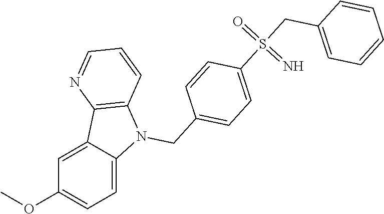 [SMILES: COc1ccc2c(c1)c1ncccc1n2Cc1ccc(S(=N)(=O)Cc2ccccc2)cc1]

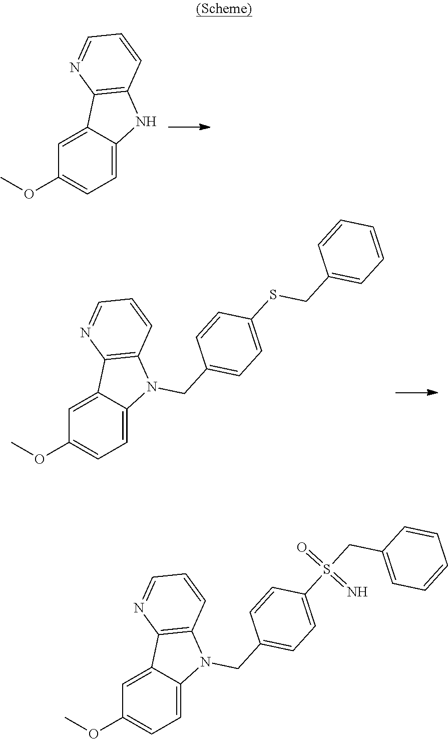 [SMILES: COc1ccc2[nH]c3cccnc3c2c1 ; COc1ccc2c(c1)c1ncccc1n2Cc1ccc(SCc2ccccc2)cc1 ; COc1ccc2c(c1)c1ncccc1n2Cc1ccc(S(=N)(=O)Cc2ccccc2)cc1]

### (Step 1) Preparation of 5-(4-(benzylthio)benzyl)-8-methoxy-5H-pyrido[3,2-b]indole

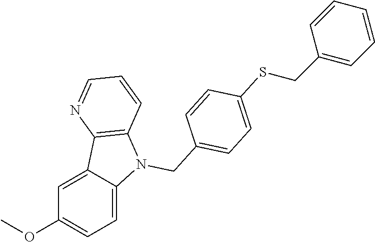 [SMILES: COc1ccc2c(c1)c1ncccc1n2Cc1ccc(SCc2ccccc2)cc1]

The compound prepared in (Step 3) of Example 5 (30.0 mg, 0.15 mmol) was dissolved in N,N-dimethylformamide (1.0 mL), and then cooled to 0° C., and 60% sodium hydride (9.10 mg, 0.13 mmol) was added to the mixture and stirred for 30 minutes. Intermediate 8 (57.7 mg, 0.20 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow solid. (20.9 mg, 34%) MS m/z: 411 [M+1]^{+ 1}H NMR (400 MHz, CDCl₃) δ ppm: 8.54 (dd, J=4.7, 1.4 Hz, 1H), 7.90 (d, J=2.6 Hz, 1H), 7.59 (dd, J=8.4, 1.5 Hz, 1H), 7.33-7.25 (m, 5H), 7.23-7.14 (m, 5H), 6.98 (d, J=8.4 Hz, 2H), 5.43 (s, 2H), 4.05 (s, 2H), 3.95 (s, 3H).

### (Step 2) Preparation of ethyl (imino) (4-((8-methoxy-5H-pyrido[3,2-b]indol-5-yl)methyl)phenyl)-l6-sulfanone (Compound 12)

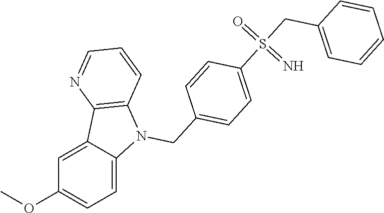 [SMILES: COc1ccc2c(c1)c1ncccc1n2Cc1ccc(S(=N)(=O)Cc2ccccc2)cc1]

The compound prepared in (Step 1) above (20.9 mg, 0.05 mmol) was dissolved in ethanol (2.00 mL), and then iodobenzene diacetate (49.2 mg, 0.15 mmol) and ammonium acetate (15.7 mg, 0.20 mmol) were added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (8.10 mg, 36%) MS m/z: 442 [M+1]^{+ 1}H NMR (400 MHz, DMSO-d₆) δ ppm: 8.59 (s, 1H), 8.25 (s, 1H), 7.81 (s, 1H), 7.65-7.57 (m, 4H), 7.34-7.19 (m, 4H), 7.13 (t, J=7.5 Hz, 2H), 6.97 (d, J=7.8 Hz, 2H), 5.83 (s, 2H), 4.52 (s, 2H), 3.89 (s, 3H).

### [Example 13] Preparation of (4-((8-methoxy-5H-pyrido[3,2-b]indol-5-yl)methyl)phenyl)phosphonic acid (Compound 13)

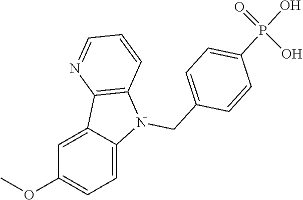 [SMILES: COc1ccc2c(c1)c1ncccc1n2Cc1ccc(P(=O)(O)O)cc1]

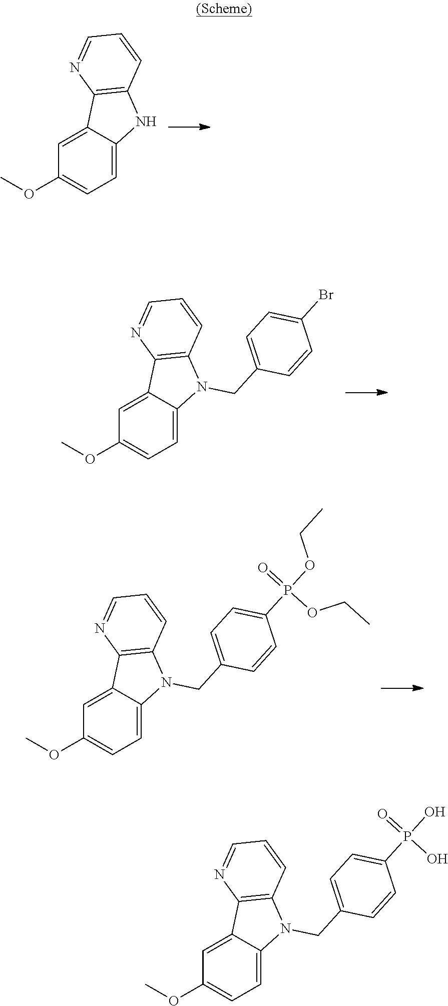

### (Step 1) Preparation of 5-(4-bromobenzyl)-8-methoxy-5H-pyrido[3,2-b]indole

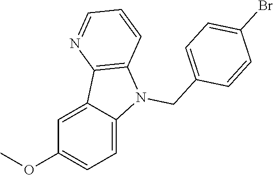 [SMILES: COc1ccc2c(c1)c1ncccc1n2Cc1ccc(Br)cc1]

The compound prepared in (Step 3) of Example 5 (40.0 mg, 0.20 mmol) was dissolved in N,N-dimethylformamide (1.00 mL), and then cooled to 0° C., and 60% sodium hydride (9.60 mg, 0.24 mmol) was added to the mixture and stirred for 30 minutes. 1-Bromo-4-(bromomethyl)benzene (60.5 mg, 0.24 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=0:10 to 3:2) to obtain the title compound as a yellow solid. (30.0 mg, 40%) MS m/z: 368 [M+1]^{+ 1}H NMR (400 MHz, CDCl₃) δ ppm: 8.55 (dd, J=4.8, 1.4 Hz, 1H), 7.89 (d, J=2.5 Hz, 1H), 7.57 (dd, J=8.4, 1.4 Hz, 1H), 7.43-7.36 (m, 2H), 7.33-7.26 (m, 2H), 7.17 (dd, J=8.9, 2.5 Hz, 1H), 6.97 (d, J=8.8 Hz, 2H), 5.44 (s, 2H), 3.95 (s, 3H).

### (Step 2) Preparation of diethyl (4-((8-methoxy-5H-pyrido[3,2-b]indol-5-yl)methyl)phenyl)phosphonate

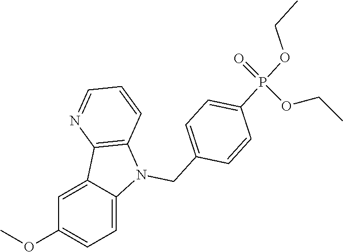 [SMILES: CCOP(=O)(OCC)c1ccc(Cn2c3ccc(OC)cc3c3ncccc32)cc1]

The compound prepared in (Step 1) above (36.0 mg, 0.10 mmol) and diethyl phosphite (14.0 μl, 0.11 mmol) were dissolved in toluene (2.00 mL), and then triethylamine (15.0 μl, 0.11 mmol) and Tetrakis(triphenylphosphine)palladium(0) (11.3 mg, 0.01 mmol) were added to the mixture and stirred at 90° C. for 3 hours. After cooling to room temperature, Precipitation was removed by filtration with diethyl ether, and then the organic layers were concentrated under reduced pressure. The residue was purified by silica gel column chromatography (ethyl acetate:n-hexane=1:1 to 10:0; methanol:methylene chloride=1:19) to obtain the title compound as a yellow liquid. (32.0 mg, 77%) MS m/z: 425 [M+1]^{+ 1}H NMR (400 MHz, CDCl₃) δ ppm: 8.56 (dd, J=4.7, 1.3 Hz, 1H), 7.93 (d, J=2.6 Hz, 1H), 7.69 (dd, J=13.0, 8.4 Hz, 2H), 7.59 (d, J=8.4 Hz, 1H), 7.31 (dd, J=8.3, 4.7 Hz, 1H), 7.28-7.26 (m, 1H), 7.22-7.15 (m, 3H), 5.53 (s, 2H), 4.18-3.97 (m, 4H), 3.95 (s, 3H), 1.29 (t, J=7.1 Hz, 6H).

### (Step 3) Preparation of (4-((8-methoxy-5H-pyrido[3,2-b]indol-5-yl)methyl)phenyl)phosphonic acid (Compound 13)

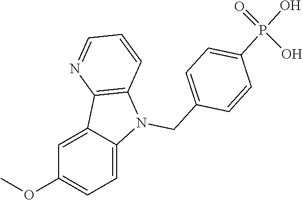 [SMILES: COc1ccc2c(c1)c1ncccc1n2Cc1ccc(P(=O)(O)O)cc1]

The compound prepared in (Step 2) above (32.0 mg, 0.08 mmol) was dissolved in methylene chloride (1.50 mL), and then bromotrimethylsilane (0.10 mL, 0.75 mmol) was added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (18.3 mg, 65%) MS m/z: 369 [M+1]^{+ 1}H NMR (400 MHz, DMSO-d₆) δ ppm: 8.63 (d, J=5.0 Hz, 1H), 8.39 (d, J=8.4 Hz, 1H), 7.84 (s, 1H), 7.75-7.63 (m, 2H), 7.55 (dd, J=12.8, 8.1 Hz, 2H), 7.29 (d, J=9.0 Hz, 1H), 7.20 (dd, J=8.1, 3.4 Hz, 2H), 5.81 (s, 2H), 3.88 (s, 3H).

### [Example 14] Preparation of (4-((8-methoxy-5H-pyrido[3,2-b]indol-5-yl)methyl)benzyl)phosphonic acid (Compound 14)

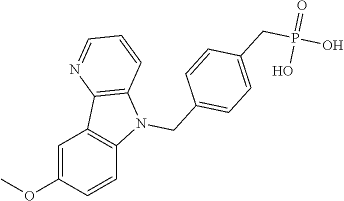 [SMILES: COc1ccc2c(c1)c1ncccc1n2Cc1ccc(CP(=O)(O)O)cc1]

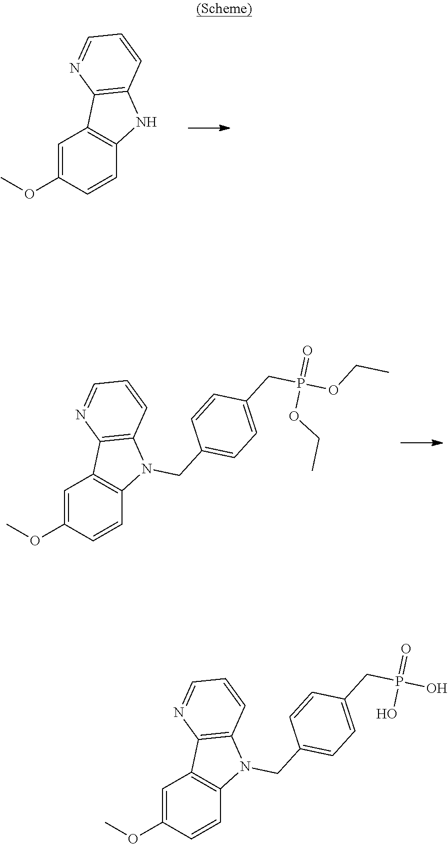

### (Step 1) Preparation of diethyl (4-((8-methoxy-5H-pyrido[3,2-b]indol-5-yl)methyl)benzyl)phosphonate

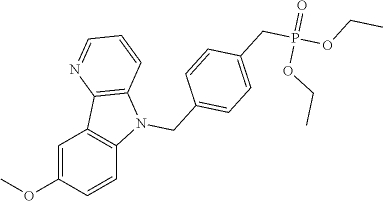 [SMILES: CCOP(=O)(Cc1ccc(Cn2c3ccc(OC)cc3c3ncccc32)cc1)OCC]

The compound prepared in (Step 3) of Example 5 (20.0 mg, 0.10 mmol) was dissolved in N,N-dimethylformamide (1.00 mL), and then cooled to 0° C., and 60% sodium hydride (8.00 mg, 0.20 mmol) was added to the mixture and stirred for 30 minutes. Intermediate 9 (35.6 mg, 0.11 mmol) dissolved in N,N-dimethylformamide (0.50 mL) was slowly added to the reaction mixture, and then stirred at 80° C. for 3 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 10:0) to obtain the title compound as a yellow solid. (12.0 mg, 27%) MS m/z: 439 [M+1]⁺

### Step 2) Preparation of (4-((8-methoxy-5H-pyrido[3,2-b]indol-5-yl)methyl)benzyl)phosphonic acid (Compound 14)

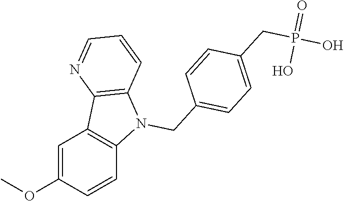 [SMILES: COc1ccc2c(c1)c1ncccc1n2Cc1ccc(CP(=O)(O)O)cc1]

The compound prepared in (Step 1) above (12.0 mg, 0.03 mmol) was dissolved in methylene chloride (1.00 mL), and then bromotrimethylsilane (36.0 μl, 0.27 mmol) was added to the mixture. The reaction mixture was stirred at room temperature for 15 hours under nitrogen. After completion of the reaction, the mixture was concentrated under reduced pressure, purified by preparative high performance liquid chromatography (prep-HPLC, 0.1% TFA acetonitrile:distilled water=5:95 to 100:0) to obtain the title compound as a yellow solid. (3.00 mg, 28%) MS m/z: 383 [M+1]^{+ 1}H NMR (400 MHz, CD₃OD) δ 8.72 (d, J=8.4 Hz, 1H), 8.66 (d, J=5.6 Hz, 1H), 7.92 (dd, J=8.5, 5.7 Hz, 1H), 7.87 (d, J=2.5 Hz, 1H), 7.77 (d, J=9.2 Hz, 1H), 7.47 (dd, J=9.2, 2.5 Hz, 1H), 7.25 (dd, J=8.1, 2.3 Hz, 2H), 7.16 (d, J=8.1 Hz, 2H), 5.79 (s, 2H), 3.97 (s, 3H), 3.08 (s, 1H), 3.03 (s, 1H).

### [Example 15] Preparation of 3-fluoro-4-((8-methoxy-5H-pyrido[3,2-b]indol-5-yl)methyl)benzenesulfonamide (Compound 15)

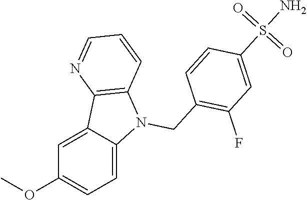 [SMILES: COc1ccc2c(c1)c1ncccc1n2Cc1ccc(S(N)(=O)=O)cc1F]

The compound prepared in (Step 3) of Example 5 (30.0 mg, 0.15 mmol) was dissolved in N,N-dimethylformamide (1.50 mL), and then cooled to 0° C., and 60% sodium hydride (12.1 mg, 0.30 mmol) was added to the mixture and stirred for 30 minutes. Intermediate 10 (48.6 mg, 0.18 mmol) dissolved in N,N-dimethylformamide (1.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (3.50 mg, 6%) MS m/z: 386 [M+1]^{+ 1}H NMR (400 MHz, DMSO-d₆) δ ppm: 8.54 (s, 1H), 8.17 (s, 1H), 7.78 (s, 1H), 7.63 (s, 1H), 7.55-7.38 (m, 5H), 7.24 (s, 1H), 7.02 (s, 1H), 5.84 (s, 2H), 3.88 (m, 3H).

### [Example 16] Preparation n of 3-chloro-4-((8-methoxy-5H-pyrido[3,2-b]indol-5-yl)methyl)benzenesulfonamide (Compound 16)

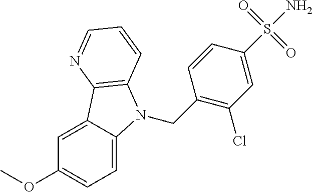 [SMILES: COc1ccc2c(c1)c1ncccc1n2Cc1ccc(S(N)(=O)=O)cc1Cl]

The compound prepared in (Step 3) of Example 5 (30.0 mg, 0.15 mmol) was dissolved in N,N-dimethylformamide (1.50 mL), and then cooled to 0° C., and 60% sodium hydride (12.1 mg, 0.30 mmol) was added to the mixture and stirred for 30 minutes. Intermediate 11 (51.6 mg, 0.18 mmol) dissolved in N,N-dimethylformamide (1.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile distilled water=5:95 to 100:0) to obtain the title compound as a yellow solid. (4.00 mg, 7%) MS m/z: 402 [M+1]^{+ 1}H NMR (400 MHz, DMSO-d₆) δ ppm: 8.55 (s, 1H), 8.08 (s, 1H), 7.93 (s, 1H), 7.80 (s, 1H), 7.53-7.41 (m, 5H), 7.20 (s, 1H), 6.58 (s, 1H), 5.83 (s, 2H), 3.88 (m, 3H).

### [Example 17] Preparation of (3-fluoro-4-((8-methoxy-5H-pyrido[3,2-b]indol-5-yl)methyl)phenyl)(imino)(methyl)-l6-sulfanone (Compound 17)

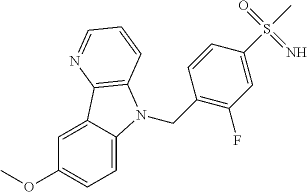 [SMILES: COc1ccc2c(c1)c1ncccc1n2Cc1ccc(S(C)(=N)=O)cc1F]

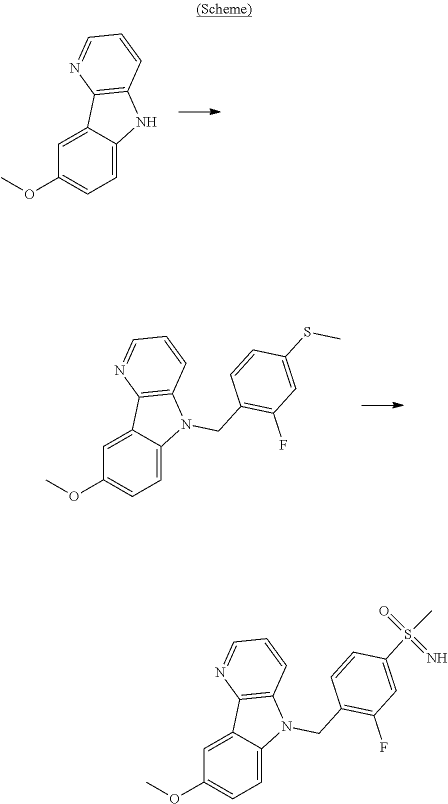

### (Step 1) Preparation of 5-(2-fluoro-4-(methylthio)benzyl)-8-methoxy-5H-pyrido[3,2-b]indole

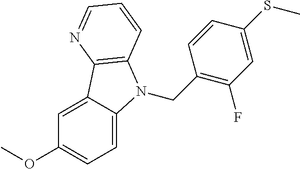

The compound prepared in (Step 3) of Example 5 (33.0 mg, 0.17 mmol) was dissolved in N,N-dimethylformamide (1.00 mL), and then cooled to 0° C., and 60% sodium hydride (7.90 mg, 0.20 mmol) was added to the mixture and stirred for 30 minutes. Intermediate 12 (50.9 mg, 0.22 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow liquid. (24.0 mg, 41%) MS m/z: 353 [M+1]^{+ 1}H NMR (400 MHz, CDCl₃) δ ppm: 8.55 (s, 1H), 7.89 (s, 1H), 7.66 (d, J=8.0 Hz, 1H), 7.35 (d, J=7.9 Hz, 2H), 7.18 (d, J=8.3 Hz, 1H), 6.97 (d, J=9.8 Hz, 1H), 6.78 (s, 1H), 6.69-6.68 (m, 1H), 5.48 (s, 2H), 3.95 (s, 3H), 2.42 (s, 3H).

### (Step 2) Preparation of (3-fluoro-4-((8-methoxy-5H-pyrido[3,2-b]indol-5-yl)methyl)phenyl)(imino)(methyl)-l6-sulfanone (Compound 17)

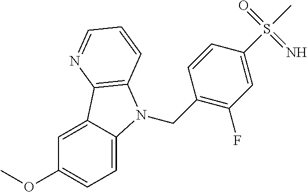 [SMILES: COc1ccc2c(c1)c1ncccc1n2Cc1ccc(S(C)(=N)=O)cc1F]

The compound prepared in (Step 1) above (24.0 mg, 0.07 mmol) was dissolved in ethanol (2.00 mL), and then iodobenzene diacetate (63.6 mg, 0.20 mmol) and ammonium acetate (19.9 mg, 0.26 mmol) were added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (18.0 mg, 68%) MS m/z: 384 [M+1]^{+ 1}H NMR (400 MHz, DMSO-d₆) δ ppm: 8.61 (s, 1H), 8.30 (s, 1H), 7.86-7.74 (m, 2H), 7.67 (d, J=10.4 Hz, 1H), 7.61 (s, 2H), 7.27 (d, J=7.4 Hz, 1H), 7.08 (s, 1H), 5.90 (s, 2H), 3.88 (s, 3H), 3.14 (s, 3H).

### [Example 18] Preparation of imino (4-((8-methoxy-5H-pyrido[3,2-b]indol-5-yl)methyl)-3-methylphenyl)(methyl)-l6-sulfanone (Compound 18)

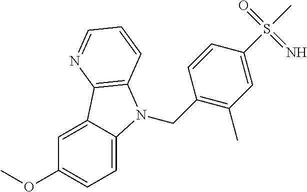 [SMILES: COc1ccc2c(c1)c1ncccc1n2Cc1ccc(S(C)(=N)=O)cc1C]

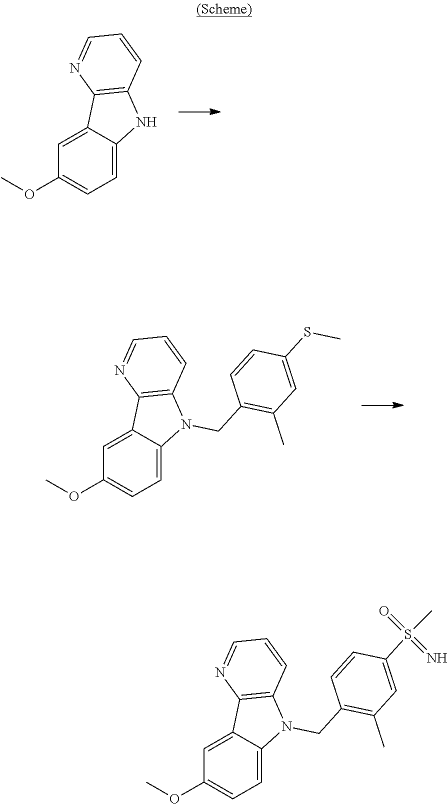

### (Step 1) Preparation of 8-methoxy-5-(2-methyl-4-(methylthio)benzyl)-5H-pyrido[3,2-b]indole

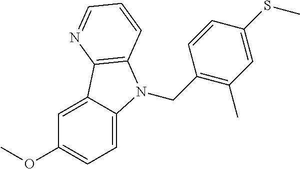

The compound prepared in (Step 3) of Example 5 (40.0 mg, 0.20 mmol) was dissolved in N,N-dimethylformamide (1.00 mL), and then cooled to 0° C., and 60% sodium hydride (12.0 mg, 0.30 mmol) was added to the mixture and stirred for 30 minutes. Intermediate 14 (56.0 mg, 0.24 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow solid. (33.5 mg, 48%) MS m/z: 349 [M+1]^{+ 1}H NMR (400 MHz, CDCl₃) δ ppm: 8.54 (dd, J=4.6, 1.4 Hz, 1H), 7.91 (d, J=2.4 Hz, 1H), 7.51 (dd, J=8.3, 1.4 Hz, 1H), 7.27 (dd, J=8.3, 4.6 Hz, 1H), 7.21 (d, J=8.9 Hz, 1H), 7.19-7.10 (m, 2H), 6.85 (dd, J=8.2, 2.2 Hz, 1H), 6.44 (d, J=8.0 Hz, 1H), 5.41 (s, 2H), 3.95 (s, 3H), 2.42 (s, 3H), 2.39 (s, 3H).

### (Step 2) Preparation of imino (4-((8-methoxy-5H-pyrido[3,2-b]indol-5-yl)methyl)-3-methylphenyl)(methyl)-l6-sulfanone (Compound 18)

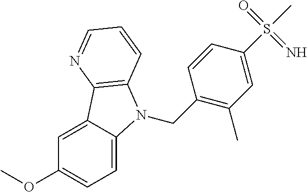 [SMILES: COc1ccc2c(c1)c1ncccc1n2Cc1ccc(S(C)(=N)=O)cc1C]

The compound prepared in (Step 1) above (33.5 mg, 0.096 mmol) was dissolved in ethanol (2.00 mL), and then iodobenzene diacetate (89.8 mg, 0.28 mmol) and ammonium acetate (28.2 mg, 0.36 mmol) were added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (36.1 mg, 98%) MS m/z: 380 [M+1]^{+ 1}H NMR (400 MHz, DMSO-d₆) δ ppm: 8.61 (dd, J=4.9, 1.3 Hz, 1H), 8.15 (d, J=8.9 Hz, 1H), 7.93 (d, J=2.4 Hz, 1H), 7.85 (d, J=2.5 Hz, 1H), 7.62-7.53 (m, 3H), 7.26 (dd, J=9.0, 2.6 Hz, 1H), 6.28 (d, J=8.4 Hz, 1H), 5.87 (s, 2H), 3.90 (s, 3H), 3.42 (s, 3H), 2.60 (s, 3H).

### [Example 19] Preparation of imino (3-methoxy-4-((8-methoxy-5H-pyrido[3,2-b]indol-5-yl)methyl)phenyl)(methyl)-l6-sulfanone (Compound 19)

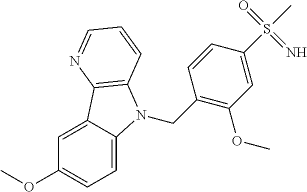 [SMILES: COc1ccc2c(c1)c1ncccc1n2Cc1ccc(S(C)(=N)=O)cc1OC]

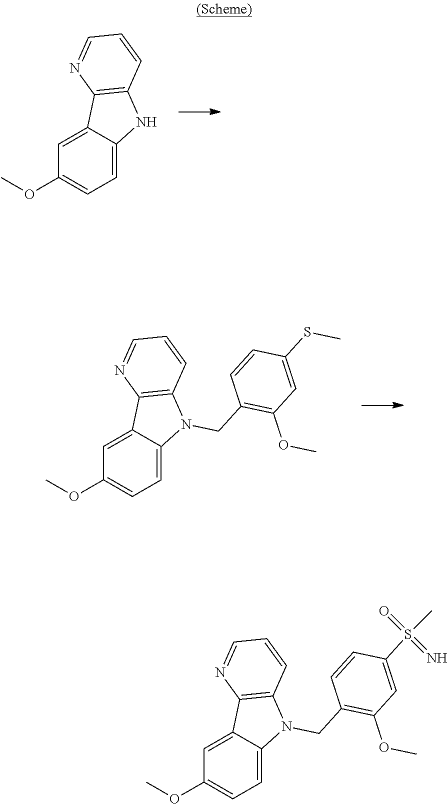

### (Step 1) Preparation of 8-methoxy-5-(2-methoxy-4-(methylthio)benzyl)-5H-pyrido[3,2-b]indole

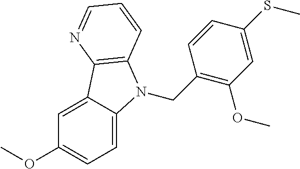

The compound prepared in (Step 3) of Example 5 (30.0 mg, 0.15 mmol) was dissolved in N,N-dimethylformamide (1.00 mL), and then cooled to 0° C., and 60% sodium hydride (9.00 mg, 0.23 mmol) was added to the mixture and stirred for 30 minutes. Intermediate 13 (48.6 mg, 0.20 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow solid. (20.0 mg, 36%) MS m/z: 365 [M+1]^{+ 1}H NMR (400 MHz, CDCl₃) δ ppm: 8.53 (dd, J=4.6, 1.4 Hz, 1H), 7.89 (d, J=2.6 Hz, 1H), 7.65 (dd, J=8.4, 1.4 Hz, 1H), 7.36-7.28 (m, 2H), 7.16 (dd, J=8.9, 2.5 Hz, 1H), 6.82 (d, J=1.8 Hz, 1H), 6.60 (dd, J=7.9, 1.8 Hz, 1H), 6.53 (d, J=8.0 Hz, 1H), 5.43 (s, 2H), 3.95 (s, 3H), 3.89 (s, 3H), 2.42 (s, 3H).

### (Step 2) Preparation of imino (3-methoxy-4-((8-methoxy-5H-pyrido[3,2-b]indol-5-yl)methyl)phenyl)(methyl)-l6-sulfanone (Compound 19)

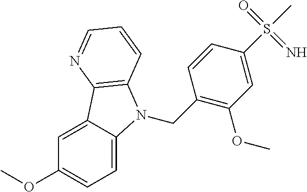 [SMILES: COc1ccc2c(c1)c1ncccc1n2Cc1ccc(S(C)(=N)=O)cc1OC]

The compound prepared in (Step 1) above (20.0 mg, 0.06 mmol) was dissolved in ethanol (2.00 mL), and then iodobenzene diacetate (51.3 mg, 0.16 mmol) and ammonium acetate (16.1 mg, 0.21 mmol) were added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (20.9 mg, 95%) MS m/z: 396 [M+1]^{+ 1}H NMR (400 MHz, DMSO-d₆) δ ppm: 8.63 (d, J=4.3 Hz, 1H), 8.31 (d, J=7.9 Hz, 1H), 7.84 (d, J=2.5 Hz, 1H), 7.64 (dd, J=8.7, 4.8 Hz, 2H), 7.57 (d, J=1.9 Hz, 1H), 7.40 (dd, J=8.0, 1.9 Hz, 1H), 7.28 (dd, J=9.0, 2.6 Hz, 1H), 6.87 (d, J=7.9 Hz, 1H), 5.77 (s, 2H), 3.94 (s, 3H), 3.88 (s, 3H), 3.34 (s, 3H).

### [Example 20] Preparation of (2-fluoro-4-((8-methoxy-5H-pyrido[3,2-b]indol-5-yl)methyl)phenyl)(imino)(methyl)-l6-sulfanone (Compound 20)

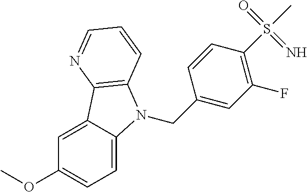 [SMILES: COc1ccc2c(c1)c1ncccc1n2Cc1ccc(S(C)(=N)=O)c(F)c1]

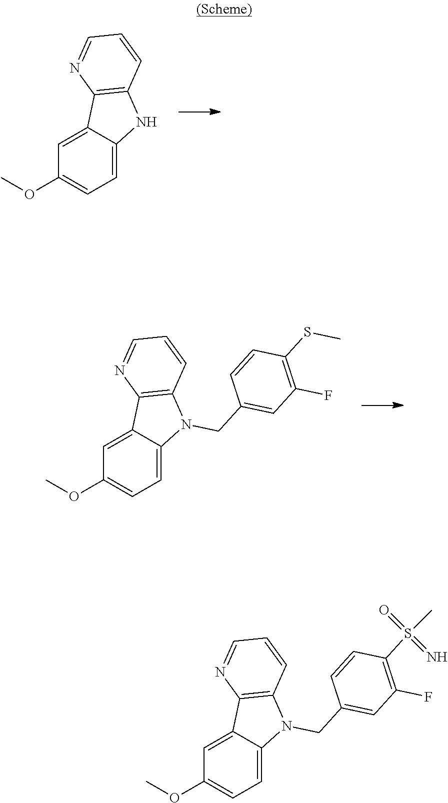

### (Step 1) Preparation of 5-(3-fluoro-4-(methylthio)benzyl)-8-methoxy-5H-pyrido[3,2-b]indole

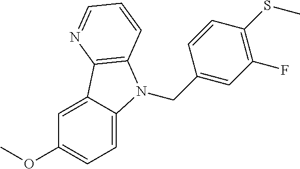

The compound prepared in (Step 3) of Example 5 (40.0 mg, 0.20 mmol) was dissolved in N,N-dimethylformamide (1.00 mL), and then cooled to 0° C., and 60% sodium hydride (10.5 mg, 0.26 mmol) was added to the mixture and stirred for 30 minutes. Intermediate 15 (61.7 mg, 0.26 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow solid. (30.0 mg, 42%) MS m/z: 353 [M+1]^{+ 1}H NMR (400 MHz, CDCl₃) δ ppm: 8.56 (s, 1H), 7.90 (s, 1H), 7.65-7.54 (m, 1H), 7.35-7.27 (m, 2H), 7.19-7.13 (m, 2H), 6.81 (m, 2H), 5.44 (s, 2H), 3.95 (s, 3H), 2.41 (s, 3H).

### (Step 2) Preparation of (2-fluoro-4-((8-methoxy-5H-pyrido[3,2-b]indol-5-yl)methyl)phenyl)(imino)(methyl)-l6-sulfanone (Compound 20)

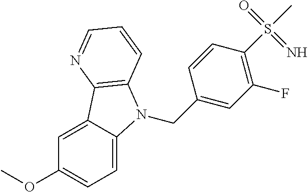 [SMILES: COc1ccc2c(c1)c1ncccc1n2Cc1ccc(S(C)(=N)=O)c(F)c1]

The compound prepared in (Step 1) above (30.0 mg, 0.09 mmol) was dissolved in ethanol (2.00 mL), and then iodobenzene diacetate (79.5 mg, 0.25 mmol) and ammonium acetate (24.9 mg, 0.32 mmol) were added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (25.4 mg, 99%) MS m/z: 384 [M+1]^{+ 1}H NMR (400 MHz, DMSO-d₆) δ ppm: 8.60 (s, 1H), 8.31 (s, 1H), 7.81 (s, 1H), 7.76 (s, 1H), 7.69 (s, 1H), 7.61 (s, 1H), 7.30 (s, 1H), 7.21 (d, J=10.1 Hz, 1H), 7.09 (s, 1H), 5.85 (s, 2H), 3.89 (s, 3H), 3.15 (s, 3H).

### [Example 21] Preparation of 4-((8-chloro-5H-pyrido[3,2-b]indol-5-yl)methyl benzenesulfonamide (Compound 21)

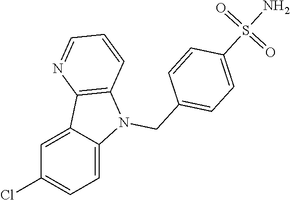 [SMILES: NS(=O)(=O)c1ccc(Cn2c3ccc(Cl)cc3c3ncccc32)cc1]

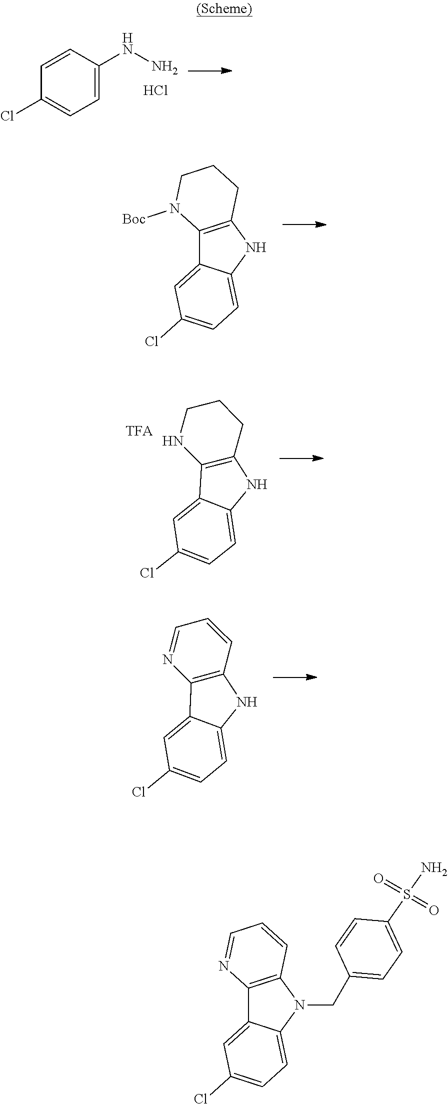 [SMILES: NNc1ccc(Cl)cc1 ; Cl ; CC(C)(C)OC(=O)N1CCCc2[nH]c3ccc(Cl)cc3c21 ; Clc1ccc2[nH]c3c(c2c1)NCCC3 ; Clc1ccc2[nH]c3cccnc3c2c1 ; NS(=O)(=O)c1ccc(Cn2c3ccc(Cl)cc3c3ncccc32)cc1]

### (Step 1) Preparation of tert-butyl 8-chloro-2,3,4,5-tetrahydro-1H-pyrido[3,2-b]indole-1-carboxylate

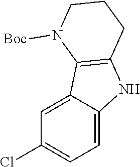 [SMILES: CC(C)(C)OC(=O)N1CCCc2[nH]c3ccc(Cl)cc3c21]

4-Chlorophenylhydrazine-hydrochloride (500 mg, 2.79 mmol) was dissolved in toluene (22.0 mL), and then tert-butyl-3-oxopiperidine-1-carboxylate (668 mg, 3.35 mmol) and propanephosphonic anhydride (0.34 mL, 0.56 mmol) were added to the mixture sequentially. The reaction mixture was stirred at 90° C. for 4 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (350 mg, 34%) MS m/z: 307 [M+1]^{+ 1}H NMR (400 MHz, CDCl₃) δ 7.78 (s, 1H), 7.64 (s, 1H), 7.15-7.09 (m, 1H), 7.07-7.03 (m, 1H), 3.75 (dt, J=7.8, 2.2 Hz, 2H), 2.85-2.77 (m, 2H), 2.11-2.00 (m, 2H), 1.54 (s, 9H).

### (Step 2) Preparation of 8-chloro-2,3,4,5-tetrahydro-1H-pyrido[3,2-b]indole-trifluoroacetic acid

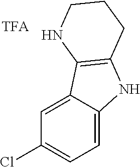 [SMILES: Clc1ccc2[nH]c3c(c2c1)NCCC3 ; "TFA"]

The compound prepared in (Step 1) above (350 mg, 1.69 mmol) was dissolved in methylene chloride (4.00 mL), and then trifluoroacetic acid (8.00 mL) was added to the mixture. The reaction mixture was stirred at room temperature for 4 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a red solid. (245 mg, 70%) MS m/z: 207 [M+1]⁺

### (Step 3) Preparation of 8-chloro-5H-pyrido[3,2-b]indole

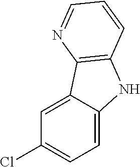 [SMILES: Clc1ccc2[nH]c3cccnc3c2c1]

The compound prepared in (Step 2) above (245 mg, 1.18 mmol) was dissolved in dimethyl sulfoxide (5.00 mL), and then stirred at 90° C. for 12 hours under nitrogen. After cooling to room temperature, distilled water and an aqueous sodium bicarbonate solution were added, and extracted twice with ethyl acetate. The residue was purified by preparative high performance liquid chromatography (prep-HPLC, acetonitrile:distilled water=5:95 to 100:0) to obtain the title compound as a yellow solid. (51.0 mg, 22%) MS m/z: 203 [M+1]⁺

### (Step 4) Preparation of 4-((8-chloro-5H-pyrido[3,2-b]indol-5-yl)methyl)benzenesulfonamide (Compound 21)

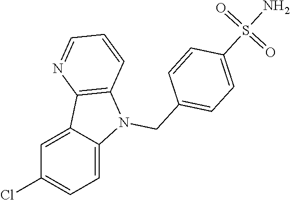 [SMILES: NS(=O)(=O)c1ccc(Cn2c3ccc(Cl)cc3c3ncccc32)cc1]

The compound prepared in (Step 3) above (20.0 mg, 0.10 mmol) was dissolved in N,N-dimethylformamide (1.00 mL), and then cooled to 0° C., and 60% sodium hydride (11.8 mg, 0.30 mmol) was added to the mixture and stirred for 30 minutes. Intermediate 1 (32.0 mg, 0.13 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (3.60 mg, 10%) MS m/z: 372 [M+1]^{+ 1}H NMR (400 MHz, CD₃OD) δ 8.66 (dd, J=5.3, 1.3 Hz, 1H), 8.43 (dd, J=8.4, 1.2 Hz, 1H), 8.39 (dd, J=1.9, 0.8 Hz, 1H), 7.85-7.80 (m, 2H), 7.78 (dd, J=8.5, 5.3 Hz, 1H), 7.74-7.65 (m, 2H), 7.35-7.28 (m, 2H), 5.87 (s, 2H).

### [Example 22] Preparation of 4-((8-(trifluoromethoxy)-5H-pyrido[3,2-b]indol-5-yl)methyl benzenesulfonamide (Compound 22)

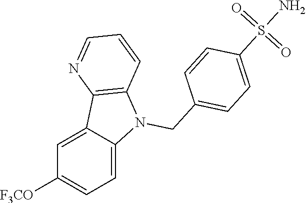 [SMILES: NS(=O)(=O)c1ccc(Cn2c3ccc(OC(F)(F)F)cc3c3ncccc32)cc1]

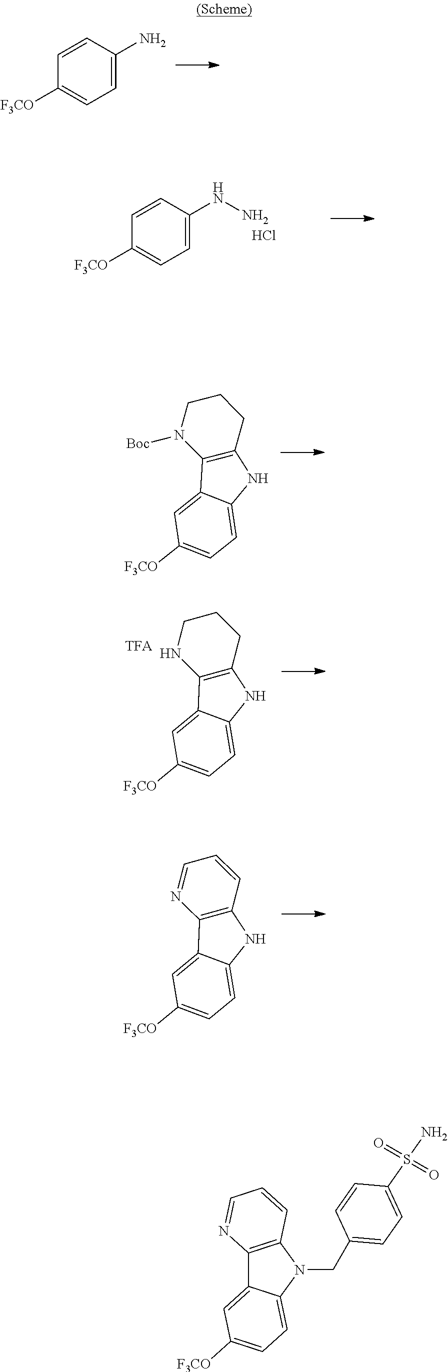

### (Step 1) Preparation of (4-(trifluoromethoxy)phenyl) hydrazine-Hydrochloride

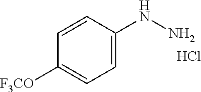 [SMILES: NNc1ccc(OC(F)(F)F)cc1 ; Cl]

4-(Trifluoromethoxy) aniline (800 mg, 4.52 mmol) was dissolved in 6N HCl (9.00 mL), and then sodium nitrite (467 mg, 6.78 mmol) dissolved in a saturated saline solution (3.00 mL) was slowly added to the mixture at −20° C. The reaction mixture was stirred at −20° C. for 30 minutes under nitrogen, and then tin (II) chloride dihydrate (3.60 g, 15.8 mmol) dissolved in 2.00 mL of conc. HCl was added slowly. After stirring at 0° C. for 2 hours, a mixed solution of ethanol:ethyl ether=3:7 was added to the reaction mixture, and further stirred for 1 hour. The resulting solid was filtered, washed with ethyl ether, and then dried to obtain the title compound (800 mg, 81%) as a white solid. MS m/z: 193 [M+1]⁺

### (Step 2) Preparation of tert-butyl 8-(trifluoromethoxy)-2,3,4,5-tetrahydro-1H-pyrido[3,2-b]indole-1-carboxylate

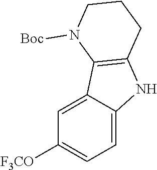 [SMILES: CC(C)(C)OC(=O)N1CCCc2[nH]c3ccc(OC(F)(F)F)cc3c21]

The compound prepared in (Step 1) above (400 mg, 2.08 mmol) was dissolved in toluene (8.00 mL), and then tert-butyl-3-oxopiperidine-1-carboxylate (456 mg, 2.30 mmol) and propanephosphonic anhydride (0.30 mL, 0.42 mmol) were added to the mixture sequentially. The reaction mixture was stirred at 90° C. for 4 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (137 mg, 18%) MS m/z: 357 [M+1]⁺

### (Step 3) Preparation of 8-(trifluoromethoxy)-2,3,4,5-tetrahydro-1H-pyrido[3,2-b]indole-trifluoroacetic acid

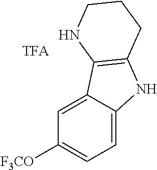 [SMILES: FC(F)(F)Oc1ccc2[nH]c3c(c2c1)NCCC3 ; "TFA"]

The compound prepared in (Step 2) above (137 mg, 0.38 mmol) was dissolved in methylene chloride (3.00 mL), and then trifluoroacetic acid (3.00 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (98.0 mg, 99%) MS m/z: 257 [M+1]⁺

### (Step 4) Preparation of 8-(trifluoromethoxy)-5H-pyrido[3,2-b]indole

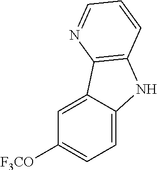 [SMILES: FC(F)(F)Oc1ccc2[nH]c3cccnc3c2c1]

The compound prepared in (Step 3) above (98.0 mg, 0.38 mmol) was dissolved in dimethyl sulfoxide (2.00 mL), and then stirred at 90° C. for 12 hours under nitrogen. After cooling to room temperature, distilled water and an aqueous sodium bicarbonate solution were added, and extracted twice with ethyl acetate. The residue was purified by preparative high performance liquid chromatography (prep-HPLC, acetonitrile:distilled water=5:95 to 100:0) to obtain the title compound as a yellow solid. (48.0 mg, 50%) MS m/z: 253 [M+1]⁺

### (Step 5) Preparation of 4-((8-(trifluoromethoxy)-5H-pyrido[3,2-b]indol-5-yl)methyl)benzenesulfonamide (Compound 22)

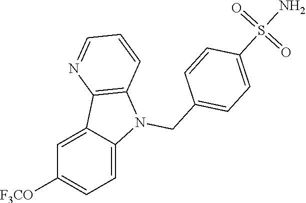 [SMILES: NS(=O)(=O)c1ccc(Cn2c3ccc(OC(F)(F)F)cc3c3ncccc32)cc1]

The compound prepared in (Step 4) above (10.0 mg, 0.04 mmol) was dissolved in N,N-dimethylformamide (1.00 mL), and then cooled to 0° C., and 60% sodium hydride (5.00 mg, 0.12 mmol) was added to the mixture and stirred for 30 minutes. Intermediate 1 (14.5 mg, 0.06 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (2.10 mg, 13%) MS m/z: 422 [M+1]^{+ 1}H NMR (400 MHz, CD₃OD) δ 8.65 (s, 1H), 8.34 (d, J=11.5 Hz, 2H), 7.86-7.70 (m, 4H), 7.60 (d, J=9.2 Hz, 1H), 7.36-7.29 (m, 2H), 5.88 (d, J=3.4 Hz, 2H).

### [Example 23] Preparation of imino(methyl) (4-((6-methyl-9H-pyrido[3,4-b]indol-9-yl)methyl)phenyl)-l6-sulfanone (Compound 23)

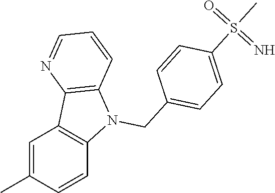 [SMILES: Cc1ccc2c(c1)c1ncccc1n2Cc1ccc(S(C)(=N)=O)cc1]

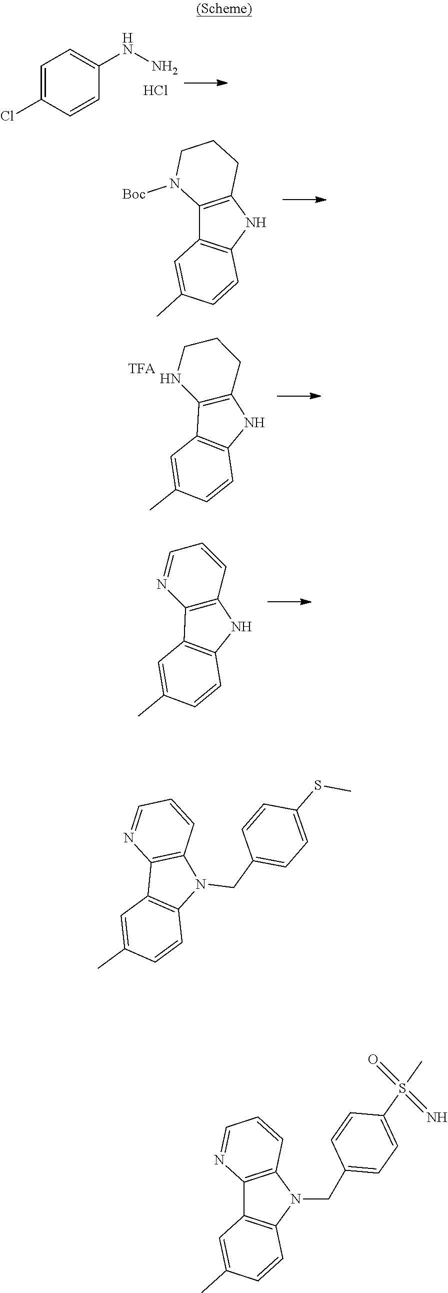

### (Step 1) Preparation of tert-butyl 8-methyl-2,3,4,5-tetrahydro-1H-pyrido[3,2-b]indole-1-carboxylate

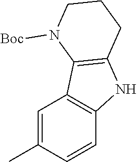 [SMILES: Cc1ccc2[nH]c3c(c2c1)N(C(=O)OC(C)(C)C)CCC3]

4-Methylphenylhydrazine-hydrochloride (1.00 g, 6.30 mmol) was dissolved in toluene (30.0 mL), and then tert-butyl-3-oxopiperidine-1-carboxylate (1.03 g, 6.30 mmol) and propanephosphonic anhydride (0.80 mL, 1.26 mmol) were added to the mixture sequentially. The reaction mixture was stirred at 90° C. for 4 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:2) to obtain the title compound as a yellow solid. (630 mg, 35%) MS m/z: 287 [M+1]^{+ 1}H NMR (400 MHz, CDCl₃) δ 7.58 (s, 1H), 7.44 (s, 1H), 7.10 (t, J=10.4 Hz, 1H), 6.93 (d, J=8.2 Hz, 1H), 3.79-3.71 (m, 2H), 2.80 (t, J=6.7 Hz, 2H), 2.43 (s, 3H), 2.11-2.00 (m, 2H), 1.54 (s, 9H).

### (Step 2) Preparation of 8-methyl-2,3,4,5-tetrahydro-1H-pyrido[3,2-b]indole-trifluoroacetic acid

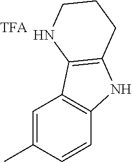

The compound prepared in (Step 1) above (280 mg, 0.98 mmol) was dissolved in methylene chloride (4.00 mL), and then trifluoroacetic acid (3.00 mL) was added to the mixture. The reaction mixture was stirred at room temperature for 3 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a red solid. (130 mg, 71%) MS m/z: 187 [M+1]⁺

### (Step 3) Preparation of 8-methyl-5H-pyrido[3,2-b]indole

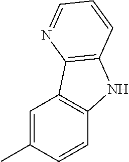

The compound prepared in (Step 2) above (130 mg, 0.70 mmol) was dissolved in toluene (3.00 mL), and then Pd/C (50 wt %, 65.0 mg) was added to the mixture. The reaction mixture was stirred at 100° C. for 15 hours. It was cooled to room temperature, and precipitation was removed by filtration using Celite, and then the filtrate was concentrated under reduced pressure. The residue was purified by preparative high performance liquid chromatography (prep-HPLC, acetonitrile:distilled water=5:95 to 100:0) to obtain the title compound as a green solid. (100 mg, 79%) MS m/z: 183 [M+1]⁺

### (Step 4) Preparation of 8-methyl-5-(4-(methylthio)benzyl)-5H-pyrido[3,2-b]indole

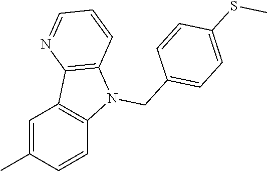

The compound prepared in (Step 3) above (27.0 mg, 0.15 mmol) was dissolved in N,N-dimethylformamide (1.00 mL), and then cooled to 0° C., and 60% sodium hydride (17.8 mg, 0.45 mmol) was added to the mixture and stirred for 30 minutes. Intermediate 6 (32.1 mg, 0.15 mmol) dissolved in N,N-dimethylformamide (0.50 mL) was slowly added to the reaction mixture, and then stirred at 0° C. for 3 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:3) to obtain the title compound as a yellow solid. (30.0 mg, 64%) MS m/z: 319 [M+1]⁺

### (Step 5) Preparation of imino(methyl) (4-((8-methyl-5H-pyrido[3,2-b]indol-5-yl)methyl)phenyl)-l6-sulfanone (Compound 23)

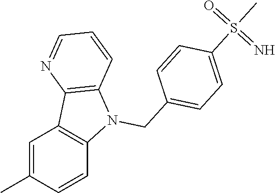 [SMILES: Cc1ccc2c(c1)c1ncccc1n2Cc1ccc(S(C)(=N)=O)cc1]

The compound prepared in (Step 4) above (30.0 mg, 0.09 mmol) was dissolved in ethanol (1.00 mL), and then iodobenzene diacetate (88.0 mg, 0.27 mmol) and ammonium acetate (28.3 mg, 0.37 mmol) were added to the mixture. The reaction mixture was stirred at room temperature for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (15.0 mg, 46%) MS m/z: 350 [M+1]^{+ 1}H NMR (400 MHz, CD₃OD) δ 8.69 (d, J=5.5 Hz, 1H), 8.65 (d, J=8.4 Hz, 1H), 8.24 (s, 1H), 8.04 (d, J=8.5 Hz, 2H), 7.91 (dd, J=8.5, 5.6 Hz, 1H), 7.66 (s, 2H), 7.51 (d, J=8.4 Hz, 2H), 6.00 (s, 2H), 3.54 (s, 3H), 2.59 (s, 3H).

### [Example 24] Preparation of (4-((8-ethoxy-5H-pyrido[3,2-b]indol-5-yl)methyl)phenyl)(imino)(methyl)-l6-sulfanone (Compound 24)

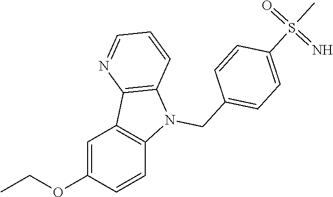 [SMILES: CCOc1ccc2c(c1)c1ncccc1n2Cc1ccc(S(C)(=N)=O)cc1]

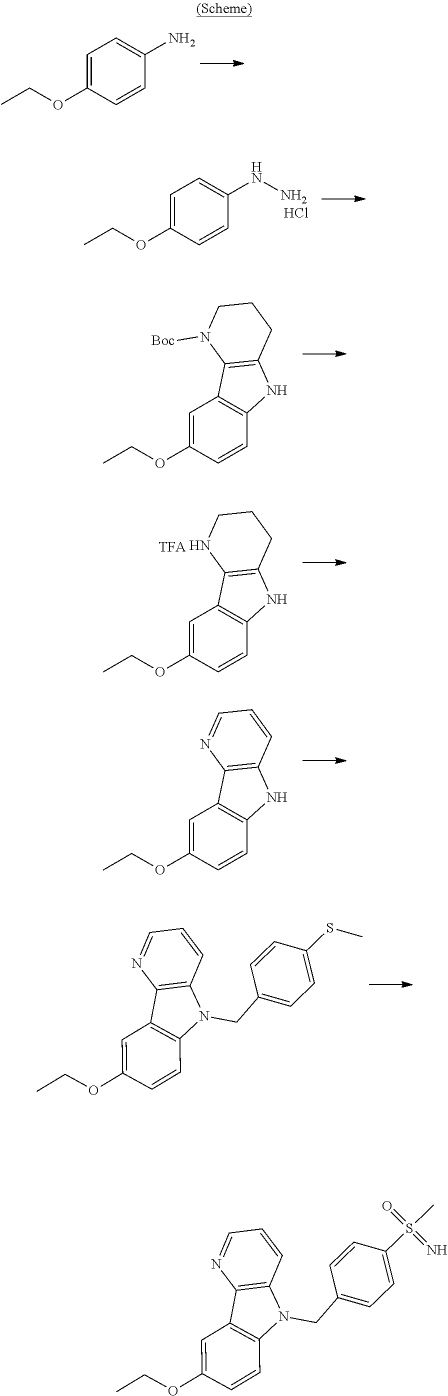

### (Step 1) Preparation of (4-ethoxyphenyl) hydrazine-hydrochloride

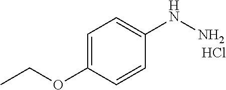

4-Ethoxyaniline (840 mg, 6.12 mmol) was dissolved in 6N HCl (5.00 mL), and then sodium nitrite (592 mg, 8.57 mmol) dissolved in 2.00 mL of a saturated saline solution was slowly added to the mixture at −20° C. The reaction mixture was stirred at −20° C. for 30 minutes under nitrogen, and then tin (II) chloride dihydrate (2.30 g, 12.3 mmol) dissolved in conc. HCl (2.00 mL) was added slowly. After stirring at 0° C. for 1 hour, a mixed solution of ethanol:ethyl ether=3:7 was added to the reaction mixture, and further stirred for 1 hour. The resulting solid was filtered, washed with ethyl ether, and then dried to obtain the title compound (530 mg, 57%) as a pale pink solid. MS m/z: 189 [M+1]⁺

### (Step 2) Preparation of tert-butyl 8-ethoxy-2,3,4,5-tetrahydro-1H-pyrido[3,2-b]indole-1-carboxylate

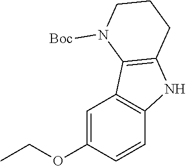 [SMILES: CCOc1ccc2[nH]c3c(c2c1)N(C(=O)OC(C)(C)C)CCC3]

The compound prepared in (Step 1) above (500 mg, 2.65 mmol) was dissolved in toluene (10.0 mL), and then tert-butyl-3-oxopiperidine-1-carboxylate (528 mg, 2.65 mmol) and propanephosphonic anhydride (0.33 mL, 0.53 mmol) were added to the mixture sequentially. The reaction mixture was stirred at 90° C. for 4 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (101 mg, 12%) MS m/z: 317 [M+1]⁺

### (Step 3) Preparation of 8-ethoxy-2,3,4,5-tetrahydro-1H-pyrido[3,2-b]indole-trifluoroacetic acid

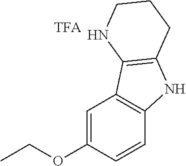 [SMILES: CCOc1ccc2[nH]c3c(c2c1)NCCC3]

The compound prepared in (Step 2) above (100 mg, 0.32 mmol) was dissolved in methylene chloride (2.00 mL), and then trifluoroacetic acid (1.00 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a red solid. (68.5 mg) MS m/z: 217 [M+1]⁺

### (Step 4) Preparation of 8-ethoxy-5H-pyrido[3,2-b]indole

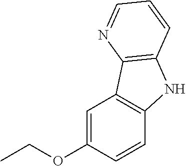 [SMILES: CCOc1ccc2[nH]c3cccnc3c2c1]

The compound prepared in (Step 3) above (68.5 mg, 0.32 mmol) was dissolved in toluene (2.00 mL), and then Pd/C (50 wt %, 34.0 mg) was added to the mixture. The mixture was stirred at 100° C. for 15 hours under nitrogen. It was cooled to room temperature, and precipitation was removed by filtration using Celite, and then the filtrate was concentrated under reduced pressure. The residue was purified by preparative high performance liquid chromatography (prep-HPLC, acetonitrile:distilled water=5:95 to 100:0) to obtain the title compound as a green solid. (65.0 mg, 97%) MS m/z: 213 [M+1]⁺

### (Step 5) Preparation of 8-ethoxy-5-(4-(methylthio)benzyl)-5H-pyrido[3,2-b]indole

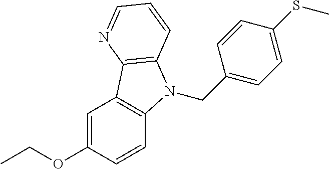 [SMILES: CCOc1ccc2c(c1)c1ncccc1n2Cc1ccc(SC)cc1]

The compound prepared in (Step 4) above (31.0 mg, 0.15 mmol) was dissolved in N,N-dimethylformamide (1.00 mL), and then cooled to 0° C., and 60% sodium hydride (12.0 mg, 0.29 mmol) was added to the mixture and stirred for 30 minutes. Intermediate 6 (31.7 mg, 0.15 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (13.0 mg, 26%) MS m/z: 349 [M+1]⁺

### (Step 6) Preparation of (4-((8-ethoxy-5H-pyrido[3,2-b]indol-5-yl)methyl)phenyl)(imino)(methyl)-l6-sulfanone (Compound 24)

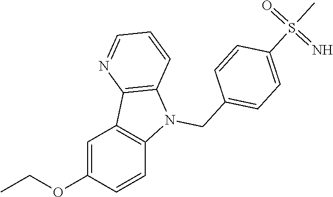 [SMILES: CCOc1ccc2c(c1)c1ncccc1n2Cc1ccc(S(C)(=N)=O)cc1]

The compound prepared in (Step 5) above (13.0 mg, 0.04 mmol) was dissolved in ethanol (1.00 mL), and then iodobenzene diacetate (34.8 mg, 0.11 mmol) and ammonium acetate (10.9 mg, 0.14 mmol) were added to the mixture. The reaction mixture was stirred at room temperature for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (8.20 mg, 55%) MS m/z: 380 [M+1]^{+ 1}H NMR (400 MHz, CD₃OD) δ 8.66 (d, J=5.4 Hz, 1H), 8.57 (d, J=8.5 Hz, 1H), 8.02 (d, J=8.4 Hz, 2H), 7.89 (d, J=2.3 Hz, 1H), 7.84 (dd, J=8.6, 5.4 Hz, 1H), 7.66 (d, J=9.1 Hz, 1H), 7.48 (d, J=8.4 Hz, 2H), 7.41 (dd, J=9.1, 2.5 Hz, 1H), 5.97 (s, 2H), 4.20 (q, J=7.0 Hz, 2H), 3.45 (s, 3H), 1.51-1.43 (m, 3H).

### [Example 25] Preparation of 4-((6-fluoro-8-methoxy-5H-pyrido[3,2-b]indol-5-yl)methyl)benzenesulfonamide (Compound 25)

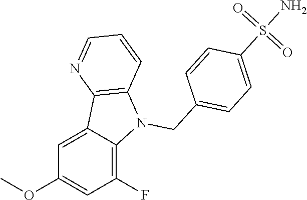 [SMILES: COc1cc(F)c2c(c1)c1ncccc1n2Cc1ccc(S(N)(=O)=O)cc1]

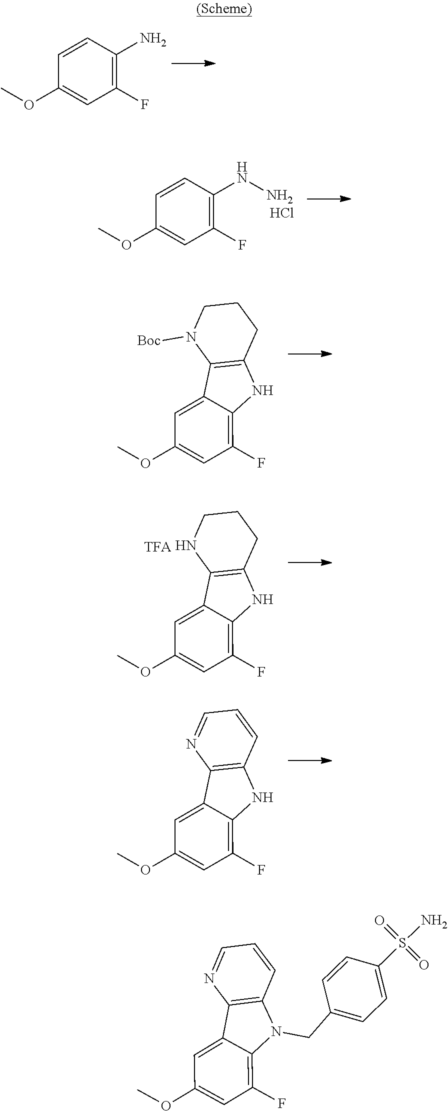

### (Step 1) Preparation of (2-fluoro-4-methoxyphenyl) hydrazine-hydrochloride

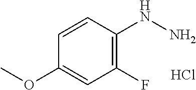

2-Fluoro 4-methoxyaniline (1.00 g, 7.09 mmol) was dissolved in 1:1 mixed solution (12.0 mL) of conc. HCl:distilled water, and then sodium nitrite (538 mg, 7.79 mmol) dissolved in 5.00 mL of a saturated saline solution was slowly added to the mixture at −20° C. The reaction mixture was stirred at −20° C. for 30 minutes under nitrogen, and then tin (II) chloride dihydrate (3.18 g, 14.2 mmol) dissolved in 4.00 mL of conc. HCl was added slowly. After stirring at 0° C. for 1 hour, a mixed solution of ethanol:ethyl ether=3:7 was added to the reaction mixture, and further stirred for 2 hours. The resulting solid was filtered, washed with ethyl ether, and then dried to obtain the title compound (582 mg, 53%) as a gray solid. MS m/z: 157 [M+1]⁺

### (Step 2) Preparation of tert-butyl 6-fluoro-8-methoxy-2,3,4,5-tetrahydro-1H-pyrido[3,2-b]indole-1-carboxylate

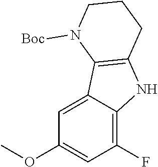 [SMILES: COc1cc(F)c2[nH]c3c(c2c1)N(C(=O)OC(C)(C)C)CCC3]

The compound prepared in (Step 1) above (100 mg, 0.52 mmol) was dissolved in toluene (63.0 mL), and then tert-butyl-3-oxopiperidine-1-carboxylate (124 mg, 0.62 mmol) and propanephosphonic anhydride (53.0 μl, 0.10 mmol) were added to the mixture sequentially. The reaction mixture was stirred at 90° C. for 6 hours under nitrogen. After cooling to room extracted twice with ethyl acetate. The organic layers were dried over anhydrous sodium sulfate, and then concentrated under reduced pressure. The residue was purified by silica gel column chromatography (ethyl acetate:n-hexane=1:2) to obtain the title compound as a yellow solid. (34.9 mg, 21%) MS m/z: 321 [M+1]⁺

### (Step 3) Preparation of 6-fluoro-8-methoxy-2,3,4,5-tetrahydro-1H-pyrido[3,2-b]indole-trifluoroacetic acid

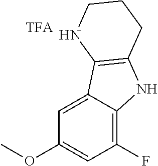 [SMILES: COc1cc(F)c2[nH]c3c(c2c1)NCCC3]

The compound prepared in (Step 2) above (34.9 mg, 0.11 mmol) was dissolved in methylene chloride (1.10 mL), and then trifluoroacetic acid (0.50 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (34.0 mg, 100%) MS m/z: 221 [M+1]⁺

### (Step 4) Preparation of 6-fluoro-8-methoxy-5H-pyrido[3,2-b]indole

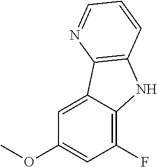 [SMILES: COc1cc(F)c2[nH]c3cccnc3c2c1]

The compound prepared in (Step 3) above (34.0 mg, 0.11 mmol) was dissolved in dimethyl sulfoxide (1.00 mL), and then stirred at 90° C. for 12 hours under nitrogen. After cooling to room temperature, distilled water and an aqueous sodium bicarbonate solution were added, and extracted twice with ethyl acetate. The residue was purified by preparative high performance liquid chromatography (prep-HPLC, acetonitrile:distilled water=5:95 to 100:0) to obtain the title compound as a yellow solid. (10.6 mg, 45%) MS m/z: 217 [M+1]⁺

### (Step 5) Preparation of 4-((6-fluoro-8-methoxy-5H-pyrido[3,2-b]indol-5-yl)methyl)benzenesulfonamide (Compound 25)

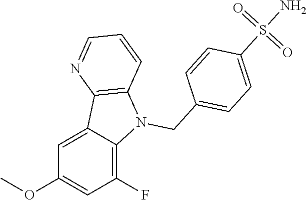 [SMILES: COc1cc(F)c2c(c1)c1ncccc1n2Cc1ccc(S(N)(=O)=O)cc1]

The compound prepared in (Step 4) above (10.6 mg, 0.05 mmol) was dissolved in N,N-dimethylformamide (0.50 mL), and then cooled to 0° C., and 60% sodium hydride (3.9 mg, 0.10 mmol) was added to the mixture and stirred for 30 minutes. Intermediate 1 (14.7 mg, 0.06 mmol) dissolved in N,N-dimethylformamide (0.20 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (7.00 mg, 37%) MS m/z: 386 [M+1]^{+ 1}H NMR (400 MHz, CD₃OD) δ 8.61 (s, 1H), 8.38 (d, J=8.3 Hz, 1H), 7.80 (d, J=7.6 Hz, 2H), 7.72 (d, J=9.5 Hz, 2H), 7.31-7.23 (m, 2H), 7.13 (d, J=14.8 Hz, 1H), 5.90 (s, 2H), 3.94 (s, 3H).

### [Example 26] Preparation of 4-((6-methoxy-8-methyl-9H-pyrido[3,4-b]indol-9-yl)methyl)benzenesulfonamide (Compound 26)

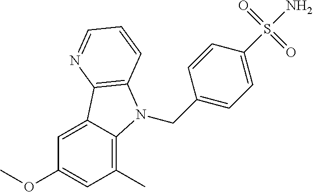 [SMILES: COc1cc(C)c2c(c1)c1ncccc1n2Cc1ccc(S(N)(=O)=O)cc1]

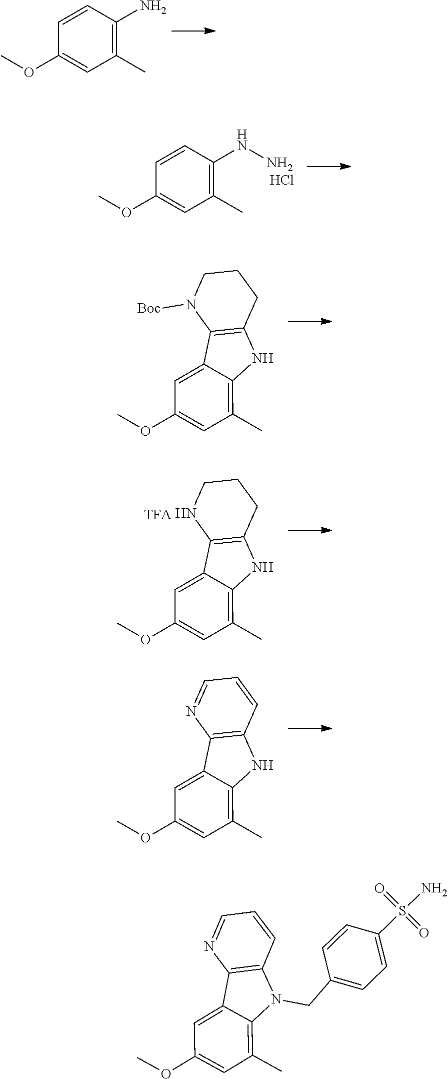

### (Step 1) Preparation of (4-methoxy-2-methylphenyl) hydrazine-hydrochloride

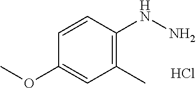

4-Methoxy-2-methylaniline (2.00 g, 14.6 mmol) was dissolved in 1:1 mixed solution (20.0 mL) of conc. HCl:distilled water, and then sodium nitrite (1.11 g, 16.0 mmol) dissolved in 5.00 mL of a saturated saline solution was slowly added to the mixture at −20° C. The reaction mixture was stirred at −20° C. for 30 minutes under nitrogen, and then tin (II) chloride dihydrate (6.58 g, 29.2 mmol) dissolved in 5.00 mL of conc. HCl was added slowly. After stirring at 0° C. for 1 hour, a mixed solution of ethanol:ethyl ether=3:7 was added to the reaction mixture, and further stirred for 2 hours. The resulting solid was filtered, washed with ethyl ether, and then dried to obtain the title compound (1.85 g, 83%) as a gray solid. MS m/z: 151 [M+1]⁺

### (Step 2) Preparation of tert-butyl 8-methoxy-6-methyl-2,3,4,5-tetrahydro-1H-pyrido[3,2-b]indole-1-carboxylate

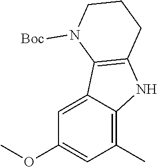

The compound prepared in (Step 1) above (300 mg, 1.59 mmol) was dissolved in toluene (15.9 mL), and then tert-butyl-3-oxopiperidine-1-carboxylate (380 mg, 1.91 mmol) and propanephosphonic anhydride (202 mL, 0.32 mmol) were added to the mixture sequentially. The reaction mixture was stirred at 90° C. for 6 hours under nitrogen. After cooling to room extracted twice with ethyl acetate. The organic layers were dried over anhydrous sodium sulfate, and then concentrated under reduced pressure. The residue was purified by silica gel column chromatography (ethyl acetate:n-hexane=1:2) to obtain the title compound as a yellow solid. (38.9 mg, 7%) MS m/z: 317 [M+1]⁺

### (Step 3) Preparation of 8-methoxy-6-methyl-2,3,4,5-tetrahydro-1H-pyrido[3,2-b]indole-trifluoroacetic acid

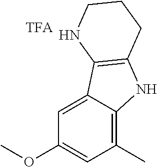

The compound prepared in (Step 2) above (38.9 mg, 0.123 mmol) was dissolved in methylene chloride (1.00 mL), and then trifluoroacetic acid (0.50 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (38.7 mg, 100%) MS m/z: 217 [M+1]⁺

### (Step 4) Preparation of 8-methoxy-6-methyl-5H-pyrido[3,2-b]indole

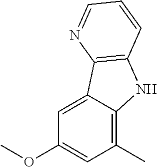

The compound prepared in (Step 3) above (38.7 mg, 0.12 mmol) was dissolved in dimethyl sulfoxide (0.20 mL), and then stirred at 90° C. for 12 hours under nitrogen. After cooling to room temperature, distilled water and an aqueous sodium bicarbonate solution were added, and extracted twice with ethyl acetate. The residue was purified by preparative high performance liquid chromatography (prep-HPLC, acetonitrile:distilled water=5:95 to 100:0) to obtain the title compound as a yellow solid. (13.5 mg, 52%) MS m/z: 213 [M+1]⁺

### (Step 5) Preparation of 4-((8-methoxy-6-methyl-5H-pyrido[3,2-b]indol-5-yl)methyl)benzenesulfonamide (Compound 26)

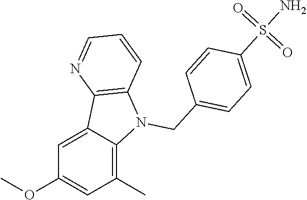 [SMILES: COc1cc(C)c2c(c1)c1ncccc1n2Cc1ccc(S(N)(=O)=O)cc1]

The compound prepared in (Step 4) above (13.5 mg, 0.06 mmol) was dissolved in N,N-dimethylformamide (0.60 mL), and then cooled to 0° C., and 60% sodium hydride (5.10 mg, 0.13 mmol) was added to the mixture and stirred for 30 minutes. Intermediate 1 (19.1 mg, 0.08 mmol) dissolved in N,N-dimethylformamide (0.10 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (3.9 mg, 16%) MS m/z: 382 [M+1]^{+ 1}H NMR (400 MHz, CD₃OD) δ 8.53 (s, 1H), 8.20 (s, 1H), 7.80 (d, J=7.6 Hz, 2H), 7.75 (s, 1H), 7.62 (s, 1H), 7.11-7.09 (m, 3H), 6.02 (s, 2H), 3.92 (s, 3H), 2.59 (s, 3H).

### [Example 27] Preparation of 4-((6-chloro-8-methoxy-5H-pyrido[3,2-b]indol-5-yl)methyl)benzenesulfonamide (Compound 27)

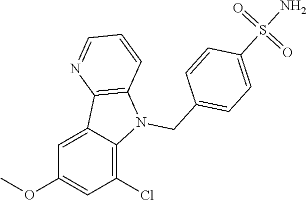 [SMILES: COc1cc(Cl)c2c(c1)c1ncccc1n2Cc1ccc(S(N)(=O)=O)cc1]

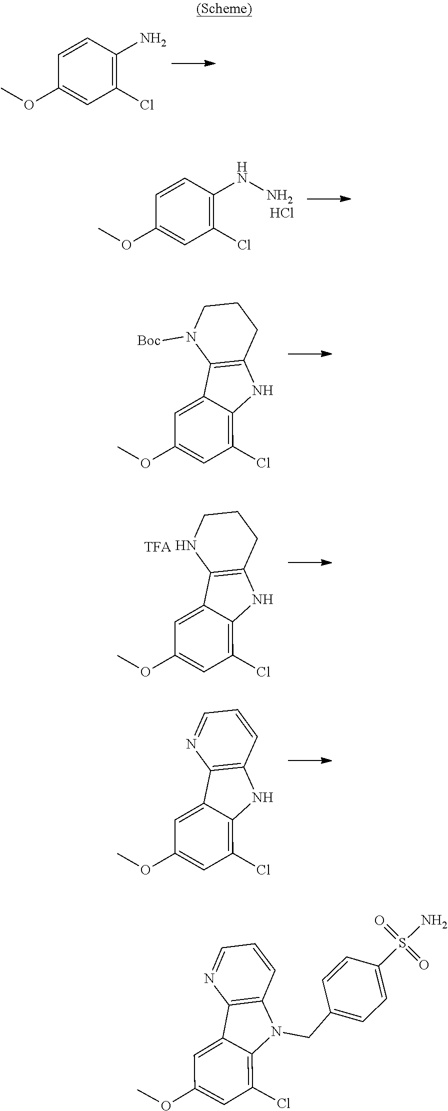

### (Step 1) Preparation of (2-chloro-4-methoxyphenyl) hydrazine-hydrochloride

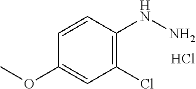

(2-Chloro-4-methoxyphenyl) hydrazine (800 mg, 5.08 mmol) was dissolved in 6N HCl (9.00 mL), and then sodium nitrite (525 mg, 7.61 mmol) dissolved in a saturated saline solution (3.00 mL) was slowly added to the mixture at −20° C. The reaction mixture was stirred at −20° C. for 30 minutes under nitrogen, and then tin (II) chloride dihydrate (4.00 g, 17.8 mmol) dissolved in 2.00 mL of conc. HCl was added slowly. After stirring at 0° C. for 1 hour, a mixed solution of ethanol:ethyl ether=3:7 was added to the reaction mixture, and further stirred for 1 hour. The resulting solid was filtered, washed with ethyl ether, and then dried to obtain the title compound (303 mg, 35%) as a white solid. MS m/z: 173 [M+1]⁺

### (Step 2) Preparation of tert-butyl 6-chloro-8-methoxy-2,3,4,5-tetrahydro-1H-pyrido[3,2-b]indole-1-carboxylate

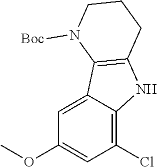 [SMILES: COc1cc(Cl)c2[nH]c3c(c2c1)N(C(=O)OC(C)(C)C)CCC3]

The compound prepared in (Step 1) above (300 mg, 1.74 mmol) was dissolved in toluene (7.00 mL), and then tert-butyl-3-oxopiperidine-1-carboxylate (492 mg, 2.09 mmol) and propanephosphonic anhydride (0.23 mL, 0.35 mmol) were added to the mixture sequentially. The reaction mixture was stirred at 90° C. for 6 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (90.0 mg, 15%) MS m/z: 337 [M+1]⁺

### (Step 3) Preparation of 6-chloro-8-methoxy-2,3,4,5-tetrahydro-1H-pyrido[3,2-b]indole-trifluoroacetic acid

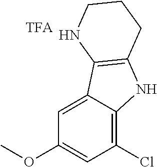 [SMILES: COc1cc(Cl)c2[nH]c3c(c2c1)NCCC3]

The compound prepared in (Step 2) above (90.0 mg, 0.27 mmol) was dissolved in methylene chloride (2.00 mL), and then trifluoroacetic acid (2.00 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (51.0 mg, 80%) MS m/z: 237 [M+1]⁺

### (Step 4) Preparation of 6-chloro-8-methoxy-5H-pyrido[3,2-b]indole

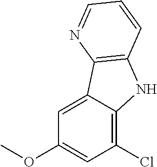 [SMILES: COc1cc(Cl)c2[nH]c3cccnc3c2c1]

The compound prepared in (Step 3) above (51.0 mg, 0.22 mmol) was dissolved in dimethyl sulfoxide (2.00 mL), and then stirred at 90° C. for 12 hours under nitrogen. After cooling to room temperature, distilled water and an aqueous sodium bicarbonate solution were added thereto, and extracted twice with ethyl acetate. The residue was purified by preparative high performance liquid chromatography (prep-HPLC, acetonitrile:distilled water=5:95 5:95 to 100:0) to obtain the title compound as a yellow solid. (21.0 mg, 41%) MS m/z: 233 [M+1]⁺

### (Step 5) Preparation of 4-((6-chloro-8-methoxy-5H-pyrido[3,2-b]indol-5-yl)methyl)benzenesulfonamide (Compound 27)

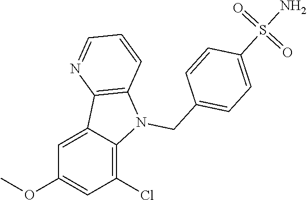 [SMILES: COc1cc(Cl)c2c(c1)c1ncccc1n2Cc1ccc(S(N)(=O)=O)cc1]

The compound prepared in (Step 4) above (11.0 mg, 0.05 mmol) was dissolved in N,N-dimethylformamide (0.50 mL), and then cooled to 0° C., and 60% sodium hydride (6.00 mg, 0.14 mmol) was added to the mixture and stirred for 30 minutes. Intermediate 1 (17.7 mg, 0.07 mmol) dissolved in N,N-dimethylformamide (0.50 mL) was slowly added to the reaction mixture, and then stirred at 0° C. for 2 hours under nitrogen. After completion of the reaction, distilled water was added thereto slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (1.60 mg, 8%) MS m/z: 402 [M+1]^{+ 1}H NMR (400 MHz, CD₃OD) δ 8.65 (s, 1H), 8.34 (d, J=8.5 Hz, 1H), 7.91 (s, 1H), 7.85-7.69 (m, 3H), 7.35 (s, 1H), 7.19 (q, J=2.8 Hz, 2H), 6.20 (s, 2H), 3.96 (t, J=2.4 Hz, 3H).

### [Example 28] Preparation of 4-((7,8-dimethoxy-5H-pyrido[3,2-b]indol-5-yl)methyl)benzenesulfonamide (Compound 28)

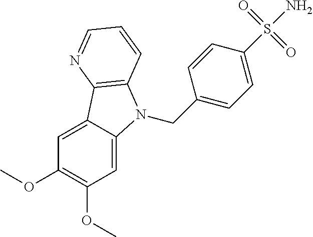 [SMILES: COc1cc2c3ncccc3n(Cc3ccc(S(N)(=O)=O)cc3)c2cc1OC]

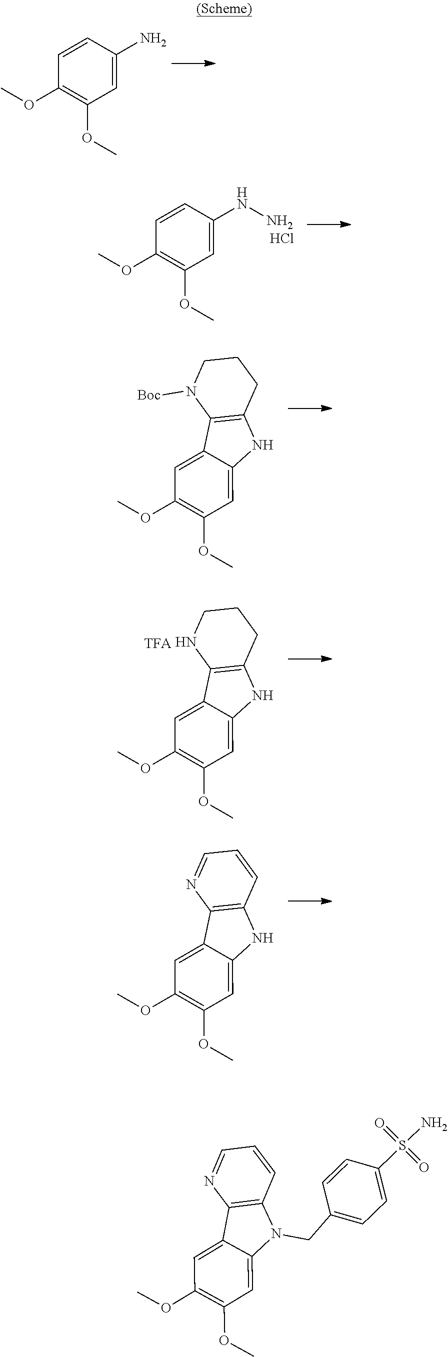

### (Step 1) Preparation of (3,4-dimethoxyphenyl) hydrazine-hydrochloride

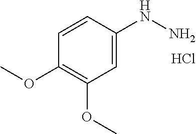

3,4-Dimethoxyaniline (1.00 g, 6.52 mmol) was dissolved in 9.00 mL of 6N HCl, and then sodium nitrite (675 mg, 6.79 mmol) dissolved in 2.00 mL of a saturated saline solution was slowly added to the mixture at −20° C. The reaction mixture was stirred at −20° C. for 30 minutes under nitrogen, and then tin (II) chloride dihydrate (4.42 g, 19.6 mmol) dissolved in 2.00 mL of conc. HCl was added slowly. After stirring at 0° C. for 1 hour, a mixed solution of ethanol:ethyl ether=3:7 was added to the reaction mixture, and further stirred for 1 hour. The resulting solid was filtered, washed with ethyl ether, and then dried to obtain the title compound (713 mg, 53%) as a white solid. MS m/z: 205 [M+1]^{+ 1}H NMR (400 MHz, DMSO-d₆) δ 10.24 (s, 3H), 7.02 (d, J=8.6 Hz, 1H), 6.98 (s, 1H), 6.70 (d, J=8.4 Hz, 1H), 3.88 (s, 3H), 3.84 (s, 3H)

### (Step 2) Preparation of tert-butyl 7,8-dimethoxy-2,3,4,5-tetrahydro-1H-pyrido[3,2-b]indole-1-carboxylate

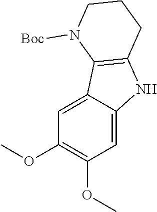 [SMILES: COc1cc2[nH]c3c(c2cc1OC)N(C(=O)OC(C)(C)C)CCC3]

The compound prepared in (Step 1) above (713 mg, 3.48 mmol) was dissolved in toluene (10.0 mL), and then tert-butyl-3-oxopiperidine-1-carboxylate (833 mg, 4.18 mmol) and propanephosphonic anhydride (0.35 mL, 0.69 mmol) were added to the mixture sequentially. The reaction mixture was stirred at 90° C. for 3 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=4:6) to obtain the title compound as a yellow solid. (492 mg, 42%) MS m/z: 333 [M+1]^{+ 1}H NMR (400 MHz, CDCl₃) δ 7.47 (s, 1H), 7.14 (s, 1H), 6.77 (s, 1H), 3.91 (dd, J=14.2, 2.4 Hz, 6H), 3.75 (d, J=5.3 Hz, 2H), 2.80 (s, 2H), 2.04 (d, J=2.6 Hz, 2H), 1.53 (d, J=2.1 Hz, 9H).

### (Step 3) Preparation of 7,8-dimethoxy-2,3,4,5-tetrahydro-1H-pyrido[3,2-b]indole-trifluoroacetic acid

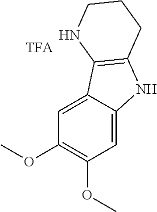 [SMILES: COc1cc2[nH]c3c(c2cc1OC)NCCC3]

The compound prepared in (Step 2) above (492 mg, 1.48 mmol) was dissolved in methylene chloride (5.00 mL), and then trifluoroacetic acid (2.00 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (512 mg, 100%) MS m/z: 233 [M+1]⁺

### (Step 4) Preparation of 7,8-dimethoxy-5H-pyrido[3,2-b]indole

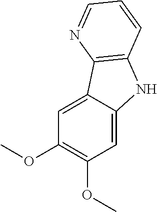 [SMILES: COc1cc2[nH]c3cccnc3c2cc1OC]

The compound prepared in (Step 3) above (512 mg, 1.48 mmol) was dissolved in dimethyl sulfoxide (1.00 mL), and then the reaction mixture was stirred at 80° C. for 24 hours under nitrogen. The reaction mixture was purified by preparative high performance liquid chromatography (prep-HPLC, acetonitrile:distilled water=5:95 to 100:0) to obtain the title compound as a yellow solid. (40.0 mg, 12%) MS m/z: 229 [M+1]⁺

### (Step 5) Preparation of 4-((7,8-dimethoxy-5H-pyrido[3,2-b]indol-5-yl)methyl)benzenesulfonamide (Compound 28)

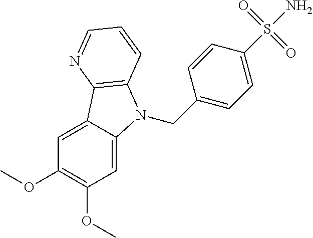 [SMILES: COc1cc2c3ncccc3n(Cc3ccc(S(N)(=O)=O)cc3)c2cc1OC]

The compound prepared in (Step 4) above (40.0 mg, 0.17 mmol) was dissolved in N,N-dimethylformamide (1.00 mL), and then cooled to 0° C., and 60% sodium hydride (14.0 mg, 0.35 mmol) was added to the mixture and stirred for 30 minutes. Intermediate 1 (65.0 mg, 0.26 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (1.00 mg, 1%) MS m/z: 398 [M+1]^{+ 1}H NMR (400 MHz, CD₃OD) δ 8.56 (s, 2H), 7.80 (m, 4H), 7.41-7.27 (m, 3H), 5.93 (d, J=2.6 Hz, 2H), 4.13-3.76 (m, 6H)

### [Example 29] Preparation of 4-((7-fluoro-8-methoxy-5H-pyrido[3,2-b]indol-5-yl)methyl)benzenesulfonamide (Compound 29)

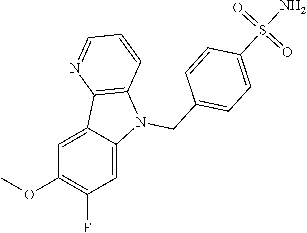 [SMILES: COc1cc2c3ncccc3n(Cc3ccc(S(N)(=O)=O)cc3)c2cc1F]

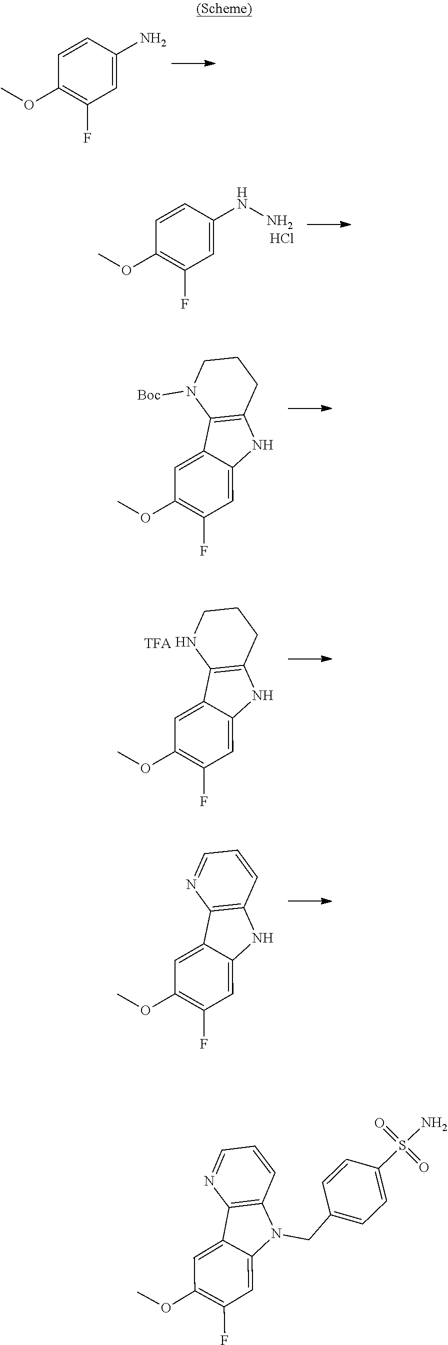

### (Step 1) Preparation of (3-fluoro-4-methoxyphenyl) hydrazine-hydrochloride

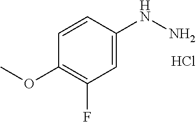

3-Fluoro-4-methoxyaniline (1.00 g, 7.08 mmol) was dissolved in 9.00 mL of 6N HCl, and then sodium nitrite (733 mg, 10.6 mmol) dissolved in 2.00 mL of a saturated saline solution was slowly added to the mixture at −20° C. The reaction mixture was stirred at −20° C. for 30 minutes under nitrogen, and then tin (II) chloride dihydrate (4.80 g, 21.3 mmol) dissolved in 2.00 mL of conc. HCl was added slowly. After stirring at 0° C. for 1 hour, a mixed solution of ethanol:ethyl ether=3:7 was added to the reaction mixture, and further stirred for 1 hour. The resulting solid was filtered, washed with ethyl ether, and then dried to obtain the title compound (1.20 g, 88%) as a white solid. MS m/z: 193 [M+1]^{+ 1}H NMR (400 MHz, DMSO-d₆) δ 10.07 (s, 3H), 8.08 (s, 1H), 7.12 (td, J=9.0, 5.3 Hz, 1H), 6.95 (dd, J=13.1, 2.7 Hz, 1H), 6.79 (d, J=9.4 Hz, 1H), 3.78 (s, 3H).

### (Step 2) Preparation of tert-butyl 7-fluoro-8-methoxy-2,3,4,5-tetrahydro-1H-pyrido[3,2-b]indole-1-carboxylate

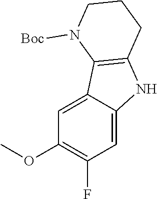 [SMILES: COc1cc2c3c([nH]c2cc1F)CCCN3C(=O)OC(C)(C)C]

The compound prepared in (Step 1) above (1.20 g, 6.22 mmol) was dissolved in toluene (10.0 mL), and then tert-butyl-3-oxopiperidine-1-carboxylate (1.49 mg, 7.47 mmol) and propanephosphonic anhydride (0.64 mL, 1.24 mmol) were added to the mixture sequentially. The reaction mixture was stirred at 90° C. for 3 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=2:8) to obtain the title compound as a yellow solid. (760 mg, 38%) MS m/z: 321 [M+1]^{+ 1}H NMR (400 MHz, CDCl₃) δ 7.53 (d, J=5.5 Hz, 1H), 7.21 (d, J=8.1 Hz, 1H), 6.98 (d, J=11.3 Hz, 1H), 3.93 (s, 3H), 3.80-3.71 (m, 2H), 2.81 (t, J=6.7 Hz, 2H), 2.11-1.99 (m, 2H), 1.56 (s, 9H)

### (Step 3) Preparation of 7-fluoro-8-methoxy-2,3,4,5-tetrahydro-1H-pyrido[3,2-b]indole-trifluoroacetic acid

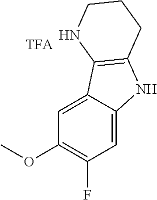 [SMILES: COc1cc2c3c([nH]c2cc1F)CCCN3]

The compound prepared in (Step 2) above (760 mg, 2.37 mmol) was dissolved in methylene chloride (5.00 mL), and then trifluoroacetic acid (2.00 mL) was added to the mixture. The reaction mixture was stirred at room temperature for 2 hours under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (792 mg, 99%) MS m/z: 221 [M+1]⁺

### (Step 4) Preparation of 7-fluoro-8-methoxy-5H-pyrido[3,2-b]indole

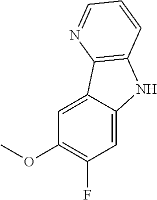 [SMILES: COc1cc2c(cc1F)[nH]c1cccnc12]

The compound prepared in (Step 3) above (392 mg, 2.37 mmol) was dissolved in toluene (15.0 mL), and then Pd/C (10 wt %, 370 mg) was added to the mixture. The reaction mixture was stirred at 120° C. for 25 hours under nitrogen. Precipitation was removed by filtration using Celite, and then the filtrate was concentrated under reduced pressure. The residue was purified by preparative high performance liquid chromatography (prep-HPLC, acetonitrile:distilled water=5:95 to 100:0) to obtain the title compound as a yellow solid. (370 mg, 72%) MS m/z: 217 [M+1]^{+ 1}H NMR (400 MHz, CDCl₃) δ 8.55 (d, J=4.4 Hz, 1H), 8.34, (bs, 1H), 7.89 (d, J=8.4 Hz, 1H), 7.72 (d, J=8.0 Hz, 1H), 7.31 (dd, J=4.8, 8.0 Hz, 1H), 7.22 (d, J=11.2 Hz, 1H), 3.97 (s, 3H).

### (Step 5) Preparation of 4-((7-fluoro-8-methoxy-5H-pyrido[3,2-b]indol-5-yl)methyl)benzenesulfonamide (Compound 29)

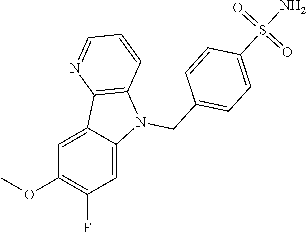 [SMILES: COc1cc2c3ncccc3n(Cc3ccc(S(N)(=O)=O)cc3)c2cc1F]

The compound prepared in (Step 4) above (80.0 mg, 0.37 mmol) was dissolved in N,N-dimethylformamide (1.00 mL), and then cooled to 0° C., and 60% sodium hydride (29.0 mg, 0.74 mmol) was added to the mixture and stirred for 30 minutes. Intermediate 1 (138 mg, 0.55 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (36.0 mg, 25%) MS m/z: 386 [M+1]^{+ 1}H NMR (400 MHz, CD₃OD) δ 8.65 (dd, J=5.5, 1.1 Hz, 1H), 8.55 (dd, J=8.4, 1.1 Hz, 1H), 8.03 (d, J=8.1 Hz, 1H), 7.85-7.79 (m, 3H), 7.63 (d, J=11.3 Hz, 1H), 7.38-7.28 (m, 2H), 5.86 (s, 2H), 4.04 (s, 3H).

### [Example 30] Preparation of (4-((7-fluoro-8-methoxy-5H-pyrido[3,2-b]indol-5-yl)methyl)phenyl)(imino)(methyl)-l6-sulfanone (Compound 30)

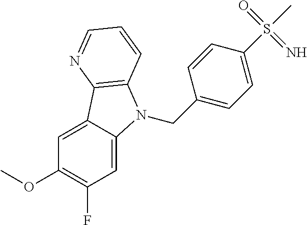 [SMILES: COc1cc2c3ncccc3n(Cc3ccc(S(C)(=N)=O)cc3)c2cc1F]

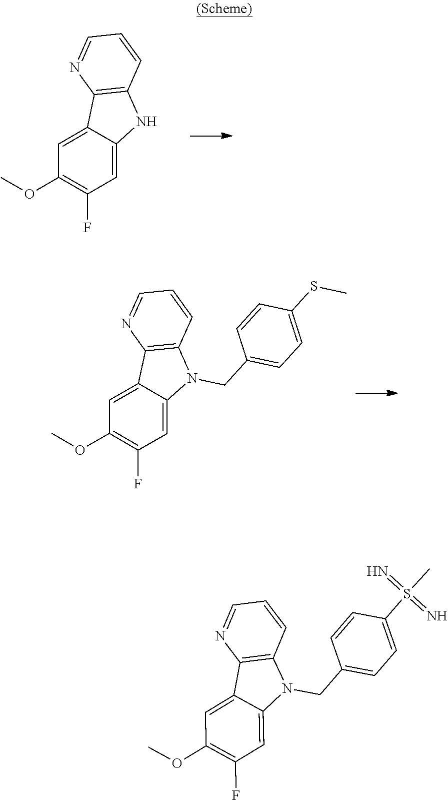

### (Step 1) Preparation of 7-fluoro-8-methoxy-5-(4-(methylthio)benzyl)-5H-pyrido[3,2-b]indole

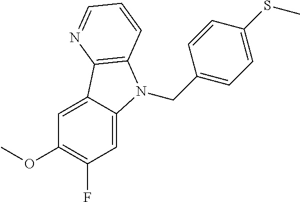

The compound prepared in (Step 4) of Example 29 (50.0 mg, 0.23 mmol) was dissolved in N,N-dimethylformamide (1.00 mL), and then cooled to 0° C., and 60% sodium hydride (18.0 mg, 0.46 mmol) was added to the mixture and stirred for 30 minutes. Intermediate 6 (55.0 mg, 0.25 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:3) to obtain the title compound as a yellow solid. (50.0 mg, 60%) MS m/z: 353 [M+1]^{+ 1}H NMR (400 MHz, CDCl₃) δ 8.54 (dd, J=4.7, 1.3 Hz, 1H), 7.96 (d, J=8.3 Hz, 1H), 7.62 (dd, J=8.3, 1.4 Hz, 1H), 7.30 (dd, J=8.3, 4.6 Hz, 1H), 7.19-7.09 (m, 3H), 7.01 (d, J=8.6 Hz, 2H), 5.40 (s, 2H), 4.02 (s, 3H), 2.43 (s, 3H).

### (Step 2) Preparation of (4-((7-fluoro-8-methoxy-5H-pyrido[3,2-b]indol-5-yl)methyl)phenyl)(imino)(methyl)-l6-sulfanone (Compound 30)

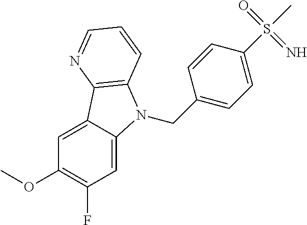 [SMILES: COc1cc2c3ncccc3n(Cc3ccc(S(C)(=N)=O)cc3)c2cc1F]

The compound prepared in (Step 1) above (50.0 mg, 0.14 mmol) was dissolved in ethanol (1.00 mL), and then iodobenzene diacetate (132 mg, 0.41 mmol) and ammonium acetate (41.0 mg, 0.54 mmol) were added to the mixture. The reaction mixture was stirred at room temperature for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (53.0 mg, 96%) Compound 30 was purified by preparative high performance liquid chromatography (prep-HPLC, chiralpak IC (5*100 mm, 5 μm), hexane:methanol:ethanol=40:30:30) to obtain 30a and 30b, which are white optical isomers, respectively.

30a: [α]D23+3° (c 0.1, CHCl3), 30b: [α]D23−3° (c 0.1, CHCl3). MS m/z: 384 [M+1]^{+ 1}H NMR (400 MHz, CD₃OD) δ 8.72 (dd, J=5.6, 1.1 Hz, 1H), 8.66 (dd, J=8.4, 1.1 Hz, 1H), 8.09 (dt, J=8.4, 2.0 Hz, 3H), 7.89 (dd, J=8.5, 5.6 Hz, 1H), 7.62 (d, J=11.1 Hz, 1H), 7.54 (d, J=8.8 Hz, 2H), 6.01 (s, 2H), 4.05 (s, 3H), 3.70 (s, 3H).

### [Example 31] Preparation of (3-fluoro-4-(7-fluoro-8-methoxy-5H-pyrido[3,2-b]indol-5-yl)methyl)phenyl)(imino)(methyl)-l6-sulfanone (Compound 31)

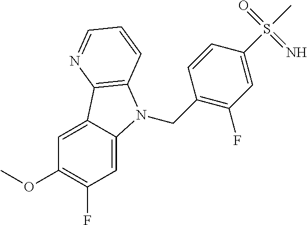 [SMILES: COc1cc2c3ncccc3n(Cc3ccc(S(C)(=N)=O)cc3F)c2cc1F]

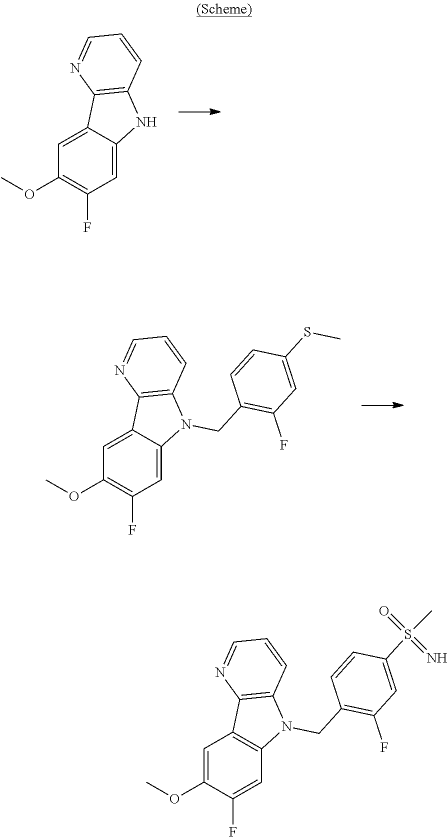

### (Step 1) Preparation of 7-fluoro-5-(2-fluoro-4-(methylthio)benzyl)-8-methoxy-5H-pyrido[3,2-b]indole

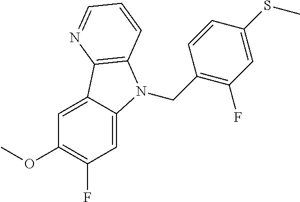

The compound prepared in (Step 4) of Example 29 (50.0 mg, 0.23 mmol) was dissolved in N,N-dimethylformamide (1.00 mL), and then cooled to 0° C., and 60% sodium hydride (18.0 mg, 0.46 mmol) was added to the mixture and stirred for 30 minutes. Intermediate 12 (59.0 mg, 0.25 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=2:8) to obtain the title compound as a yellow solid. (50.0 mg, 58%) MS m/z: 371 [M+1]⁺

### (Step 2) Preparation of (3-fluoro-4-((7-fluoro-8-methoxy-5H-pyrido[3,2-b]indol-5-yl)methyl)phenyl)(imino)(methyl)-l6-sulfanone (Compound 31)

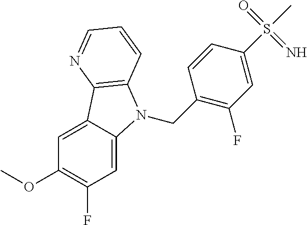 [SMILES: COc1cc2c3ncccc3n(Cc3ccc(S(C)(=N)=O)cc3F)c2cc1F]

The compound prepared in (Step 1) above (42.0 mg, 0.11 mmol) was dissolved in ethanol (1.00 mL), and then iodobenzene diacetate (106 mg, 0.33 mmol) and ammonium acetate (33.0 mg, 0.43 mmol) were added to the mixture. The reaction mixture was stirred at room temperature for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (45.0 mg, 99%) Compound 31 was purified by preparative high performance liquid chromatography (prep-HPLC, chiralpak IC (5*100 mm, 5 μm), hexane:methanol:ethanol=40:30:30) to obtain 31a and 31b, which are white optical isomers, respectively.

31a: [α]D23+9° (c 0.12, CHCl3), 3b: [α]D23−7° (c 0.12, CHCl3). MS m/z: 402 [M+1]^{+ 1}H NMR (400 MHz, CD₃OD) δ 8.75-8.67 (m, 2H), 8.07 (d, J=8.1 Hz, 1H), 7.98-7.88 (m, 2H), 7.85-7.77 (m, 1H), 7.68 (d, J=11.1 Hz, 1H), 7.37-7.28 (m, 1H), 6.01 (s, 2H), 4.04 (s, 3H), 3.50 (s, 3H).

### [Example 32] Preparation of (4-((8-fluoro-7-methoxy-5H-pyrido[3,2-b]indol-5-yl)methyl)phenyl)(imino)(methyl)-l6-sulfanone (Compound 32)

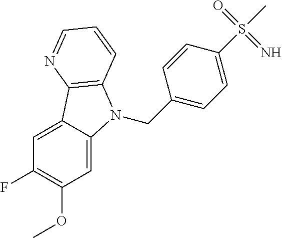 [SMILES: COc1cc2c(cc1F)c1ncccc1n2Cc1ccc(S(C)(=N)=O)cc1]

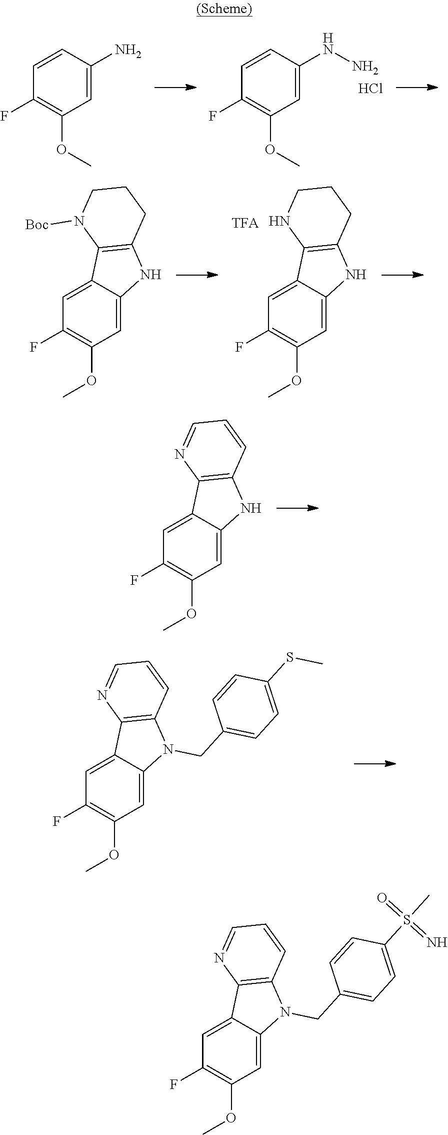

### (Step 1) Preparation of (4-fluoro-3-methoxyphenyl) hydrazine-hydrochloride

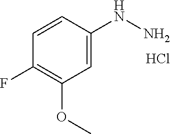

4-Fluoro-3-methoxyaniline (1.00 g, 7.08 mmol) was dissolved in 4.00 mL of 6N HCl, and then sodium nitrite (733 mg, 10.6 mmol) dissolved in 2.00 mL of a saturated saline solution was slowly added to the mixture at −20° C. The reaction mixture was stirred at −20° C. for 30 minutes under nitrogen, and then tin (II) chloride dihydrate (4.80 g, 21.3 mmol) dissolved in 2.00 mL of conc. HCl was added slowly. After stirring at 0° C. for 1 hour, the solid produced in the reaction mixture was filtered, washed with conc. hydrochloric acid, and then dried to obtain the title compound (1.10 g, 80%) as a white solid. MS m/z: 193 [M+1]^{+ 1}H NMR (400 MHz, DMSO-d₆) δ 9.99 (s, 2H), 8.08 (s, 1H), 7.15 (dd, J=11.4, 8.8 Hz, 1H), 6.85 (dd, J=7.4, 2.7 Hz, 1H), 6.49 (dt, J=8.6, 3.1 Hz, 1H), 3.81 (s, 3H).

### (Step 2) Preparation of tert-butyl 8-fluoro-7-methoxy-2,3,4,5-tetrahydro-1H-pyrido[3,2-b]indole-1-carboxylate

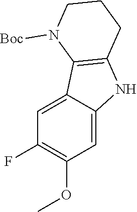 [SMILES: COc1cc2[nH]c3c(c2cc1F)N(C(=O)OC(C)(C)C)CCC3]

The compound prepared in (Step 1) above (200 mg, 1.03 mmol) was dissolved in toluene (5.00 mL), and then tert-butyl-3-oxopiperidine-1-carboxylate (248 mg, 1.24 mmol) and propanephosphonic anhydride (0.20 mL, 0.21 mmol) were added to the mixture sequentially. The reaction mixture was stirred at 90° C. for 3 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=3:7) to obtain the title compound as a pale yellow solid. (95.0 mg, 28%) MS m/z: 321 [M+1]^{+ 1}H NMR (400 MHz, CDCl₃) δ 7.66 (s, 1H), 7.46-7.29 (m, 1H), 6.76 (d, J=7.1 Hz, 1H), 3.88 (s, 3H), 3.79-3.70 (m, 2H), 2.78 (t, J=6.8 Hz, 2H), 2.04 (q, J=3.3 Hz, 2H), 1.53 (s, 9H).

### (Step 3) Preparation of 8-fluoro-7-methoxy-2,3,4,5-tetrahydro-1H-pyrido[3,2-b]indole-trifluoroacetic acid

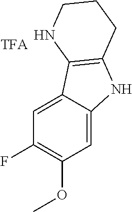 [SMILES: COc1cc2[nH]c3c(c2cc1F)NCCC3]

The compound prepared in (Step 2) above (95.0 mg, 0.29 mmol) was dissolved in methylene chloride (1.00 mL), and then trifluoroacetic acid (1.00 mL) was added to the mixture. The reaction mixture was stirred at room temperature for 1 hour under nitrogen. After completion of the reaction, the mixture was concentrated under reduced pressure. The mixture was dissolved in ethyl ether, and then n-hexane was added. The resulting solid was filtered, washed with a mixed solution of ethyl ether/n-hexane, and dried to obtain the title compound as a brown solid. (95.0 mg, 99%) MS m/z: 221 [M+1]⁺

### (Step 4) Preparation of 8-fluoro-7-methoxy-5H-pyrido[3,2-b]indole

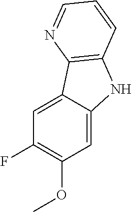 [SMILES: COc1cc2[nH]c3cccnc3c2cc1F]

The compound prepared in (Step 3) above (95.0 mg, 0.29 mmol) was dissolved in toluene (5.00 mL), and then Pd/C (10 wt %, 50.0 mg) was added to the mixture. The reaction mixture was stirred at 120° C. for 18 hours under nitrogen. Precipitation was removed by filtration using Celite, and then the filtrate was concentrated under reduced pressure. The residue was purified by preparative high performance liquid chromatography (prep-HPLC, acetonitrile:distilled water=5:95 to 100:0) to obtain the title compound as a yellow solid. (40.0 mg, 65%) MS m/z: 217 [M+1]⁺

### (Step 5) Preparation of 8-fluoro-7-methoxy-5-(4-(methylthio)benzyl)-5H-pyrido[3,2-b]indole

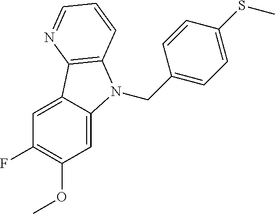 [SMILES: COc1cc2c(cc1F)c1ncccc1n2Cc1ccc(SC)cc1]

The compound prepared in (Step 4) above (40.0 mg, 0.18 mmol) was dissolved in N,N-dimethylformamide (1.00 mL), and then cooled to 0° C., and 60% sodium hydride (15.0 mg, 0.37 mmol) was added to the mixture and stirred for 30 minutes. Intermediate 6 (44.0 mg, 0.20 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=2:3) to obtain the title compound as a pale yellow solid. (25.0 mg, 39%) MS m/z: 353 [M+1]^{+ 1}H NMR (400 MHz, CD₃OD) δ 8.39 (dd, J=4.8, 1.4 Hz, 1H), 7.97 (d, J=10.9 Hz, 1H), 7.88 (dd, J=8.3, 1.4 Hz, 1H), 7.37 (dd, J=8.3, 4.8 Hz, 1H), 7.23 (d, J=6.9 Hz, 1H), 7.21-7.14 (m, 2H), 7.07 (d, J=8.4 Hz, 2H), 5.60 (s, 2H), 3.95 (s, 3H), 2.40 (s, 3H).

### (Step 6) Preparation of (4-((8-fluoro-7-methoxy-5H-pyrido[3,2-b]indol-5-yl)methyl)phenyl)(imino)(methyl)-l6-sulfanone (Compound 32)

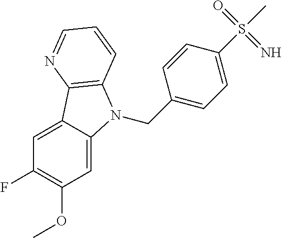 [SMILES: COc1cc2c(cc1F)c1ncccc1n2Cc1ccc(S(C)(=N)=O)cc1]

The compound prepared in (Step 5) above (25.0 mg, 0.07 mmol) was dissolved in ethanol (1.00 mL), and then iodobenzene diacetate (56.0 mg, 0.20 mmol) and ammonium acetate (20.0 mg, 0.26 mmol) were added to the mixture. The reaction mixture was stirred at room temperature for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (19.5 mg, 71%) MS m/z: 384 [M+1]^{+ 1}H NMR (400 MHz, CD₃OD) δ 8.66 (d, J=5.8 Hz, 1H), 8.58 (d, J=8.5 Hz, 1H), 8.15-8.04 (m, 3H), 7.83 (dd, J=8.4, 5.7 Hz, 1H), 7.57-7.51 (m, 2H), 7.46 (d, J=6.8 Hz, 1H), 6.06 (s, 2H), 4.02 (s, 3H), 3.60 (s, 3H).

### [Example 33] Preparation of 4-((6-methoxy-3-methyl-1,2,3,4-tetrahydro-9H-pyrido[3,4-b]indol-9-yl)methyl)benzene sulfonamide (Compound 33)

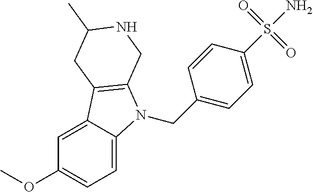 [SMILES: COc1ccc2c(c1)c1c(n2Cc2ccc(S(N)(=O)=O)cc2)CNC(C)C1]

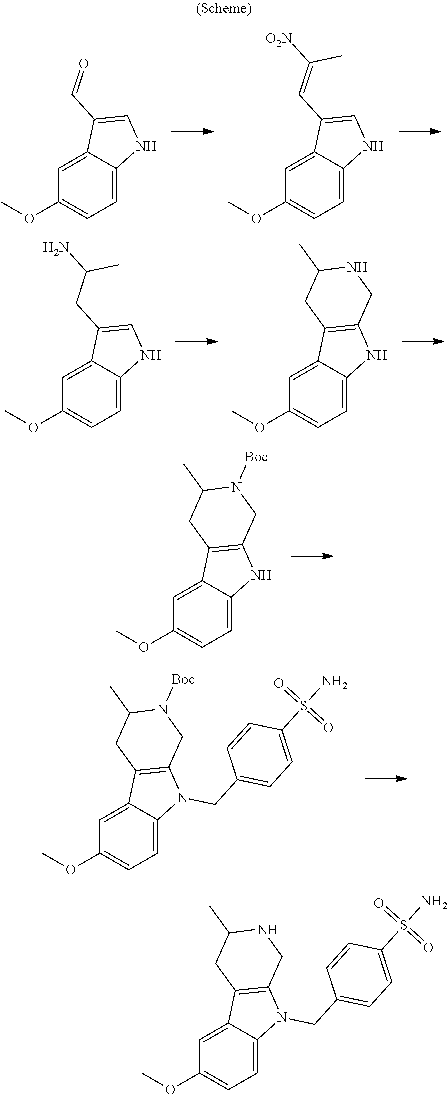

### (Step 1) Preparation of (E)-5-methoxy-3-(2-nitroprop-1-en-1-yl)-1H-indole

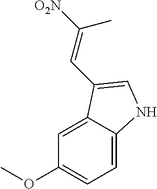 [SMILES: COc1ccc2[nH]cc(/C=C(\C)[N+](=O)[O-])c2c1]

Nitroethane (257 mg, 3.42 mmol) and ammonium acetate (329 mg, 4.27 mmol) were sequentially added to 5-methoxy-1H-indole-3-carbaldehyde (500 mg, 2.85 mmol). The reaction mixture was stirred at 90° C. for 6 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:1 to 3:1) to obtain the title compound as a white solid. (410 mg, 75%) MS m/z: 233 [M+1]^{+ 1}H NMR (400 MHz, DMSO-d₆) δ 12.11 (s, 1H), 8.50 (s, 1H), 7.97 (s, 1H), 7.47-7.33 (m, 2H), 6.92-6.84 (m, 1H), 3.85 (s, 3H), 2.52 (s, 3H).

### (Step 2) Preparation of 1-(5-methoxy-1H-indol-3-yl) propan-2-amine

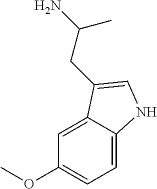

The compound prepared in (Step 1) above (410 mg, 1.75 mmol) was dissolved in tetrahydrofuran (3.00 mL), and then lithium aluminum hydride (133 mg, 3.52 mmol) was added to the mixture. The reaction mixture was stirred at 80° C. for 10 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=5:1 to 9:1) to obtain the title compound as a white solid. (280 mg, 55%) MS m/z: 205 [M+1]^{+ 1}H NMR (400 MHz, DMSO-d₆) δ 8.51 (s, 1H), 7.28-7.21 (m, 1H), 7.05-6.95 (m, 1H), 6.88-6.81 (m, 1H), 3.84 (s, 3H), 3.50 (br s, 2H), 3.29 (s, 1H), 2.91-2.63 (m, 2H), 1.18 (s, 3H).

### (Step 3) Preparation of 6-methoxy-3-methyl-2,3,4,9-tetrahydro-1H-pyrido[3,4-b]indole

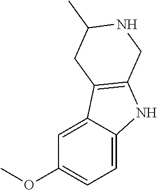 [SMILES: COc1ccc2[nH]c3c(c2c1)CC(C)NC3]

Formaldehyde (5.0 mL) was added to the compound prepared in (Step 2) above (100 mg, 0.49 mmol). The reaction mixture was stirred at 80° C. for 5 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=5:1 to 7:1) to obtain the title compound as a white solid. (33.0 mg) MS m/z: 217 [M+1]⁺

### (Step 4) Preparation of tert-butyl 6-methoxy-3-methyl-1,3,4,9-tetrahydro-2H-pyrido[3,4-b]indole-2-carboxylate

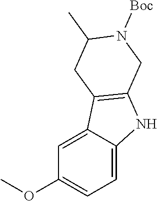 [SMILES: COc1ccc2[nH]c3c(c2c1)CC(C)N(C(=O)OC(C)(C)C)C3]

The compound prepared in (Step 3) above (33.0 mg, 0.15 mmol) was dissolved in methylene chloride (3.00 mL), and then N,N-diisopropylethylamine (53.0 μl, 0.30 mmol) and di-tert-butyl dicarbonate (36.1 mg, 0.16 mmol) were added to the mixture sequentially. The reaction mixture was stirred at room temperature for 8 hours under nitrogen. After completion of the reaction, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=3:1 to 5:1) to obtain the title compound as a white solid. (13.0 mg, 48%) MS m/z: 317 [M+1]⁺

### (Step 5) Preparation of tert-butyl 6-methoxy-3-methyl-9-(4-sulfamoylbenzyl)-1,3,4,9-tetrahydro-2H-pyrido[3,4-b]indole-2-carboxylate

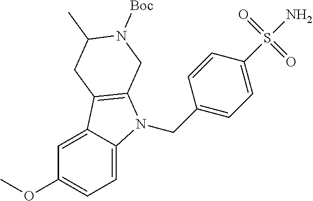 [SMILES: COc1ccc2c(c1)c1c(n2Cc2ccc(S(N)(=O)=O)cc2)CN(C(=O)OC(C)(C)C)C(C)C1]

The compound prepared in (Step 4) above (13.0 mg, 0.04 mmol) was dissolved in N,N-dimethylformamide (1.00 mL), and then cooled to 0° C., and 60% sodium hydride (2.00 mg, 0.08 mmol) was added to the mixture and stirred for 30 minutes. Intermediate 1 (12.1 mg, 0.05 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (methylene chloride:methanol=20:1→9:1) to obtain the title compound as a yellow solid. (8.50 mg, 43%) MS m/z: 486 [M+1]⁺

### (Step 6) Preparation of 4-((6-methoxy-3-methyl-1,2,3,4-tetrahydro-9H-pyrido[3,4-b]indol-9-yl)methyl)benzene sulfonamide (Compound 33)

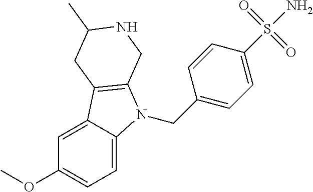 [SMILES: COc1ccc2c(c1)c1c(n2Cc2ccc(S(N)(=O)=O)cc2)CNC(C)C1]

The compound prepared in (Step 5) above (8.00 mg, 0.02 mmol) was dissolved in methylene chloride (1.50 mL), and then trifluoroacetic acid (1.50 mL) was added to the mixture. The reaction mixture was stirred at room temperature for 2 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (4.10 mg, 5%) MS m/z: 386 [M+1]^{+ 1}H NMR (400 MHz, DMSO-d₆) δ 7.74 (d, J=8.4 Hz, 2H), 7.32 (s, 3H), 7.17 (d, J=8.4 Hz, 2H), 7.02 (s, 1H), 6.79-6.72 (m, 1H), 5.45 (s, 2H), 3.76 (s, 3H), 3.75-3.74 (m, 1H), 3.01 (s, 2H), 2.71 (s, 2H), 1.43 (d, J=6.4 Hz, 3H).

### [Example 34] Preparation of 4-((3-methoxy-5,6,7,8-tetrahydro-9H-pyrrolo[2,3-b:5,4-c′]dipyridin-9-yl)methyl)benzene sulfonamide (Compound 34)

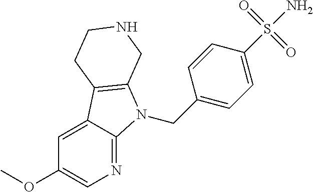 [SMILES: COc1cnc2c(c1)c1c(n2Cc2ccc(S(N)(=O)=O)cc2)CNCC1]

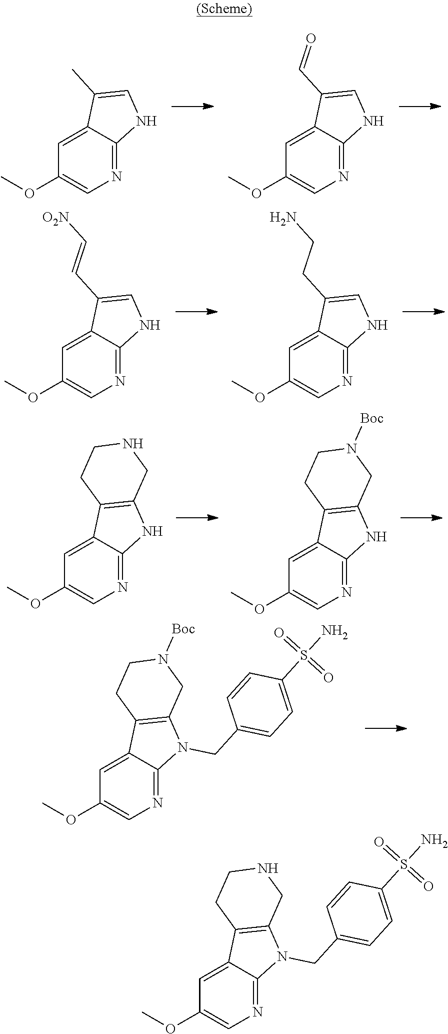

### (Step 1) Preparation of 5-methoxy-1H-pyrrolo[2,3-b]pyridine-3-carbaldehyde

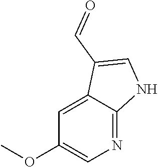

Phosphoryl chloride (51.7 g, 337 mmol) and N,N-dimethylformamide (30.0 mL) were sequentially added to the mixture at 0° C., and then stirred for 30 minutes. 5-Methoxy-1H-pyrrolo[2,3-b]pyridine (5.00 g, 33.7 mmol) dissolved in N,N-dimethylformamide (30.0 mL) was slowly added to the reaction mixture, and then stirred at 80° C. for 3 hours. After completion of the reaction, it was cooled to room temperature, and 2M aqueous sodium hydroxide solution was added (pH=9). The reaction mixture was extracted 3 times with ethyl acetate. Ethyl acetate was added to the mixture, and the resulting solid was filtered and dried to obtain the title compound as a yellow solid. (2.68 g, 45%) MS m/z: 304 [M+1]^{+ 1}H NMR (400 MHz, DMSO-d₆) & 12.60 (br s, 1H), 9.89 (s, 1H), 8.40 (s, 1H), 8.11 (d, J=2.9 Hz, 1H), 7.91 (d, J=2.9 Hz, 1H), 3.86 (s, 3H).

### (Step 2) Preparation of (E)-5-methoxy-3-(2-nitrovinyl)-1H-pyrrolo[2,3-b]pyridine

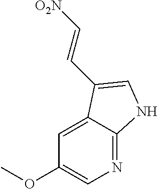 [SMILES: COc1cnc2[nH]cc(/C=C/[N+](=O)[O-])c2c1]

The compound prepared in (Step 1) above (1.68 g, 9.54 mmol) was dissolved in nitromethane (20.0 mL), and then ammonium acetate (2.83 g, 36.7 mmol) was added to the mixture. The mixture was stirred at 85° C. for 1 hour under nitrogen. After completion of the reaction, distilled water was added, and extracted with methylene chloride. The organic layers were dried over anhydrous sodium sulfate, and then concentrated under reduced pressure. The title compound was obtained as a brown solid without further purification. (1.80 g, 86%) MS m/z: 220 [M+1]^{+ 1}H NMR (400 MHz, DMSO-d₆) δ 8.37 (d, J=13.5 Hz, 1H), 8.32 (s, 1H), 8.16 (d, J=13.5 Hz, 1H), 8.08 (d, J=2.6 Hz, 1H), 7.97 (d, J=2.6 Hz, 1H), 3.91 (s, 3H).

### (Step 3) Preparation of 2-(5-methoxy-1H-pyrrolo[2,3-b]pyridin-3-yl) ethan-1-amine

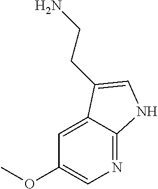

The compound prepared in (Step 2) above (2.50 g, 11.4 mmol) was dissolved in tetrahydrofuran (20.0 mL), and then lithium aluminum hydride (2.60 g, 68.4 mmol) was slowly added to the mixture at 0° C. The reaction mixture was stirred at room temperature for 5 hours under nitrogen. After completion of the reaction, distilled water and 15% aqueous sodium hydroxide solution (2.6 mL) were added, and then extracted twice with a mixed solution of methylene chloride:methanol=5:1. The organic layers were dried over anhydrous sodium sulfate, and then concentrated under reduced pressure. The title compound was obtained as a brown oil without further purification. (2.00 g) MS m/z: 192 [M+1]^{+ 1}H NMR (400 MHz, DMSO-d₆) δ 11.66 (br s, 1H), 8.04 (br d, J=2.5 Hz, 3H), 7.86 (d, J=2.4 Hz, 1H), 7.41 (d, J=2.3 Hz, 1H), 3.87 (s, 3H), 3.12-2.97 (m, 4H).

### (Step 4) Preparation of 3-methoxy-6,7,8,9-tetrahydro-5H-pyrrolo[2,3-b:5,4-c′]dipyridine

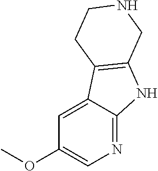

The compound prepared in (Step 3) above (200 mg, 1.04 mmol) was dissolved in acetic acid (4.00 mL) and methanol (0.40 mL), and then formaldehyde solution (452 μl, 1.04 mmol) was added to the mixture. The reaction mixture was stirred at 65° C. for 4 hours under nitrogen. After cooling to room temperature, the mixture was concentrated under reduced pressure to obtain the title compound as a yellow solid without further purification. (75.0 mg, 53%) MS m/z: 204 [M+1]⁺

### (Step 5) Preparation of tert-butyl 3-methoxy-5,6,8,9-tetrahydro-7H-pyrrolo[2,3-b:5,4-c′]dipyridine-7-carboxylate

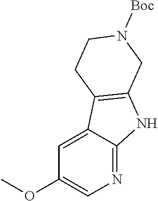 [SMILES: COc1cnc2[nH]c3c(c2c1)CCN(C(=O)OC(C)(C)C)C3]

The compound prepared in (Step 4) above (75.0 mg, 0.37 mmol) was dissolved in methylene chloride (2.00 mL), and then cooled to 0° C., and triethylamine (80.0 μl, 0.55 mmol) and di-tert-butyl dicarbonate (93.0 uL, 0.41 mmol) were added to the mixture and stirred at room temperature for 1 hour under nitrogen. After completion of the reaction, distilled water was added slowly, and extracted twice with methylene chloride. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (30.0 mg, 26%) MS m/z: 304 [M+1]⁺

### (Step 6) Preparation of tert-butyl 3-methoxy-9-(4-sulfamoylbenzyl)-5,6,8,9-tetrahydro-7H-pyrrolo[2,3-b:5,4-c′]dipyridine-7-carboxylate

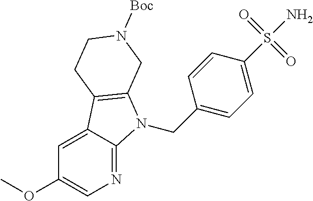 [SMILES: COc1cnc2c(c1)c1c(n2Cc2ccc(S(N)(=O)=O)cc2)CN(C(=O)OC(C)(C)C)CC1]

The compound prepared in (Step 5) above (30.0 mg, 0.10 mmol) was dissolved in N,N-dimethylformamide (1.00 mL), and then cooled to 0° C., and 60% sodium hydride (11.9 mg, 0.30 mmol) was added to the mixture and stirred for 30 minutes. Intermediate 1 (27.2 mg, 0.11 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:3) to obtain the title compound as a yellow solid. (25.0 mg, 53%) MS m/z: 473 [M+1]⁺

### (Step 7) Preparation of 4-((3-methoxy-5,6,7,8-tetrahydro-9H-pyrrolo[2,3-b:5,4-c′]dipyridin-9-yl)methyl)benzene sulfonamide (Compound 34)

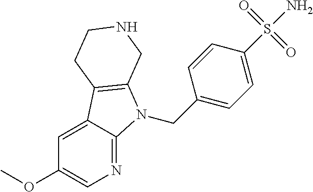 [SMILES: COc1cnc2c(c1)c1c(n2Cc2ccc(S(N)(=O)=O)cc2)CNCC1]

The compound prepared in (Step 6) above (25.0 mg, 0.05 mmol) was dissolved in methylene chloride (0.50 mL), and then trifluoroacetic acid (0.50 mL) was added to the mixture. The reaction mixture was stirred at room temperature for 2 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (2.30 mg, 12%) MS m/z: 373 [M+1]^{+ 1}H NMR (400 MHz, CD₃OD) δ 8.04 (s, 1H), 7.82 (s, 2H), 7.60 (s, 1H), 7.24 (s, 2H), 5.55 (s, 2H), 4.34 (s, 2H), 3.90 (s, 3H), 3.58 (s, 2H), 3.10 (s, 2H).

### [Example 35] Preparation of 4-((7-methoxy-1,2,3,4-tetrahydro-5H-pyrido[4,3-b]indol-5-yl)methyl)benzene sulfonamide (Compound 35)

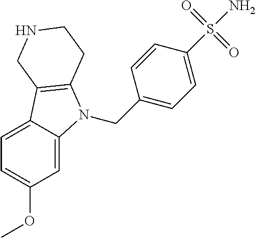 [SMILES: COc1ccc2c3c(n(Cc4ccc(S(N)(=O)=O)cc4)c2c1)CCNC3]

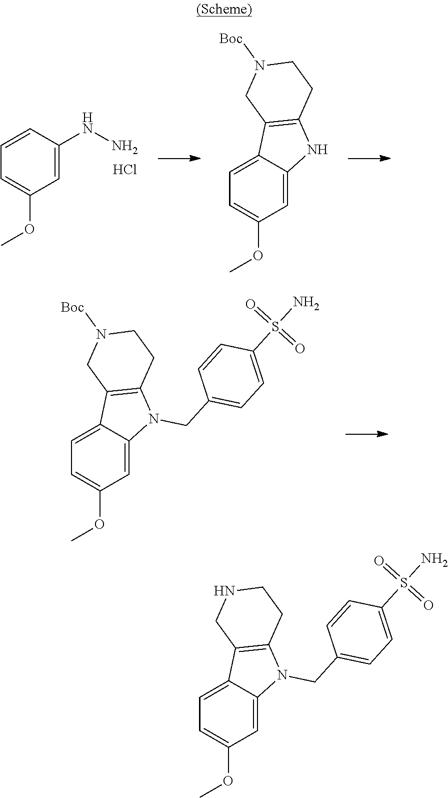

### (Step 1) Preparation of tert-butyl 7-methoxy-1,3,4,5-tetrahydro-2H-pyrido[4,3-b]indole-2-carboxylate

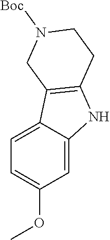 [SMILES: COc1ccc2c3c([nH]c2c1)CCN(C(=O)OC(C)(C)C)C3]

(3-Methoxyphenyl) hydrazine-hydrochloride (2.00 g, 11.5 mmol) was dissolved in toluene (5.00 mL), and then tert-butyl-4-oxopiperidine-1-carboxylate (2.70 g, 13.8 mmol) and propanephosphonic anhydride (2.50 g, 17.2 mmol) were added to the mixture sequentially. The reaction mixture was stirred at 90° C. for 6 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:4 to 1:2) to obtain the title compound as a yellow solid. (180 mg) MS m/z: 303 [M+1]⁺

### (Step 2) Preparation of tert-butyl 7-methoxy-5-(4-sulfamoylbenzyl)-1,3,4,5-tetrahydro-2H-pyrido[4,3-b]indole-2-carboxylate

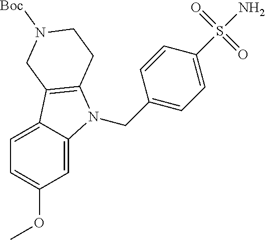 [SMILES: COc1ccc2c3c(n(Cc4ccc(S(N)(=O)=O)cc4)c2c1)CCN(C(=O)OC(C)(C)C)C3]

The compound prepared in (Step 1) above (20.0 mg, 0.07 mmol) was dissolved in N,N-dimethylformamide (1.50 mL), and then cooled to 0° C., and 60% sodium hydride (3.20 mg, 0.13 mmol) was added to the mixture and stirred for 30 minutes. Intermediate 1 (19.5 mg, 0.08 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (methanol:methylene chloride=5: 95→10:90) to obtain the title compound as a yellow solid. (5.10 mg, 16%) MS m/z: 472 [M+1]⁺

### (Step 3) Preparation of 4-((7-methoxy-1,2,3,4-tetrahydro-5H-pyrido[4,3-b]indol-5-yl)methyl)benzenesulfonamide (Compound 35)

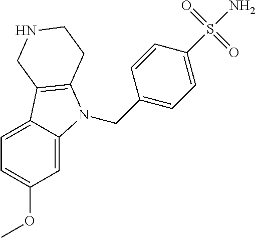 [SMILES: COc1ccc2c3c(n(Cc4ccc(S(N)(=O)=O)cc4)c2c1)CCNC3]

The compound prepared in (Step 2) above (5.00 mg, 0.01 mmol) was dissolved in methylene chloride (2.00 mL), and then trifluoroacetic acid (2.00 mL) was added to the mixture. The reaction mixture was stirred at room temperature for 2 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (1.50 mg, 40%) MS m/z: 372 [M+1]⁺

### [Example 36] Preparation of 4-((8-methoxy-1,2,3,4-tetrahydro-5H-pyrido[4,3-b]indol-5-yl)methyl)benzene sulfonamide (Compound 36)

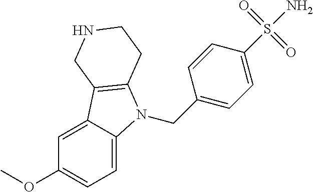 [SMILES: COc1ccc2c(c1)c1c(n2Cc2ccc(S(N)(=O)=O)cc2)CCNC1]

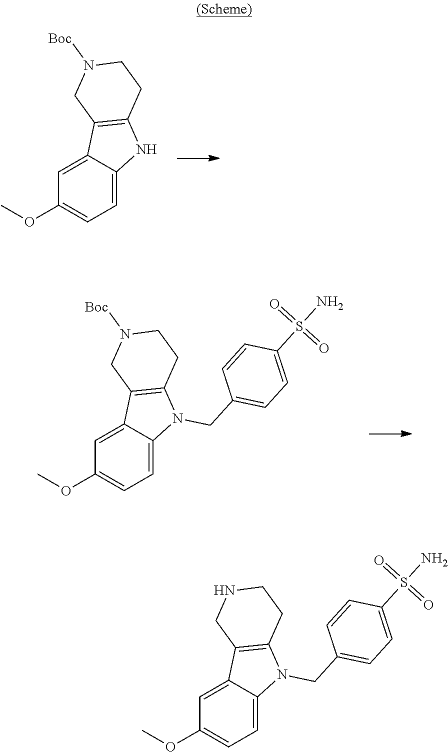 [SMILES: COc1ccc2[nH]c3c(c2c1)CN(C(=O)OC(C)(C)C)CC3 ; COc1ccc2c(c1)c1c(n2Cc2ccc(S(N)(=O)=O)cc2)CCN(C(=O)OC(C)(C)C)C1 ; COc1ccc2c(c1)c1c(n2Cc2ccc(S(N)(=O)=O)cc2)CCNC1]

### (Step 1) Preparation of tert-butyl 8-methoxy-5-(4-sulfamoylbenzyl)-1,3,4,5-tetrahydro-2H-pyrido[4,3-b]indole-2-carboxylate

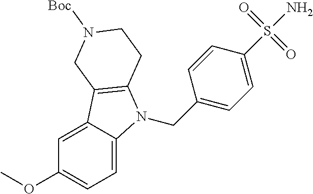 [SMILES: COc1ccc2c(c1)c1c(n2Cc2ccc(S(N)(=O)=O)cc2)CCN(C(=O)OC(C)(C)C)C1]

The compound prepared in (Step 1) of Example 4 (180 mg, 0.60 mmol) was dissolved in N,N-dimethylformamide (3.00 mL), and then cooled to 0° C., and 60% sodium hydride (90.0 mg, 2.25 mmol) was added to the mixture and stirred for 30 minutes. Intermediate 1 (178 mg, 0.71 mmol) was slowly added to the reaction mixture, and then stirred at 0° C. for 2 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=2:3 to 3:2) to obtain the title compound as a yellow solid. (128 mg, 46%) MS m/z: 472 [M+1]^{+ 1}H NMR (400 MHz, CDCl₃) δ ppm: 7.81 (d, J=8.0 Hz, 2H), 7.08 (d, J=8.0 Hz, 2H), 7.04 (d, J=8.9 Hz, 1H), 6.95 (s, 1H), 6.79 (d, J=8.6 Hz, 1H), 5.28 (s, 2H), 4.79 (s, 2H), 4.65 (s, 2H), 3.86-3.80 (m, 5H), 2.68 (s, 2H), 1.50 (s, 9H).

### (Step 2) Preparation of 4-((8-methoxy-1,2,3,4-tetrahydro-5H-pyrido[4,3-b]indol-5-yl)methyl)benzenesulfonamide (Compound 36)

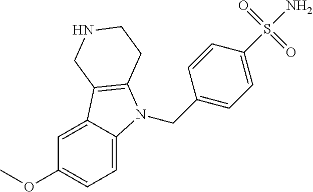 [SMILES: COc1ccc2c(c1)c1c(n2Cc2ccc(S(N)(=O)=O)cc2)CCNC1]

The compound prepared in (Step 1) above (128 mg, 0.27 mmol) was dissolved in methylene chloride (1.00 mL), and then trifluoroacetic acid (1.00 mL) was added to the mixture. The reaction mixture was stirred at room temperature for 2 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (48.0 mg, 47%) MS m/z: 372 [M+1]^{+ 1}H NMR (400 MHz, DMSO-d₆) δ ppm: 9.03 (s, 1H), 7.75 (d, J=8.0 Hz, 2H), 7.45-7.30 (m, 3H), 7.20 (d, J=8.1 Hz, 2H), 7.09 (s, 1H), 6.79 (d, J=8.9 Hz, 1H), 5.48 (s, 2H), 4.35 (s, 2H), 3.78 (s, 3H), 2.99 (s, 2H).

### [Example 37] Preparation of 4-((7-methoxy-1,2,3,4-tetrahydro-5H-pyrido[3,2-b]indol-5-yl)methyl)benzene sulfonamide (Compound 37)

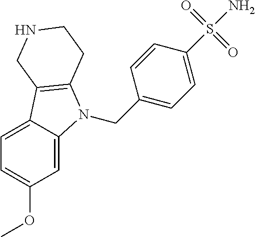 [SMILES: COc1ccc2c3c(n(Cc4ccc(S(N)(=O)=O)cc4)c2c1)CCNC3]

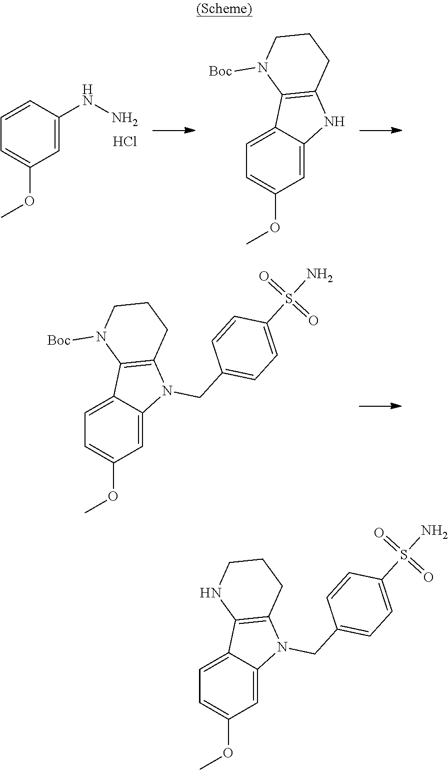

### (Step 1) Preparation of tert-butyl 7-methoxy-2,3,4,5-tetrahydro-1H-pyrido[3,2-b]indole-1-carboxylate

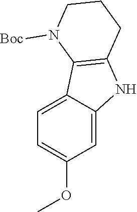 [SMILES: COc1ccc2c3c([nH]c2c1)CCCN3C(=O)OC(C)(C)C]

1,3-Dimethylurea (8.81 g, 100 mmol) and L-(+)-tartaric acid (4.50 g, 30.0 mmol) were mixed, and then the reaction mixture was stirred at 80° C. for 2 hours under nitrogen. (3-Methoxyphenyl) hydrazine-hydrochloride (1.87 g, 11.0 mmol) and tert-butyl-3-oxopiperidine-1-carboxylate (2.00 mg, 10.0 mmol) were sequentially added to the reaction mixture, and then stirred at 80° C. for 2 hours. After cooling to room temperature, distilled water and an aqueous sodium bicarbonate solution were added, and extracted twice with methylene chloride. The organic layers were dried over anhydrous sodium sulfate, and then concentrated under reduced pressure. The residue was purified by silica gel column chromatography (ethyl acetate:n-hexane=0:10 to 2:8) to obtain the title compound as a yellow solid. (471 mg, 15%) MS m/z: 303 [M+1]^{+ 1}H NMR (400 MHz, CDCl₃) δ 7.56-7.48 (m, 2H), 6.75 (s, 2H), 3.86 (s, 3H), 3.76-3.75 (m, 2H), 2.81-2.78 (m, 2H), 2.07-2.04 (m, 2H), 1.52 (s, 9H)

### (Step 2) Preparation of tert-butyl 7-methoxy-5-(4-sulfamoylbenzyl)-2,3,4,5-tetrahydro-1H-pyrido[3,2-b]indole-1-carboxylate

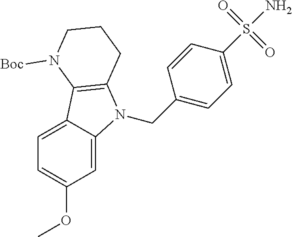 [SMILES: COc1ccc2c3c(n(Cc4ccc(S(N)(=O)=O)cc4)c2c1)CCCN3C(=O)OC(C)(C)C]

The compound prepared in (Step 1) above (100 mg, 0.33 mmol) was dissolved in N,N-dimethylformamide (2.00 mL), and then cooled to 0° C., and 60% sodium hydride (26.0 mg, 0.66 mmol) was added to the mixture and stirred for 30 minutes. Intermediate 1 (90.0 mg, 0.36 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=3:7) to obtain the title compound as a yellow solid. (64.0 mg, 41%) MS m/z: 472 [M+1]⁺

### (Step 3) Preparation of 4-((7-methoxy-1,2,3,4-tetrahydro-5H-pyrido[3,2-b]indol-5-yl)methyl)benzenesulfonamide (Compound 37)

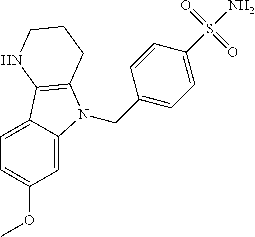 [SMILES: COc1ccc2c3c(n(Cc4ccc(S(N)(=O)=O)cc4)c2c1)CCCN3]

The compound prepared in (Step 2) above (64.0 mg, 0.13 mmol) was dissolved in methylene chloride (2.00 mL), and then hydrogen chloride (4M 1,4-dioxane solution, 1.00 mL) was added to the mixture. The reaction mixture was stirred at room temperature for 1 hour under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (33.0 mg, 52%) MS m/z: 372 [M+1]^{+ 1}H NMR (400 MHz, CD₃OD) δ 7.83 (d, J=7.6 Hz, 2H), 7.50-7.41 (m, 1H), 7.19 (d, J=8.4 Hz, 2H), 6.94 (s, 1H), 6.86-6.82 (m, 1H), 5.47 (s, 2H), 3.77 (s, 3H). 3.58 (s, 2H), 2.83 (s, 2H), 2.46 (s, 2H)

### [Example 38] Preparation of 4-((8-methoxy-1,2,3,4-tetrahydro-5H-pyrido[3,2-b]indol-5-yl)methyl)benzene sulfonamide (Compound 38)

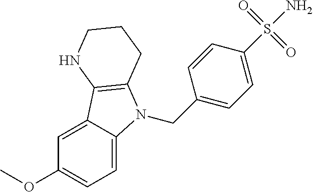 [SMILES: COc1ccc2c(c1)c1c(n2Cc2ccc(S(N)(=O)=O)cc2)CCCN1]

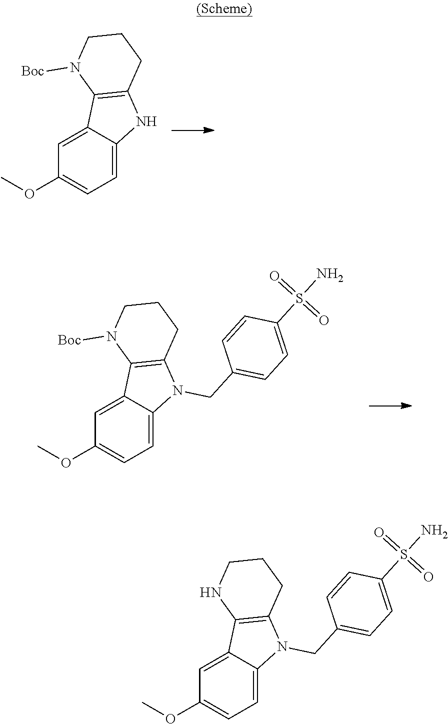 [SMILES: COc1ccc2[nH]c3c(c2c1)N(C(=O)OC(C)(C)C)CCC3 ; COc1ccc2c(c1)c1c(n2Cc2ccc(S(N)(=O)=O)cc2)CCCN1C(=O)OC(C)(C)C ; COc1ccc2c(c1)c1c(n2Cc2ccc(S(N)(=O)=O)cc2)CCCN1]

### (Step 1) Preparation of tert-butyl 8-methoxy-5-(4-sulfamoylbenzyl)-2,3,4,5-tetrahydro-1H-pyrido[3,2-b]indole-1-carboxylate

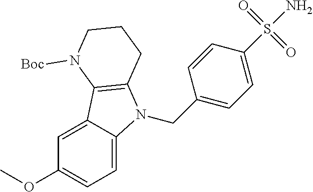 [SMILES: COc1ccc2c(c1)c1c(n2Cc2ccc(S(N)(=O)=O)cc2)CCCN1C(=O)OC(C)(C)C]

The compound prepared in (Step 1) of Example 5 (200 mg, 0.66 mmol) was dissolved in N,N-dimethylformamide (2.00 mL), and then cooled to 0° C., and 60% sodium hydride (52 mg, 1.32 mmol) was added to the mixture and stirred for 30 minutes. Intermediate 1 (330 mg, 1.32 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=2:8) to obtain the title compound as a yellow solid. (22.0 mg, 7%) MS m/z: 472 [M+1]⁺

### (Step 2) Preparation of 4-((8-methoxy-1,2,3,4-tetrahydro-5H-pyrido[3,2-b]indol-5-yl)methyl)benzenesulfonamide (Compound 38)

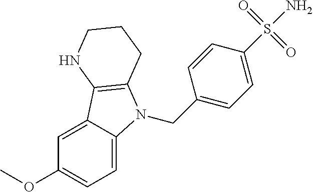 [SMILES: COc1ccc2c(c1)c1c(n2Cc2ccc(S(N)(=O)=O)cc2)CCCN1]

The compound prepared in (Step 1) above (11.0 mg, 0.02 mmol) was dissolved in methylene chloride (1.00 mL), and then 4N hydrogen chloride dioxane solution (1.00 mL) was added to the mixture. The reaction mixture was stirred at room temperature for 2 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (6.00 mg, 68%) MS m/z: 372 [M+1]^{+ 1}H NMR (400 MHz, CD₃OD) δ 7.78 (d, J=7.4 Hz, 2H), 7.10 (d, J=7.7 Hz, 3H), 7.00 (s, 1H), 6.72 (s, 1H), 5.33 (s, 2H), 3.82 (d=J=1.62 Hz, 3H), 3.23 (s, 2H), 2.69 (s, 2H), 2.02 (s, 2H).

### [Example 39] Preparation of 3-((8-methoxy-1,2,3,4-tetrahydro-5H-pyrido[3,2-b]indol-5-yl)methyl)benzene sulfonamide (Compound 39)

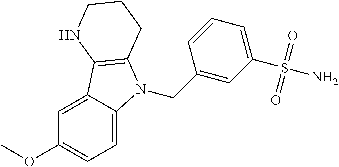 [SMILES: COc1ccc2c(c1)c1c(n2Cc2cccc(S(N)(=O)=O)c2)CCCN1]

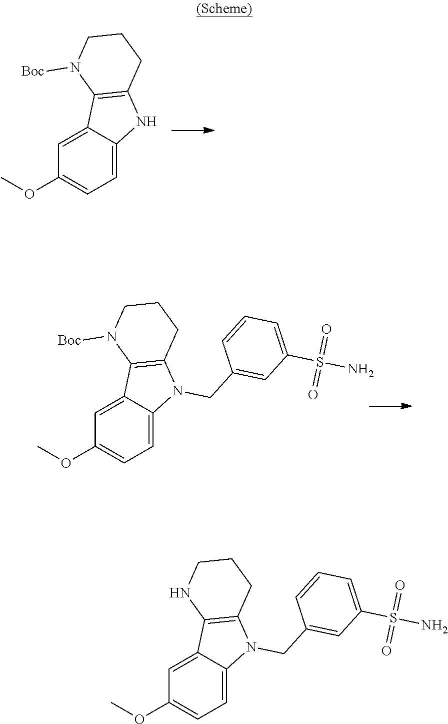 [SMILES: COc1ccc2[nH]c3c(c2c1)N(C(=O)OC(C)(C)C)CCC3 ; COc1ccc2c(c1)c1c(n2Cc2cccc(S(N)(=O)=O)c2)CCCN1C(=O)OC(C)(C)C ; COc1ccc2c(c1)c1c(n2Cc2cccc(S(N)(=O)=O)c2)CCCN1]

### (Step 1) Preparation of tert-butyl 8-methoxy-5-(3-sulfamoylbenzyl)-2,3,4,5-tetrahydro-1H-pyrido[3,2-b]indole-1-carboxylate

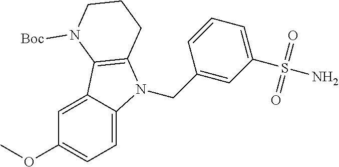 [SMILES: COc1ccc2c(c1)c1c(n2Cc2cccc(S(N)(=O)=O)c2)CCCN1C(=O)OC(C)(C)C]

The compound prepared in (Step 1) of Example 5 (50.0 mg, 0.17 mmol) was dissolved in N,N-dimethylformamide (1.00 mL), and then cooled to 0° C., and 60% sodium hydride (19.8 mg, 0.50 mmol) was added thereto and stirred for 30 minutes. 3-(Bromomethyl)benzenesulfonamide (45.0 mg, 0.18 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:8) to obtain the title compound as a yellow solid. (22.0 mg, 28%) MS m/z: 472 [M+1]⁺

### (Step 2) Preparation of 3-((8-methoxy-1,2,3,4-tetrahydro-5H-pyrido[3,2-b]indol-5-yl)methyl)benzenesulfonamide (Compound 39)

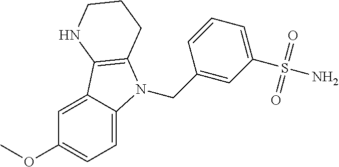 [SMILES: COc1ccc2c(c1)c1c(n2Cc2cccc(S(N)(=O)=O)c2)CCCN1]

The compound prepared in (Step 1) above (22.0 mg, 0.05 mmol) was dissolved in methylene chloride (0.50 mL), and then trifluoroacetic acid (0.50 mL) was added to the mixture. The reaction mixture was stirred at room temperature for 3 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (5.00 mg, 29%) MS m/z: 372 [M+1]^{+ 1}H NMR (400 MHz, CD₃OD) δ 7.64 (d, J=8.8 Hz, 2H), 7.40-7.34 (m, 1H), 7.30 (t, J=7.6 Hz, 1H), 7.13 (dd, J=9.2, 2.6 Hz, 1H), 6.84 (d, J=2.6 Hz, 1H), 6.74 (d, J=9.2 Hz, 1H), 4.14-3.95 (m, 2H), 3.69 (s, 3H), 3.58 (d, J=13.6 Hz, 1H), 3.26 (d, J=13.7 Hz, 1H), 2.59 (ddd, J=12.7, 8.2, 3.8 Hz, 1H), 2.28 (tdd, J=16.9, 10.6, 6.3 Hz, 1H), 2.06-1.92 (m, 1H), 1.83 (dt, J=13.2, 9.1 Hz, 1H).

### [Example 40] Preparation of 4-((8-methyl-1,2,3,4-tetrahydro-5H-pyrido[3,2-b]indol-5-yl)methyl benzenesulfonamide (Compound 40)

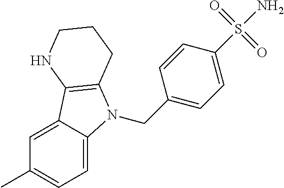 [SMILES: Cc1ccc2c(c1)c1c(n2Cc2ccc(S(N)(=O)=O)cc2)CCCN1]

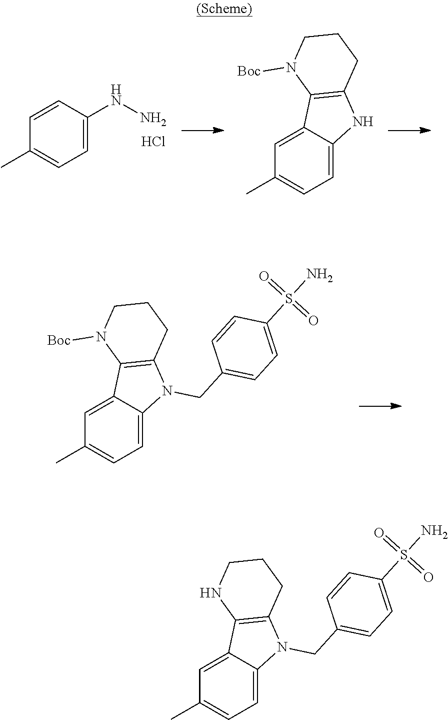

### (Step 1) Preparation of tert-butyl 8-methyl-2,3,4,5-tetrahydro-1H-pyrido[3,2-b]indole-1-carboxylate

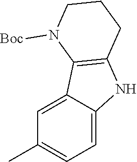 [SMILES: Cc1ccc2[nH]c3c(c2c1)N(C(=O)OC(C)(C)C)CCC3]

p-Tolylhydrazine-hydrochloride (400 mg, 2.52 mmol) was dissolved in toluene (25.0 mL), and then tert-butyl-3-oxopiperidine-1-carboxylate (603 mg, 3.03 mmol) and propanephosphonic anhydride (0.30 mL, 0.50 mmol) were added to the mixture sequentially. The reaction mixture was stirred at 90° C. for 6 hours under nitrogen. After cooling to room temperature, distilled water and an aqueous sodium bicarbonate solution were added, and extracted twice with ethyl acetate. The organic layers were dried over anhydrous sodium sulfate, and then concentrated under reduced pressure. The residue was purified by silica gel column chromatography (ethyl acetate:n-hexane=1:2) to obtain the title compound as a yellow solid. (320 mg, 44%) MS m/z: 287 [M+1]⁺

### (Step 2) Preparation of tert-butyl 8-methyl-5-(4-sulfamoylbenzyl)-2,3,4,5-tetrahydro-1H-pyrido[3,2-b]indole-1-carboxylate

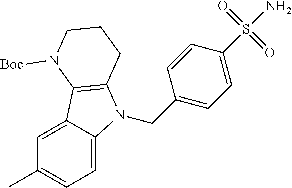 [SMILES: Cc1ccc2c(c1)c1c(n2Cc2ccc(S(N)(=O)=O)cc2)CCCN1C(=O)OC(C)(C)C]

The compound prepared in (Step 1) above (50.0 mg, 0.18 mmol) was dissolved in N,N-dimethylformamide (2.40 mL), and then cooled to 0° C., and 60% sodium hydride (14.0 mg, 0.35 mmol) was added to the mixture and stirred for 30 minutes. Intermediate 1 (52.4 mg, 0.21 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:3) to obtain the title compound as a white solid. (17.1 mg, 21%) MS m/z: 456 [M+1]⁺

### (Step 3) Preparation of 4-((8-methyl-1,2,3,4-tetrahydro-5H-pyrido[3,2-b]indol-5-yl)methyl)benzenesulfonamide (Compound 40)

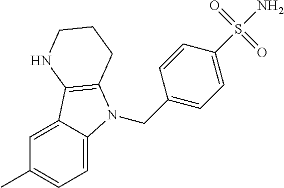 [SMILES: Cc1ccc2c(c1)c1c(n2Cc2ccc(S(N)(=O)=O)cc2)CCCN1]

The compound prepared in (Step 2) above (17.1 mg, 0.04 mmol) was dissolved in methylene chloride (0.80 mL), and then trifluoroacetic acid (0.40 mL) was added to the mixture. The reaction mixture was stirred at room temperature for 2 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a pink solid. (10.6 mg, 79%) MS m/z: 356 [M+1]^{+ 1}H NMR (400 MHz, CD₃OD) δ 7.86-7.74 (m, 2H), 7.35 (s, 1H), 7.30 (d, J=9.0 Hz, 1H), 7.23-7.12 (m, 2H), 7.08 (d, J=9.1 Hz, 1H), 5.48 (s, 2H), 3.60 (d, J=5.9 Hz, 2H), 2.85 (t, J=5.9 Hz, 2H), 2.45 (s, 3H), 2.36-2.16 (m, 2H).

### [Example 41] Preparation of 4-((8-methoxy-1-oxo-1,2,3,4-tetrahydro-5H-pyrido[4,3-b]indol-5-yl)methyl)benzene sulfonamide (Compound 41)

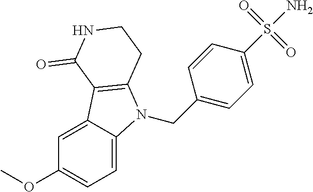 [SMILES: COc1ccc2c(c1)c1c(n2Cc2ccc(S(N)(=O)=O)cc2)CCNC1=O]

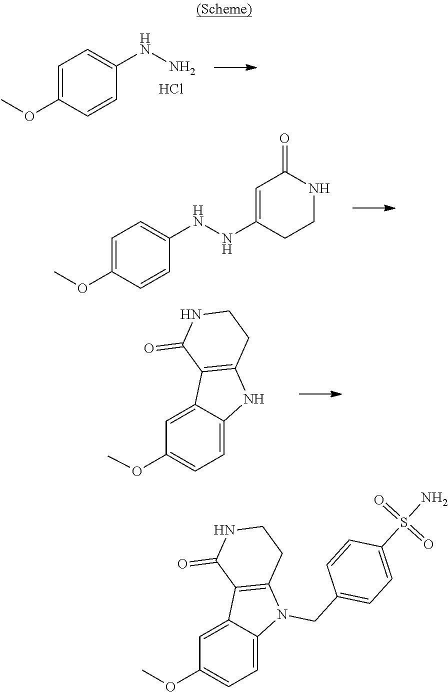

### (Step 1) Preparation of 4-(2-(4-methoxyphenyl) hydrazineyl)-5,6-dihydropyridin-2 (1H)-one

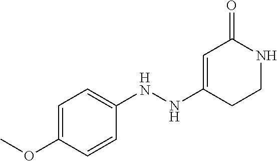 [SMILES: COc1ccc(NNC2=CC(=O)NCC2)cc1]

Piperidine-2,4-dione (6.48 g, 57.3 mmol) was dissolved in ethanol (80 mL), and then 4-methoxyhydrazine-hydrochloride (10.0 g, 57.3 mmol) was added to the mixture. The reaction mixture was stirred at room temperature for 1 hour under nitrogen. After completion of the reaction, the mixture was concentrated under reduced pressure. The title compound was obtained as a brown solid without further purification. (9.80 g) MS 1 m/z: 234 [M+1]⁺

### (Step 2) Preparation of 8-methoxy-2,3,4,5-tetrahydro-1H-pyrido[4,3-b]indol-1-one

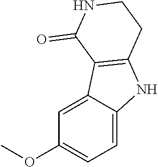 [SMILES: COc1ccc2[nH]c3c(c2c1)C(=O)NCC3]

The compound prepared in (Step 1) above (8.80 g, 37.7 mmol) was slowly dissolved in a mixed solution (44.0 mL) of sulfuric acid:distilled water=3:1 at 0° C., and then stirred at room temperature for 3 hours. After completion of the reaction, distilled water and 2 M aqueous sodium hydroxide solution (200 mL) were slowly added, and extracted twice with a mixed solution of methylene chloride:methanol=10:1. The organic layers were dried over anhydrous sodium sulfate, and then concentrated under reduced pressure. The residue was purified by silica gel column chromatography (methylene chloride:methanol=100:1 to 10:1) to obtain the title compound as a yellow solid. (1.06 g, 13%) MS m/z: 217 [M+1]^{+ 1}H NMR (400 MHz, DMSO-d₆) δ 11.47 (s, 1H), 7.35 (d, J=2.5 Hz, 1H), 7.25 (d, J=8.7 Hz, 1H), 6.96 (br s, 1H), 6.72 (dd, J=2.6, 8.8 Hz, 1H), 3.74 (s, 3H), 3.43 (dt, J=2.3, 6.9 Hz, 2H), 2.92 (t, J=6.9 Hz, 2H).

### (Step 3) Preparation of 4-((8-methoxy-1-oxo-1,2,3,4-tetrahydro-5H-pyrido[4,3-b]indol-5-yl)methyl)benzene sulfonamide (Compound 41)

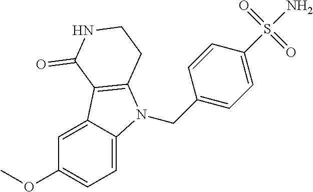 [SMILES: COc1ccc2c(c1)c1c(n2Cc2ccc(S(N)(=O)=O)cc2)CCNC1=O]

The compound prepared in (Step 2) above (50.0 mg, 0.23 mmol) was dissolved in N,N-dimethylformamide (1.00 mL), and then cooled to 0° C., and 60% sodium hydride (18.0 mg, 0.46 mmol) was added to the mixture and stirred for 30 minutes. Intermediate 1 (86.0 mg, 0.34 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (2.80 mg, 3%) MS m/z: 386 [M+1]^{+ 1}H NMR (400 MHz, CD₃OD) δ 7.92 (d, J=8.0 Hz, 1H), 7.87 (d, J=8.3 Hz, 1H), 7.62 (d, J=9.2 Hz, 2H), 7.26 (t, J=9.7 Hz, 2H), 6.83 (d, J=9.5 Hz, 1H), 5.54 (s, 2H), 3.87 (s, 3H), 3.65 (s, 2H), 3.03 (s, 2H)

### [Example 42] Preparation of 4-((7-methoxy-1-oxo-1,2,3,4-tetrahydro-5H-pyrido[4,3-b]indol-5-yl)methyl)benzene sulfonamide (Compound 42)

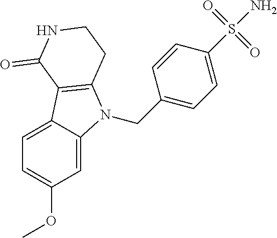 [SMILES: COc1ccc2c3c(n(Cc4ccc(S(N)(=O)=O)cc4)c2c1)CCNC3=O]

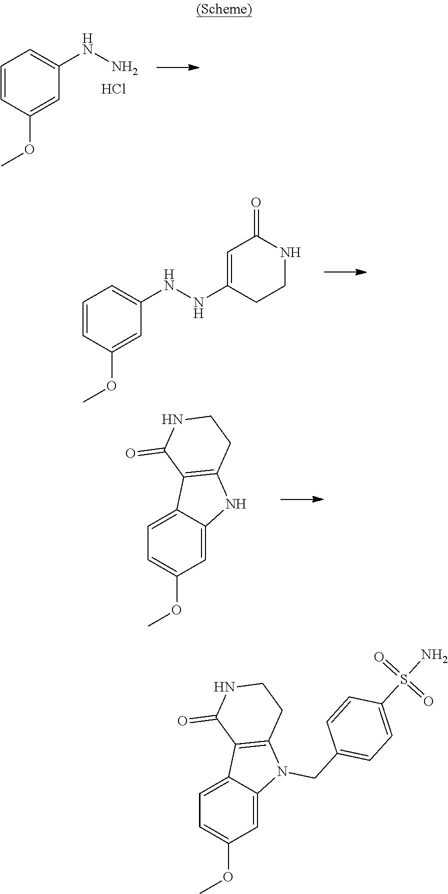

### (Step 1) Preparation of 4-(2-(3-methoxyphenyl) hydrazineyl)-5,6-dihydropyridin-2 (1H)-one

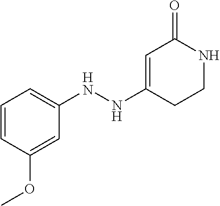 [SMILES: COc1cccc(NNC2=CC(=O)NCC2)c1]

Piperidine-2,4-dione (10.0 g, 88.4 mmol) was dissolved in ethanol (80 mL), and then 3-methoxyhydrazine-hydrochloride (15.4 g, 88.4 mmol) was added to the mixture. The reaction mixture was stirred at room temperature for 1 hour under nitrogen. After completion of the reaction, the mixture was concentrated under reduced pressure. The title compound was obtained as a yellow solid without further purification. (20.0 g) MS m/z: 234 [M+1]⁺

### (Step 2) Preparation of 7-methoxy-2,3,4,5-tetrahydro-1H-pyrido[4,3-b]indol-1-one

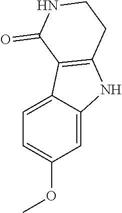 [SMILES: COc1ccc2c3c([nH]c2c1)CCNC3=O]

The compound prepared in (Step 1) above (10.0 g, 42.9 mmol) was slowly dissolved in a mixed solution (45.0 mL) of sulfuric acid:distilled water=3:1 at 0° C., and then stirred at room temperature for 3 hours. After completion of the reaction, distilled water and 2 M aqueous sodium hydroxide solution (200 mL) were slowly added to the mixture, and extracted twice with a mixed solution of methylene chloride:methanol=10:1. The organic layers were dried over anhydrous sodium sulfate, and then concentrated under reduced pressure. The residue was purified by silica gel column chromatography (methylene chloride:methanol=100:1 to 10:1) to obtain the title compound as a yellow solid. (354 mg, 4%) MS m/z: 217 [M+1]^{+ 1}H NMR (400 MHz, DMSO-d₆) δ 11.42 (s, 1H), 7.70 (d, J=8.6 Hz, 1H), 6.93 (br s, 1H), 6.88 (d, J=2.1 Hz, 1H), 6.73 (dd, J=2.3, 8.6 Hz, 1H), 3.76 (s, 3H), 3.44 (dt, J=2.4, 6.9 Hz, 2H), 2.91 (t, J=6.9 Hz, 2H).

### (Step 3) Preparation of 4-((7-methoxy-1-oxo-1,2,3,4-tetrahydro-5H-pyrido[4,3-b]indol-5-yl)methyl)benzene sulfonamide (Compound 42)

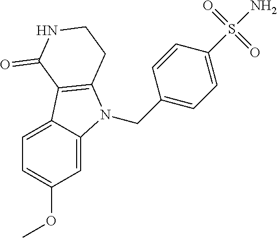 [SMILES: COc1ccc2c3c(n(Cc4ccc(S(N)(=O)=O)cc4)c2c1)CCNC3=O]

The compound prepared in (Step 2) above (50.0 mg, 0.23 mmol) was dissolved in N,N-dimethylformamide (2.40 mL), and then cooled to 0° C., and 60% sodium hydride (18.5 mg, 0.46 mmol) was added to the mixture and stirred for 30 minutes. Intermediate 1 (69.4 mg, 0.28 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a white solid. (16.2 mg, 18%) MS m/z: 386 [M+1]^{+ 1}H NMR (400 MHz, CD₃OD) δ 7.96-7.73 (m, 3H), 7.30-7.10 (m, 2H), 6.92-6.75 (m, 2H), 5.50 (s, 2H), 3.75 (s, 3H), 3.59 (t, J=7.2 Hz, 2H), 2.96 (t, J=7.1 Hz, 2H).

### [Example 43] Preparation of 4-((7-methoxy-1-oxo-1,2-dihydro-5H-pyrido[4,3-b]indol-5-yl)methyl)benzene sulfonamide (Compound 43)

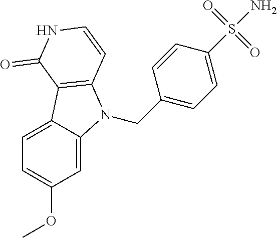 [SMILES: COc1ccc2c3c(=O)[nH]ccc3n(Cc3ccc(S(N)(=O)=O)cc3)c2c1]

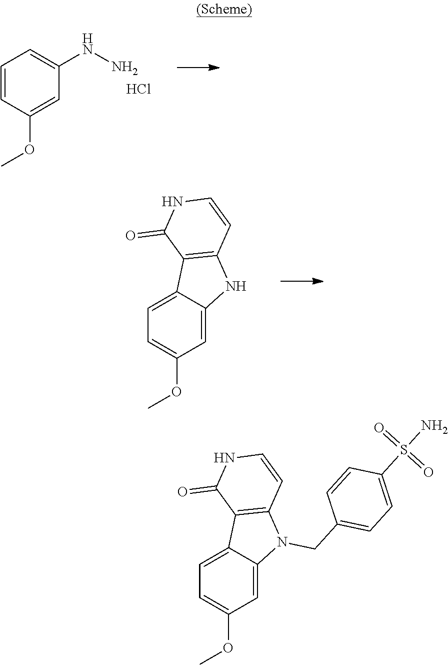

### (Step 1) Preparation of 7-methoxy-2,5-dihydro-1H-pyrido[4,3-b]indol-1-one

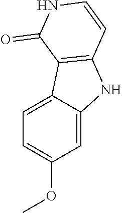 [SMILES: COc1ccc2c(c1)[nH]c1cc[nH]c(=O)c12]

(3-Methoxyphenyl) hydrazine-hydrochloride (1.12 g, 8.10 mmol) was dissolved in diphenyl ether (50.0 mL), and then 2,4-dihydroxypyridine (300 mg, 2.70 mmol) was added to the mixture sequentially. The reaction mixture was stirred at 180° C. for 1 hour and then at 240° C. for 3 hours under nitrogen. After completion of the reaction, it was cooled to room temperature, and then the residue was purified by silica gel column chromatography (ethyl acetate:n-hexane=0:10 to methanol:methylene chloride=1:9) to obtain the title compound as a yellow solid. (196 mg, 34%) MS m/z: 215 [M+1]^{+ 1}H NMR (400 MHz, CD₃OD) δ 8.08 (d, J=8.4 Hz, 1H), 7.31 (s, 1H), 7.01 (s, 1H), 6.89 (d, J=8.3 Hz, 1H), 6.67 (d, J=4.5 Hz, 1H), 3.86 (s, 3H).

### (Step 2) Preparation of 4-((7-methoxy-1-oxo-1,2-dihydro-5H-pyrido[4,3-b]indol-5-yl)methyl)benzenesulfonamide (Compound 43)

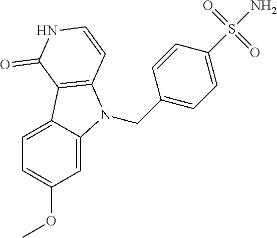 [SMILES: COc1ccc2c3c(=O)[nH]ccc3n(Cc3ccc(S(N)(=O)=O)cc3)c2c1]

The compound prepared in (Step 1) above (52.0 mg, 0.24 mmol) was dissolved in N,N-dimethylformamide (1.00 mL), and then cooled to 0° C., and 60% sodium hydride (19.0 mg, 0.48 mmol) was added to the mixture and stirred for 30 minutes. Intermediate 1 (72.0 mg, 0.29 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (3.70 mg, 3%) MS m/z: 384 [M+1]^{+ 1}H NMR (400 MHz, CD₃OD) δ 8.06 (s, 1H), 7.87 (s, 2H), 7.58 (s, 1H), 7.45 (s, 2H), 7.02 (s, 1H), 6.90 (s, 1H), 6.72 (s, 1H), 5.41 (s, 2H), 3.88 (s, 3H)

### [Example 44] Preparation of 4-((8-methoxy-1-oxo-1,2-dihydro-5H-pyridazino[4,5-b]indol-5-yl)methyl)benzene sulfonamide (Compound 44)

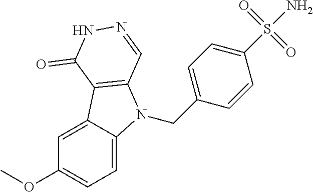 [SMILES: COc1ccc2c(c1)c1c(=O)[nH]ncc1n2Cc1ccc(S(N)(=O)=O)cc1]

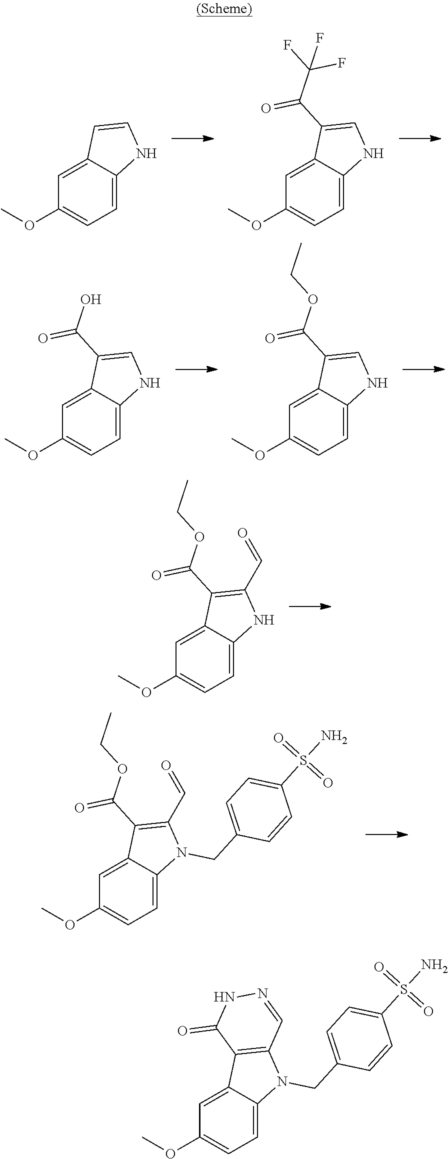

### (Step 1) Preparation of 2,2,2-trifluoro-1-(5-methoxy-1H-indol-3-yl) ethan-1-one

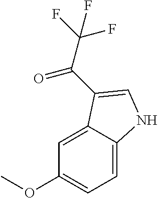 [SMILES: COc1ccc2[nH]cc(C(=O)C(F)(F)F)c2c1]

5-Methoxyindole (3.00 g, 20.4 mmol) was dissolved in N,N-dimethylformamide (15.0 mL), and then trifluoroacetic anhydride (TFAA, 1.60 mL, 30.6 mmol) was added to the mixture at 0° C. The reaction mixture was stirred at room temperature for 16 hours under nitrogen. After completion of the reaction, distilled water was added to the mixture, and the resulting solid was filtered and dried to obtain the title compound as a white solid. (6.40 g, 129%) MS m/z: 244 [M+1]^{+ 1}H NMR (400 MHz, DMSO-d₆) δ ppm: 12.60 (s, 1H), 8.40 (s, 1H), 7.68 (s, 1H), 7.49 (d, J=8.9 Hz, 1H), 6.97 (d, J=8.8 Hz, 1H), 3.81 (s, 3H).

### (Step 2) Preparation of 5-methoxy-1H-indole-3-carboxylic acid

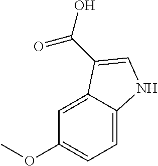

The compound prepared in (Step 1) above (6.40 g) was dissolved in 5 N aqueous sodium hydroxide solution (20.0 mL), and then tetrahydrofuran (5.00 mL) was added to the mixture. The reaction mixture was stirred at 90° C. for 16 hours under nitrogen. The reaction mixture was concentrated under reduced pressure. After cooling to room temperature, distilled water was added slowly, and extracted twice with ethyl acetate. The water layers were adjusted to pH 2 or less by adding 1 N aqueous hydrogen chloride solution thereto, and the resulting solid was filtered and dried to obtain the title compound as a white solid. (2.90 g, 76%) MS m/z: 192 [M+1]^{+ 1}H NMR (400 MHz, DMSO-d₆) δ ppm: 11.66 (s, 1H), 7.92 (d, J=2.5 Hz, 1H), 7.49 (d, J=2.6 Hz, 1H), 7.35 (d, J=8.8 Hz, 1H), 6.82 (dd, J=8.8, 2.5 Hz, 1H), 3.78 (s, 3H).

### (Step 3) Preparation of ethyl 5-methoxy-1H-indole-3-carboxylate

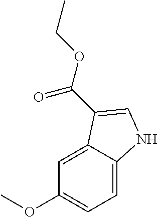 [SMILES: CCOC(=O)c1c[nH]c2ccc(OC)cc12]

The compound prepared in (Step 2) above (1.30 g, 6.80 mmol) was dissolved in methanol:distilled water (10.0 mL, 9:1), and then cesium carbonate (2.10 g, 6.46 mmol) was added to the mixture and stirred at room temperature for 1 hour. After completion of the reaction, it was concentrated under reduced pressure, and iodoethane (0.49 mL, 6.12 mmol) was added and stirred for additional 16 hours. Distilled water was slowly added to the reaction mixture, and extracted twice with ethyl acetate. The organic layers were dried over anhydrous sodium sulfate, and then concentrated under reduced pressure. The residue was purified by silica gel column chromatography (ethyl acetate:n-hexane=0:10 to 2:3) to obtain the title compound as a white solid. (1.10 g, 74%) MS m/z: 220 [M+1]^{+ 1}H NMR (400 MHz, DMSO-d₆) δ ppm: 11.78 (s, 1H), 7.98 (d, J=2.9 Hz, 1H), 7.48 (d, J=2.6 Hz, 1H), 7.35 (d, J=8.9 Hz, 1H), 6.82 (dd, J=8.8, 2.6 Hz, 1H), 4.24 (q, J=7.1 Hz, 2H), 3.78 (s, 3H), 1.31 (t, J=7.1 Hz, 3H).

### (Step 4) Preparation of ethyl 2-formyl-5-methoxy-1H-indole-3-carboxylate

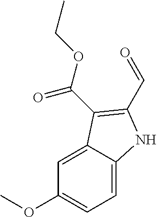 [SMILES: CCOC(=O)c1c(C=O)[nH]c2ccc(OC)cc12]

Phosphoryl chloride (0.39 mL, 4.20 mmol) and N,N-dimethylformamide (0.28 mL, 3.65 mmol) were sequentially added thereto at 0° C. under nitrogen, and then stirred for 1 minutes. The reaction mixture was dissolved in methylene chloride (5.00 mL), and the compound prepared in (Step 3) above (400 mg, 1.82 mmol) was slowly added to the mixture, and then stirred at room temperature for 16 hours. After completion of the reaction, it was concentrated under reduced pressure, and 10 mL of an aqueous saturated sodium acetate solution was added and stirred for additional 12 hours. The reaction mixture was extracted twice with methylene chloride. The organic layers were dried over anhydrous sodium sulfate, and then concentrated under reduced pressure. The residue was purified by silica gel column chromatography (ethyl acetate:n-hexane=0:10 to 2:3) to obtain the title compound as a white solid. (80.0 mg, 18%) MS m/z: 248 [M+1]^{+ 1}H NMR (400 MHz, DMSO-d₆) δ ppm: 12.07 (s, 1H), 10.66 (s, 1H), 8.09 (d, J=3.4 Hz, 1H), 7.66 (dd, J=8.9, 3.4 Hz, 1H), 7.10 (d, J=9.0 Hz, 1H), 4.05-4.00 (m, 2H), 3.85 (s, 3H), 1.16 (t, J=4.8 Hz, 3H).

### (Step 5) Preparation of ethyl 2-formyl-5-methoxy-1-(4-sulfamoylbenzyl)-1H-indole-3-carboxylate

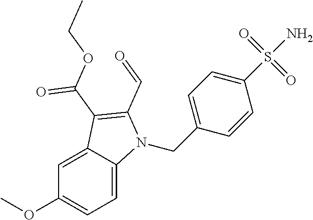 [SMILES: CCOC(=O)c1c(C=O)n(Cc2ccc(S(N)(=O)=O)cc2)c2ccc(OC)cc12]

The compound prepared in (Step 4) above (25.0 mg, 0.10 mmol) was dissolved in N,N-dimethylformamide (1.00 mL), and then cooled to 0° C., and 60% sodium hydride (4.90 mg, 0.12 mmol) was added to the mixture and stirred for 30 minutes. Intermediate 1 (30.3 mg, 0.12 mmol) was slowly added to the reaction mixture, and then stirred at 0° C. for 2 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:4 to 9:1) to obtain the title compound as a yellow solid. (23.0 mg, 55%) MS m/z: 417 [M+1]^{+ 1}H NMR (400 MHz, CD₃OD) δ ppm: 10.77 (s, 1H), 8.19 (s, 1H), 7.86 (d, J=8.0 Hz, 2H), 7.60 (d, J=9.2 Hz, 1H), 7.34 (d, J=8.1 Hz, 2H), 7.10 (d, J=9.1 Hz, 1H), 5.60 (s, 2H), 4.32 (q, J=6.7 Hz, 2H), 3.92 (s, 3H), 1.37 (t, J=7.1 Hz, 3H).

### (Step 6) Preparation of 4-((8-methoxy-1-oxo-1,2-dihydro-5H-pyridazino[4,5-b]indol-5-yl)methyl)benzene sulfonamide (Compound 44)

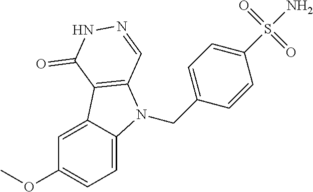 [SMILES: COc1ccc2c(c1)c1c(=O)[nH]ncc1n2Cc1ccc(S(N)(=O)=O)cc1]

The compound prepared in (Step 5) above (23.0 mg, 0.055 mmol) was dissolved in acetic acid (0.50 mL) and ethanol (0.50 mL), and then hydrazine-monohydrate (8.00 μl, 0.17 mmol) was added to the mixture. The reaction mixture was stirred at 90° C. for 3 hours under nitrogen. After completion of the reaction, it was concentrated under reduced pressure. The residue was purified by preparative high performance liquid chromatography (prep-HPLC, 0.1% TFA acetonitrile:distilled water=5:95 to 100:0) to obtain the title compound as a yellow solid. (4.70 mg, 17%) MS m/z: 499 [M+1]^{+ 1}H NMR (400 MHz, DMSO-d₆) δ ppm: 10.18 (s, 1H), 8.07 (s, 1H), 7.77 (d, J=7.6 Hz, 2H), 7.63 (s, 1H), 7.47-7.27 (m, 5H), 6.93 (d, J=8.7 Hz, 1H), 5.50 (s, 2H), 3.81 (s, 3H).

### [Example 45] Preparation of 4-((7-methoxy-1-oxo-1,2-dihydro-5H-pyridazino[4,5-b]indol-5-yl)methyl)benzene sulfonamide (Compound 45)

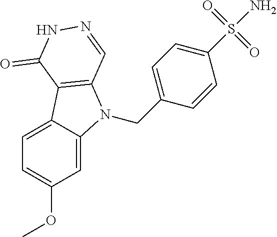 [SMILES: COc1ccc2c3c(=O)[nH]ncc3n(Cc3ccc(S(N)(=O)=O)cc3)c2c1]

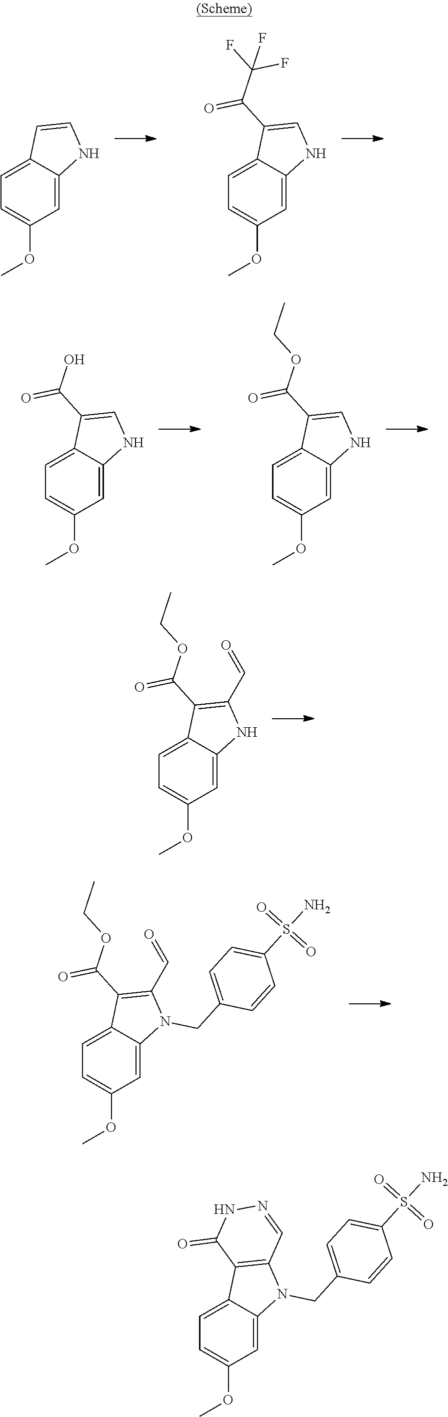

### (Step 1) Preparation of 2,2,2-trifluoro-1-(6-methoxy-1H-indol-3-yl) ethan-1-one

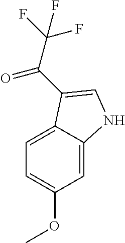 [SMILES: COc1ccc2c(C(=O)C(F)(F)F)c[nH]c2c1]

6-Methoxyindole (3.00 g, 20.38 mmol) was dissolved in N,N-dimethylformamide (15.0 mL), and then trifluoroacetic anhydride (TFAA, 1.60 mL, 30.6 mmol) was added to the mixture at 0° C. The reaction mixture was stirred at room temperature for 16 hours under nitrogen. After completion of the reaction, distilled water was added to the mixture, and the resulting solid was filtered and dried to obtain the title compound as a white solid. (7.00 g, 141%) MS m/z: 244 [M+1]^{+ 1}H NMR (400 MHz, DMSO-d₆) δ ppm: 12.50 (s, 1H), 8.36 (s, 1H), 8.02 (d, J=8.7 Hz, 1H), 7.06 (s, 1H), 6.95 (d, J=8.6 Hz, 1H), 3.81 (s, 3H).

### (Step 2) Preparation of 6-methoxy-1H-indole-3-carboxylic acid

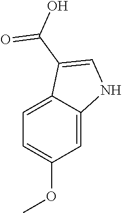

The compound prepared in (Step 1) above (7.00 g) was dissolved in 5 N aqueous sodium hydroxide solution (20.0 mL), and then tetrahydrofuran (5.00 mL) was added to the mixture. The reaction mixture was stirred at 90° C. for 16 hours under nitrogen. The reaction mixture was concentrated under reduced pressure. After cooling to room temperature, distilled water was added slowly, and extracted twice with ethyl acetate. The water layers were adjusted to pH 2 or less by adding 1 N aqueous hydrogen chloride solution, and the resulting solid was filtered and dried to obtain the title compound as a white solid. (3.50 g, 90%) MS m/z: 192 [M+1]^{+ 1}H NMR (400 MHz, DMSO-d₆) δ ppm: 11.84 (br, 1H), 11.57 (s, 1H), 7.90-7.72 (m, 2H), 6.92 (s, 1H), 6.78 (d, J=8.7, 1H), 3.76 (s, 3H).

### (Step 3) Preparation of ethyl 6-methoxy-1H-indole-3-carboxylate

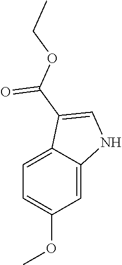 [SMILES: CCOC(=O)c1c[nH]c2cc(OC)ccc12]

The compound prepared in (Step 2) above (500 mg, 2.61 mmol) was dissolved in methanol:distilled water (10.0 mL, 9:1), and then cesium carbonate (810 mg, 2.48 mmol) was added to the mixture and stirred at room temperature for 1 hour. After completion of the reaction, it was concentrated under reduced pressure, and iodoethane (0.19 mL, 2.35 mmol) was added and stirred for additional 16 hours. Distilled water was slowly added to the reaction mixture, and extracted twice with ethyl acetate. The organic layers were dried over anhydrous sodium sulfate, and then concentrated under reduced pressure. The residue was purified by silica gel column chromatography (ethyl acetate:n-hexane=0:10 to 2:3) to obtain the title compound as a white solid. (372 mg, 64%) MS m/z: 220 [M+1]^{+ 1}H NMR (400 MHz, CDCl₃) δ ppm: 8.37 (br, 1H), 8.06 (d, J=8.8 Hz, 1H), 7.83 (s, 1H), 7.01-6.93 (m, 1H), 6.90 (s, 1H), 4.49-4.29 (m, 2H), 3.88 (s, 3H), 1.44 (t, J=7.2 Hz, 3H).

### (Step 4) Preparation of ethyl 2-formyl-6-methoxy-1H-indole-3-carboxylate

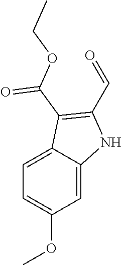 [SMILES: CCOC(=O)c1c(C=O)[nH]c2cc(OC)ccc12]

Phosphoryl chloride (0.39 mL, 4.20 mmol) and N,N-dimethylformamide (0.28 mL, 3.65 mmol) were sequentially added to the mixture at 0° C. under nitrogen, and then stirred for 1 minutes. The compound prepared in (Step 3) above (400 mg, 1.82 mmol) dissolved in methylene chloride (5.20 mL) was slowly added to the reaction mixture, and then stirred at room temperature for 16 hours. After completion of the reaction, it was concentrated under reduced pressure, and 10.0 mL of an aqueous saturated sodium acetate solution was added and stirred for additional 12 hours. The reaction mixture was extracted twice with methylene chloride. The organic layers were dried over anhydrous sodium sulfate, and then concentrated under reduced pressure. The residue was purified by silica gel column chromatography (ethyl acetate:n-hexane=1:9 to 2:3) to obtain the title compound as a white solid. (330 mg, 37%) MS m/z: 248 [M+1]⁺

### (Step 5) Preparation of ethyl 2-formyl-6-methoxy-1-(4-sulfamoylbenzyl)-1H-indole-3-carboxylate

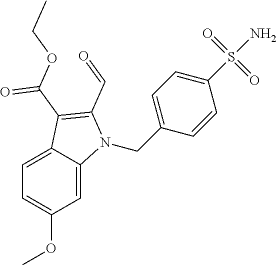 [SMILES: CCOC(=O)c1c(C=O)n(Cc2ccc(S(N)(=O)=O)cc2)c2cc(OC)ccc12]

The compound prepared in (Step 4) above (50.0 mg, 0.20 mmol) was dissolved in N,N-dimethylformamide (2.00 mL), and then cooled to 0° C., and 60% sodium hydride (16.1 mg, 0.40 mmol) was added to the mixture and stirred for 30 minutes. Intermediate 1 (60.7 mg, 0.24 mmol) dissolved in N,N-dimethylformamide (0.30 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:8 to 1:2) to obtain the title compound as a yellow solid. (35.0 mg, 42%) MS m/z: 417 [M+1]⁺

### (Step 6) Preparation of 4-((7-methoxy-1-oxo-1,2-dihydro-5H-pyridazino[4,5-b]indol-5-yl)methyl)benzene sulfonamide (Compound 45)

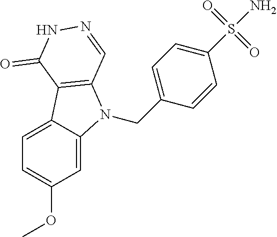 [SMILES: COc1ccc2c3c(=O)[nH]ncc3n(Cc3ccc(S(N)(=O)=O)cc3)c2c1]

The compound prepared in (Step 5) above (35.0 mg, 0.08 mmol) was dissolved in acetic anhydride (0.40 mL), and then hydrazine-monohydrate (12.0 μl, 0.25 mmol) was added to the mixture. The reaction mixture was stirred at 80° C. for 3 hours under nitrogen. After completion of the reaction, it was concentrated under reduced pressure. The residue was purified by preparative high performance liquid chromatography (prep-HPLC, 0.1% TFA acetonitrile:distilled water=5:95 to 100:0) to obtain the title compound as a yellow solid. (4.60 mg, 14%) MS m/z: 385 [M+1]^{+ 1}H NMR (400 MHz, CD₃OD) δ 8.53 (s, 1H), 8.22 (d, J=9.3 Hz, 1H), 7.94-7.73 (m, 2H), 7.33 (d, J=5.4 Hz, 2H), 7.17 (s, 1H), 7.07 (d, J=8.6 Hz, 1H), 5.86 (s, 2H), 3.88 (s, 3H).

### [Example 46] Preparation of 7-methoxy-5-(4-(S-methylsulfonimidoyl)benzyl)-2,5-dihydro-1H-pyridazino[4,5-b]indol-1-one (Compound 46)

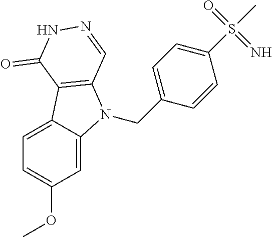 [SMILES: COc1ccc2c3c(=O)[nH]ncc3n(Cc3ccc(S(C)(=N)=O)cc3)c2c1]

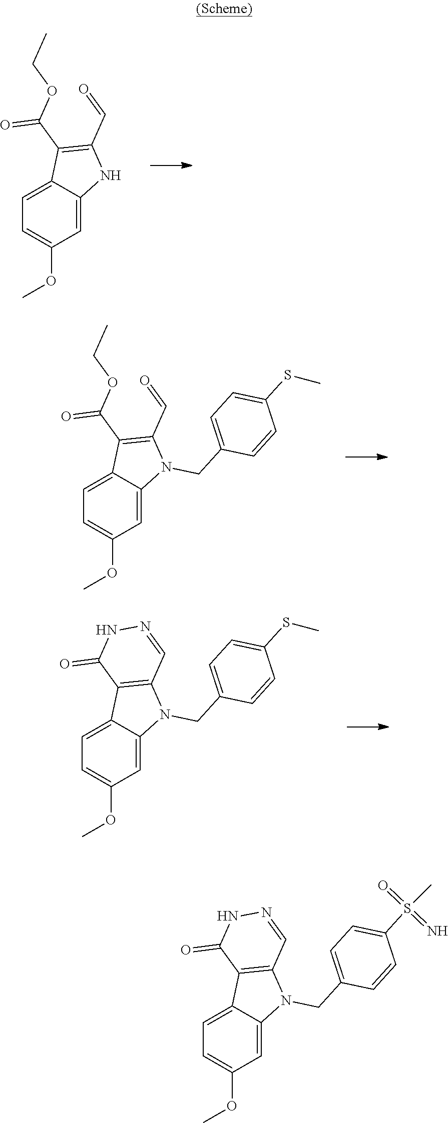 [SMILES: CCOC(=O)c1c(C=O)[nH]c2cc(OC)ccc12 ; CCOC(=O)c1c(C=O)n(Cc2ccc(SC)cc2)c2cc(OC)ccc12 ; COc1ccc2c3c(=O)[nH]ncc3n(Cc3ccc(SC)cc3)c2c1 ; COc1ccc2c3c(=O)[nH]ncc3n(Cc3ccc(S(C)(=N)=O)cc3)c2c1]

### (Step 1) Preparation of ethyl 2-formyl-6-methoxy-1-(4-(methylthio)benzyl)-1H-indole-3-carboxylate

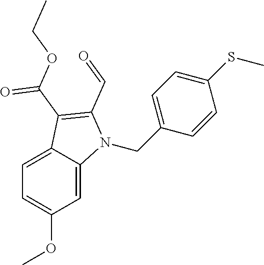 [SMILES: CCOC(=O)c1c(C=O)n(Cc2ccc(SC)cc2)c2cc(OC)ccc12]

The compound prepared in (Step 4) of Example 45 (50.0 mg, 0.20 mmol) was dissolved in N,N-dimethylformamide (2.00 mL), and then cooled to 0° C., and 60% sodium hydride (16.2 mg, 0.40 mmol) was added to the mixture and stirred for 30 minutes. Intermediate 6 (87.8 mg, 0.40 mmol) dissolved in N,N-dimethylformamide (0.50 mL) was slowly added to the reaction mixture, and then stirred at 0° C. for 1 hour under nitrogen. After completion of the reaction, distilled water was added thereto slowly, and extracted twice with ethyl acetate. The organic layers were dried over anhydrous sodium sulfate, and then concentrated under reduced pressure. The residue was purified by silica gel column chromatography (ethyl acetate:n-hexane=1:8 to 1:4) to obtain the title compound as a yellow solid. (75.0 mg, 97%) MS m/z: 384 [M+1]⁺

### (Step 2) Preparation of 7-methoxy-5-(4-(methylthio)benzyl)-2,5-dihydro-1H-pyridazino[4,5-b]indol-1-one

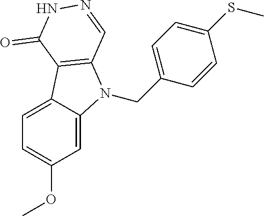 [SMILES: COc1ccc2c3c(=O)[nH]ncc3n(Cc3ccc(SC)cc3)c2c1]

The compound prepared in (Step 1) above (75.0 mg, 0.20 mmol) was dissolved in acetic anhydride (2.00 mL), and then hydrazine-monohydrate (0.03 mL, 0.59 mmol) was added to the mixture. The reaction mixture was stirred at 80° C. for 3 hours under nitrogen. After completion of the reaction, it was concentrated under reduced pressure. The residue was purified by preparative high performance liquid chromatography (prep-HPLC, 0.1% TFA acetonitrile:distilled water=5:95 to 100:0) to obtain the title compound as a yellow solid. (38.0 mg, 55%) MS m/z: 352 [M+1]⁺

### (Step 3) Preparation of 7-methoxy-5-(4-(S-methylsulfonimidoyl)benzyl)-2,5-dihydro-1H-pyridazino[4,5-b]indol-1-one (Compound 46)

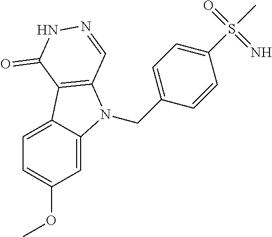 [SMILES: COc1ccc2c3c(=O)[nH]ncc3n(Cc3ccc(S(C)(=N)=O)cc3)c2c1]

The compound prepared in (Step 2) above (20.0 mg, 0.06 mmol) was dissolved in ethanol (1.70 mL), and then iodobenzene diacetate (53.2 mg, 0.17 mmol) and ammonium acetate (16.7 mg, 0.22 mmol) were added to the mixture. The reaction mixture was stirred at room temperature for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a white solid. (4.60 mg, 21%) MS m/z: 383 [M+1]^{+ 1}H NMR (400 MHz, CD₃OD) δ 8.49 (s, 1H), 8.22 (d, J=7.6 Hz, 1H), 8.10 (d, J=5.6 Hz, 2H), 7.52 (d, J=3.6 Hz, 2H), 7.12 (s, 1H), 7.07 (d, J=7.2 Hz, 1H), 5.95 (s, 2H), 3.86 (s, 3H), 2.03 (s, 3H).

### [Example 47] Preparation of 4-((7-methoxy-5H-pyridazino[4,5-b]indol-5-yl)methyl)benzenesulfonamide (Compound 47)

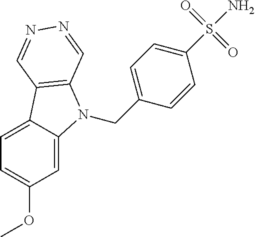 [SMILES: COc1ccc2c3cnncc3n(Cc3ccc(S(N)(=O)=O)cc3)c2c1]

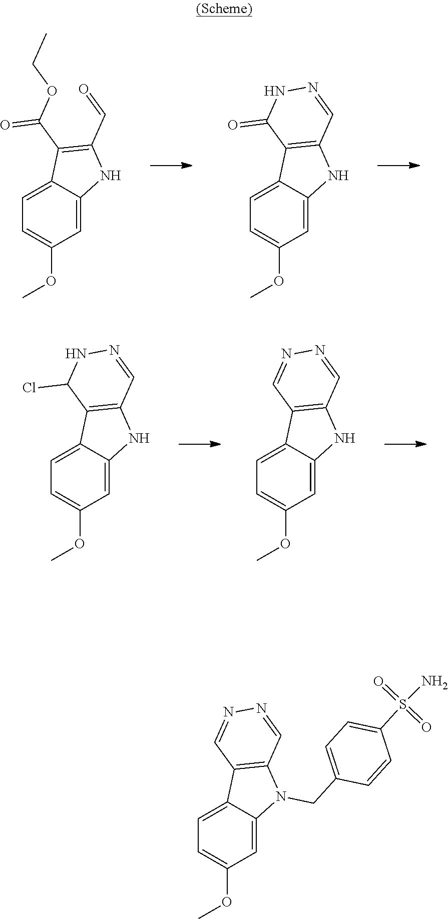 [SMILES: CCOC(=O)c1c(C=O)[nH]c2cc(OC)ccc12 ; COc1ccc2c(c1)[nH]c1cn[nH]c(=O)c12 ; COc1ccc2c3c([nH]c2c1)C=NNC3Cl ; COc1ccc2c(c1)[nH]c1cnncc12 ; COc1ccc2c3cnncc3n(Cc3ccc(S(N)(=O)=O)cc3)c2c1]

### (Step 1) Preparation of 7-methoxy-2,5-dihydro-1H-pyridazino[4,5-b]indol-1-one

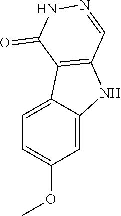 [SMILES: COc1ccc2c(c1)[nH]c1cn[nH]c(=O)c12]

The compound prepared in (Step 4) of Example 45 (75.0 mg, 0.30 mmol) was dissolved in acetic anhydride (3.00 mL), and then hydrazine-monohydrate (44.0 μl, 0.91 mmol) was added to the mixture. The reaction mixture was stirred at 80° C. for 3 hours under nitrogen. After completion of the reaction, it was cooled to room temperature and concentrated under reduced pressure. The residue was purified by preparative high performance liquid chromatography (prep-HPLC, 0.1% TFA acetonitrile:distilled water=5:95 to 100:0) to obtain the title compound as a yellow solid. (12.1 mg, 19%) MS m/z: 216 [M+1]⁺

### (Step 2) Preparation of 1-chloro-7-methoxy-2,5-dihydro-1H-pyridazino[4,5-b]indole

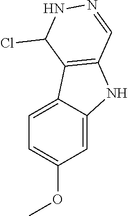 [SMILES: COc1ccc2c3c([nH]c2c1)C=NNC3Cl]

The compound prepared in (Step 1) above (12.1 mg, 0.06 mmol) was dissolved in trichlorophosphate (1.00 mL), and then stirred at 60° C. for 3 hours under nitrogen. After completion of the reaction, it was cooled to room temperature, and then distilled water was added to the mixture slowly, and extracted twice with ethyl acetate. The organic layers were dried over anhydrous sodium sulfate, and then concentrated under reduced pressure. The title compound was obtained as a white solid without further purification. (13.1 mg, 99%) MS m/z: 244 [M+1]⁺

### (Step 3) Preparation of 7-methoxy-5H-pyridazino[4,5-b]indole

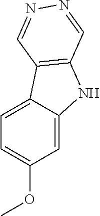

The compound prepared in (Step 2) above (13.1 mg, 0.06 mmol) was dissolved in methanol (1.10 mL), and then ammonium formate (14.0 mg, 0.22 mmol) and 10% Pd/C (5.00 mg) were added to the mixture. The reaction mixture was stirred at room temperature for 12 hours under nitrogen. Precipitation was removed by filtration using Celite, and then the filtrate was concentrated under reduced pressure. The title compound was obtained as a white solid without further purification. (11.2 mg, 100%) MS m/z: 200 [M+1]⁺

### (Step 4) Preparation of 4-((7-methoxy-5H-pyridazino[4,5-b]indol-5-yl)methyl)benzenesulfonamide (Compound 47)

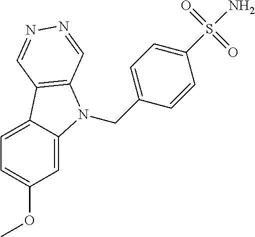 [SMILES: COc1ccc2c3cnncc3n(Cc3ccc(S(N)(=O)=O)cc3)c2c1]

The compound prepared in (Step 3) above (11.2 mg, 0.06 mmol) was dissolved in N,N-dimethylformamide (0.60 mL), and then cooled to 0° C., and 60% sodium hydride (4.50 mg, 0.11 mmol) was added to the mixture and stirred for 30 minutes. Intermediate 1 (21.1 mg, 0.08 mmol) dissolved in N,N-dimethylformamide (0.30 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (1.70 mg, 8%) MS m/z: 369 [M+1]^{+ 1}H NMR (400 MHz, CD₃OD) δ 9.99 (s, 1H), 9.64 (s, 1H), 8.34 (d, J=5.2 Hz, 1H), 7.86 (d, J=4.4 Hz, 2H), 7.38 (s, 2H), 7.30 (s, 1H), 7.21 (d, J=6.4 Hz, 1H), 6.06 (s, 2H), 3.99 (s, 3H).

### [Example 48] Preparation of 4-((7-methoxy-9H-pyrimido[4,5-b]indol-9-yl)methyl)benzenesulfonamide (Compound 48)

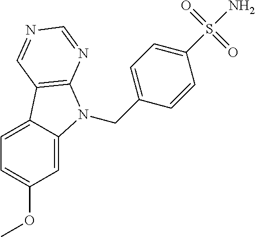 [SMILES: COc1ccc2c3cncnc3n(Cc3ccc(S(N)(=O)=O)cc3)c2c1]

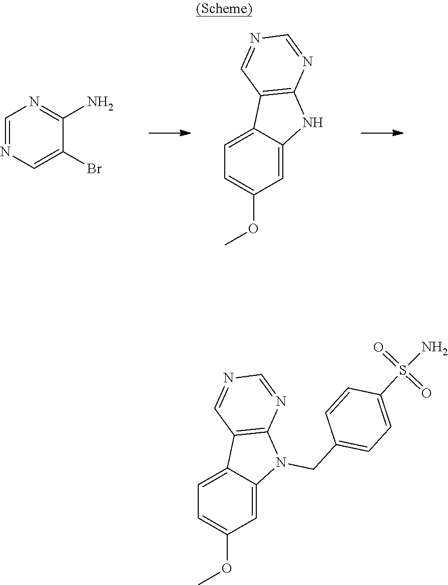 [SMILES: Nc1ncncc1Br ; COc1ccc2c(c1)[nH]c1ncncc12 ; COc1ccc2c3cncnc3n(Cc3ccc(S(N)(=O)=O)cc3)c2c1]

### (Step 1) Preparation of 7-methoxy-9H-pyrimido[4,5-b]indole

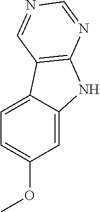

4-Amino-5-bromopyrimidine (500 mg, 2.87 mmol) was dissolved in 1,4-dioxane (6.00 mL), and then [1,1-bis(diphenylphosphino)ferrosine]dichloropalladium (165 mg, 0.29 mmol), xantphos (166 mg, 0.29 mmol) and sodium tert-butoxide (828 mg, 8.62 mmol) were added to the mixture sequentially and stirred at 110° C. for 24 hours. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:1) to obtain the title compound as a pale yellow solid. (220 mg, 38%) MS m/z: 200 [M+1]^{+ 1}H NMR (400 MHz, CDCl₃) δ 9.74 (s, 1H), 9.21 (d, J=5.3 Hz, 1H), 8.99 (d, J=5.6 Hz, 1H), 7.99 (s, 1H), 7.11-6.84 (m, 2H), 3.93 (d, J=1.3 Hz, 3H).

### (Step 2) Preparation of 4-((7-methoxy-9H-pyrimido[4,5-b]indol-9-yl)methyl)benzenesulfonamide (Compound 48)

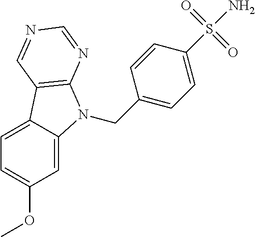 [SMILES: COc1ccc2c3cncnc3n(Cc3ccc(S(N)(=O)=O)cc3)c2c1]

The compound prepared in (Step 1) above (50.0 mg, 0.25 mmol) was dissolved in N,N-dimethylformamide (1.00 mL), and then cooled to 0° C., and 60% sodium hydride (20.0 mg, 0.50 mmol) was added to the mixture and stirred for 30 minutes. Intermediate 1 (95.0 mg, 0.38 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (49.0 mg, 51%) MS m/z: 369 [M+1]^{+ 1}H NMR (400 MHz, CD₃OD) δ 9.58-9.43 (m, 1H), 9.30-9.12 (m, 1H), 8.38-8.21 (m, 1H), 7.91-7.77 (m, 2H), 7.63-7.43 (m, 2H), 7.34-7.24 (m, 1H), 7.24-7.13 (m, 1H), 6.02-5.86 (m, 2H), 3.91 (t, J=3.2 Hz, 3H).

### [Example 49] Preparation of 4-((6-methoxy-9H-pyrido[2,3-b]indol-9-yl)methyl)benzenesulfonamide (Compound 49)

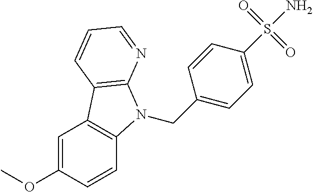 [SMILES: COc1ccc2c(c1)c1cccnc1n2Cc1ccc(S(N)(=O)=O)cc1]

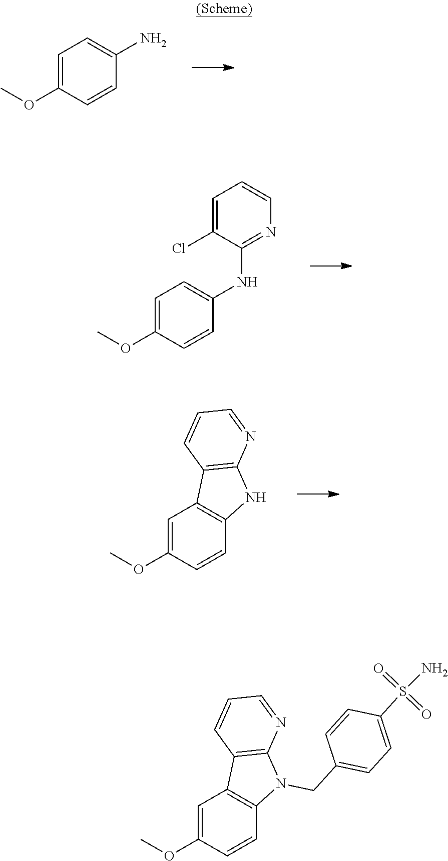

### (Step 1) Preparation of 3-chloro-N-(4-methoxyphenyl)pyridin-2-amine

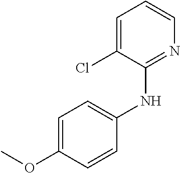 [SMILES: COc1ccc(Nc2ncccc2Cl)cc1]

The compounds 4-methoxyaniline (500 mg, 4.05 mmol) and 2,3-dichloropyridine (500 mg, 3.34 mmol) were dissolved in toluene (10.0 mL), and then palladium acetate (75.8 mg, 0.34 mmol), BINAP (421 mg, 0.68 mmol) and potassium carbonate (1.60 g, 11.8 mol) were added to the mixture and stirred at 100° C. for 18 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 10:0) to obtain the title compound as a yellow solid. (540 mg, 68%). MS m/z: 235 [M+1]+ 1H NMR (400 MHz, CDCl3) δ 8.07 (dd, J=4.9, 1.4 Hz, 1H), 7.57-7.51 (m, 1H), 7.51-7.45 (m, 2H), 6.93-6.88 (m, 2H), 6.80 (s, 1H), 6.69-6.63 (m, 1H), 3.81 (d, J=0.8 Hz, 3H).

### (Step 2) Preparation of 6-methoxy-9H-pyrido[2,3-b]indole

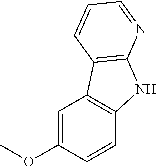

The compound (35.0 mg, 0.15 mmol) prepared in (Step 1) above was dissolved in N,N-dimethylformamide (1.0 mL), and then palladium acetate (3.30 mg, 0.01 mmol) and potassium carbonate (31.0 mg, 0.22 mmol) were added to the mixture and stirred at 135° C. for 18 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 10:0) to obtain the title compound as a yellow solid. (5.0 mg, 17%). MS m/z: 199 [M+1]+ 1H NMR (400 MHz, CDCl3) δ 9.08 (s, 1H), 8.45 (dd, J=5.0, 1.6 Hz, 1H), 8.30 (dd, J=7.8, 1.6 Hz, 1H), 7.54 (d, J=2.6 Hz, 1H), 7.42 (d, J=8.8 Hz, 1H), 7.15 (ddd, J=13.9, 8.3, 3.8 Hz, 2H), 3.93 (s, 3H).

### (Step 3) Preparation of 4-((6-methoxy-9H-pyrido[2,3-b]indol-9-yl)methyl)benzenesulfonamide (Compound 49)

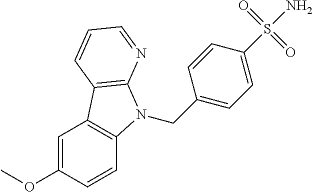 [SMILES: COc1ccc2c(c1)c1cccnc1n2Cc1ccc(S(N)(=O)=O)cc1]

The compound (19.0 mg, 0.096 mmol) prepared in (Step 2) above was dissolved in N,N-dimethylformamide (1.00 mL), and then cooled to 0° C., and 60% sodium hydride (7.80 mg, 0.11 mmol) was added to the mixture and stirred for 30 minutes. Intermediate 1 (28.8 mg, 0.12 mmol) dissolved in N,N-dimethylformamide (0.50 mL) was slowly added to the reaction mixture, and then stirred at 0° C. for 2 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:4 to 10:0) to obtain the title compound as a yellow solid. (2.9 mg, 8%). MS m/z: 368 [M+1]+1H NMR (400 MHz, CD3OD) δ 8.62 (dd, J=7.8, 1.5 Hz, 1H), 8.45 (d, J=5.3 Hz, 1H), 7.82 (d, J=8.4 Hz, 2H), 7.78 (d, J=2.9 Hz, 1H), 7.40 (d, J=9.0 Hz, 1H), 7.34 (dd, J=7.8, 5.1 Hz, 3H), 7.15 (dd, J=8.9, 2.6 Hz, 1H), 5.81 (s, 2H), 3.93 (d, J=1.0 Hz, 3H).

### [Example 50] Preparation of 4-((8-methoxy-5H-pyridazino[4,5-b]indol-5-yl)methyl)benzenesulfonamide (Compound 50)

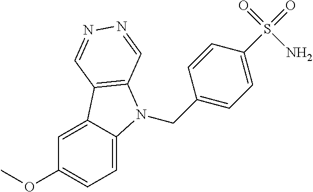 [SMILES: COc1ccc2c(c1)c1cnncc1n2Cc1ccc(S(N)(=O)=O)cc1]

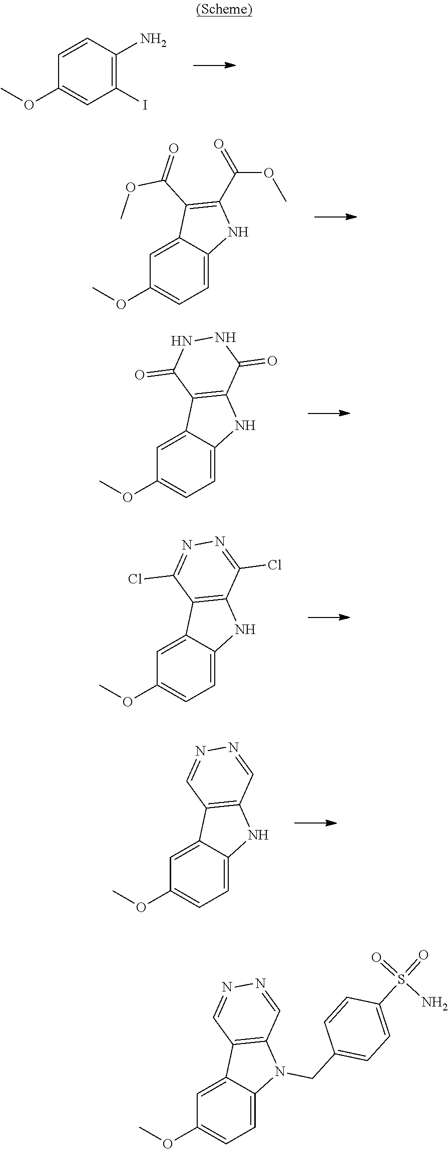

### (Step 1) Preparation of dimethyl 5-methoxy-1H-indole-2,3-dicarboxylate

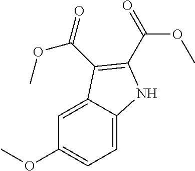

2-Iodo-4-methoxyaniline (5.0 g, 20.1 mmol) and dimethyl acetylene dicarboxylate (3.14 g, 22.1 mmol) were dissolved in dimethyl sulfoxide (50.0 mL), and then tris (dibenzylideneacetone) dipalladium (0) (919 mg, 1.00 mmol) and triethylamine (8.38 mL, 60.2 mmol) were added to the mixture and stirred at 90° C. for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:ether=1:100 to 2:5) to obtain the title compound as a yellow solid. (960 mg, 18%). MS m/z: 264 [M+1]+1H NMR (400 MHz, CDCl3) δ 9.19 (br s, 1H), 7.49 (d, J=2.4 Hz, 1H), 7.33 (d, J=9.2 Hz, 1H), 7.05 (dd, J=9.2, 2.4 Hz, 1H), 3.99 (s, 3H), 3.99 (s, 3H), 3.89 (s, 3H).

### (Step 2) Preparation of 8-methoxy-2,3-dihydro-1H-pyridazino[4,5-b]indole-1,4 (5H)-dione

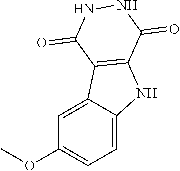 [SMILES: COc1ccc2[nH]c3c(=O)[nH][nH]c(=O)c3c2c1]

The compound (960 mg, 3.65 mmol) prepared in (Step 1) above was dissolved in ethanol (5.7 mL), and then hydrazine monohydrate (5.82 g, 114 mmol, 98% pure) was added to the mixture slowly. The mixture was stirred at 80° C. for 16 hours under nitrogen. After completion of the reaction, it was concentrated under reduced pressure. The title compound was obtained as a yellow solid without further purification. (970 mg). MS m/z: 232 [M+1]+ 1H NMR (400 MHz, CDCl3) δ 7.56 (d, J=2.4 Hz, 1H), 7.49 (d, J=8.8 Hz, 1H), 7.04 (dd, J=8.8, 2.4 Hz, 1H), 3.82 (s, 3H).

### (Step 3) Preparation of 1,4-dichloro-8-methoxy-5H-pyridazino[4,5-b]indole

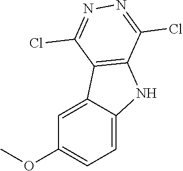 [SMILES: COc1ccc2[nH]c3c(Cl)nnc(Cl)c3c2c1]

The compound (970 mg, 4.20 mmol) prepared in (Step 2) above was cooled to 0° C., and then phosphoryl chloride (5.00 mL) was added to the mixture slowly. The mixture was stirred at 110° C. for 16 hours under nitrogen. The reaction was completed by slowly adding aqueous sodium carbonate solution, and then the resulting solid was filtered to obtain the title compound as a brown solid. (870 mg, 77%). MS m/z: 268 [M+1]+ 1H NMR (400 MHz, DMSO-d6) δ 7.80 (d, J=2.4 Hz, 1H), 7.73 (d, J=8.8 Hz, 1H) 7.42 (dd, J=8.8, 2.4 Hz, 1H), 3.91 (s, 3H).

### (Step 4) Preparation of 8-methoxy-5H-pyridazino[4,5-b]indole

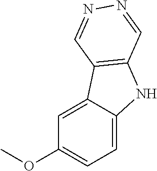

The compound (870 mg, 3.25 mmol) prepared in (Step 3) above was dissolved in methanol (30.0 mL), and then Pd/C (300 mg, 10% pure, 50% wet) was added to the mixture. The mixture was stirred at room temperature for 2 hours under hydrogen. After completion of the reaction, it was filtrated through a Celite pad to remove impurities, and then the solvent was removed from the filtrate under reduced pressure. The residue was purified by silica gel column chromatography (methanol:methylene chloride=1:10) to obtain the title compound as a brown solid. (403 mg, 62%). MS m/z: 200 [M+1]+ 1H NMR (400 MHz, DMSO-d6) δ 12.44-13.76 (m, 1H), 10.21 (s, 1H), 9.64 (s, 1H), 8.05 (d, J=2.4 Hz, 1H), 7.79 (d, J=8.8 Hz, 1H), 7.40 (dd, J=8.8, 2.4 Hz, 1H), 3.90 (s, 3H)

### (Step 5) Preparation of 4-((6-methoxy-9H-pyrido[2,3-b]indol-9-yl)methyl)benzenesulfonamide (Compound 49)

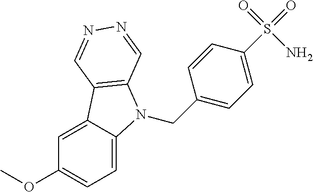 [SMILES: COc1ccc2c(c1)c1cnncc1n2Cc1ccc(S(N)(=O)=O)cc1]

The compound (50.0 mg, 0.251 mmol) prepared in (Step 4) above was dissolved in N,N-dimethylformamide (2.50 mL), and then cooled to 0° C., and 60% sodium hydride (20.1 mg, 0.50 mmol) was added to the mixture and stirred for 30 minutes. Intermediate 1 (75.3 mg, 0.30 mmol) dissolved in N,N-dimethylformamide (0.50 mL) was slowly added to the reaction mixture, and then stirred at 0° C. for 2 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (5.6 mg, 6%). MS m/z: 369 [M+1]+ 1H NMR (400 MHz, CD3OD) δ 10.21 (s, 1H), 10.01 (s, 1H), 8.00-7.95 (m, 3H), 7.84 (d, J=9.2 Hz, 1H), 7.73 (d, J=8.0 Hz, 2H), 7.57 (dd, J=2.2, 9.0 Hz, 1H), 6.14 (s, 2H), 3.98 (s, 3H)

### [Example 51] Preparation of 4-((8-methoxy-5H-pyrimido[5,4-b]indol-5-yl)methyl benzenesulfonamide (Compound 51)

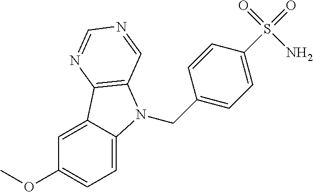 [SMILES: COc1ccc2c(c1)c1ncncc1n2Cc1ccc(S(N)(=O)=O)cc1]

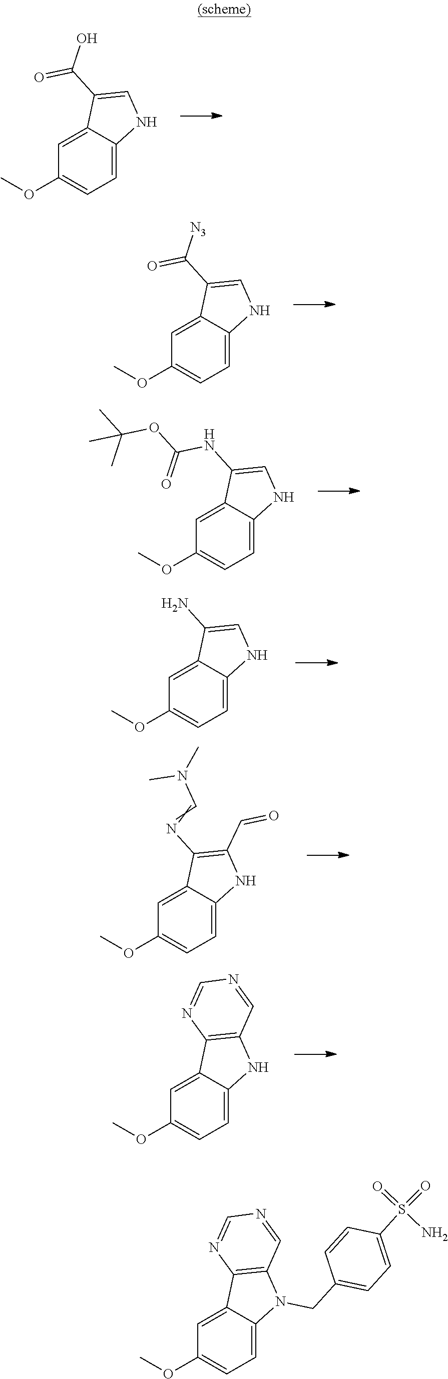

### (Step 1) Preparation of 5-methoxy-1H-indole-3-carbonyl azide

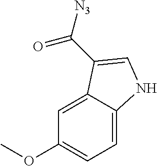 [SMILES: COc1ccc2[nH]cc(C(=O)N=[N+]=[N-])c2c1]

5-Methoxyindole-3-carboxylic acid (1.00 g, 5.23 mmol) was dissolved in tetrahydrofuran (17.0 mL), and then diphenylphosphoryl azide (1.18 mL, 5.49 mmol) and triethylamine (0.77 mL, 5.49 mmol) were added to the mixture, and stirred at room temperature for 24 hours. After completion of the reaction, the reaction mixture was concentrated under reduced pressure. The residue was purified by silica gel column chromatography (ethyl acetate:n-hexane=2:3) to obtain the title compound as a white solid. (870 mg, 77%). MS m/z: 217 [M+1]+ 1H NMR (400 MHz, CDCl3) δ ppm: 12.11 (s, 1H), 8.12 (s, 1H), 7.53 (d, J=2.6 Hz, 1H), 7.42 (d, J=8.9 Hz, 1H), 6.89 (dd, J=8.9, 2.6 Hz, 1H), 3.80 (s, 3H)

### (Step 2) Preparation of tert-butyl (5-methoxy-1H-indol-3-yl) carbamate

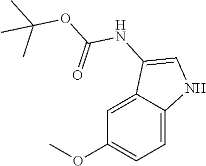 [SMILES: COc1ccc2[nH]cc(NC(=O)OC(C)(C)C)c2c1]

The compound (870 mg, 4.02 mmol) prepared in (Step 1) above was dissolved in tert-butanol (30.0 mL), and then the reaction mixture was stirred at 80° C. for 16 hours under nitrogen. After cooling to room temperature, the reaction mixture was concentrated under reduced pressure. The residue was purified by silica gel column chromatography (ethyl acetate:n-hexane=0:10 to 1:3) to obtain the title compound as a green solid. (589 mg, 56%). MS m/z: 263 [M+1]+ 1H NMR (400 MHz, DMSO-d6) δ ppm: 10.49 (s, 1H), 8.99 (s, 1H), 7.34 (d, J=16.4 Hz, 2H), 7.17 (d, J=8.8 Hz, 1H), 6.69 (dd, J=8.8, 2.5 Hz, 1H), 3.73 (s, 3H), 1.49 (s, 9H).

### (Step 3) Preparation of 5-methoxy-1H-indol-3-amine

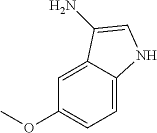

The compound (100 mg, 0.38 mmol) prepared in (Step 2) above was dissolved in methylene chloride (1.50 mL), and then trifluoroacetic acid (0.14 mL, 1.91 mmol) was added to the mixture, and stirred at room temperature for 2 hours under nitrogen. After completion of the reaction, the reaction mixture was concentrated under reduced pressure to remove the solvent, and a brown mixture (140 mg) was obtained without further purification. MS m/z: 163 [M+1]+

### (Step 4) Preparation of N′-(2-formyl-5-methoxy-1H-indol-3-yl)-N,N-dimethylformimidamide

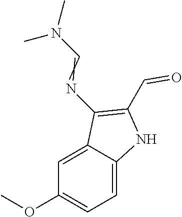 [SMILES: COc1ccc2[nH]c(C=O)c(N=CN(C)C)c2c1]

Phosphoryl chloride (0.18 mL, 1.96 mmol) was dissolved in methylene chloride (1.00 mL) at 0° C. under nitrogen, and then N,N-dimethylformamide (0.15 mL, 1.96 mmol) was added to the mixture and stirred for 30 minutes. The compound (140 mg) prepared in (Step 3) above, dissolved in methylene chloride (3.00 mL), was added slowly, and then stirred at room temperature for 24 hours. After completion of the reaction, the reaction mixture was placed in an Erlenmeyer flask filled with ice, and an aqueous sodium bicarbonate solution was added, and then extracted with ethyl acetate three times. The organic layers were dried over anhydrous sodium sulfate, and then concentrated under reduced pressure to obtain the title compound. The residue was purified by preparative high performance liquid chromatography (prep-HPLC, 0.1% TFA acetonitrile:distilled water=5:95 to 100:0) to obtain the title compound as a yellow solid. (7.5 mg). MS m/z: 246 [M+1]+

### (Step 5) Preparation of 8-methoxy-5H-pyrimido[5,4-b]indole

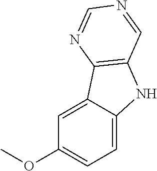

The compound (7.50 mg, 0.03 mmol) prepared in (Step 4) above was dissolved in ethanol (0.50 mL), and then an aqueous ammonia solution (0.30 mL) was added to the mixture. The reaction mixture was stirred at 80° C. for 2 hours under nitrogen. After completion of the reaction, it was concentrated under reduced pressure. The residue was purified by silica gel column chromatography (methanol:methylene chloride=1:19) to obtain the title compound as a yellow solid. (4.8 mg). MS m/z: 200 [M+1]+ 1H NMR (400 MHz, DMSO-d6) δ ppm: 11.70 (s, 1H), 9.08 (s, 1H), 9.01 (s, 1H), 7.71 (d, J=2.9 Hz, 1H), 7.61 (d, J=8.9 Hz, 1H), 7.33 (dd, J=8.6, 2.2 Hz, 1H), 3.89 (s, 3H).

### (Step 6) Preparation of 4-((8-methoxy-5H-pyrimido[5,4-b]indol-5-yl)methyl)benzenesulfonamide (Compound 51)

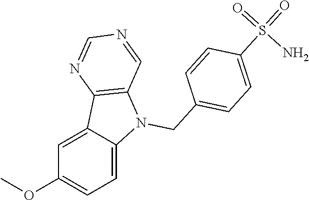 [SMILES: COc1ccc2c(c1)c1ncncc1n2Cc1ccc(S(N)(=O)=O)cc1]

The compound (4.80 mg, 0.024 mmol) prepared in (Step 5) above was dissolved in N,N-dimethylformamide (0.40 mL), and then cooled to 0° C., and 60% sodium hydride (1.70 mg, 0.043 mmol) was added to the mixture and stirred for 10 minutes. Intermediate 1 (15.1 mg, 0.06 mmol) dissolved in N,N-dimethylformamide (0.40 mL) was slowly added to the reaction mixture, and then stirred at 0° C. for 1 hour under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (3.4 mg, 38%). MS m/z: 369 [M+1]+ 1H NMR (400 MHz, DMSO-d6) δ ppm: 9.28 (s, 1H), 9.06 (s, 1H), 7.78 (d, J=2.5 Hz, 1H), 7.74 (d, J=3.4 Hz, 1H), 7.71 (d, J=4.1 Hz, 2H), 7.40-7.32 (m, 3H), 7.28 (s, 2H), 5.86 (s, 2H), 3.89 (s, 3H).

### [Example 52] Preparation of 4-((7-methoxy-5H-pyrido[4,3-b]indol-5-yl)methyl)benzenesulfonamide (Compound 52)

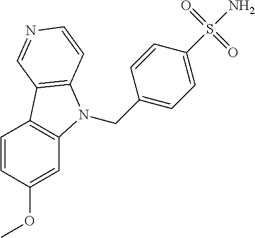 [SMILES: COc1ccc2c3cnccc3n(Cc3ccc(S(N)(=O)=O)cc3)c2c1]

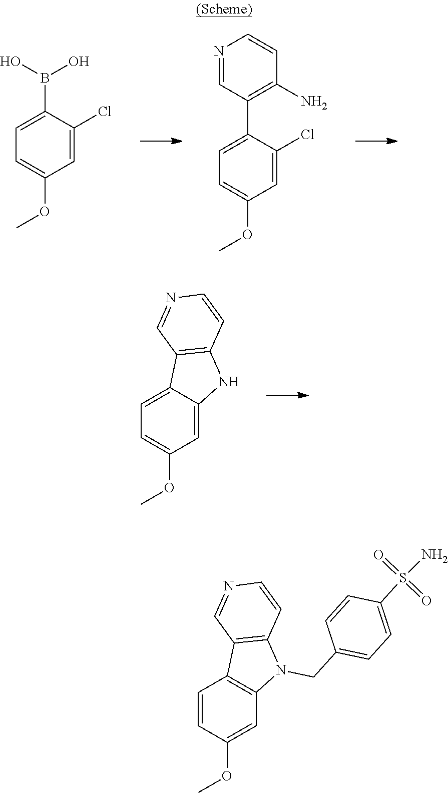

### (Step 1) Preparation of 3-(2-chloro-4-methoxyphenyl)pyridin-4-amine

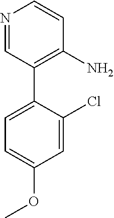 [SMILES: COc1ccc(-c2cnccc2N)c(Cl)c1]

(2-Chloro-4-methoxyphenyl) boronic acid (2.0 g, 11.6 mmol) and 4-bromopyridin-4-amine (2.59 g, 13.0 mmol) were dissolved in a mixed solution of toluene (30.0 mL), ethanol (20.0 mL) and water (10.0 mL), and then sodium carbonate (7.35 g, 69.4 mmol) and tetrakis (triphenylphosphine) palladium (0) (1.34 g, 1.16 mmol) were added to the mixture. The reaction mixture was stirred at 100° C. for 6 hours under nitrogen. After completion of the reaction, the reaction mixture was concentrated under reduced pressure. The residue was purified by silica gel column chromatography (ethyl acetate:ether=1:100 to 1:3) to obtain the title compound as a yellow solid. (1.1 g, 40%). MS m/z: 234.7 [M+1]+ 1H NMR (400 MHz, DMSO-d6) δ ppm: 8.23 (d, J=5.6 Hz, 1H), 8.10 (s, 1H), 7.23 (d, J=8.4 Hz, 1H), 7.08 (d, J=2.4 Hz, 1H), 6.92 (dd, J=8.4, 2.5 Hz, 1H), 6.63 (d, J=5.6 Hz, 1H), 4.15 (s, 2H), 3.86 (s, 3H).

### (Step 2) Preparation of 7-methoxy-5H-pyrido[4,3-b]indole

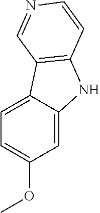

The compound (1.0 g, 4.26 mmol) prepared in (Step 1) above was dissolved in dimethyl sulfoxide (10.0 mL), and then potassium tert-butoxide (1.43 g, 12.8 mmol) was added to the mixture. The reaction mixture was stirred at 130° C. for 4 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (methanol:methylene chloride=1:100 to 1:20) to obtain the title compound as a pale brown solid. (893 mg, 52%). MS m/z: 199.0 [M+1]+ 1H NMR (400 MHz, CDCl3) δ ppm: 9.54 (br s, 1H), 9.24 (s, 1H), 8.47 (d, J=5.6 Hz, 1H), 8.01 (d, J=8.4 Hz, 1H), 7.33 (d, J=6.0 Hz, 1H), 6.92-6.99 (m, 2H), 3.90 (s, 3H).

### (Step 3) Preparation of 4-((7-methoxy-5H-pyrido[4,3-b]indol-5-yl)methyl)benzenesulfonamide (Compound 52)

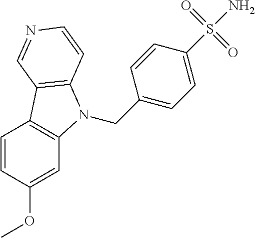 [SMILES: COc1ccc2c3cnccc3n(Cc3ccc(S(N)(=O)=O)cc3)c2c1]

The compound (50.0 mg, 0.25 mmol) prepared in (Step 2) above was dissolved in N,N-dimethylformamide (1.00 mL), and then cooled to 0° C., and 60% sodium hydride (15.0 mg, 0.38 mmol) was added to the mixture and stirred for 30 minutes. Intermediate 1 (126.2 mg, 0.50 mmol) dissolved in N,N-dimethylformamide (1.0 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (methanol:methylene chloride=1:19), and then purified by preparative high performance liquid chromatography (prep-HPLC, 0.1% TFA acetonitrile:distilled water=5:95 to 100:0) to obtain the title compound as a white solid. (15.6 mg, 16%). MS m/z: 368 [M+1]+ 1H NMR (400 MHz, DMSO-d6) δ ppm: 14.86 (s, 1H), 9.68 (s, 1H), 8.70 (d, J=6.6 Hz, 1H), 8.40 (d, J=8.8 Hz, 1H), 8.20 (d, J=6.8 Hz, 1H), 7.76 (d, J=8.4 Hz, 2H), 7.47 (d, J=2.8 Hz, 1H), 7.39 (d, J=8.3 Hz, 2H), 7.33 (s, 2H), 7.18 (dd, J=8.7, 2.3 Hz, 1H), 5.99 (s, 2H), 3.89 (s, 3H).

### [Example 53] Preparation of 4-((7-methoxy-5H-pyrido[3,2-b]indol-5-yl)methyl)benzenesulfonamide (Compound 53)

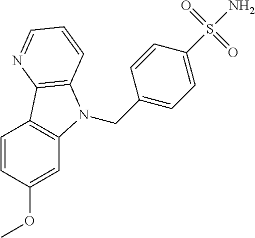 [SMILES: COc1ccc2c3ncccc3n(Cc3ccc(S(N)(=O)=O)cc3)c2c1]

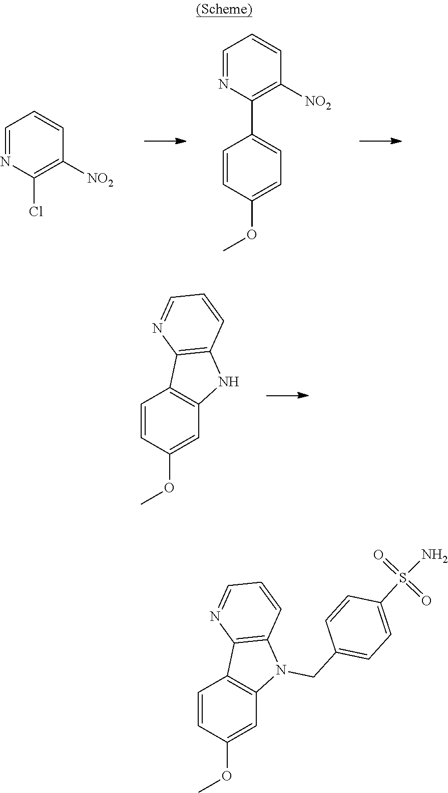 [SMILES: O=[N+]([O-])c1cccnc1Cl ; COc1ccc(-c2ncccc2[N+](=O)[O-])cc1 ; COc1ccc2c(c1)[nH]c1cccnc12 ; COc1ccc2c3ncccc3n(Cc3ccc(S(N)(=O)=O)cc3)c2c1]

### (Step 1) Preparation of 2-(4-methoxyphenyl)-3-nitropyridine

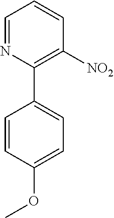 [SMILES: COc1ccc(-c2ncccc2[N+](=O)[O-])cc1]

2-Chloro-3-nitropyridine (1.00 g, 6.31 mmol), 4-methoxyphenylboronic acid (1.25 g, 8.20 mmol), tetrakis(triphenylphosphine) palladium (0) (729 mg, 0.63 mmol) and potassium carbonate (2.18 g, 15.77 mmol) were dissolved in N,N-dimethylformamide (4.00 mL) and distilled water (0.40 mL), and then stirred at 90° C. for 30 minutes using a microwave reactor. After cooling to room temperature, precipitation was removed by filtration using Celite, and then the filtrate was diluted with distilled water and extracted with ethyl acetate three times. The organic layers were dried over anhydrous magnesium sulfate, and then concentrated under reduced pressure. The residue was purified by silica gel column chromatography (ethyl acetate:hexane=3:7) to obtain the title compound as a yellow solid. (1.13 g, 78%). 1H NMR (400 MHz, CDCl3) δ ppm: 8.83 (d, J=4.8 Hz, 1H), 8.09 (d, J=8.1 Hz, 1H), 7.54 (d, J=8.8 Hz, 2H), 7.38 (dd, J=8.1, 4.8 Hz, 1H), 6.99 (d, J=8.9 Hz, 2H), 3.86 (s, 3H).

### (Step 2) Preparation of 7-methoxy-5H-pyrido[3,2-b]indole

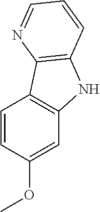

The compound (200 mg, 0.87 mmol) prepared in (step 1) above, 1,2-bis(diphenylphosphino) ethane (380 mg, 0.96 mmol) and toluene (1.00 mL) were added to a pressure flask and stirred at 140° C. for 5 hours. After cooling to room temperature, distilled water was added slowly, and extracted with ethyl acetate three times. The organic layers were dried over anhydrous sodium sulfate, and then concentrated under reduced pressure. The residue was purified by silica gel column chromatography (ethyl acetate:n-hexane=6:4) to obtain the title compound as a white solid. (107 mg, 62%). MS m/z: 199 [M+1]+ 1H NMR (400 MHz, DMSO-d6) δ ppm: 11.26 (s, 1H), 8.37 (d, J=4.8 Hz, 1H), 8.03 (d, J=8.5 Hz, 1H), 7.79 (d, J=8.1 Hz, 1H), 7.28 (dd, J=8.2, 4.7 Hz, 1H), 7.02 (d, J=2.4 Hz, 1H), 6.85 (dd, J=8.6, 2.3 Hz, 1H), 3.87 (s, 3H).

### (Step 3) Preparation of 4-((7-methoxy-5H-pyrido[3,2-b]indol-5-yl)methyl)benzenesulfonamide (Compound 53)

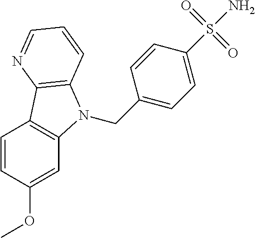 [SMILES: COc1ccc2c3ncccc3n(Cc3ccc(S(N)(=O)=O)cc3)c2c1]

The compound (50.0 mg, 0.25 mmol) prepared in (Step 2) above was dissolved in N,N-dimethylformamide (1.00 mL), and then cooled to 0° C., and 60% sodium hydride (18.2 mg, 0.46 mmol) was added to the mixture and stirred for 10 minutes. Intermediate 1 (158 mg, 0.63 mmol) dissolved in N,N-dimethylformamide (0.50 mL) was slowly added to the reaction mixture, and then stirred at 0° C. for 1 hour under nitrogen. After completion of the reaction, distilled water was slowly added to the reaction mixture, and extracted twice with ethyl acetate. The organic layers were dried over anhydrous sodium sulfate, and then concentrated under reduced pressure. The residue was purified by silica gel column chromatography (ethyl acetate:n-hexane=1:9 to 3:7), and then purified by preparative high performance liquid chromatography (prep-HPLC, 0.1% TFA acetonitrile:distilled water=5:95 to 100:0) to obtain the title compound as a white solid. (18.1 mg, 19%). MS m/z: 368 [M+1]+ 1H NMR (400 MHz, DMSO-d6) δ ppm: 8.62 (d, J=5.3 Hz, 1H), 8.38 (d, J=8.0 Hz, 1H), 8.28 (d, J=8.8 Hz, 1H), 7.74 (d, J=8.4 Hz, 2H), 7.70-7.59 (m, 1H), 7.41-7.33 (m, 3H), 7.30 (s, 2H), 7.05 (d, J=8.8 Hz, 1H), 5.89 (s, 2H), 3.90 (s, 3H).

### [Example 54] Preparation of 4-((9-fluoro-8-methoxy-5H-pyrido[3,2-b]indol-5-yl)methyl)benzenesulfonamide (Compound 54)

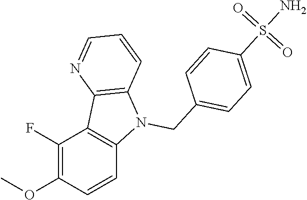 [SMILES: COc1ccc2c(c1F)c1ncccc1n2Cc1ccc(S(N)(=O)=O)cc1]

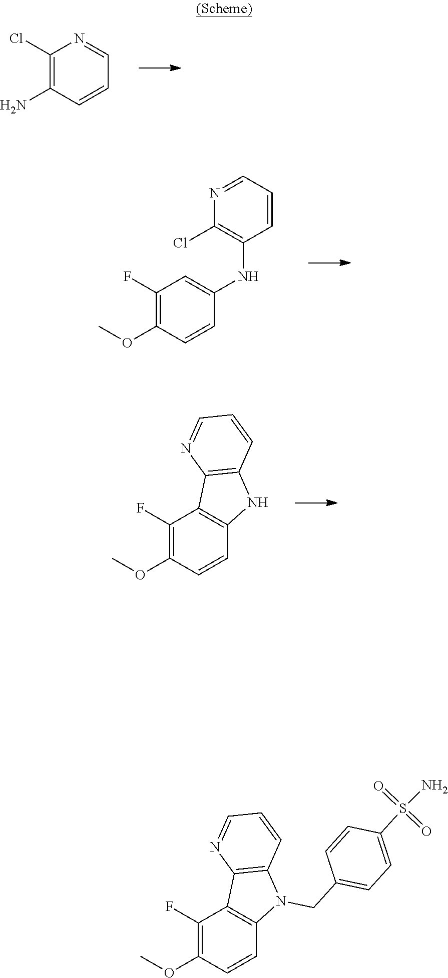 [SMILES: Nc1cccnc1Cl ; COc1ccc(Nc2cccnc2Cl)cc1F ; COc1ccc2[nH]c3cccnc3c2c1F ; COc1ccc2c(c1F)c1ncccc1n2Cc1ccc(S(N)(=O)=O)cc1]

### (Step 1) Preparation of 2-chloro-N-(3-fluoro-4-methoxyphenyl)pyridin-3-amine

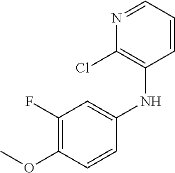 [SMILES: COc1ccc(Nc2cccnc2Cl)cc1F]

2-Chloro-3-iodopyridine (2.50 g, 10.4 mmol) and 3-fluoro-4-methoxyaniline (1.77 g, 12.5 mmol) were dissolved in toluene (15.0 mL), and then palladium diacetate (0) (117 mg, 0.522 mmol), 2,2′-bis(diphenylphosphino)-1,1′-binaphthyl (650 mg, 1.04 mmol) and calcium carbonate (6.8 g, 20.9 mmol) were added to the mixture sequentially. The reaction mixture was stirred at 100° C. for 16 hours under nitrogen. After cooling to room temperature, precipitation was removed by filtration using Celite, and then the filtrate was diluted with distilled water and extracted with ethyl acetate three times. The organic layers were dried over anhydrous magnesium sulfate, and then concentrated under reduced pressure. The residue was purified by silica gel column chromatography (ethyl acetate:n-hexane=1:8 to 1:1) to obtain the title compound as a white solid. (1.4 g, 58%). MS m/z: 253 [M+1]+

### (Step 2) Preparation of 9-fluoro-8-methoxy-5H-pyrido[3,2-b]indole

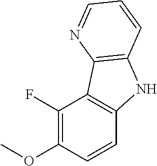 [SMILES: COc1ccc2[nH]c3cccnc3c2c1F]

The compound (1.40 g, 5.54 mmol) prepared in (Step 1) above was dissolved in N,N-dimethylacetamide (10.0 mL), and then bis(triphenylphosphine) palladium (II) dichloride (311 mg, 0.440 mmol) and sodium acetate (1.14 g, 13.9 mmol) were added to the mixture sequentially. The reaction mixture was stirred at 180° C. for 16 hours under nitrogen. After cooling to room temperature, precipitation was removed by filtration using Celite, and then the filtrate was diluted with distilled water and extracted with ethyl acetate three times. The organic layers were dried over anhydrous magnesium sulfate, and then concentrated under reduced pressure. The residue was purified by silica gel column chromatography (ethyl acetate:n-hexane=1:8 to 1:1) to obtain the title compound as a white solid. (140 mg, 12%). MS m/z: 217 [M+1]+

### (Step 3) Preparation of 4-((9-fluoro-8-methoxy-5H-pyrido[3,2-b]indol-5-yl)methyl)benzenesulfonamide (Compound 54)

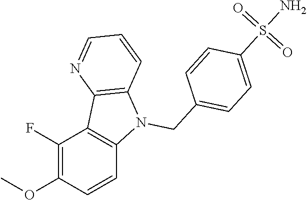 [SMILES: COc1ccc2c(c1F)c1ncccc1n2Cc1ccc(S(N)(=O)=O)cc1]

The compound (30.0 mg, 0.14 mmol) prepared in (Step 2) above was dissolved in N,N-dimethylformamide (0.50 mL), and then cooled to 0° C., and 60% sodium hydride (11.1 mg, 0.28 mmol) was added to the mixture and stirred for 30 minutes. Intermediate 1 (41.6 mg, 0.17 mmol) dissolved in N,N-dimethylformamide (0.50 mL) was slowly added to the reaction mixture, and then stirred at 0° C. for 3 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:5 to 10:0) to obtain the title compound as a yellow solid. (11.0 mg, 20%). MS m/z: 386 [M+1]+ 1H NMR (400 MHz, CD3OD) δ 8.67-8.58 (m, 2H), 7.92 (dd, J=8.6, 5.6 Hz, 1H), 7.83 (d, J=8.5 Hz, 2H), 7.66 (t, J=8.6 Hz, 1H), 7.51 (d, J=9.0 Hz, 1H), 7.34 (d, J=8.3 Hz, 2H), 5.89 (s, 2H), 4.01 (s, 3H).

### [Example 55] Preparation of imino (4-((8-methoxy-5H-pyrido[3,2-b]indol-5-yl)methyl)-3-(trifluoromethyl)phenyl)(methyl)-l6-sulfanone (Compound 55)

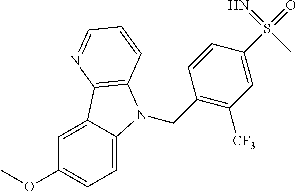

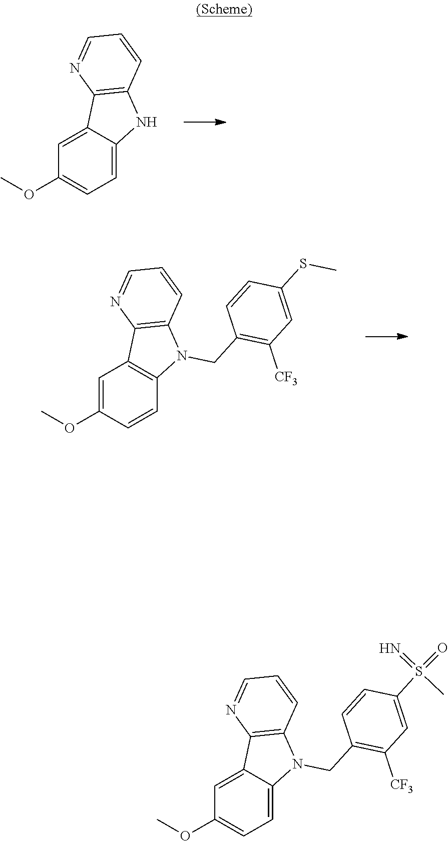

### (Step 1) Preparation of 8-methoxy-5-(4-(methylthio)-2-(trifluoromethyl)benzyl)-5H-pyrido[3,2-b]indole

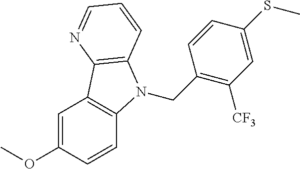

The compound (40.0 mg, 0.20 mmol) prepared in (Step 3) of Example 5 was dissolved in N,N-dimethylformamide (1.00 mL), and then cooled to 0° C., and 60% sodium hydride (12.0 mg, 0.30 mmol) was added to the mixture and stirred for 30 minutes. Intermediate 17 (74.8 mg, 0.26 mmol) dissolved in N,N-dimethylformamide (0.50 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow liquid. (59.0 mg, 73%). MS m/z: 403 [M+1]+ 1H NMR (400 MHz, CDCl3) δ ppm: 8.57 (d, J=4.6 Hz, 1H), 7.92 (d, J=2.5 Hz, 1H), 7.60 (s, 1H), 7.54 (d, J=9.6 Hz, 1H), 7.32 (dd, J=8.3, 4.6 Hz, 1H), 7.24-7.14 (m, 2H), 7.06 (d, J=10.5 Hz, 1H), 6.41 (d, J=8.5 Hz, 1H), 5.64 (s, 2H), 3.96 (s, 3H), 2.44 (s, 3H).

### (Step 2) Preparation of imino (4-((8-methoxy-5H-pyrido[3,2-b]indol-5-yl)methyl)-3-(trifluoromethyl)phenyl)(methyl)-l6-sulfanone (Compound 55)

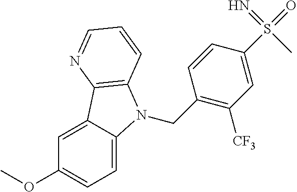

The compound (59.0 mg, 0.15 mmol) prepared in (Step 1) above was dissolved in ethanol (2.00 mL), and then iodobenzene diacetate (142 mg, 0.44 mmol) and ammonium acetate (45.2 mg, 0.59 mmol) were added to the mixture. The reaction mixture was stirred at room temperature for 18 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (48.5 mg, 76%). MS m/z: 434 [M+1]+ 1H NMR (400 MHz, DMSO-d6) δ ppm: δ 8.66 (d, J=5.0 Hz, 1H), 8.32 (s, 1H), 8.21 (d, J=8.5 Hz, 1H), 7.95 (d, J=8.3 Hz, 1H), 7.88 (s, 1H), 7.64 (dd, J=8.4, 4.8 Hz, 1H), 7.46 (d, J=9.0 Hz, 1H), 7.28 (d, J=8.9 Hz, 1H), 6.63 (d, J=8.1 Hz, 1H), 6.01 (s, 2H), 3.90 (s, 3H), 3.17 (s, 3H).

### [Example 56] Preparation of cyclopropyl (4-((7-fluoro-8-methoxy-5H-pyrido[3,2-b]indol-5-yl)methyl)phenyl) (imino)-l6-sulfanone (Compound 56)

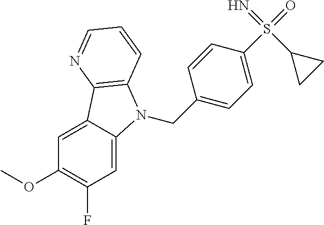 [SMILES: COc1cc2c3ncccc3n(Cc3ccc(S(=N)(=O)C4CC4)cc3)c2cc1F]

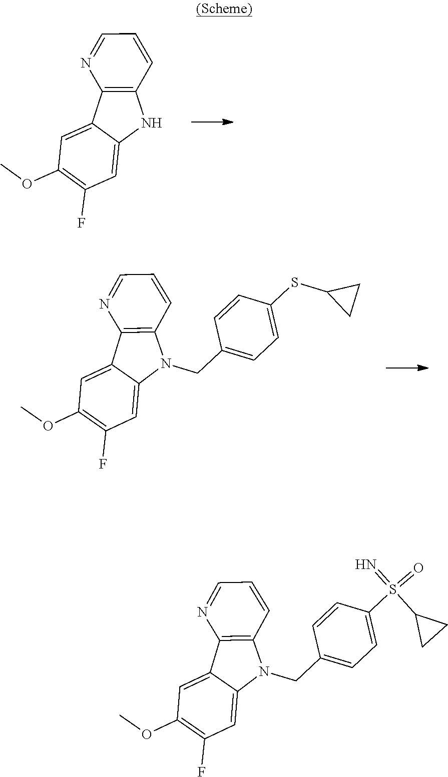 [SMILES: COc1cc2c(cc1F)[nH]c1cccnc12 ; COc1cc2c3ncccc3n(Cc3ccc(SC4CC4)cc3)c2cc1F ; COc1cc2c3ncccc3n(Cc3ccc(S(=N)(=O)C4CC4)cc3)c2cc1F]

### (Step 1) Preparation of 5-(4-(cyclopropylthio)benzyl)-7-fluoro-8-methoxy-5H-pyrido[3,2-b]indole

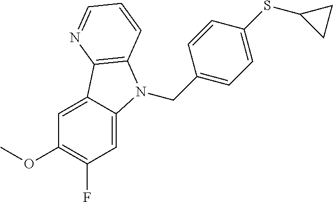 [SMILES: COc1cc2c3ncccc3n(Cc3ccc(SC4CC4)cc3)c2cc1F]

The compound (67.0 mg, 0.31 mmol) prepared in (Step 4) of Example 29 was dissolved in N,N-dimethylformamide (1.00 mL), and then cooled to 0° C., and 60% sodium hydride (18.6 mg, 0.46 mmol) was added to the mixture and stirred for 10 minutes. Intermediate 16 (150 mg, 0.62 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=1:9 to 1:1) to obtain the title compound as a yellow liquid. (65.0 mg, 55%). MS m/z: 379 [M+1]+ 1H NMR (400 MHz, CDCl3) δ ppm: δ 8.54 (d, J=4.6 Hz, 1H), 7.97 (d, J=8.4 Hz, 1H), 7.63 (d, J=8.4 Hz, 1H), 7.29 (s, 1H), 7.27 (d, J=3.3 Hz, 2H), 7.14 (d, J=11.4 Hz, 1H), 7.01 (d, J=8.3 Hz, 2H), 5.41 (s, 2H), 4.02 (s, 3H), 2.12 (td, J=7.6, 3.9 Hz, 1H), 1.03 (q, J=6.4 Hz, 2H), 0.71-0.60 (m, 2H).

### (Step 2) Preparation of imino (4-((8-methoxy-5H-pyrido[3,2-b]indol-5-yl)methyl)-3-(trifluoromethyl)phenyl)(methyl)-l6-sulfanone (Compound 56)

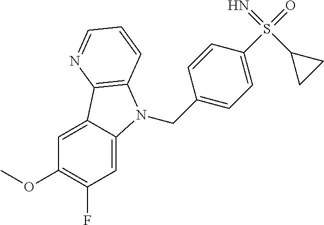 [SMILES: COc1cc2c3ncccc3n(Cc3ccc(S(=N)(=O)C4CC4)cc3)c2cc1F]

The compound (185 mg, 0.39 mmol) prepared in (Step 1) above was dissolved in ethanol (4.00 mL), and then iodobenzene diacetate (630 mg, 1.96 mmol) and ammonium acetate (181 mg, 2.34 mmol) were added to the mixture. The reaction mixture was stirred at 40° C. for 16 hours under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=3:7 to 10:0, methanol:methylene chloride=1:9) to obtain the title compound as a white solid. (147 mg, 91%). Compound 56 was purified by preparative high performance liquid chromatography (prep-HPLC, chiralpak IC (5*100 mm, 5 μm), hexane:methanol:ethanol=40:30:30) to obtain 56a and 56b, which are white optical isomers, respectively. 56a: [α]D20−20.9° (c 0.1, CHCl3)*56b: [α]D20+23.9° (c 0.1, CHCl3) MS m/z: 410 [M+1]+ 1H NMR (400 MHz, DMSO-d6) δ ppm: δ 8.58 (s, 1H), 7.99 (d, J=8.4 Hz, 1H), 7.89 (d, J=8.4 Hz, 2H), 7.64-7.57 (m, 1H), 7.33 (dd, J=8.3, 4.7 Hz, 1H), 7.23 (d, J=8.3 Hz, 2H), 7.09 (d, J=11.1 Hz, 1H), 5.53 (s, 2H), 4.03 (s, 3H), 2.49 (tt, J=8.0, 4.8 Hz, 1H), 1.36 (dq, J=11.9, 4.9 Hz, 1H), 1.16 (dq, J=10.1, 4.9 Hz, 1H), 1.04 (p, J=8.0 Hz, 1H), 0.90 (p, J=8.1 Hz, 1H).

### [Example 57] Preparation of (4-((7-fluoro-8-methoxy-5H-pyrido[3,2-b]indol-5-yl)methyl)phenyl)phosphonic acid (Compound 57)

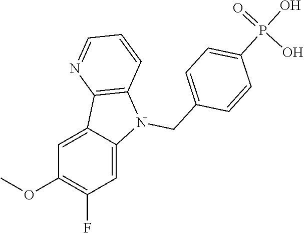 [SMILES: COc1cc2c3ncccc3n(Cc3ccc(P(=O)(O)O)cc3)c2cc1F]

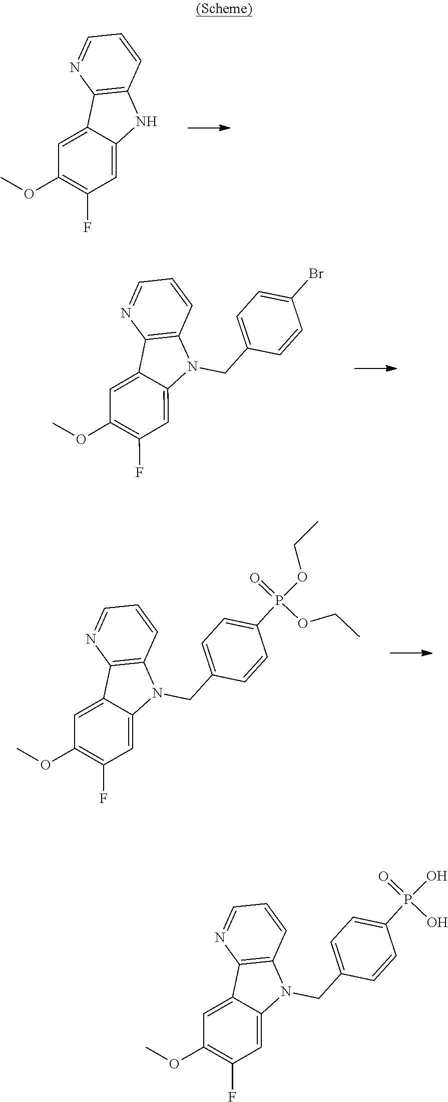

### (Step 1) Preparation of 5-(4-bromobenzyl)-7-fluoro-8-methoxy-5H-pyrido[3,2-b]indole

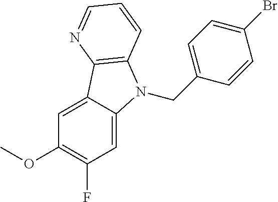 [SMILES: COc1cc2c3ncccc3n(Cc3ccc(Br)cc3)c2cc1F]

The compound (115 mg, 0.53 mmol) prepared in (Step 4) of Example 29 was dissolved in N,N-dimethylformamide (1.00 mL), and then cooled to 0° C., and 60% sodium hydride (44.7 mg, 1.12 mmol) was added to the mixture and stirred for 10 minutes. 1-Bromo-4-(bromomethyl)benzene (266 mg, 1.06 mmol) dissolved in N,N-dimethylformamide (1.00 mL) was slowly added to the reaction mixture, and then stirred at 0° C. for 1 hour under nitrogen. After completion of the reaction, distilled water was added slowly, and extracted twice with ethyl acetate. The organic layers were dried over anhydrous sodium sulfate, and then concentrated under reduced pressure. The residue was purified by silica gel column chromatography (ethyl acetate:n-hexane=0:10 to 1:1) to obtain the title compound as a yellow solid. (155 mg, 75%). MS m/z: 386 [M+1]+ 1H NMR (400 MHz, CDCl3) δ ppm: 8.55 (d, J=4.8 Hz, 1H), 7.97 (d, J=8.4 Hz, 1H), 7.59 (d, J=8.3 Hz, 1H), 7.41 (d, J=8.4 Hz, 2H), 7.31 (dd, J=8.3, 4.7 Hz, 1H), 7.10 (d, J=11.1 Hz, 1H), 6.96 (d, J=8.5 Hz, 2H), 5.40 (s, 2H), 4.02 (s, 3H).

### (Step 2) Preparation of diethyl (4-((7-fluoro-8-methoxy-5H-pyrido[3,2-b]indol-5-yl)methyl)phenyl)phosphonate

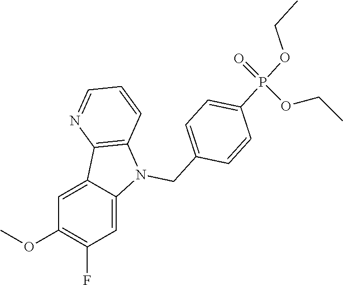 [SMILES: CCOP(=O)(OCC)c1ccc(Cn2c3cc(F)c(OC)cc3c3ncccc32)cc1]

The compound (155 mg, 0.40 mmol) prepared in (Step 1) above and diethyl phosphite (0.10 mL, 0.80 mmol) were dissolved in toluene (5.00 mL), and then triethylamine (0.11 mL, 0.80 mmol) and tetrakis(triphenylphosphine) palladium (0) (46.5 mg, 0.040 mmol) were added to the mixture and stirred at 90° C. for 3 hours. After cooling to room temperature, precipitation was removed by filtration with diethyl ether, and then the organic layers were concentrated under reduced pressure. The residue was purified by silica gel column chromatography (ethyl acetate:n-hexane=1:1 to 10:0, methanol:methylene chloride=1:19) to obtain the title compound as a yellow liquid. (164 mg, 92%). MS m/z: 443 [M+1]+ 1H NMR (400 MHz, CDCl3) δ ppm: 8.56 (d, J=4.6 Hz, 1H), 7.98 (d, J=8.4 Hz, 1H), 7.73 (dd, J=13.0, 8.1 Hz, 2H), 7.59 (d, J=8.3 Hz, 1H), 7.31 (dd, J=8.3, 4.7 Hz, 1H), 7.17 (dd, J=8.1, 3.9 Hz, 2H), 7.09 (d, J=11.1 Hz, 1H), 5.49 (s, 2H), 4.18-4.01 (m, 7H), 1.29 (t, J=7.1 Hz, 6H).

### (Step 3) Preparation of (4-((7-fluoro-8-methoxy-5H-pyrido[3,2-b]indol-5-yl)methyl)phenyl)phosphonic acid (Compound 57)

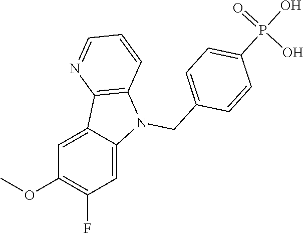 [SMILES: COc1cc2c3ncccc3n(Cc3ccc(P(=O)(O)O)cc3)c2cc1F]

The compound (146 mg, 0.26 mmol) prepared in (Step 2) above was dissolved in methylene chloride (2.50 mL), and then bromotrimethylsilane (0.35 mL, 2.64 mmol) was added to the mixture. The reaction mixture was stirred at room temperature for 16 hours under nitrogen. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (62.0 mg, 60%). MS m/z: 387 [M+1]+ 1H NMR (400 MHz, DMSO-d6) δ ppm: 8.48 (d, J=4.6 Hz, 1H), 8.02 (d, J=7.3 Hz, 1H), 7.92 (d, J=8.5 Hz, 1H), 7.74 (d, J=12.0 Hz, 1H), 7.58 (dd, J=12.6, 8.1 Hz, 2H), 7.41 (dd, J=8.3, 4.7 Hz, 1H), 7.21 (dd, J=8.2, 3.4 Hz, 2H), 5.72 (s, 2H), 3.97 (s, 3H).

### [Example 58] Preparation of (difluoro (4-((8-methoxy-5H-pyrido[3,2-b]indol-5-yl)methyl)phenyl methyl)phosphonic acid (Compound 58)

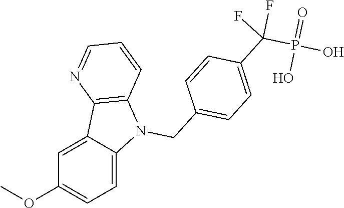 [SMILES: COc1ccc2c(c1)c1ncccc1n2Cc1ccc(C(F)(F)P(=O)(O)O)cc1]

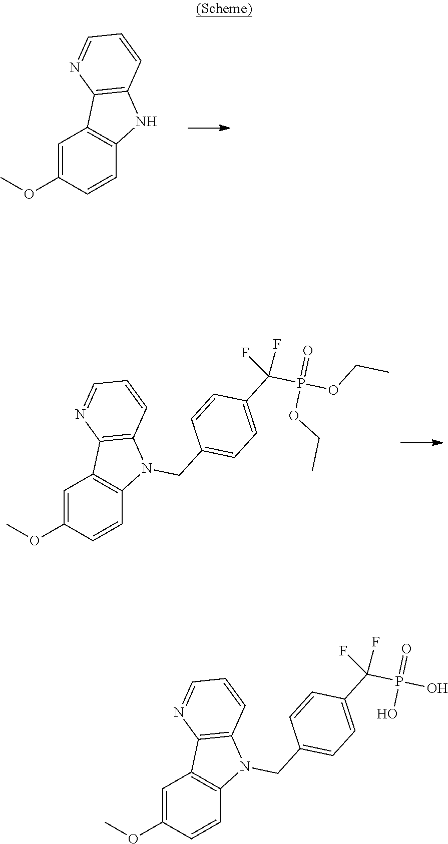

### (Step 1) Preparation of diethyl (difluoro (4-((8-methoxy-5H-pyrido[3,2-b]indol-5-yl)methyl)phenyl)methyl)phosphonate

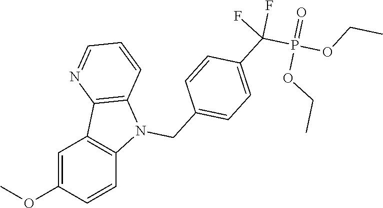 [SMILES: CCOP(=O)(OCC)C(F)(F)c1ccc(Cn2c3ccc(OC)cc3c3ncccc32)cc1]

The compound (10.0 mg, 0.05 mmol) prepared in (Step 3) of Example 5 was dissolved in N,N-dimethylformamide (0.50 mL), and then cooled to 0° C., and 60% sodium hydride (6.00 mg, 0.15 mmol) was added to the mixture and stirred for 30 minutes. Intermediate 18 (46.0 mg, 0.13 mmol) dissolved in N,N-dimethylformamide (0.5 mL) was slowly added to the reaction mixture, and then stirred at 0° C. for 2 hours under nitrogen. After completion of the reaction, distilled water was added slowly, and extracted twice with ethyl acetate. The organic layers were dried over anhydrous sodium sulfate, and then concentrated under reduced pressure. The title compound was obtained as a yellow solid without further purification. (6.8 mg, 29%). MS m/z: 475 [M+1]+

### (Step 2) Preparation of (difluoro (4-((8-methoxy-5H-pyrido[3,2-b]indol-5-yl)methyl)phenyl)methyl)phosphonic acid (Compound 58)

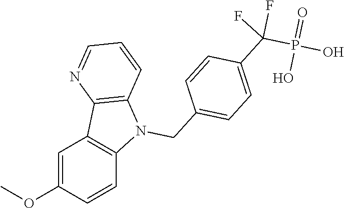 [SMILES: COc1ccc2c(c1)c1ncccc1n2Cc1ccc(C(F)(F)P(=O)(O)O)cc1]

The compound (6.5 mg, 0.01 mmol) prepared in (Step 1) above was dissolved in methylene chloride (1.0 mL), and then bromotrimethylsilane (22.0 mg, 0.14 mmol) was added to the mixture. The reaction mixture was stirred at room temperature for 4 hours under nitrogen. After completion of the reaction, the mixture was concentrated under reduced pressure, and purified by preparative high performance liquid chromatography (prep-HPLC, acetonitrile:distilled water=5:95 to 100:0) to obtain the title compound as a yellow solid. (3.6 mg, 59%). MS m/z: 419 [M+1]+ 1H NMR (400 MHz, DMSO-d6) δ 8.57 (d, J=5.0 Hz, 1H), 8.28 (d, J=7.5 Hz, 1H), 7.83 (s, 1H), 7.70 (d, J=9.0 Hz, 1H), 7.59 (d, J=8.1 Hz, 1H), 7.45 (d, J=8.0 Hz, 2H), 7.26 (t, J=10.4 Hz, 3H), 5.78 (s, 2H), 3.89 (s, 3H).

### [Example 59] Preparation of isopropyl ((4-((8-methoxy-5H-pyrido[3,2-b]indol-5-yl)methyl)phenyl) (phenoxy)phosphoryl)-L-alaninate (Compound 59)

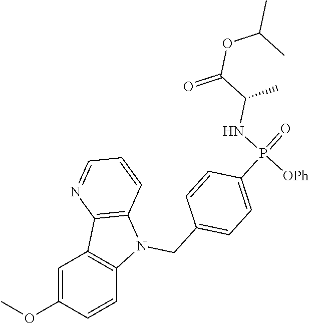 [SMILES: COc1ccc2c(c1)c1ncccc1n2Cc1ccc(P(=O)(N[C@@H](C)C(=O)OC(C)C)Oc2ccccc2)cc1]

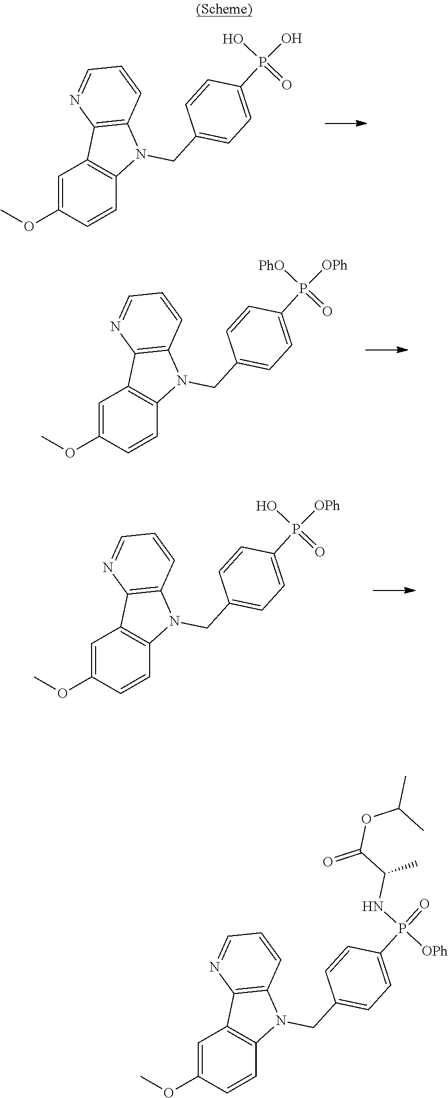 [SMILES: COc1ccc2c(c1)c1ncccc1n2Cc1ccc(P(=O)(O)O)cc1 ; COc1ccc2c(c1)c1ncccc1n2Cc1ccc(P(=O)(Oc2ccccc2)Oc2ccccc2)cc1 ; COc1ccc2c(c1)c1ncccc1n2Cc1ccc(P(=O)(O)Oc2ccccc2)cc1 ; COc1ccc2c(c1)c1ncccc1n2Cc1ccc(P(=O)(N[C@@H](C)C(=O)OC(C)C)Oc2ccccc2)cc1]

### (Step 1) Preparation of diphenyl (4-((8-methoxy-5H-pyrido[3,2-b]indol-5-yl)methyl)phenyl)phosphonate

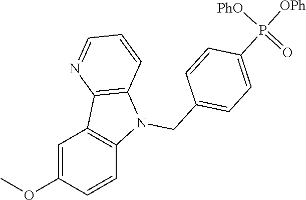 [SMILES: COc1ccc2c(c1)c1ncccc1n2Cc1ccc(P(=O)(Oc2ccccc2)Oc2ccccc2)cc1]

Compound 13 (200 mg, 0.54 mmol) was dissolved in methylene chloride (4.00 mL), and then N,N-dimethylformamide (2 drops) was added thereto. Oxalyl chloride (0.14 mL, 1.63 mmol) dissolved in methylene chloride (1.00 mL) was slowly added to the reaction mixture over 10 minutes, and then stirred at 40° C. for 1 hour under nitrogen. The reaction mixture was concentrated under reduced pressure, and then the residue was dissolved in methylene chloride (5.00 mL), and phenol (138 mg, 1.47 mmol) was added to the mixture at 0° C. N,N-Diisopropylethylamine (0.30 mL, 1.74 mmol) was added thereto slowly, and then stirred at room temperature for 18 hours. After completion of the reaction, the reaction mixture was concentrated under reduced pressure. The residue was purified by silica gel column chromatography (ethyl acetate:n-hexane=0:10 to 2:3) to obtain the title compound as a pale yellow liquid. (124 mg, 43%). MS m/z: 521 [M+1]+ 1H NMR (400 MHz, CDCl3) δ ppm: δ 8.57 (d, J=4.6 Hz, 1H), 7.94-7.83 (m, 3H), 7.58 (d, J=8.3 Hz, 1H), 7.32 (dd, J=8.3, 4.7 Hz, 1H), 7.30-7.27 (m, 3H), 7.25 (d, J=2.3 Hz, 2H), 7.24-7.18 (m, 3H), 7.18-7.10 (m, 6H), 5.55 (s, 2H), 3.96 (s, 3H).

### (Step 2) Preparation of phenyl hydrogen (4-((8-methoxy-5H-pyrido[3,2-b]indol-5-yl)methyl)phenyl)phosphonate

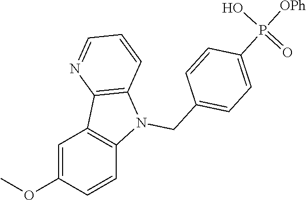 [SMILES: COc1ccc2c(c1)c1ncccc1n2Cc1ccc(P(=O)(O)Oc2ccccc2)cc1]

The compound (124 mg, 0.24 mmol) prepared in (Step 1) above was dissolved in tetrahydrofuran (2.00 mL), and then 2 N aqueous sodium hydroxide solution (0.20 mL) was added to the mixture. The reaction mixture was stirred at room temperature for 18 hours. After completion of the reaction, the reaction mixture was concentrated under reduced pressure. The residue was dissolved in tetrahydrofuran and acetonitrile, and then dried over anhydrous sodium sulfate, and concentrated under reduced pressure to obtain the title compound as a white solid. (108 mg, 87%). MS m/z: 445 [M+1]+ 1H NMR (400 MHz, DMSO-d6) δ ppm: 8.61 (d, J=4.4 Hz, 1H), 8.31 (s, 1H), 7.82 (s, 1H), 7.73-7.58 (m, 4H), 7.33-7.21 (m, 5H), 7.14-7.00 (m, 3H), 5.82 (s, 2H), 3.88 (s, 3H).

### (Step 3) Preparation of isopropyl ((4-((8-methoxy-5H-pyrido[3,2-b]indol-5-yl)methyl)phenyl) (phenoxy)phosphoryl)-L-alaninate (Compound 59)

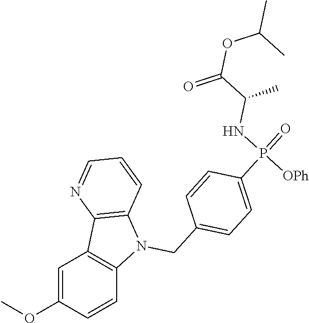 [SMILES: COc1ccc2c(c1)c1ncccc1n2Cc1ccc(P(=O)(N[C@@H](C)C(=O)OC(C)C)Oc2ccccc2)cc1]

The compound (40.0 mg, 0.09 mmol) prepared in (Step 2) above was dissolved in methylene chloride (2.00 mL), and then N,N-dimethylformamide (2 drops) was added to the mixture. Oxalyl chloride (0.015 mL, 0.18 mmol) dissolved in methylene chloride (0.50 mL) was slowly added to the reaction mixture, and then stirred at 40° C. for 1 hour under nitrogen. After completion of the reaction, the reaction mixture was concentrated under reduced pressure. The residue was dissolved in methylene chloride (2.00 mL), and L-alanine isopropyl ester hydrochloride (45.3 mg, 0.27 mmol) and N,N-diisopropylethylamine (0.063 mL, 0.45 mmol) dissolved in methylene chloride (0.50 mL) at 0° C. were added to the mixture slowly. The reaction mixture was stirred at room temperature for 16 hours, and concentrated under reduced pressure. The residue was purified by preparative high performance liquid chromatography (prep-HPLC, 0.1% TFA acetonitrile:distilled water=5:95 to 100:0) to obtain the title compound as a yellow solid. (14.8 mg, 28%). MS m/z: 558 [M+1]+ 1H NMR (400 MHz, CDCl3,) δ ppm: 8.71 (d, J=5.4 Hz, 1H), 8.23 (s, 1H), 8.04 (d, J=8.4 Hz, 1H), 7.88-7.81 (m, 2H), 7.66 (dd, J=8.4, 5.4 Hz, 1H), 7.37 (s, 2H), 7.30-7.26 (m, 2H), 7.21-7.09 (m, 5H), 5.64 (s, 2H), 4.93-4.85 (m, 1H), 3.98-3.93 (m, 4H), 3.65 (dt, J=36.4, 10.3 Hz, 1H), 1.26 (d, J=9.9 Hz, 3H), 1.17-1.07 (m, 6H).

### [Example 60] Preparation of (((((tert-butoxycarbonyl)oxy) methoxy) (4-((8-methoxy-5H-pyrido[3,2-b]indol-5-yl)methyl)phenyl)phosphoryl)oxy) methyl pivalate (Compound 60)

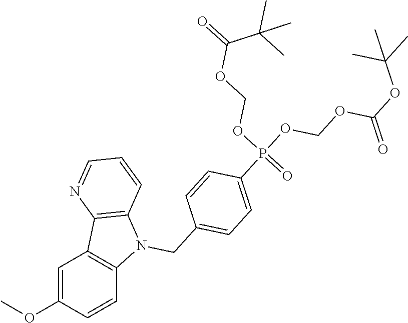 [SMILES: COc1ccc2c(c1)c1ncccc1n2Cc1ccc(P(=O)(OCOC(=O)OC(C)(C)C)OCOC(=O)C(C)(C)C)cc1]

Compound 13 (30.0 mg, 0.08 mmol) was dissolved in N-methyl-2-pyrrolidone (0.50 mL), and then triethylamine (0.034 mL, 0.240 mmol) and chloromethyl pivalate (27.0 mg, 0.180 mmol) were added to the mixture sequentially. The mixture was stirred at 60° C. for 6 hours under nitrogen. After completion of the reaction, distilled water was added slowly, and extracted twice with ethyl acetate. The organic layers were dried over anhydrous sodium sulfate, and then concentrated under reduced pressure. The residue was purified by preparative high performance liquid chromatography (prep-HPLC, 0.1% TFA acetonitrile:distilled water=5:95 to 100:0) to obtain the title compound as a yellow solid. (19.6 mg, 40%). MS m/z: 597 [M+1]+ 1H NMR (400 MHz, CD3OD) δ 8.61 (d, J=5.6 Hz, 1H), 8.48 (d, J=8.4 Hz, 1H), 7.87 (d, J=2.0 Hz, 1H), 7.78 (dd, J=5.6, 8.4 Hz, 1H), 7.72-7.64 (m, 3H), 7.40-7.32 (m, 3H), 5.85 (s, 2H), 5.69 (S, 2H), 5.65 (s, 2H), 3.95 (s, 3H), 0.98 (s, 18H)

### [Example 61] Preparation of (1S)-1-(((((S)-1-((ethoxycarbonyl)oxy)ethyl) amino) (4-((8-methoxy-5H-pyrido[3,2-b]indol-5-yl)methyl)phenyl)phosphoryl) amino)ethyl propionate (Compound 61)

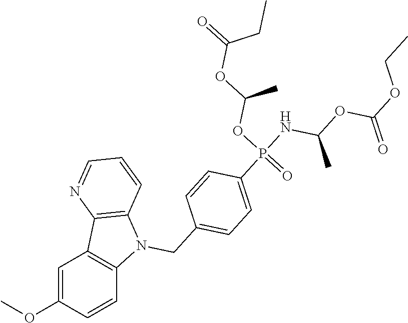 [SMILES: CCOC(=O)O[C@@H](C)NP(=O)(O[C@H](C)OC(=O)CC)c1ccc(Cn2c3ccc(OC)cc3c3ncccc32)cc1]

Compound 13 (50.0 mg, 0.14 mmol) was dissolved in pyridine (0.700 mL), and then triethylamine (0.114 mL, 0.815 mmol), ethyl (2S)-2-aminopropionic acid (63.6 mg, 0.543 mmol), 2,2′-dithiodipyridine (179 mg, 0.815 mmol) and triphenylphosphine (213 mg, 0.815 mmol) were added to the mixture sequentially. The mixture was stirred at 60° C. for 6 hours under nitrogen. After completion of the reaction, the mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (45.6 mg, 59%). MS m/z: 567 [M+1]+ 1H NMR (400 MHz, CD3OD) δ 8.60 (s, 1H), 8.40 (d, J=7.6 Hz, 1H), 7.90 (s, 1H), 7.76-7.64 (m, 4H), 7.36-7.27 (m, 3H), 5.81 (s, 2H), 4.29 (dd, J=7.2, 14.4 Hz, 2H), 4.08 (dd, J=6.6, 13.8 Hz, 2H), 3.95 (s, 3H), 3.92-3.83 (m, 2H), 1.52 (d, J=7.2 Hz, 3H), 1.28 (d, J=9.6 Hz, 3H), 1.28 (t, J=9.6 Hz, 3H), 1.01 (t, J=7.0 Hz, 3H)

### [Example 62] Preparation of isopropyl ((4-((7-fluoro-8-methoxy-5H-pyrido[3,2-b]indol-5-yl)methyl)phenyl) (phenoxy)phosphoryl)-L-alaninate (Compound 62)

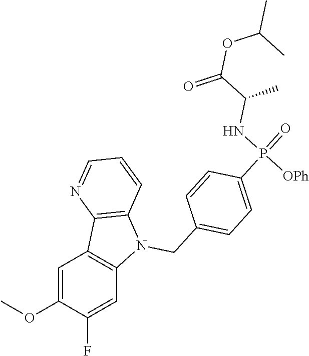 [SMILES: COc1cc2c3ncccc3n(Cc3ccc(P(=O)(N[C@@H](C)C(=O)OC(C)C)Oc4ccccc4)cc3)c2cc1F]

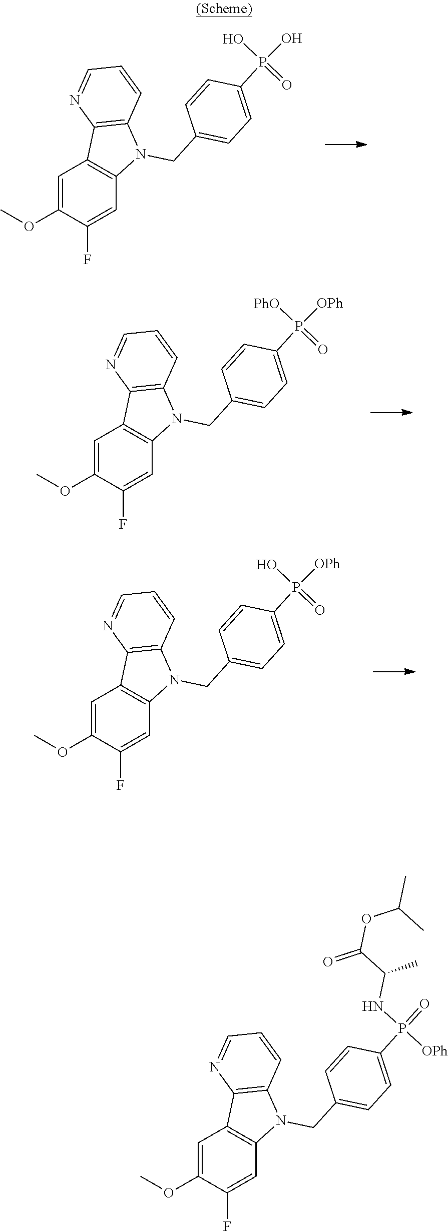 [SMILES: COc1cc2c3ncccc3n(Cc3ccc(P(=O)(O)O)cc3)c2cc1F ; COc1cc2c3ncccc3n(Cc3ccc(P(=O)(Oc4ccccc4)Oc4ccccc4)cc3)c2cc1F ; COc1cc2c3ncccc3n(Cc3ccc(P(=O)(O)Oc4ccccc4)cc3)c2cc1F ; COc1cc2c3ncccc3n(Cc3ccc(P(=O)(N[C@@H](C)C(=O)OC(C)C)Oc4ccccc4)cc3)c2cc1F]

### (Step 1) Preparation of diphenyl (4-((7-fluoro-8-methoxy-5H-pyrido[3,2-b]indol-5-yl)methyl)phenyl)phosphonate

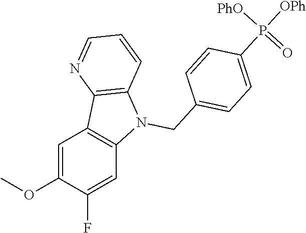 [SMILES: COc1cc2c3ncccc3n(Cc3ccc(P(=O)(Oc4ccccc4)Oc4ccccc4)cc3)c2cc1F]

Compound 57 (177 mg) was dissolved in methylene chloride (2.00 mL), and then N,N-dimethylformamide (2 drops) was added to the mixture. Oxalyl chloride (0.094 mL, 1.10 mmol) dissolved in methylene chloride (1.00 mL) was slowly added to the reaction mixture over 10 minutes, and then stirred at 40° C. for 1 hour under nitrogen, and the reaction mixture was concentrated under reduced pressure. The mixture was dissolved in methylene chloride (3.00 mL), and phenol (93.1 mg, 0.99 mmol) was added at 0° C. And then, N,N-Diisopropylethylamine (0.20 mL, 1.17 mmol) was added slowly, and stirred at room temperature for 18 hours. After completion of the reaction, the reaction mixture was concentrated under reduced pressure. The residue was purified by silica gel column chromatography (ethyl acetate:n-hexane=0:10 to 2:3) to obtain the title compound as a pale yellow liquid. (140 mg). MS m/z: 539 [M+1]+

### (Step 2) Preparation of phenyl hydrogen (4-((7-fluoro-8-methoxy-5H-pyrido[3,2-b]indol-5-yl)methyl)phenyl)phosphonate

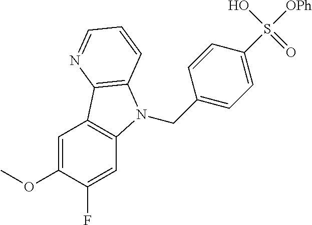

The compound (140 mg) prepared in (Step 1) above was dissolved in tetrahydrofuran (1.50 mL), and then an aqueous sodium hydroxide solution (0.20 mL) was added to the mixture. The reaction mixture was stirred at room temperature for 18 hours. After completion of the reaction, the reaction mixture was concentrated under reduced pressure. The residue was purified by preparative high performance liquid chromatography (prep-HPLC, 0.1% TFA acetonitrile:distilled water=5:95 to 100:0) to obtain the title compound as a yellow solid. (101 mg). MS m/z: 463 [M+1]+ 1H NMR (400 MHz, DMSO-d6) δ ppm: 8.53 (d, J=4.6 Hz, 1H), 8.09 (d, J=8.3 Hz, 1H), 7.95 (d, J=8.6 Hz, 1H), 7.75 (d, J=11.9 Hz, 1H), 7.67 (dd, J=13.1, 8.2 Hz, 2H), 7.47 (dd, J=8.3, 4.6 Hz, 1H), 7.31-7.20 (m, 4H), 7.13-7.01 (m, 3H), 5.76 (s, 2H), 3.97 (s, 3H).

### (Step 3) Preparation of isopropyl ((4-((7-fluoro-8-methoxy-5H-pyrido[3,2-b]indol-5-yl)methyl)phenyl) (phenoxy)phosphoryl)-L-alaninate (Compound 62)

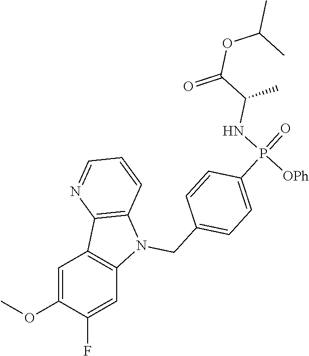 [SMILES: COc1cc2c3ncccc3n(Cc3ccc(P(=O)(N[C@@H](C)C(=O)OC(C)C)Oc4ccccc4)cc3)c2cc1F]

The compound (101 mg, 0.22 mmol) prepared in (Step 2) above was dissolved in methylene chloride (4.00 mL), and then N,N-dimethylformamide (2 drops) was added to the mixture. Oxalyl chloride (0.037 mL, 0.44 mmol) dissolved in methylene chloride (1.00 mL) was slowly added to the reaction mixture, and then stirred at 40° C. for 1 hour under nitrogen. After completion of the reaction, the reaction mixture was concentrated under reduced pressure. The mixture was dissolved in methylene chloride (4.00 mL), and L-alanine isopropyl ester hydrochloride (110 mg, 0.66 mmol) and N,N-diisopropylethylamine (0.15 mL, 1.09 mmol) dissolved in methylene chloride (1.0 mL) at 0° C. were added slowly. The reaction mixture was stirred at room temperature for 16 hours, and concentrated under reduced pressure. The residue was purified by preparative high performance liquid chromatography (prep-HPLC, 0.1% TFA acetonitrile:distilled water=5:95 to 100:0) to obtain the title compound as a yellow solid. (19.1 mg, 15%). MS m/z: 576 [M+1]+ 1H NMR (400 MHz, DMSO-d6) δ ppm: 8.60 (d, J=4.9 Hz, 1H), 8.26 (d, J=6.6 Hz, 1H), 7.99 (d, J=8.5 Hz, 1H), 7.81 (d, J=11.8 Hz, 1H), 7.78-7.68 (m, 2H), 7.57 (dd, J=8.3, 4.9 Hz, 1H), 7.33-7.26 (m, 4H), 7.23-7.06 (m, 3H), 6.02-5.87 (m, 1H), 5.80 (s, 2H), 4.69-4.60 (m, 1H), 3.97 (s, 3H), 1.10 (d, J=7.3 Hz, 3H), 1.02-0.85 (m, 6H).

### Example 3: ENPP-1 Enzyme Assay with pNP-TMP Substrate

In order to measure the ENPP-1 inhibitory activity of the compounds according to the present disclosure, the following tests were performed.

ENPP-1 hydrolyzes a variety of substrates, including phosphodiester bonds of nucleotides and nucleotide sugars and pyrophosphate bonds of nucleotides and nucleotide sugars. p-Nitrophenyl 5′-thymidine monophosphate (p-Nph-5′-TMP) is an artificial substrate for 5′-adenosine monophosphate (AMP), which is hydrolyzed by the human recombinant ENPP-1 enzyme. In the above reaction, ENPP-1 enzymatic activity is monitored through the formation of yellow p-nitrophenolate, which absorbs at 405 nm.

First, the reaction mixture contains 100 mM Tris-HCl (pH 9.0), 150 mM NaCl, 5 mM NaCl, and 1% DMSO. Serially diluted ENPP-1 inhibitors (test compound concentrations ranging from 10 μM to 0.5 nM) are reacted with 12.5 ng of ENPP-1 (Biovendor, catalog number RD172124100) and 720 μM substrate pNP-TMP at 37° C. for 60 minutes. All assays include a DMSO control without ENPP-1 inhibitor that gives maximum absorbance.

After 60 minutes of this reaction, the luminescence signal is measured using an Epoch™ Microplate Spectrophotometer (BioTek Instruments Inc.). Inhibition (%) is calculated using the following formula: Sample OD405 nM/Max OD405 nM×100%. % residual ENPP-1 enzyme activity versus IC50 values for ENPP-1 inhibitor concentrations were determined by fitting inhibition curves using the 4-parameter method of GraphPad Prism® software [GraphPad version 9.3.1 for Windows, GraphPad Software, La Jolla California USA, www.graphpad.com].

Serially diluted samples of one compound were tested twice or more, and the average IC50 value for each compound was calculated, and the experimental results are shown in Table 1 below.

TABLE 1

| Compound | pNP-TMP assay |
|---|---|
| No. | activity |
| 1 | A |
| 2 | A |
| 3 | A |
| 4 | A |
| 5 | A |
| 6 | A |
| 7 | B |
| 8 | A |
| 9 | A |
| 10a | A |
| 10b | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | B |
| 25 | A |
| 26 | A |
| 27 | A |
| 28 | A |
| 29 | A |
| 30a | A |
| 30b | A |
| 31 | A |
| 32 | A |
| 33 | C |
| 34 | C |
| 35 | A |
| 36 | B |
| 37 | A |
| 38 | A |
| 39 | C |
| 40 | B |
| 41 | A |
| 42 | A |
| 43 | A |
| 44 | A |
| 45 | A |
| 46 | A |
| 47 | A |
| 48 | A |
| 49 | A |
| 50 | B |
| 51 | A |
| 52 | A |
| 53 | A |
| 55 | C |
| 56a | A |
| 56b | A |
| 57 | A |
| 58 | A |
| IC50 (μM) | A < 1 μM, 1 μM < B < 10 μM, C > 10 μM |

### Example 4: ENPP-1 Enzyme Assay with cGAMP Substrate

2′3′-cGAMP is a natural substrate specifically hydrolyzed by ENPP-1, and produces 5′-adenosine monophosphate (AMP) and 5′-guanosine monophosphate (GMP). AMP and GMP produced from the reaction are monitored for ENPP-1 enzyme activity through an AMP²/GMP² ENPP-1 Assay kit (Bellbrook Labs, catalog number 3015).

First, the reaction mixture contains 25 mM Tris-HCl (pH 7.4), 5 mM MgCl₂, 0.01% Brij-35, and 1% DMSO. Serially diluted ENPP-1 inhibitors (test compound concentrations ranging from 10 μM to 0.5 nM) are reacted with 200 pM ENPP-1 (Biovendor, catalog number RD172124100) and 10 μM substrate 2′3′-cGAMP at 37° C. for 30 minutes. All assays include a DMSO control without ENPP-1 inhibitor that provides maximal AMP and GMP production. After 30 minutes of this reaction, AMP and GMP production is detected as a measure of ENPP-1 enzymatic activity using the Transcreener® AMP²/GMP² ENPP-1 Assay kit. The detection reagent is a reagent containing 16 nM AMP²/GMP² Antibody-Tb, 25 mM Tris-HCl and 60.1 nM AMP/GMP HiLyte647 Tracer, which is added by 5 μl, mixed well, and incubated at 25° C. for 2 hours.

The luminescence signal is measured using a SPARK Cyto plate reader (Tecan). Inhibition (%) is calculated using the following formula: (Max 615:665 ratio-615:665 ratio)/Max 615:665 ratio×100%. % residual ENPP-1 enzyme activity versus IC50 values for ENPP-1 inhibitor concentrations were determined by fitting inhibition curves using the 4-parameter method of GraphPad Prism® software [GraphPad version 9.3.1 for Windows, GraphPad Software, La Jolla California USA, www.graphpad.com].

Serially diluted samples of one compound were tested twice or more, and the average IC50 value for each compound was calculated, and the experimental results are shown in Table 2 below.

TABLE 2

| Compound | cGAMP assay |
|---|---|
| No. | activity |
| 1 | B |
| 2 | A |
| 3 | A |
| 4 | A |
| 5 | A |
| 6 | A |
| 7 | C |
| 8 | A |
| 9 | C |
| 10a | A |
| 10b | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | B |
| 23 | A |
| 24 | A |
| 25 | B |
| 26 | A |
| 27 | B |
| 28 | A |
| 29 | A |
| 30a | A |
| 30b | A |
| 31 | A |
| 32 | A |
| 33 | — |
| 34 | C |
| 35 | B |
| 36 | C |
| 37 | C |
| 38 | A |
| 39 | — |
| 40 | — |
| 41 | B |
| 42 | A |
| 43 | B |
| 44 | A |
| 45 | A |
| 46 | A |
| 47 | B |
| 48 | B |
| 49 | B |
| 50 | — |
| 51 | A |
| 52 | B |
| 53 | A |
| 54 | A |
| 55 | B |
| 56b | A |
| 55 | A |
| 57 | A |
| 58 | B |
| IC50 (μM) | A < 1 μM, 1 μM < B < 10 μM, C > 10 μM |

Although the examples of the present disclosure have been described above, those of ordinary skill in the technical art to which the present disclosure pertains will understand that the present disclosure may be implemented in other specific forms without changing the technical spirit or essential characteristics thereof. Therefore, it should be understood that the examples described above are illustrative and not restrictive in all respects.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by Formula I or a pharmaceutically acceptable salt thereof: 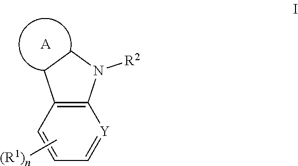
wherein
each R¹ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
A is an aryl, heteroaryl, or heterocyclyl;
Y is CH, CR¹, or N;
R² is aralkyl, heteroaralkyl, heterocyclylalkyl, or cycloalkylalkyl; and
n is 0, 1, 2, 3, or 4.

2. The compound of claim 1, wherein A is a heteroaryl (e.g., pyridinyl, pyrimidinyl, or pyridazinyl, preferably pyridinyl).

3. The compound of claim 1, wherein A is a heterocyclyl (e.g., piperidinyl, piperidonyl, tetrahydropyridazinonyl,

4. The compound of claim 1, wherein the compound has a structure represented by Formula Ia or a pharmaceutically acceptable salt thereof: 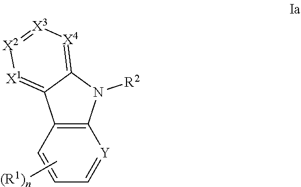
wherein,
X¹ is CR³ or N;
X² is CR⁴ or N;
X³ is CR⁵ or N;
X⁴ is CR⁶ or N; and
R³, R⁴, R⁵, and R⁶ are each independently selected from H, alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, and sulfonamido.

5. The compound of claim 4, wherein X¹ is N.

6. The compound of claim 4, wherein X¹ is CR³.

7. The compound of claim 6, wherein R³ is H.

8. The compound of any one of claims 4-7, wherein X² is N.

9. The compound of any one of claims 4-7, wherein X² is CR⁴.

10. The compound of claim 9, wherein R⁴ is H.

11. The compound of any one of claims 4-10, wherein X³ is N.

12. The compound of any one of claims 4-10, wherein X³ is CR⁷.

13. The compound of claim 12, wherein R⁵ is H.

14. The compound of any one of claims 4-13, wherein X⁴ is N.

15. The compound of any one of claims 4-13, wherein X⁴ is CR⁶.

16. The compound of claim 15, wherein R⁶ is H.

17. The compound of any one of claims 1-12, wherein n is 1.

18. The compound of any one of claims 1-12, wherein n is 2.

19. The compound of any one of claims 1-16, wherein Y is CH.

20. The compound of any one of claims 1-16, wherein Y is N.

21. The compound of any one of claims 1-17 and 19, wherein the compound has a structure represented by Formula Ib or a pharmaceutically acceptable salt thereof:

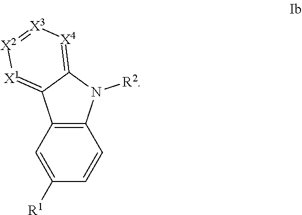

22. The compound of any one of claims 1-17 and 19, wherein the compound has a structure represented by Formula Ic or a pharmaceutically acceptable salt thereof:

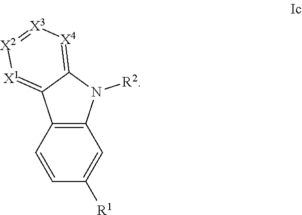

23. The compound of any one of claims 1-16, 18, and 19, wherein the compound has a structure represented by Formula Id or a pharmaceutically acceptable salt thereof: 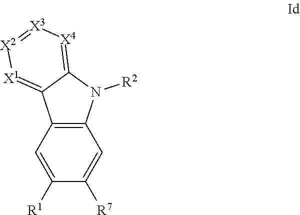
wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.

24. The compound of any one of claims 1-16, 18, and 19, wherein the compound has a structure represented by Formula Ie or a pharmaceutically acceptable salt thereof: 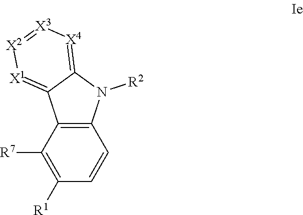
wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.

25. The compound of any one of claims 1-16, 18, and 19, wherein the compound has a structure represented by Formula If or a pharmaceutically acceptable salt thereof: 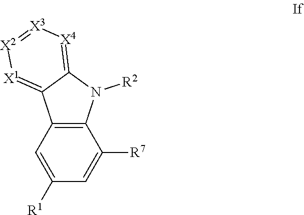
wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.

26. The compound of any one of claims 1-25, wherein R² is aralkyl (e.g., phenylalkyl, such as phenylmethylenyl or phenylethylenyl, preferably phenylmethylenyl).

27. The compound of any one of claims 1-25, wherein R² is heterocyclylalkyl (e.g., piperidinylmethylenyl).

28. The compound of any one of claims 1-17 and 19, wherein the compound has a structure represented by Formula Ila or a pharmaceutically acceptable salt thereof: 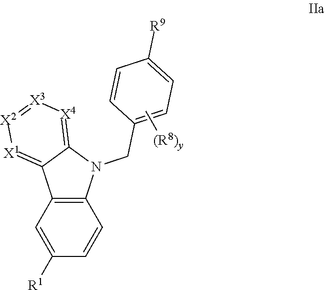
wherein,
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

29. The compound of any one of claims 1-17 and 19, wherein the compound has a structure represented by Formula IIb or a pharmaceutically acceptable salt thereof: 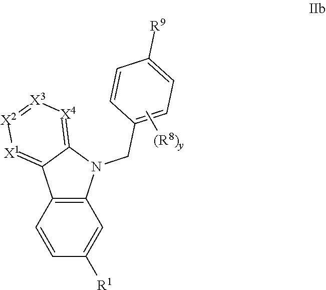
wherein,
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

30. The compound of any one of claims 1-16, 18, and 19, wherein the compound has a structure represented by Formula IIc or a pharmaceutically acceptable salt thereof: 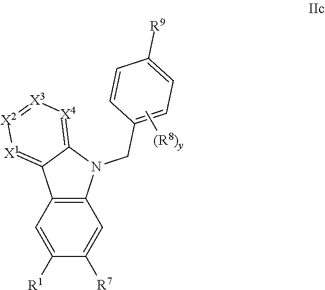
wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

31. The compound of any one of claims 1-16, 18, and 19, wherein the compound has a structure represented by Formula IId or a pharmaceutically acceptable salt thereof: 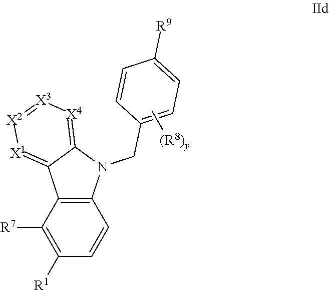
wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclic, alkylsulfoxidyl, alkylsulfonyl, or sulfonamido.
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 1, 2, 3, or 4.

32. The compound of any one of claims 1-16, 18, and 19, wherein the compound has a structure represented by Formula Ile or a pharmaceutically acceptable salt thereof: 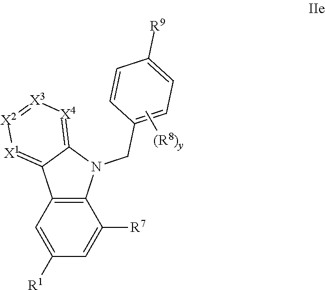
wherein,
R⁷ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitro, azido, cycloalkyl, heterocyclyl, alkylsulfoxidyl, alkylsulfonyl, or sulfonamido.
each R⁸ is independently alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, amino, amido, cyano, nitros, azido, cycloalkyl, heterocyclyl, alkylsulfoxidyl, alkylsulfonyl, or sulfonamido;
R⁹ is alkyl, alkoxy, alkenyl, alkynyl, aralkyl, heteroaralkyl, aryl, heteroaryl, halo, haloalkyl, hydroxyl, carboxyl, acyl, ester, thioester, phosphoryl, phosphoramidityl, phosphoramidatyl, amino, amido, cyano, nitro, azido, cycloalkyl, heterocyclyl, alkylsulfoxidyl, alkylsulfonyl, sulfonamido, iminosulfanonyl, or sulfamidyl; and
y is 0, 1, 2, 3, or 4.

33. The compound of any one of claims 1-32, wherein R¹ is alkyl (e.g., methyl).

34. The compound of any one of claims 1-32, wherein R¹ is alkoxy (e.g., methoxy, trifluoromethoxy, or ethoxy, preferably methoxy).

35. The compound of any one of claims 1-32, wherein R¹ is halo (e.g., fluoro or chloro).

36. The compound of any one of claims 23-35, wherein R⁷ is alkyl (e.g., methyl).

37. The compound of any one of claims 23-35, wherein R⁷ is alkoxy (e.g., methoxy).

38. The compound of any one of claims 23-35, wherein R⁷ is halo (e.g., fluoro or chloro).

39. The compound of any one of claims 28-38, wherein y is 0.

40. The compound of any one of claims 28-38, wherein y is 1.

41. The compound of any one of claims 28-40, wherein R⁸ is halo (e.g., fluoro or chloro).

42. The compound of any one of claims 28-40, wherein R⁸ is alkyl (e.g., methyl or trifluoromethyl).

43. The compound of any one of claims 28-40, wherein R¹⁰ is alkoxy (e.g., methoxy).

44. The compound of any one of claims 28-43, wherein R⁹ is sulfonamido (e.g., methylsulfonamido).

45. The compound of any one of claims 28-43, wherein R⁹ is iminosulfanonyl (e.g., (imino)(methyl)iminosulfanonyl, (imino) (ethyl)iminosulfanonyl, (imino) (benzyl)iminosulfanonyl, or imino) (cyclopropyl)iminosulfanonyl).

46. The compound of any one of claims 28-43, wherein R⁹ is sulfamidyl (e.g., (imino)(methyl)iminosulfanonyl).

47. The compound of any one of claims 28-43, wherein R⁹ is phosphoryl.

48. The compound of any one of claims 28-43, wherein R⁹ is alkyl (e.g., phosphorylmethyl or phosphoryldifluoromethyl).

49. The compound of any one of claims 28-43, wherein R⁹ is phosphoramidityl.

50. The compound of any one of claims 28-43, wherein R⁹ is phosphoramidatyl.

51. The compound of claim 1, wherein the compound is selected from 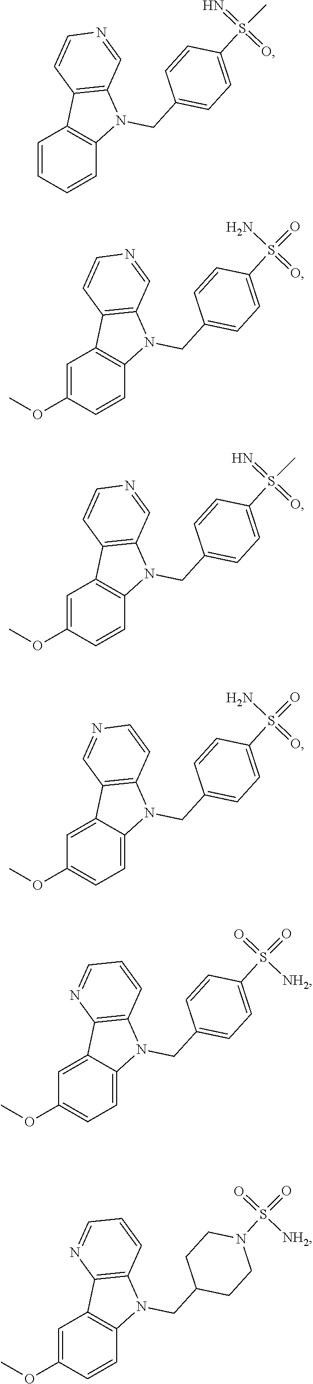 [SMILES: CS(=N)(=O)c1ccc(Cn2c3ccccc3c3ccncc32)cc1 ; COc1ccc2c(c1)c1ccncc1n2Cc1ccc(S(N)(=O)=O)cc1 ; COc1ccc2c(c1)c1ccncc1n2Cc1ccc(S(C)(=N)=O)cc1 ; COc1ccc2c(c1)c1cnccc1n2Cc1ccc(S(N)(=O)=O)cc1 ; COc1ccc2c(c1)c1ncccc1n2Cc1ccc(S(N)(=O)=O)cc1 ; COc1ccc2c(c1)c1ncccc1n2CC1CCN(S(N)(=O)=O)CC1] 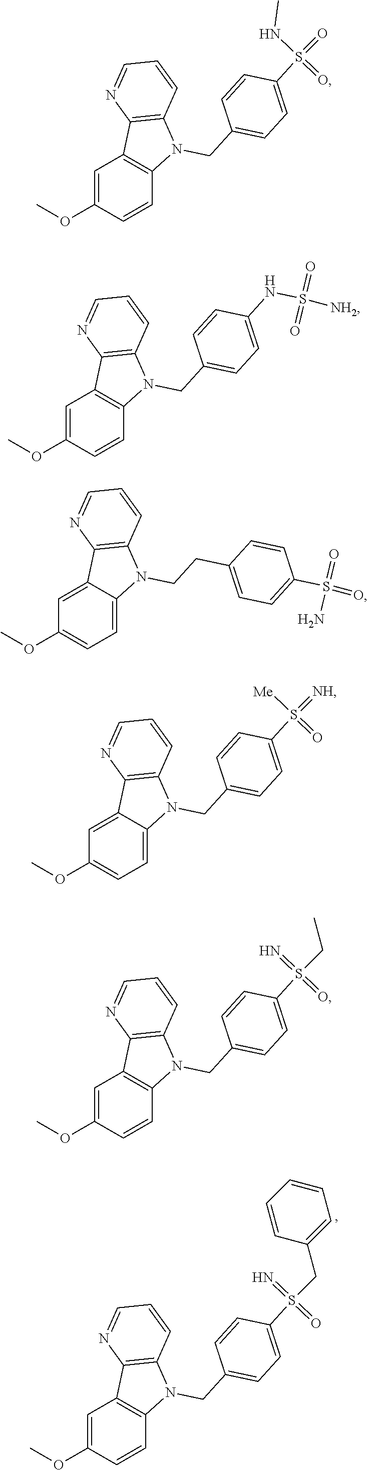 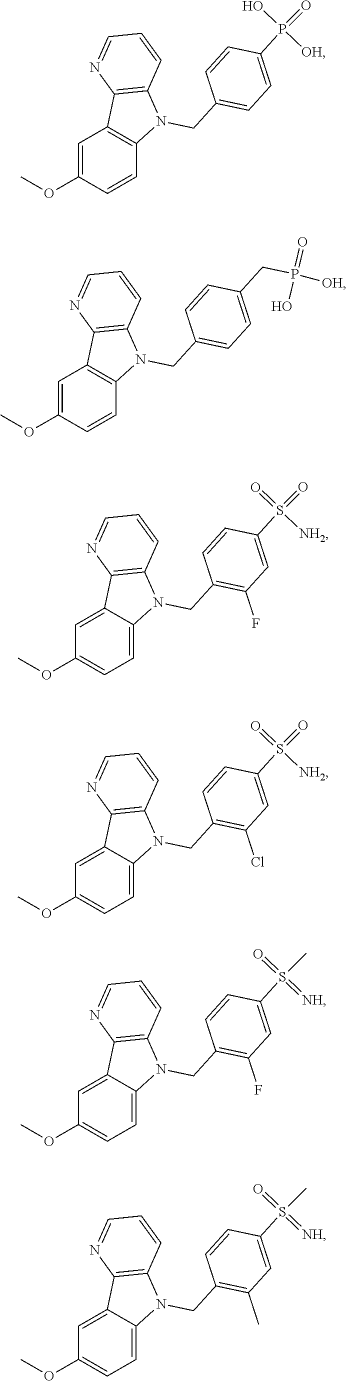 [SMILES: COc1ccc2c(c1)c1ncccc1n2Cc1ccc(P(=O)(O)O)cc1 ; COc1ccc2c(c1)c1ncccc1n2Cc1ccc(CP(=O)(O)O)cc1 ; COc1ccc2c(c1)c1ncccc1n2Cc1ccc(S(N)(=O)=O)cc1F ; COc1ccc2c(c1)c1ncccc1n2Cc1ccc(S(N)(=O)=O)cc1Cl ; COc1ccc2c(c1)c1ncccc1n2Cc1ccc(S(C)(=N)=O)cc1F ; COc1ccc2c(c1)c1ncccc1n2Cc1ccc(S(C)(=N)=O)cc1C] 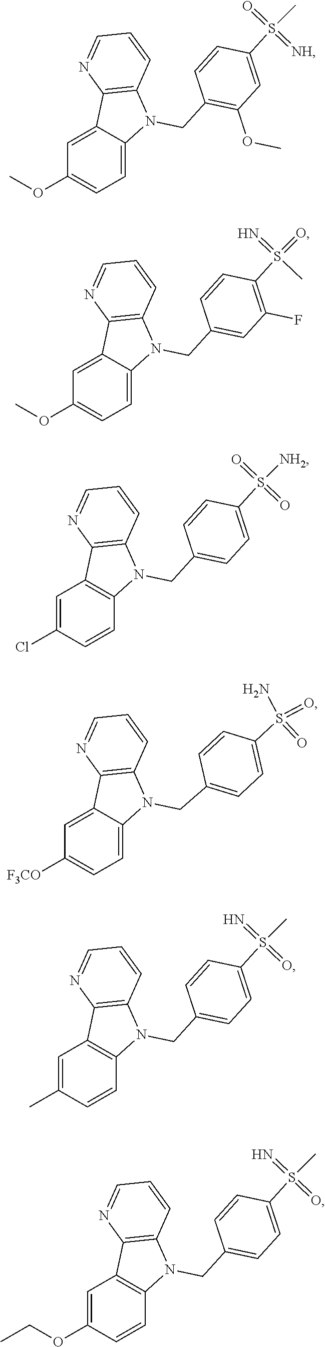 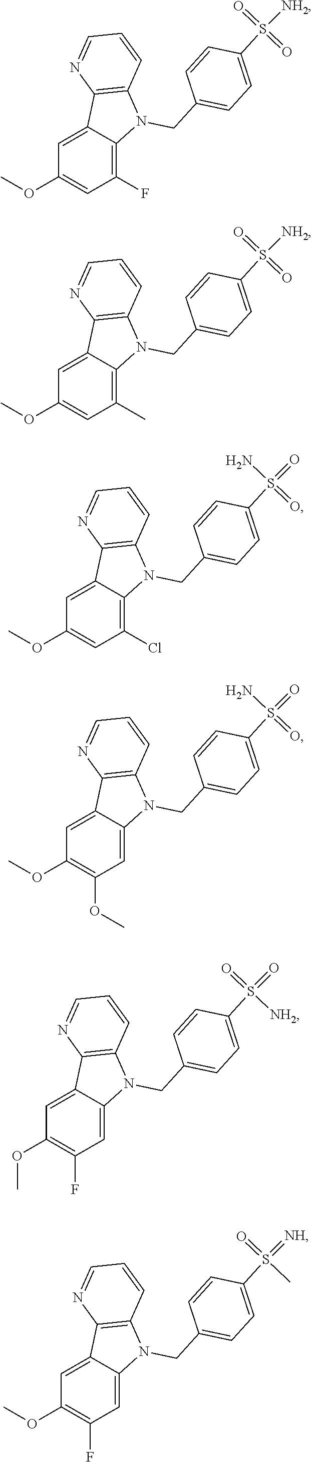 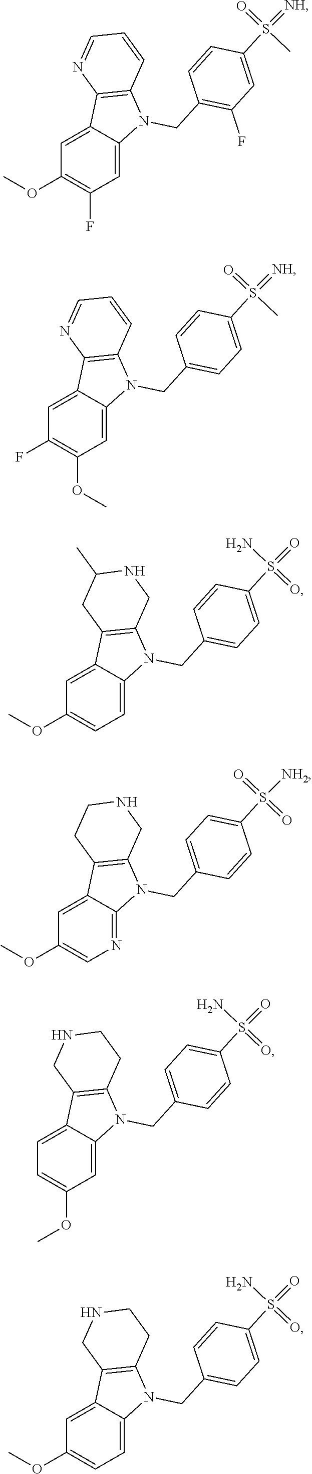 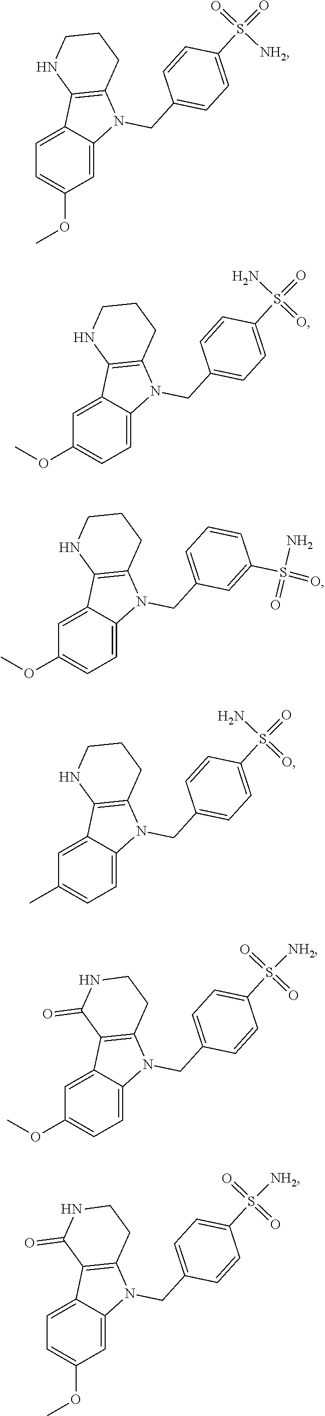 [SMILES: COc1ccc2c3c(n(Cc4ccc(S(N)(=O)=O)cc4)c2c1)CCCN3 ; COc1ccc2c(c1)c1c(n2Cc2ccc(S(N)(=O)=O)cc2)CCCN1 ; COc1ccc2c(c1)c1c(n2Cc2cccc(S(N)(=O)=O)c2)CCCN1 ; Cc1ccc2c(c1)c1c(n2Cc2ccc(S(N)(=O)=O)cc2)CCCN1 ; COc1ccc2c(c1)c1c(n2Cc2ccc(S(N)(=O)=O)cc2)CCNC1=O ; COc1ccc2c3c(n(Cc4ccc(S(N)(=O)=O)cc4)c2c1)CCNC3=O] 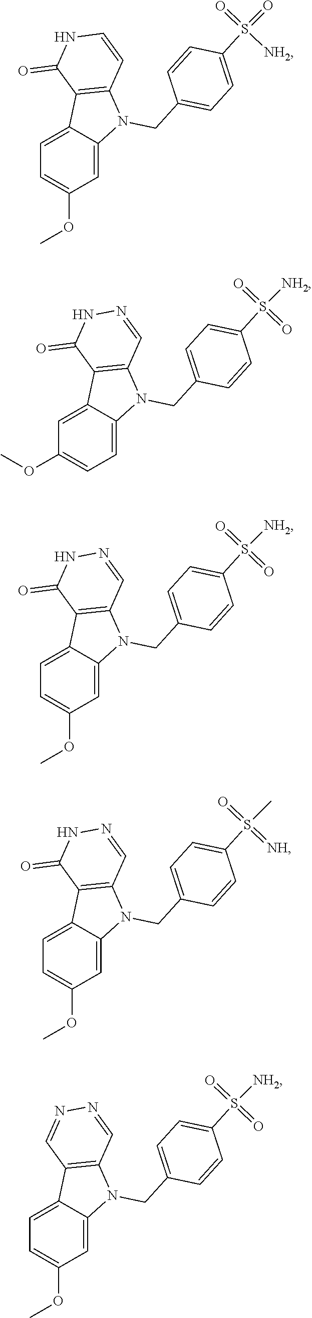 [SMILES: COc1ccc2c3c(=O)[nH]ccc3n(Cc3ccc(S(N)(=O)=O)cc3)c2c1 ; COc1ccc2c(c1)c1c(=O)[nH]ncc1n2Cc1ccc(S(N)(=O)=O)cc1 ; COc1ccc2c3c(=O)[nH]ncc3n(Cc3ccc(S(N)(=O)=O)cc3)c2c1 ; COc1ccc2c3c(=O)[nH]ncc3n(Cc3ccc(S(C)(=N)=O)cc3)c2c1 ; COc1ccc2c3cnncc3n(Cc3ccc(S(N)(=O)=O)cc3)c2c1] 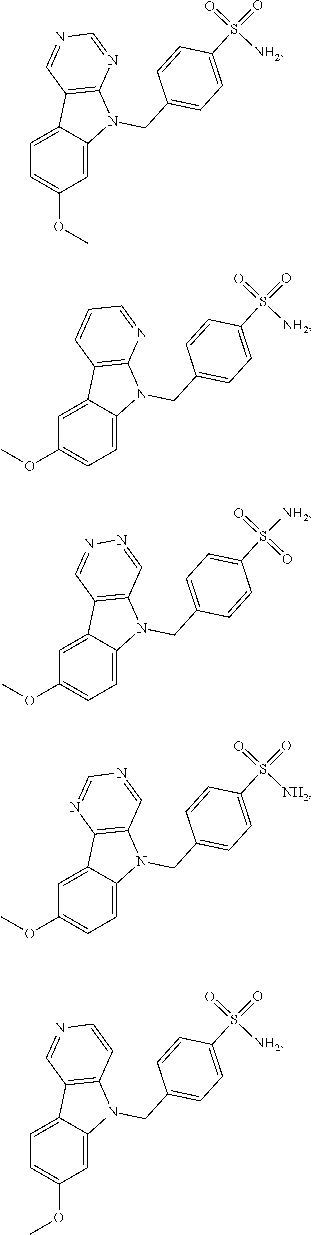 [SMILES: COc1ccc2c3cncnc3n(Cc3ccc(S(N)(=O)=O)cc3)c2c1 ; COc1ccc2c(c1)c1cccnc1n2Cc1ccc(S(N)(=O)=O)cc1 ; COc1ccc2c(c1)c1cnncc1n2Cc1ccc(S(N)(=O)=O)cc1 ; COc1ccc2c(c1)c1ncncc1n2Cc1ccc(S(N)(=O)=O)cc1 ; COc1ccc2c3cnccc3n(Cc3ccc(S(N)(=O)=O)cc3)c2c1] 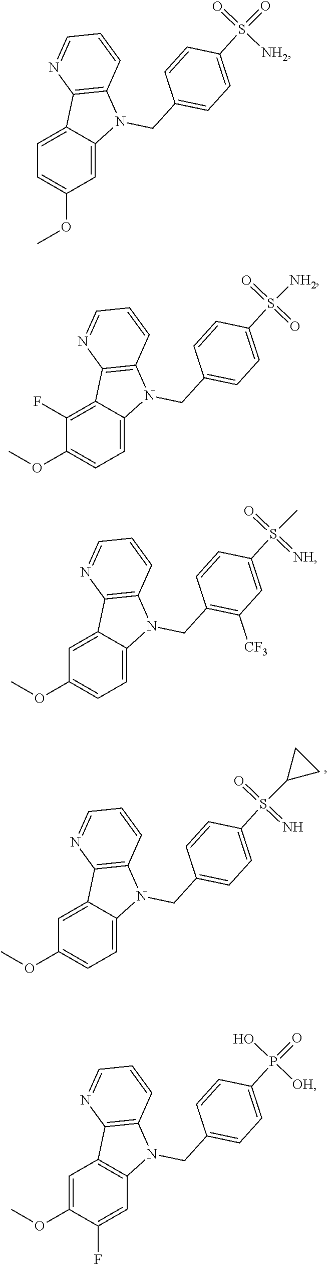 [SMILES: COc1ccc2c3ncccc3n(Cc3ccc(S(N)(=O)=O)cc3)c2c1 ; COc1ccc2c(c1F)c1ncccc1n2Cc1ccc(S(N)(=O)=O)cc1 ; COc1ccc2c(c1)c1ncccc1n2Cc1ccc(S(C)(=N)=O)cc1C(F)(F)F ; COc1ccc2c(c1)c1ncccc1n2Cc1ccc(S(=N)(=O)C2CC2)cc1 ; COc1cc2c3ncccc3n(Cc3ccc(P(=O)(O)O)cc3)c2cc1F] 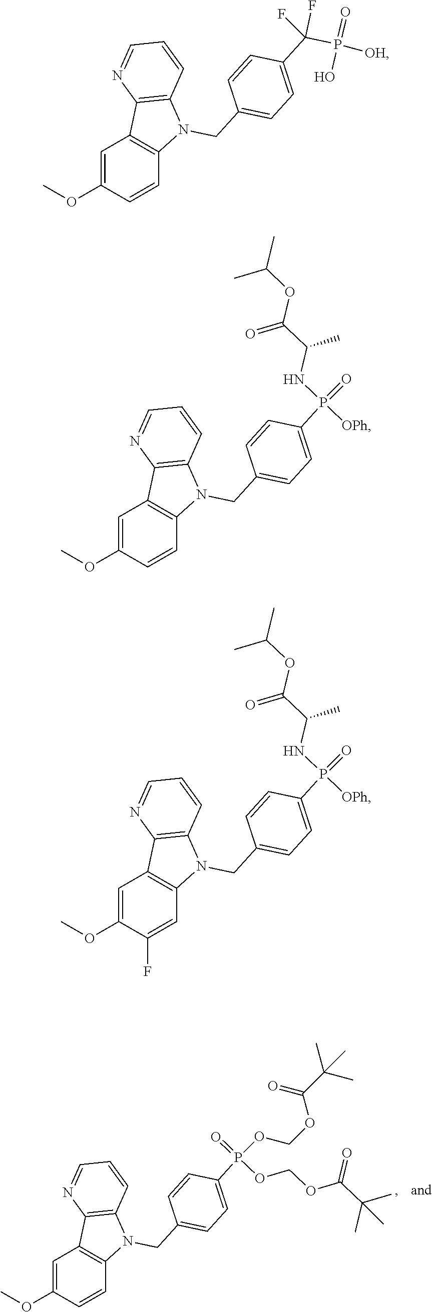 [SMILES: COc1ccc2c(c1)c1ncccc1n2Cc1ccc(C(F)(F)P(=O)(O)O)cc1 ; COc1ccc2c(c1)c1ncccc1n2Cc1ccc(P(=O)(N[C@@H](C)C(=O)OC(C)C)Oc2ccccc2)cc1 ; COc1cc2c3ncccc3n(Cc3ccc(P(=O)(N[C@@H](C)C(=O)OC(C)C)Oc4ccccc4)cc3)c2cc1F ; COc1ccc2c(c1)c1ncccc1n2Cc1ccc(P(=O)(OCOC(=O)C(C)(C)C)OCOC(=O)C(C)(C)C)cc1] 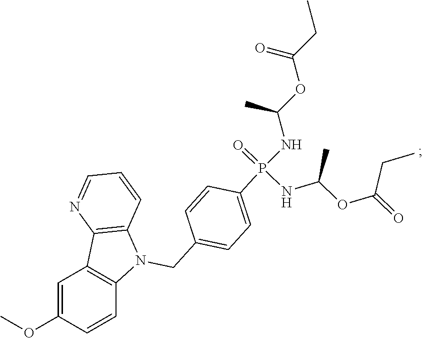 [SMILES: CCC(=O)O[C@H](C)NP(=O)(N[C@@H](C)OC(=O)CC)c1ccc(Cn2c3ccc(OC)cc3c3ncccc32)cc1]
or a pharmaceutically acceptable salt thereof.

52. A compound represented by following Formula 1, a hydrate thereof, a solvate thereof, an isomer thereof, or a pharmaceutically acceptable salt thereof: 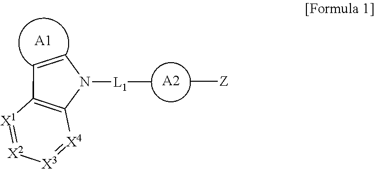
wherein,
A1 is a substituted or unsubstituted C3-30 cycloalkyl ring, a substituted or unsubstituted C2-30 heterocycloalkyl ring, a substituted or unsubstituted C6-30 aryl ring, or a substituted or unsubstituted C3-30 heteroaryl ring, fused to an adjacent ring;
A2 is a substituted or unsubstituted C3-20 cycloalkylene group, a substituted or unsubstituted C2-20 heterocycloalkylene group, a substituted or unsubstituted C6-30 arylene group, or a substituted or unsubstituted C3-30 heteroarylene group;
X₁ to X₄ are each independently CR or N;
R is hydrogen, a hydroxy group, a halogen group, a C1-10 alkyl group, a C1-10 alkoxy group, a C6-20 aryl group, a C3-20 heteroaryl group, a C3-10 cycloalkyl group, a C3-10 heterocycloalkyl group, an amino group, a nitro group, an amide group, a carboxyl acid group, a nitrile group, a urea group, or a sulfonamide group;
L₁ is a C1-10 alkylene group or a C2-10 alkenylene group;
Z is represented by 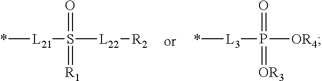
R₁ is O or NR₅;
R₂ is hydrogen, a hydroxy group, a cyano group, a C1-C10 alkyl group, a C1-10 alkoxy group, a C6-C12 aryl group, a C3-C10 cycloalkyl group, a C2-C12 heterocycloalkyl group, —C(═O)OR₆, or —NR₈R₉;
L₂₁, L₂₂ and L₃ are each independently a single bond, a substituted or unsubstituted C1-C5 alkylene group, or —NR₇—;
R₃ to R₇ are each independently hydrogen or a C1-C5 alkyl group;
R₈ and R₉ are each independently hydrogen, —C(═O)R₁₀ (with the proviso that R₁₀ is a C1-C5 alkyl group), or -Boc (tert-butoxycarbonyl); and
* is a point connected to A2 in Formula 1.

53. The compound according to claim 52, a hydrate thereof, a solvate thereof, an isomer thereof, or a pharmaceutically acceptable salt thereof, wherein A1 in Formula 1 is represented by following Formula 2 or Formula 3: 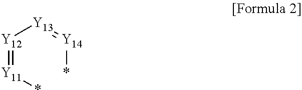
wherein,
Y₁₁ to Y₁₄ are each independently CRₐ or N;
Rₐ is hydrogen, a hydroxy group, a halogen group, a C1-10 alkyl group, a C1-10 alkoxy group, a C6-20 aryl group, a C3-20 heteroaryl group, a C3-10 cycloalkyl group, a C3-10 heterocycloalkyl group, an amino group, a nitro group, an amide group, a carboxyl acid group, a nitrile group, a urea group, or a sulfonamide group; and
* is a point connected to each of two adjacent carbon atoms on the fused ring to which A1 is attached, and 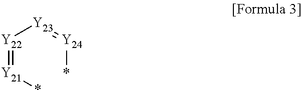
wherein,
Y₂₁ to Y₂₄ are each independently CR_{b}R_{c}, —C(═O)—, or NR_{d};
R_{b} to R_{d} are each independently hydrogen, a hydroxy group, a halogen group, a C1-10 alkyl group, a C1-10 alkoxy group, a C6-20 aryl group, a C3-20 heteroaryl group, a C3-10 cycloalkyl group or a C3-10 heterocycloalkyl group, an amino group, a nitro group, an amide group, a carboxyl acid group, a nitrile group, a urea group, or a sulfonamide group; and
* is a point connected to each of two adjacent carbon atoms on the fused ring to which A1 is attached.

54. The compound according to claim 52, a hydrate thereof, a solvate thereof, an isomer thereof, or a pharmaceutically acceptable salt thereof,
wherein, A1 in Formula 1 is a substituted or unsubstituted C3-10 cycloalkyl ring, a substituted or unsubstituted C2-10 heterocycloalkyl ring, a substituted or unsubstituted C6-10 aryl ring, or a substituted or unsubstituted C3-10 heteroaryl ring, fused to an adjacent ring;
A2 is a substituted or unsubstituted C3-10 cycloalkylene group, a substituted or unsubstituted C2-10 heterocycloalkylene group, a substituted or unsubstituted C6-10 arylene group, or a substituted or unsubstituted C3-10 heteroarylene group;
X₁ to X₄ are each independently CR or N;
R is hydrogen, a hydroxy group, a halogen group, a C1-10 alkyl group, a C1-10 alkoxy group, a C6-10 aryl group, a C3-10 heteroaryl group, a C3-10 cycloalkyl group, a C3-10 heterocycloalkyl group, an amino group, a nitro group, an amide group, a carboxyl acid group, a nitrile group, a urea group, or a sulfonamide group; and
L₁ is a C1-10 alkylene group.

55. The compound according to claim 52, a hydrate thereof, a solvate thereof, an isomer thereof, or a pharmaceutically acceptable salt thereof,
wherein, A1 in Formula 1 is a substituted or unsubstituted C5 heterocycloalkyl ring, a substituted or unsubstituted C6 aryl ring, or a substituted or unsubstituted C4-5 heteroaryl ring, fused to an adjacent ring;
A2 is a substituted or unsubstituted C5 heterocycloalkylene group, a substituted or unsubstituted C6 arylene group, or a substituted or unsubstituted C5 heteroarylene group,
X₁ to X₄ are each independently CR or N;
R is hydrogen, a halogen group, a C1-2 alkyl group, or a C1-2 alkoxy group; and
L₁ is a C1-2 alkylene group.

56. The compound according to claim 53, a hydrate thereof, a solvate thereof, an isomer thereof, or a pharmaceutically acceptable salt thereof, wherein, Y11 to Y14 are each independently CRa or N; and
Rₐ is hydrogen, a hydroxy group, a C1-10 alkyl group, or a C1-10 alkoxy group.

57. The compound according to claim 53, a hydrate thereof, a solvate thereof, an isomer thereof, or a pharmaceutically acceptable salt thereof, wherein, Y₂₁ to Y₂₄ are each independently CR_{b}R_{c}, —C(═O)—, or NR_{d}; and
R_{b} to R_{d} are each independently hydrogen, a hydroxy group, a C1-10 alkyl group, or a C1-10 alkoxy group.

58. The compound according to claim 53, a hydrate thereof, a solvate thereof, an isomer thereof, or a pharmaceutically acceptable salt thereof, wherein, Z is 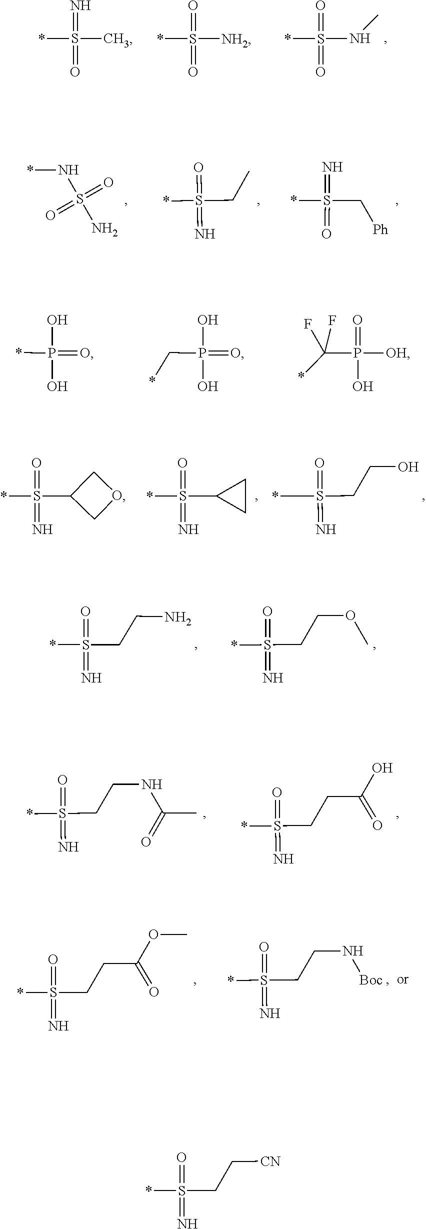
(with the proviso that * is a point connected to A2 in Formula 1).

59. The compound according to claim 52, a hydrate thereof, a solvate thereof, an isomer thereof, or a pharmaceutically acceptable salt thereof, wherein the compound is selected from compounds having the following structure:

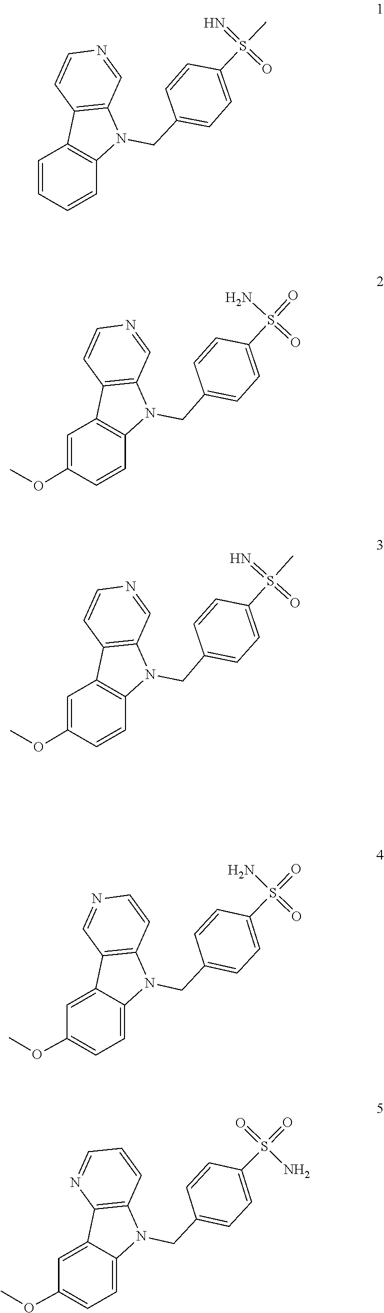 [SMILES: CS(=N)(=O)c1ccc(Cn2c3ccccc3c3ccncc32)cc1 ; COc1ccc2c(c1)c1ccncc1n2Cc1ccc(S(N)(=O)=O)cc1 ; COc1ccc2c(c1)c1ccncc1n2Cc1ccc(S(C)(=N)=O)cc1 ; COc1ccc2c(c1)c1cnccc1n2Cc1ccc(S(N)(=O)=O)cc1 ; COc1ccc2c(c1)c1ncccc1n2Cc1ccc(S(N)(=O)=O)cc1]

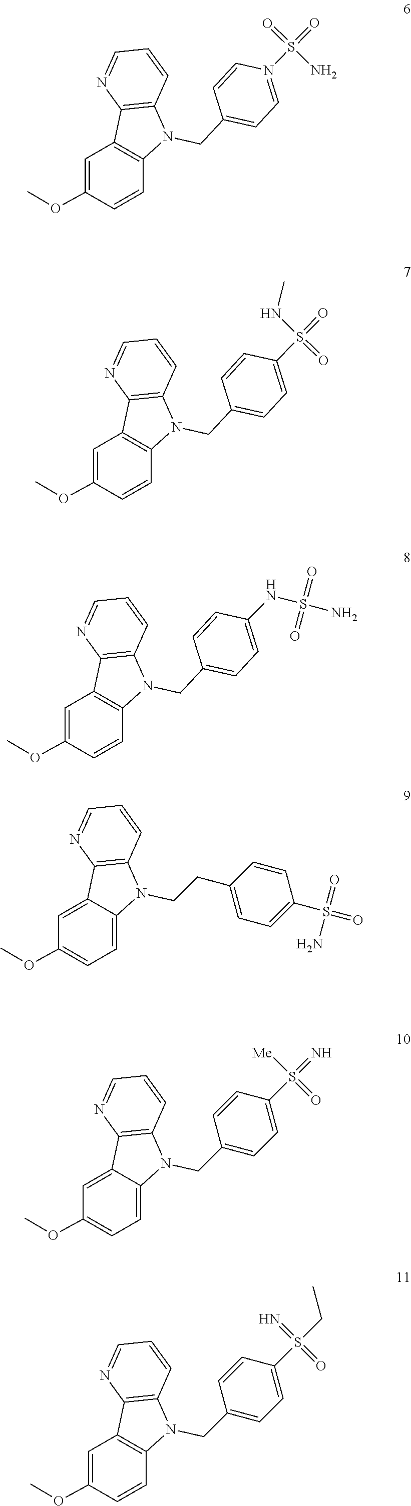

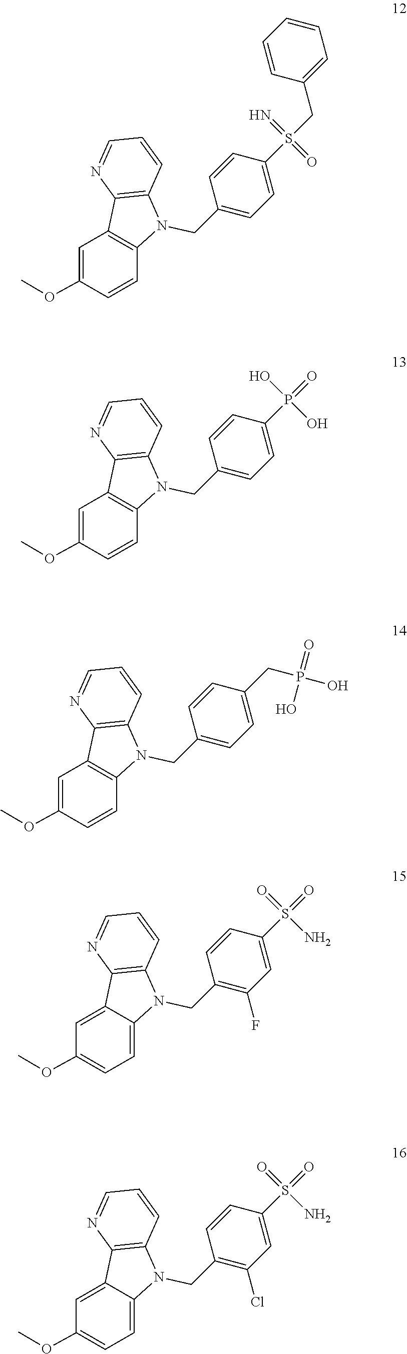 [SMILES: COc1ccc2c(c1)c1ncccc1n2Cc1ccc(S(=N)(=O)Cc2ccccc2)cc1 ; COc1ccc2c(c1)c1ncccc1n2Cc1ccc(P(=O)(O)O)cc1 ; COc1ccc2c(c1)c1ncccc1n2Cc1ccc(CP(=O)(O)O)cc1 ; COc1ccc2c(c1)c1ncccc1n2Cc1ccc(S(N)(=O)=O)cc1F ; COc1ccc2c(c1)c1ncccc1n2Cc1ccc(S(N)(=O)=O)cc1Cl]

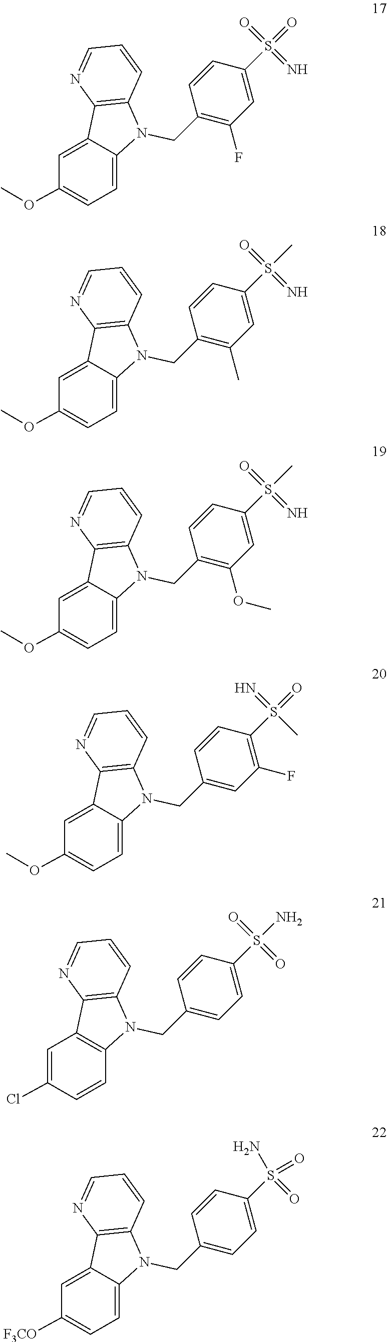

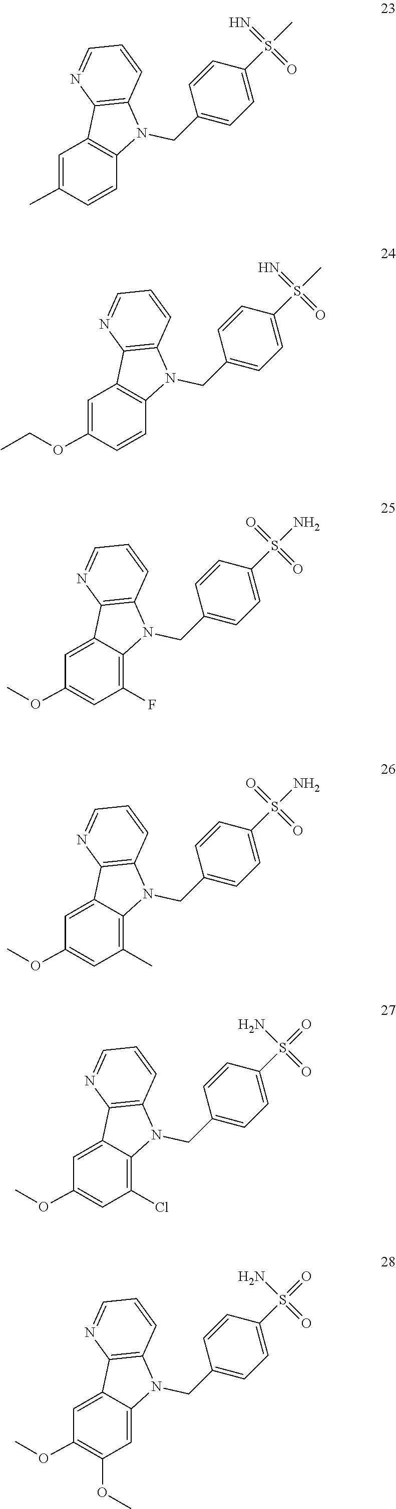

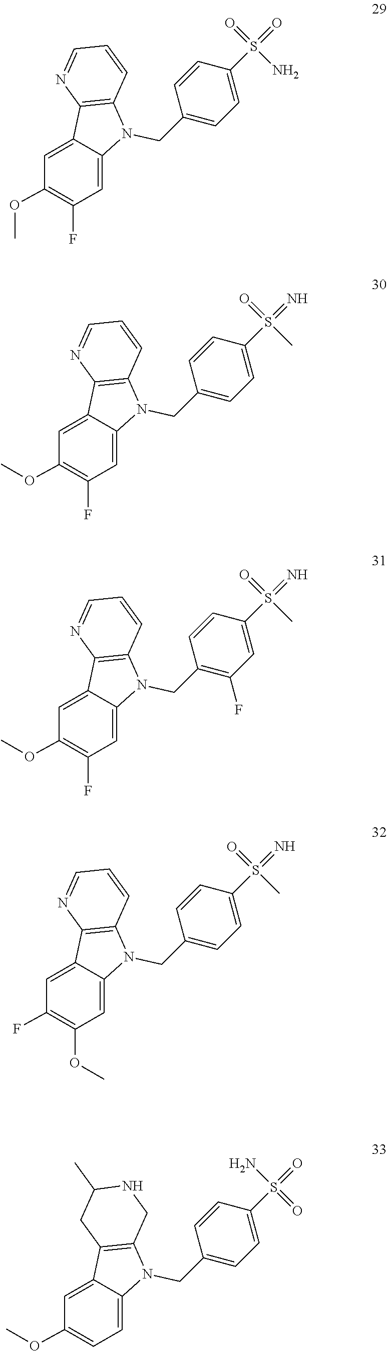 [SMILES: COc1cc2c3ncccc3n(Cc3ccc(S(N)(=O)=O)cc3)c2cc1F ; COc1cc2c3ncccc3n(Cc3ccc(S(C)(=N)=O)cc3)c2cc1F ; COc1cc2c3ncccc3n(Cc3ccc(S(C)(=N)=O)cc3F)c2cc1F ; COc1cc2c(cc1F)c1ncccc1n2Cc1ccc(S(C)(=N)=O)cc1 ; COc1ccc2c(c1)c1c(n2Cc2ccc(S(N)(=O)=O)cc2)CNC(C)C1]

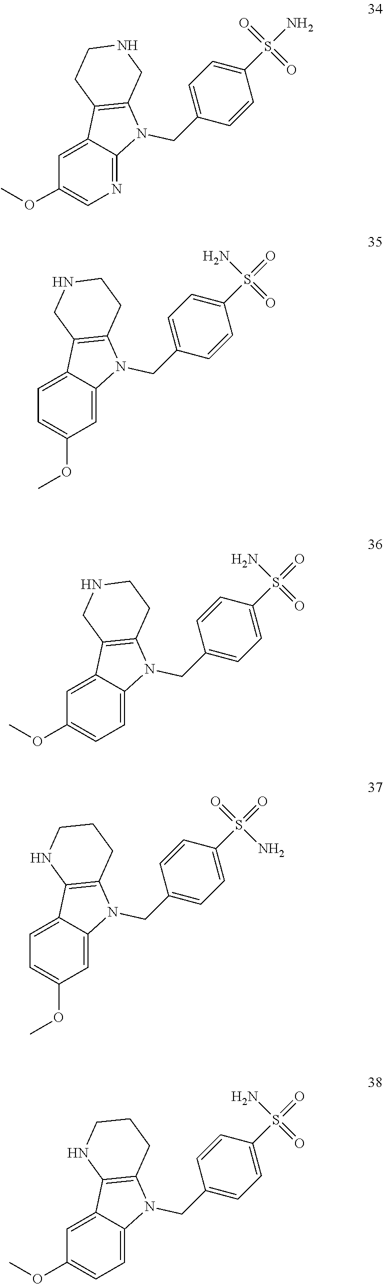 [SMILES: COc1cnc2c(c1)c1c(n2Cc2ccc(S(N)(=O)=O)cc2)CNCC1 ; COc1ccc2c3c(n(Cc4ccc(S(N)(=O)=O)cc4)c2c1)CCNC3 ; COc1ccc2c(c1)c1c(n2Cc2ccc(S(N)(=O)=O)cc2)CCNC1 ; COc1ccc2c3c(n(Cc4ccc(S(N)(=O)=O)cc4)c2c1)CCCN3 ; COc1ccc2c(c1)c1c(n2Cc2ccc(S(N)(=O)=O)cc2)CCCN1]

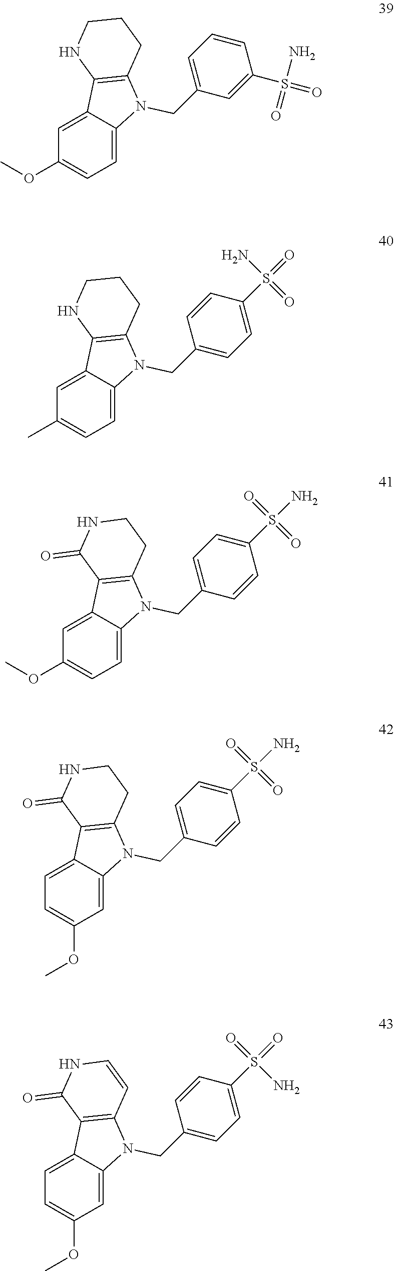 [SMILES: COc1ccc2c(c1)c1c(n2Cc2cccc(S(N)(=O)=O)c2)CCCN1 ; Cc1ccc2c(c1)c1c(n2Cc2ccc(S(N)(=O)=O)cc2)CCCN1 ; COc1ccc2c(c1)c1c(n2Cc2ccc(S(N)(=O)=O)cc2)CCNC1=O ; COc1ccc2c3c(n(Cc4ccc(S(N)(=O)=O)cc4)c2c1)CCNC3=O ; COc1ccc2c3c(=O)[nH]ccc3n(Cc3ccc(S(N)(=O)=O)cc3)c2c1]

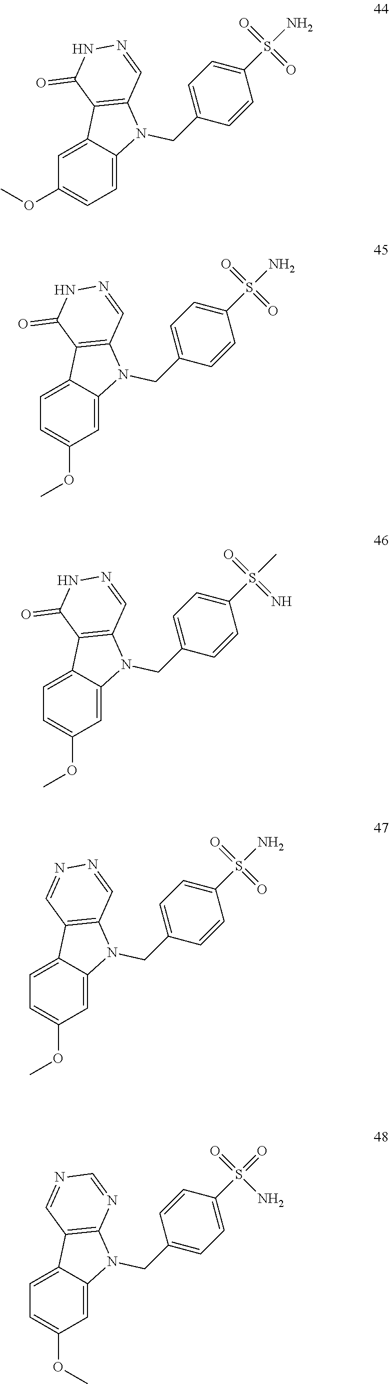 [SMILES: COc1ccc2c(c1)c1c(=O)[nH]ncc1n2Cc1ccc(S(N)(=O)=O)cc1 ; COc1ccc2c3c(=O)[nH]ncc3n(Cc3ccc(S(N)(=O)=O)cc3)c2c1 ; COc1ccc2c3c(=O)[nH]ncc3n(Cc3ccc(S(C)(=N)=O)cc3)c2c1 ; COc1ccc2c3cnncc3n(Cc3ccc(S(N)(=O)=O)cc3)c2c1 ; COc1ccc2c3cncnc3n(Cc3ccc(S(N)(=O)=O)cc3)c2c1]

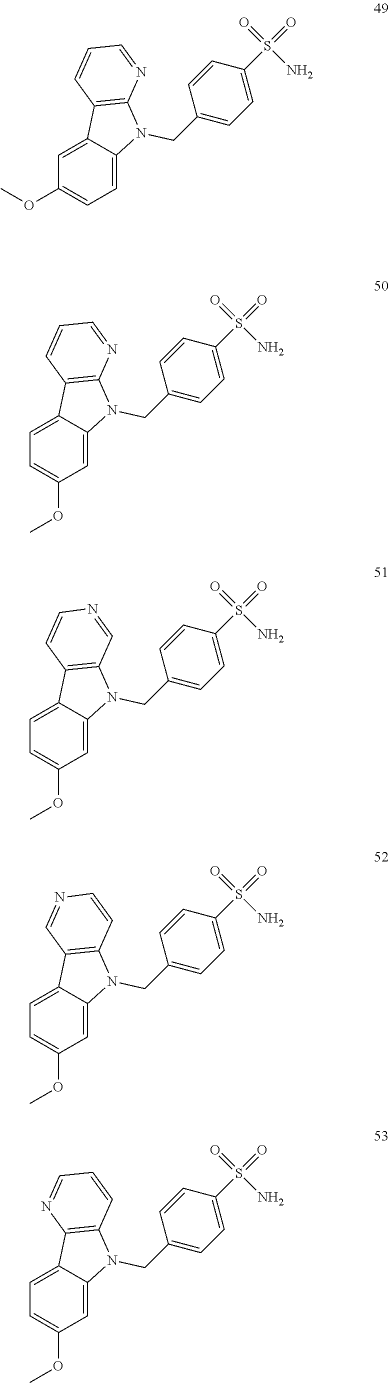 [SMILES: COc1ccc2c(c1)c1cccnc1n2Cc1ccc(S(N)(=O)=O)cc1 ; COc1ccc2c3cccnc3n(Cc3ccc(S(N)(=O)=O)cc3)c2c1 ; COc1ccc2c3ccncc3n(Cc3ccc(S(N)(=O)=O)cc3)c2c1 ; COc1ccc2c3cnccc3n(Cc3ccc(S(N)(=O)=O)cc3)c2c1 ; COc1ccc2c3ncccc3n(Cc3ccc(S(N)(=O)=O)cc3)c2c1]

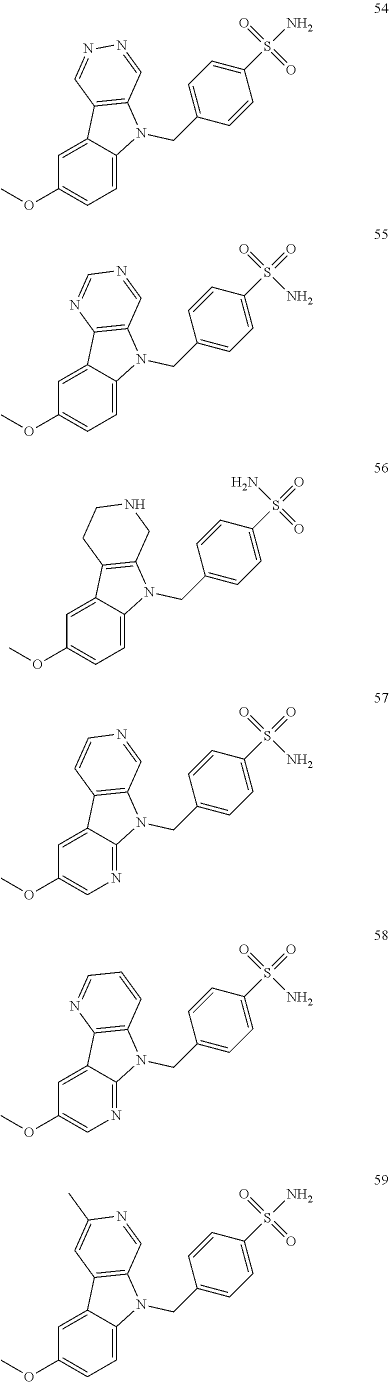 [SMILES: COc1ccc2c(c1)c1cnncc1n2Cc1ccc(S(N)(=O)=O)cc1 ; COc1ccc2c(c1)c1ncncc1n2Cc1ccc(S(N)(=O)=O)cc1 ; COc1ccc2c(c1)c1c(n2Cc2ccc(S(N)(=O)=O)cc2)CNCC1 ; COc1cnc2c(c1)c1ccncc1n2Cc1ccc(S(N)(=O)=O)cc1 ; COc1cnc2c(c1)c1ncccc1n2Cc1ccc(S(N)(=O)=O)cc1 ; COc1ccc2c(c1)c1cc(C)ncc1n2Cc1ccc(S(N)(=O)=O)cc1]

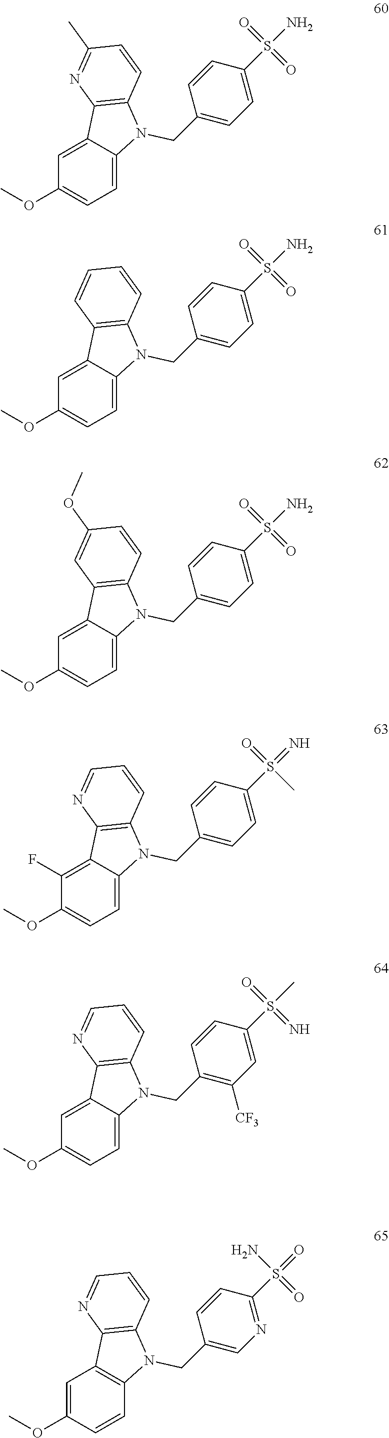

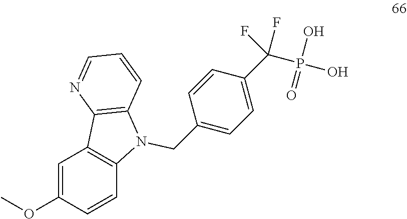 [SMILES: COc1ccc2c(c1)c1ncccc1n2Cc1ccc(C(F)(F)P(=O)(O)O)cc1]

60. The compound according to claim 52, a hydrate thereof, a solvate thereof, an isomer thereof, or a pharmaceutically acceptable salt thereof, wherein the pharmaceutically acceptable salt is a salt with an inorganic or organic acid selected from the group consisting of hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, and toluenesulfonic acid.

61. A pharmaceutical composition comprising the compound of any one of claims 1-60 and a pharmaceutically acceptable excipient.

62. A method of treating cancer in a subject in need thereof comprising administering the compound of any one of claims 1-60, or a pharmaceutically acceptable salt thereof, to the subject.

63. The method claim 62, wherein the cancer is gastric cancer, lung cancer, liver cancer, colorectal cancer, small intestine cancer, pancreatic cancer, brain cancer, bone cancer, melanoma, breast cancer, sclerosing adenosis, uterine cancer, cervical cancer, head and neck cancer, esophageal cancer, thyroid cancer, parathyroid cancer, kidney cancer, sarcoma, prostate cancer, urethral cancer, bladder cancer, hematologic malignancy (e.g., leukemia, multiple myeloma, and myelodysplastic syndrome), lymphoma (e.g., Hodgkin's lymphoma or non-Hodgkin's lymphoma), or fibroadenoma.

64. The method of claim 62 or 63, wherein the method further comprises conjointly administering an additional therapeutic agent (e.g., a cancer agent).

65. A method of treating a disease or disorder characterized by ENPP-1 in a subject in need thereof comprising administering the compound of any one of claims 1-60, or a pharmaceutically acceptable salt thereof, to the subject.

66. A pharmaceutical composition for preventing, alleviating or treating cancer, comprising the compound according to any one of claims 1-60, a hydrate thereof, a solvate thereof, an isomer thereof or a pharmaceutically acceptable salt thereof as an active ingredient.

67. An ENPP-1 inhibitor composition comprising the compound according to any one of claims 1-60, a hydrate thereof, a solvate thereof, an isomer thereof or a pharmaceutically acceptable salt thereof as an active ingredient.
